(19) United States
(12) Patent Application Publication
(10) Pub. No.: US 2025/0066856 A1
(43) Pub. Date: Feb. 27, 2025

(54) PLATFORM AND SAMPLE TYPE INDEPENDENT SINGLE SAMPLE CLASSIFIER FOR TREATMENT DECISION MAKING IN PANCREATIC DUCTAL ADENOCARCINOMA CANCER

(71) Applicant: The University of North Carolina at Chapel Hill, Chapel Hill, NC (US)

(72) Inventors: Jen Jen Yeh, Chapel Hill, NC (US); Richard Moffitt, Stony Brook, NY (US); Naim Ur Rashid, Raleigh, NC (US)

(73) Assignee: The University of North Carolina at Chapel Hill, Chapel Hill, NC (US)

(21) Appl. No.: 18/649,802

(22) Filed: Apr. 29, 2024

Related U.S. Application Data
(63) Continuation of application No. 17/601,002, filed on Oct. 1, 2021, now Pat. No. 12,000,003.
(60) Provisional application No. 62/827,473, filed on Apr. 1, 2019.

(51) Int. Cl. C12Q 1/6886; G16B 25/00; G16B 25/10

(52) U.S. Cl. CPC C12Q 1/6886; G16B 25/00; G16B 25/10; C12Q 2600/106; C12Q 2600/112; C12Q 2600/158

(57) ABSTRACT

Provided are methods for identifying pancreatic cancer subtypes in a subject and treating the same. In some embodiments, the method comprise obtaining gene expression levels for each of the following genes in the biological sample: GPR87, KRT6A, BCAR3, PTGES, ITGA3, C16orf74, S100A2, KRTS, REG4, ANXA10, GATA6, CLDN18, LGALS4, DDC, SLC40A1, CLRN3; performing pair-wise comparisons of gene expression levels for combinations of these genes; and calculating a Raw Score for the biological sample, wherein the Raw Score is indicative of the pancreatic cancer subtype in the subject. Also provided are methods for identifying differential treatment strategies for subjects diagnosed with PDAC, methods for treating PDAC patients based on the subtype of PDAC the patients have; and methods for classifying subjects diagnosed with PDAC as having a basal-like subtype or a classical subtype of PDAC.

### CROSS REFERENCE TO RELATED APPLICATIONS

This application is a continuation of U.S. patent application Ser. No. 17/601,002, filed Oct. 1, 2021 (pending), which is a U.S. National Phase Application of PCT International Patent Application No. PCT/US2020/026209 (expired), filed Apr. 1, 2020, incorporated herein by reference in its entirety and which claims the benefit of U.S. Provisional Patent Application Ser. No. 62/827,473 (expired), filed Apr. 1, 2019, the disclosure of each of which is incorporated herein by reference in its entirety.

### GOVERNMENT INTEREST

This invention was made with government support under grant numbers CA199064 and CA211000 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 191,600 byte UTF-8-encoded XML file created on Apr. 26, 2024 and entitled “421 454 2 PCT_US_CON_ST26.xml”. The Sequence Listing submitted via Patent Center is hereby incorporated by reference in its entirety.

### BACKGROUND

Recent treatment advances, including FOLFIRINOX (Conroy et al., 2011), gemcitabine plus nab-paclitaxel (Von Hoff et al., 2013), and olaparib for BRCA-mutant patients (Kindler et al., 2019), have provided patients and providers with better options. With the substantial progress in molecular subtyping for pancreatic cancer (Collisson et al., 2011; Moffitt et al., 2015; Bailey et al., 2016; Cancer Genome Atlas Research Network., 2017; Puleo et al., 2018; Maurer et al., 2019), there is now an opportunity to determine the optimal choice of therapy given a patient's molecular subtype and other biomarker information, enabling “precision medicine” approaches in pancreatic cancer (Aguirre et al., 2018; Aung et al., 2018).

Transcriptomic molecular subtyping in pancreatic cancer is currently an area of active development, where multiple subtyping schemas for pancreatic cancer have been proposed. For example, three molecular subtypes with potential clinical and therapeutic relevance were first described by Collisson and colleagues (Collisson et al., 2011), leveraging a combination of cell line, bulk, and laser capture microdissected (LCM) patient samples: Collisson (i) quasi-mesenchymal (QM-PDA), (ii) classical, and (iii) exocrine-like. A subsequent study of patients with pancreatic cancer (Bailey et al., 2016), based on more diverse pancreatic cancer histologies in addition to the most common pancreatic ductal adenocarcinoma (PDAC), found four molecular subtypes: Bailey (i) squamous, (ii) pancreatic progenitor, (iii) immunogenic, and (iv) aberrantly differentiated endocrine exocrine (ADEX). More recently, Puleo and colleagues describe five subtypes that are based on features specific to tumor cells and the local microenvironment (Puleo et al., 2018) Maurer and colleagues performed LCM of both tumor and stroma and showed the contribution of each to the three schemas above (Maurer et al., 2019) Finally, we have previously shown two tumor-intrinsic subtypes of PDAC (Moffitt et al., 2015), which we called Moffitt (i) basal-like, given the similarities with basal breast and basal bladder cancer, and (ii) classical, given the overlap with the Collisson classical subtype.

However, consensus regarding proposed subtypes for clinical decision making in PDAC has been elusive. In addition, each proposed schema utilized independent cohorts of patients to demonstrate clinical relevance. As a result, the generalizability, robustness, and relative clinical utility of each proposed subtyping schema remains unclear. Comparative evaluations of these proposed subtyping systems have been limited, partially due to the difficulty in curating and applying these diverse subtyping approaches in new datasets.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments methods for determining a subtype of a pancreatic tumor in a biological sample comprising, consisting essentially of, or consisting of pancreatic tumor cells obtained from a subject. In some embodiments, the methods comprise obtaining gene expression levels for each of the following genes in the biological sample: GPR87, KRT6A, BCAR3, PTGES, ITGA3, C16orf74, S100A2, KRT5, REG4, ANXA10, GATA6, CLDN18, LGALS4, DDC, SLC40A1, CLRN3; performing a pair-wise comparison of the gene expression levels for each of Gene Pairs 1-8 or for each of Gene Pairs A-H, wherein Gene Pairs 1-8 and Gene Pairs A-H are presented in Table 1, and calculating a Raw Score for the biological sample, wherein the calculating comprises assigning a value of 1 for each Gene Pair for which Gene A of the Gene Pair has a higher expression level than Gene B of the Gene Pair, and a value of 0 for each Gene Pair for which Gene A of the Gene Pair has a lower expression level than Gene B of the Gene Pair; multiplying each assigned value by the coefficient listed above for the corresponding Gene Pair to calculate eight individual Gene Pair scores, and adding the eight individual Gene Pair scores together along with a baseline effect to calculate a Raw Score for the biological sample, wherein the baseline effect is −6.815 for Gene Pairs 1-8 and −12.414 for Gene Pairs A-H, wherein if the calculated Raw Score is greater than or equal to 0, the tumor subtype is determined to be a basal-like subtype, and if the calculated Raw Score if less than 0, the tumor subtype is determined to be a classical subtype. In some embodiments, the method further comprises converting the Raw Score to a predicted basal-like probability (PBP) using the inverse-logit transformation

$$P B P = {e}^{Raw Score} / ( 1 + {e}^{Raw Score} ) ,$$

wherein if the PBP is greater than 0.5, the tumor subtype is determined to be a basal-like subtype and if the PBP if less than or equal to 0.5, the tumor subtype is determined to be a classical subtype. In some embodiments, the pancreatic tumor is a pancreatic ductal adenocarcinoma (PDAC). In some embodiments, the biological sample comprises a biopsy sample, optionally a fine needle biopsy aspiration or a percutaneous core needle biopsy, or comprises a frozen or archival sample derived therefrom. In some embodiments, the obtaining employs a technique selected from the group consisting of microarray analysis, RNAseq, quantitative RT-PCR, NanoString, or any combination thereof. In some embodiments, the technique comprises NanoString and employs probes comprising the SEQ ID NOs. as set forth in Table 2. In some embodiments, the subject is a human.

The presently disclosed subject matter also provides in some embodiments methods for identifying a differential treatment strategy for a subject diagnosed with pancreatic ductal adenocarcinoma (PDAC). In some embodiments, the methods comprise obtaining gene expression levels for each of the following genes in a biological sample comprising PDAC cells isolated from the subject: GPR87, KRT6A, BCAR3, PTGES, ITGA3, C16orf74, S100A2, KRT5, REG4, ANXA10, GATA6, CLDN18, LGALS4, DDC, SLC40A1, CLRN3; performing a pair-wise comparison of the gene expression levels for each of Gene Pairs 1-8 or for each of Gene Pairs A-H, wherein Gene Pairs 1-8 and Gene Pairs A-H are as defined herein above, calculating a Raw Score for the biological sample, wherein the calculating comprises assigning a value of 1 for each Gene Pair for which Gene A of the Gene Pair has a higher expression level than Gene B of the Gene Pair, and a value of 0 for each Gene Pair for which Gene A of the Gene Pair has a lower expression level than Gene B of the Gene Pair; multiplying each assigned value by the coefficient listed above for the corresponding Gene Pair to calculate eight individual Gene Pair scores, and adding the eight individual Gene Pair scores together along with a baseline effect to calculate a Raw Score for the biological sample, wherein the baseline effect is −6.815 for Gene Pairs 1-8 and −12.414 for Gene Pairs A-H, wherein if the calculated Raw Score is greater than or equal to 0, the tumor subtype is determined to be a basal-like subtype, and if the calculated Raw Score if less than 0, the tumor subtype is determined to be a classical subtype; identifying a differential treatment strategy for the subject based on the subtype assigned, wherein if the assigned subtype is a basal-like subtype, the differential treatment strategy comprises treatment with gemcitabine, optionally in combination with nab-paclitaxel; and if the assigned subtype is a classical subtype, the different treatment strategy comprises treatment with FOLFIRINOX. In some embodiments, the biological sample comprises a biopsy sample, optionally a fine needle biopsy aspiration or a percutaneous core needle biopsy, or comprises a frozen or archival sample derived therefrom. In some embodiments, the obtaining employs a technique selected from the group consisting of microarray analysis, RNAseq, quantitative RT-PCR, NanoString, or any combination thereof. In some embodiments, the technique comprises NanoString and employs probes comprising the SEQ ID NOs: identified herein above. In some embodiments, the subject is a human.

The presently disclosed subject matter also provides in some embodiments methods for treating patients diagnosed with pancreatic ductal adenocarcinoma (PDAC). In some embodiments, the methods comprise identifying a subtype of the patient's PDAC via any method disclosed herein; and treating the patient with gemcitabine, optionally in combination with nab-paclitaxel, if the assigned subtype is a basal-like subtype and treating the patient with FOLFIRINOX if the assigned subtype is classical. In some embodiments, the treating comprises at least one additional anti-PDAC treatment. In some embodiments, the at least one additional anti-PDAC treatment is surgery, radiation, administration of an additional chemotherapeutic agent, and any combination thereof. In some embodiments, the additional chemotherapeutic agent is a CCR2 inhibitor, a checkpoint inhibitor, or any combination thereof. In some embodiments, the patient is a human.

The presently disclosed subject matter also provides in some embodiments methods for classifying a subject diagnosed with pancreatic ductal adenocarcinoma (PDAC) as having a basal-like subtype or a classical subtype of PDAC. In some embodiments, the methods comprise performing a pair-wise comparison of gene expression levels for each of Gene Pairs 1-8 or for each of Gene Pairs A-H in a sample comprising PDAC cells isolated from the subject, wherein Gene Pairs 1-8 and Gene Pairs A-H are as defined herein above; and calculating a Raw Score for the sample, wherein the calculating comprises assigning a value of 1 for each Gene Pair for which Gene A of the Gene Pair has a higher expression level than Gene B of the Gene Pair, and a value of 0 for each Gene Pair for which Gene A of the Gene Pair has a lower expression level than Gene B of the Gene Pair; multiplying each assigned value by the coefficient listed above for the corresponding Gene Pair to calculate eight individual Gene Pair scores, and adding the eight individual Gene Pair scores together along with a baseline effect to calculate a Raw Score for the biological sample, wherein the baseline effect is −6.815 for Gene Pairs 1-8 and −12.414 for Gene Pairs A-H, wherein if the calculated Raw Score is greater than or equal to 0, the PDAC subtype is determined to be a basal-like subtype, and if the calculated Raw Score if less than 0, the PDAC subtype is determined to be a classical subtype. In some embodiments, the methods further comprise converting the Raw Score to a predicted basal-like probability (PBP) using the inverse-logit transformation

$$P B P = {e}^{Raw Score} / ( 1 + {e}^{Raw Score} ) ,$$

wherein if the PBP is greater than 0.5, the PDAC subtype is determined to be a basal-like subtype and if the PBP if less than or equal to 0.5, the PDAC subtype is determined to be a classical subtype. In some embodiments, the sample comprises a biopsy sample, optionally a fine needle biopsy aspiration or a percutaneous core needle biopsy, or comprises a frozen or archival sample derived therefrom. In some embodiments, the gene expression levels for each of Gene Pairs 1-8 or for each of Gene Pairs A-H in a sample are determined using a technique selected from the group consisting of microarray analysis, RNAseq, quantitative RT-PCR, NanoString, or any combination thereof. In some embodiments, the technique comprises NanoString and employs probes comprising the SEQ ID NOs: identified herein above. In some embodiments, the subject is a human.

Thus, it is an object of the presently disclosed subject matter to provide methods for classifying PDAC cancers into basal-like or classical subtypes, which in some embodiments can be used to differentially treat the PDAC cancers based on the subtype identified. An object of the presently disclosed subject matter having been stated hereinabove, and which is achieved in whole or in part by the presently disclosed subject matter, other objects will become evident as the description proceeds when taken in connection with the accompanying EXAMPLES and Figures as best described herein below

### BRIEF DESCRIPTION OF THE FIGURES

FIGS. 1A-1C are Kaplan-Meier plots showing subtype performance in predicting patient prognosis in pooled datasets from the survival group (see Table 7). Kaplan-Meier plots of OS in the context of the subtyping schemas of Collisson (FIG. 1A), Bailey (FIG. 1B), and Moffitt (FIG. 1C). Log-rank P values for overall association were determined from stratified Cox proportional hazards models, where dataset was used as a stratification factor to account for variation in baseline hazard across studies. BIC was calculated to compare the three subtyping schemas.

FIG. 2 shows the results of development and validation of the PurIST SSC classifier It provides an overview of the PurIST prediction procedure. Gene expression for genes pertaining to each PurIST TSP is first measured in a new sample. Values are assigned for each TSP given the relative expression of each gene in the TSP (1 if gene A>gene B expression in the pair, 0 otherwise). Given the set of estimated PurIST TSP coefficients, a TSP score is calculated by summing the product of each TSP and its corresponding TSP coefficient, adjusting for the model intercept. This value is finally transformed into a predicted probability of belonging to the basal-like subtype for classification (inverse logit function).

FIGS. 3A-3G show clinical relevance of PurIST SSC in datasets belonging to the treatment group. FIGS. 3A and 3B are Kaplan-Meier plots of OS in pooled datasets (FIG. 3A) belonging to the survival group minus datasets belonging to the training group and Yeh Seq FNA samples (FIG. 3B). P value and HRs for overall association were estimated by stratified Cox proportional hazards model in FIG. 3A, where dataset of origin was used as a stratification factor. FIGS. 3C and 3D are waterfall plots showing the percent change (% change) in size of tumor target lesions from baseline in the context of PurIST subtypes in the COMPASS (FIG. 3C) and Linehan trials (FIG. 3D). +20% and −30% of size change are marked by dashed lines. In FIG. 3C, gray vs. black bars denote PurIST subtype calls of the patient tumors. Patients marked with * were treated with gemcitabine/nab-paclitaxel (GP)-based therapy, and the rest were treated with modified FOLFIRINOX (m-FOLFIRINOX). In FIG. 3D, gray vs. black bars denote PurIST subtype calls of pretreatment samples. Colored tracks below to compare subtype calls for samples pre- and posttreatment of PurIST subtyping and the Moffitt schema Patients marked with * were treated with FOLFIRINOX, and the rest were treated with FOLFIRINOX+PF-04133309. FIG. 3E is a plot of correlation between the PurIST score (basal-like probability) for patient samples pre- and posttreatment in the Linehan trial. Basal-like samples are denoted with light gray triangles and classical samples are denoted with black triangles. FIGS. 3F and 3G are plots showing correlation between the percentage of change (% change) of tumors and the PurIST score (basal-like probability) derived from PurIST in basal-like (FIG. 3F) and classical samples (FIG. 3G), excluding a basal-like sample with an unstable DNA subtype.

### BRIEF DESCRIPTION OF THE SEQUENCE LISTING

SEQ ID NOs: 1-58 are exemplary biosequences corresponding to certain human gene products as disclosed herein and summarized herein below. For each of SEQ ID NOs: 1-58, the odd numbered SEQ ID NO: encodes the immediately following even numbered SEQ ID NO. as set forth in Table 3.

SEQ ID NOs: 59-102 are exemplary NanoString probes for certain gene products disclosed herein, which are as follows: ANXA10 (SEQ ID NO: 59), C16orf74 (SEQ ID NO: 60), CDH17 (SEQ ID NO: 61), DCBLD2 (SEQ ID NO: 62), DDC (SEQ ID NO: 63), GPR87 (SEQ ID NO: 64), KRT6A (SEQ ID NO: 65), KRTIS (SEQ ID NO. 66), KRT17 (SEQ ID NO. 67), LGALS4 (SEQ ID NO: 68), PLA2G10 (SEQ ID NO: 69), PTGES (SEQ ID NO: 70), REG4 (SEQ ID NO: 71), S100A2 (SEQ ID NO: 72), TFF1 (SEQ ID NO: 73), TSPAN8 (SEQ ID NO: 74), CTSE (SEQ ID NO: 75), LYZ (SEQ ID NO: 76), MUC17 (SEQ ID NO: 77), MYOIA (SEQ ID NO: 78), NR112 (SEQ ID NO: 79), PIPSKIB (SEQ ID NO: 80), BCAR3 (SEQ ID NO: 81), GATA6 (SEQ ID NO: 82), CLRN3 (SEQ ID NO: 83), CLDN18 (SEQ ID NO: 84), ITGA3 (SEQ ID NO: 85), SLC40A1 (SEQ ID NO: 86), KRT5 (SEQ ID NO: 87), RPLPO (SEQ ID NO: 88), B2M (SEQ ID NO: 89), ACTB (SEQ ID NO: 90), RPL19 (SEQ ID NO: 91), GAPDH (SEQ ID NO: 92), LDHA (SEQ ID NO: 93), PGK1 (SEQ ID NO: 94), TUBB (SEQ ID NO: 95), SDHA (SEQ ID NO: 96), CLTC (SEQ ID NO: 97), HPRTI (SEQ ID NO: 98), ABCF1 (SEQ ID NO: 99), GUSB (SEQ ID NO: 100), TBP (SEQ ID NO: 101), and ALASI (SEQ ID NO: 102).

Genes listed among SEQ ID NOs: 59-102 that are not included in those among SEQ ID NOs: 1-59 (e.g., those corresponding to SEQ ID NOs: 75-80 and 88-102) can be employed in some embodiments as internal controls for any of the gene expression techniques disclosed herein.

### DETAILED DESCRIPTION

### I. General Considerations

Molecular subtyping for pancreatic cancer has made substantial progress in recent years, facilitating the optimization of existing therapeutic approaches to improve clinical outcomes in pancreatic cancer. Disclosed herein are assessments of three major subtype classification schemas in the context of results from two clinical trials and by meta-analysis of publicly available expression data to assess statistical criteria of subtype robustness and overall clinical relevance. We then developed a single-sample classifier (SSC) using penalized logistic regression based on the most robust and replicable schema.

Demonstrated herein is that a tumor-intrinsic two-subtype schema is most robust, replicable, and clinically relevant. We developed Purity Independent Subtyping of Tumors (PurIST), a SSC with robust and highly replicable performance on a wide range of platforms and sample types. We show that PurIST subtypes have meaningful associations with patient prognosis and have significant implications for treatment response to FOLIFIRNOX.

We show that a tumor-intrinsic two-subtype schema is the most replicable and clinically robust across different subtype schemas, with basal-like subtype tumors showing resistance to FOLFIRINOX-based regimens in two independent clinical trials. Our results strongly support the need to evaluate molecular subtyping in treatment decision-making for patients with PDAC in the context of future clinical trials. We present PurIST, a clinically usable single-sample classifier that is robust and highly replicable across different gene expression platforms and sample collection types, and may be utilized in future clinical trials.

As such, present herein is a clinically usable SSC that may be used on any type of gene expression data including RNAseq, microarray, and NanoString, and on diverse sample types including FFPE, core biopsies, FNAs, and bulk frozen tumors. Although results of the association of FOLFIRINOX resistance in patients with basal-like subtype tumors is compelling, future prospective clinical trials in patients with PDAC will be needed to evaluate the utility of PurIST in treatment decision making, and in the context of different therapies. The flexibility and utility of PurIST on low-input samples such as tumor biopsies allows it to be used at the time of diagnosis to facilitate the choice of effective therapies for patients with pancreatic ductal adenocarcinoma and should be considered in the context of future clinical trials

### II. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” mean “one or more” when used in this application, including the claims. Thus, the phrase “a cell” refers to one or more cells, unless the context clearly indicates otherwise.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause, other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising.” “consisting essentially of,” and “consisting of,” where one of these three terms is used herein, the presently disclosed and claimed subject matter can include the use of either of the other two terms. For example, it is understood that the methods of the presently disclosed subject matter in some embodiments comprise the steps that are disclosed herein and/or that are recited in the claims, in some embodiments consist essentially of the steps that are disclosed herein and/or that are recited in the claims, and in some embodiments consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, the presently disclosed subject matter relates to human subjects.

Similarly, all genes, gene names, and gene products disclosed herein are intended to correspond to orthologs from any species for which the compositions and methods disclosed herein are applicable. Thus, the terms include, but are not limited to genes and gene products from humans. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are also intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about,” as used herein when referring to a measurable value such as an amount of weight, time, dose, etc., is meant to encompass variations of in some embodiments ±20%, in some embodiments±10%, in some embodiments±5%, in some embodiments #1%, and in some embodiments±0.1% from the specified amount, as such variations are appropriate to perform the disclosed methods and/or to employ the presently disclosed arrays.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

As used herein, the term “ANXA10” refers to the annexin A10 (ANXA10) gene and its transcription and translation products. Exemplary ANXA10 nucleic acid and amino acid sequences are presented in Accession Nos. NM 007193.5 and NP 009124.2 of the GENBANK® biosequence database, respectively, and are also set forth in SEQ ID NOs: 1 and 2, respectively.

As used herein, the term “BCAR3” refers to the BCAR3 adaptor protein, NSP family member (BCAR3), gene and its transcription and translation products. Exemplary BCAR3 nucleic acid and amino acid sequences are presented in Accession Nos. NM_001261408.2 and NP 001248337.1 of the GENBANK® biosequence database, and are also set forth in SEQ ID NOs: 3 and 4, respectively.

As used herein, the term “C16orf74” refers to the Homo sapiens chromosome 16 open reading frame 74 (C16orf74) gene and its transcription and translation products. Exemplary C16orf74 nucleic acid and amino acid sequences are presented in Accession Nos. NM 206967.3 and NP_996850.1 of the GENBANK® biosequence database, and are also set forth in SEQ ID NOs: 5 and 6, respectively.

As used herein, the term “CDH17” refers to the cadherin 17 (CDH17) gene and its transcription and translation products. Exemplary CDH17 nucleic acid and amino acid sequences are presented in Accession Nos. NM 004063.4 and NP 004054.3 of the GENBANK® biosequence database, and are also set forth in SEQ ID NOs: 7 and 8, respectively.

As used herein, the term “CLDN18” refers to the claudin 18 (CLDN18) gene and its transcription and translation products. Exemplary CLDN18 nucleic acid and amino acid sequences are presented in Accession Nos. NM 016369.4 and NP 057453.1 of the GENBANK® biosequence database, and are also set forth in SEQ ID NOs: 9 and 10, respectively.

As used herein, the term “CLRN3” refers to the clarin 3 (CLRN3) gene and its transcription and translation products. Exemplary CLRN3 nucleic acid and amino acid sequences are presented in Accession Nos. NM 152311.5 and NP 689524.1 of the GENBANK® biosequence database, and are also set forth in SEQ ID NOs: amino acid and 12, respectively.

As used herein, the term “CTSE” refers to the cathepsin E (CTSE) gene and its transcription and translation products. Exemplary CTSE nucleic acid and amino acid sequences are presented in Accession Nos. NM 001910.4 and NP 001901.1 of the GENBANK® biosequence database, and are also set forth in SEQ ID NOs: 13 and 14, respectively.

As used herein, the term “DCBLD2” refers to the discoidin, CUB and LCCL domain containing 2 (DCBLD2) gene and its transcription and translation products. Exemplary DCBLD2 nucleic acid and amino acid sequences are presented in Accession Nos. NM 080927.4 and NP_563615.3 of the GENBANK® biosequence database, and are also set forth in SEQ ID NOs: 15 and 16, respectively.

As used herein, the term “DDC” refers to the dopa decarboxylase (DDC) gene and its transcription and translation products. Exemplary DDC nucleic acid and amino acid sequences are presented in Accession Nos. NM_000790.4 and NP 000781.2 of the GENBANK® biosequence database, and are also set forth in SEQ ID NOs: 17 and 18, respectively.

As used herein, the term “GATA6” refers to the GATA binding protein 6 (GATA6) gene and its transcription and translation products. Exemplary GATA6 nucleic acid and amino acid sequences are presented in Accession Nos. NM 005257.6 and NP 005248.2 of the GENBANK® biosequence database, and are also set forth in SEQ ID NOs: 19 and 20, respectively.

As used herein, the term “GPR87” refers to the G protein-coupled receptor 87 (GPR87) gene and its transcription and translation products. Exemplary GPR87 nucleic acid and amino acid sequences are presented in Accession Nos. NM 023915.4 and NP 076404.3 of the GENBANK® biosequence database, and are also set forth in SEQ ID NOs: 21 and 22, respectively.

As used herein, the term “ITGA3” refers to the integrin subunit alpha 3 (ITGA3) gene and its transcription and translation products. Exemplary ITGA3 nucleic acid and amino acid sequences are presented in Accession Nos. NM_002204.4 and NP 002195.1 of the GENBANK® biosequence database, and are also set forth in SEQ ID NOs: 23 and 24, respectively.

As used herein, the term “KRTS” refers to the keratin 5 (KRTS) gene and its transcription and translation products. Exemplary KRT5 nucleic acid and amino acid sequences are presented in Accession Nos. NM_000424.4 and NP_000415.2 of the GENBANK® biosequence database, and are also set forth in SEQ ID NOs: 25 and 26, respectively.

As used herein, the term “KRT6A” refers to the keratin 6A (KRT6A) gene and its transcription and translation products. Exemplary KRT6A nucleic acid and amino acid sequences are presented in Accession Nos. NM 005554.4 and NP 005545.1 of the GENBANK® biosequence database, and are also set forth in SEQ ID NOs: 27 and 28, respectively.

As used herein, the term “KRT15” refers to the keratin 15 (KRTIS) gene and its transcription and translation products. Exemplary KRT15 nucleic acid and amino acid sequences are presented in Accession Nos. NM_002275.4 and NP 002266.3 of the GENBANK& biosequence database, and are also set forth in SEQ ID NOs: 29 and 30, respectively.

As used herein, the term “KRT17” refers to the keratin 17 (KRT17) gene and its transcription and translation products. Exemplary KRT17 nucleic acid and amino acid sequences are presented in Accession Nos. NM_000422.3 and NP_000413.1 of the GENBANK® biosequence database, and are also set forth in SEQ ID NOs: 31 and 32, respectively.

As used herein, the term “LGALS4” refers to the galectin 4 (LGALS4) gene and its transcription and translation products. Exemplary LGALS4 nucleic acid and amino acid sequences are presented in Accession Nos. NM_006149.4 and NP_006140.1 of the GENBANK® biosequence database, and are also set forth in SEQ ID NOs: 33 and 34, respectively.

As used herein, the term “LYZ” refers to the lysozome (LYZ) gene and its transcription and translation products. Exemplary LYZ nucleic acid and amino acid sequences are presented in Accession Nos. NM 000239.3 and NP_000230.1 of the GENBANK® biosequence database, and are also set forth in SEQ ID NOs: 35 and 36, respectively.

As used herein, the term “MUC17” refers to the mucin 17, cell surface associated (MUC17) gene and its transcription and translation products. Exemplary MUC17 nucleic acid and amino acid sequences are presented in Accession Nos. NM_001040105.2 and NP 001035194.1 of the GENBANK® biosequence database, and are also set forth in SEQ ID NOs: 37 and 38, respectively.

As used herein, the term “MYOIA” refers to the myosin 1A (MYOIA) gene and its transcription and translation products. Exemplary MYO1A nucleic acid and amino acid sequences are presented in Accession Nos. NM_005379.4 and NP 005370.1 of the GENBANK® biosequence database, and are also set forth in SEQ ID NOs: 39 and 40, respectively.

As used herein, the term “NR112” refers to the nuclear receptor subfamily 1 group I member 2 (NR1I2) gene and its transcription and translation products. Exemplary NR1I2 nucleic acid and amino acid sequences are presented in Accession Nos. NM_022002.2 and NP 071285.1 of the GENBANK® biosequence database, and are also set forth in SEQ ID NOs: 41 and 42, respectively.

As used herein, the term “PIP5K1B” refers to the phosphatidylinositol-4-phosphate 5-kinase, type I, beta (PIP5K1B) gene and its transcription and translation products. Exemplary PIPSKIB nucleic acid and amino acid sequences are presented in Accession Nos. NM 003558.4 and NP 003549.1 of the GENBANK® biosequence database, and are also set forth in SEQ ID NOs: 43 and 44, respectively.

As used herein, the term “PLA2G10” refers to the phospholipase A2 group X (PLA2G10) gene and its transcription and translation products. Exemplary PLA2G10 nucleic acid and amino acid sequences are presented in Accession Nos. NM 003561.3 and NP 003552.1 of the GENBANK® biosequence database, and are also set forth in SEQ ID NOs: 45 and 46, respectively.

As used herein, the term “PTGES” refers to the prostaglandin E synthase (PTGES) gene and its transcription and translation products. Exemplary PTGES nucleic acid and amino acid sequences are presented in Accession Nos. NM 004878.5 and NP 004869.1 of the GENBANK® biosequence database, and are also set forth in SEQ ID NOs: 47 and 48, respectively.

As used herein, the term “REG4” refers to the regenerating family member 4 (REG4) gene and its transcription and translation products. Exemplary REG4 nucleic acid and amino acid sequences are presented in Accession Nos. NM 032044.4 and NP 114433.1 of the GENBANK® biosequence database, and are also set forth in SEQ ID NOs: 49 and 50, respectively.

As used herein, the term “S100A2” refers to the S100 calcium binding protein A2 (S100A2) gene and its transcription and translation products. Exemplary S100A2 nucleic acid and amino acid sequences are presented in Accession Nos. NM_005978.4 and NP 005969.2 of the GENBANK® biosequence database, and are also set forth in SEQ ID NOs: 51 and 52, respectively.

As used herein, the term “SLC40A1” refers to the solute carrier family 40 member 1 (SLC40A1) gene and its transcription and translation products. Exemplary SLC40A1 nucleic acid and amino acid sequences are presented in Accession Nos. NM_014585.6 and NP 055400.1 of the GENBANK® biosequence database, and are also set forth in SEQ ID NOs: 53 and 54, respectively.

As used herein, the term “TFF1” refers to the trefoil factor 1 (TFF1) gene and its transcription and translation products. Exemplary TFF1 nucleic acid and amino acid sequences are presented in Accession Nos. NM_003225.3 and NP 003216.1 of the GENBANK® biosequence database, and are also set forth in SEQ ID NOs: 55 and 56, respectively.

As used herein, the term “TSPAN8” refers to the tetraspanin 8 (TSPAN8) gene and its transcription and translation products. Exemplary TSPAN8 nucleic acid and amino acid sequences are presented in Accession Nos. NM_004616.3 and NP_004607.1 of the GENBANK® biosequence database, and are also set forth in SEQ ID NOs: 57 and 58, respectively.

The term “isolated,” as used in the context of a nucleic acid or polypeptide (including, for example, a nucleotide sequence, a polypeptide, and/or a peptide), indicates that the nucleic acid or polypeptide exists apart from its native environment. An isolated nucleic acid or polypeptide can exist in a purified form or can exist in a non-native environment.

Further, as used for example in the context of a cell, nucleic acid, polypeptide, or peptide, the term “isolated” indicates that the cell, nucleic acid, polypeptide, or peptide exists apart from its native environment. In some embodiments, “isolated” refers to a physical isolation, meaning that the cell, nucleic acid, polypeptide, or peptide has been removed from its native environment (e.g., from a subject).

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene,” “cDNA,” and “mRNA.” Nucleic acids can be synthesized, or can be derived from any biological source, including any organism.

As used herein, the terms “peptide” and “polypeptide” refer to polymers of at least two amino acids linked by peptide bonds. Typically, “peptides” are shorter than “polypeptides,” but unless the context specifically requires, these terms are used interchangeably herein.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### II. Methods

In some embodiments, the presently disclosed subject matter relates to methods for determining a subtype of a pancreatic tumor in a biological sample comprising, consisting essentially of, or consisting of pancreatic tumor cells obtained from a subject. As used herein, the phrase “subtype of a pancreatic tumor” refers to classifications wherein the underlying nature of the pancreatic tumor and/or cells thereof are classified differentially with respect to gene expression, prognosis, treatment decisions, etc. Various subtypes for pancreatic tumors and cells thereof have been described in the literature, including those set forth in, for example, U.S. Patent Application Publication No. 2017/0233827; Moffitt et al., 2015; Bailey et al., 2016; Nywening et al., 2016; Aung et al., 2017; Cancer Genome Atlas Research Network, 2017; Connor et al., 2017; and Aguirre et al., 2018; each of which is incorporated herein by reference in its entirety.

In some embodiments, the pancreatic tumor is classified as being of the basal-like subtype or of the classical subtype. The classification with respect to basal-like vs classical can be made on the basis of the methods disclosed herein. By way of example and not limitation, a method for classifying a pancreatic tumor as being of the classical vs. the basal-like subtype can comprise obtaining gene expression levels for each of the following genes in the biological sample: GPR87, KRT6A, BCAR3, PTGES, ITGA3, C16orf74, S100A2, KRT5, REG4, ANXA10, GATA6, CLDN18, LGALS4, DDC,GENE SLC40A1, CLRN3; performing a pair-wise comparison of the gene expression levels for each of Gene Pairs 1-8 or for each of Gene Pairs A-H, wherein Gene Pairs 1-8 and Gene Pairs A-H are as shown in Table 1; and calculating a Raw Score for the biological sample. In some embodiments, the calculating comprises assigning a value of 1 for each Gene Pair for which Gene A of the Gene Pair has a higher expression level than Gene B of the Gene Pair, and a value of 0 for each Gene Pair for which Gene A of the Gene Pair has a lower expression level than Gene B of the Gene Pair; multiplying each assigned value by the coefficient listed in Table 1 for the corresponding Gene Pair to calculate eight individual Gene Pair scores, and adding the eight individual Gene Pair scores together along with a baseline effect to calculate a Raw Score for the biological sample, wherein the baseline effect is −6.815 for Gene Pairs 1-8 and −12.414 for Gene Pairs A-H (i.e., the intercepts identified in Tables 25 and 26). To assign a subtype to the biological sample, if the calculated Raw Score is greater than or equal to 0, the tumor subtype is determined to be a basal-like subtype, and if the calculated Raw Score if less than 0, the tumor subtype is determined to be a classical subtype.

In some embodiments, the Raw Score that is calculated is further converted to a predicted basal-like probability (PBP) using the inverse-logit transformation

$$P B P = {e}^{Raw Score} / ( 1 + {e}^{Raw Score} ) .$$

The PBP is another way to classify pancreatic tumor subtypes as being basal-like or classical. When a PBP is calculated, the threshold value for classifying basal-like vs. classical is slightly modified In these cases, if the PBP is greater than 0.5, the tumor subtype is determined to be a basal-like subtype, and if the PBP if less than or equal to 0.5, the tumor subtype is determined to be a classical subtype.

As used herein, the terms “biological sample” and “sample” refer to a biopsy sample, optionally a fine needle biopsy aspiration or a percutaneous core needle biopsy, or a frozen or archival sample derived therefrom, that comprises pancreatic tumor (in some embodiments, pancreatic ductal adenocarcinoma (PDAC)) cells that have been isolated from a patient with a pancreatic tumor and/or nucleic acids and/or proteins that have been isolated from such a sample. Depending on the type of gene expression analysis to be employed (discussed in more detail herein below), the sample should comprise DNA, RNA (in some embodiments messenger RNA; mRNA), or protein.

Given that the methods disclosed herein relate to pairwise comparisons of multiple genes with respect to expression levels of the corresponding gene products in the biological samples, comparisons of nucleic acid gene products or protein gene products can be employed. As would be understood by one of ordinary skill in the art, quantitative assays can be desirable to determine relative expression levels. With respect to nucleic acids, particularly mRNA gene products, a technique selected from the group consisting of microarray analysis, RNAseq, quantitative RT-PCR, NanoString, or any combination thereof can be employed. Non-limiting examples of such techniques include whole transcriptome RNAseq, targeted RNAseq, SAGE, RT-PCR (particularly QRT-PCR), cDNA microarray analyses, and NanoString analysis. Techniques for assaying gene expression levels using RT-PCR, nucleic acid and/or protein microarray hybridization, and RNA-Seq are known in the art (see e.g., U.S. Pat. Nos. 5,800,992; 6,004,755; 6,013,449; 6,020,135; 6,033,860; 6,040,138; 6,177,248; 6,251,601; 6,309,822; 7,824,856; 9,920,367; 10,227,584; each of which is incorporated by reference in its entirety. See also U.S. Patent Application Publication Nos. 2010/0120097; 2011/0189679; 2014/0113333; 2015/0307874; each of which is incorporated by reference in its entirety.

In some embodiments, the assay involves use of NanoString. The basic NanoString technology is described in PCT International Patent Application Publication No. WO 2019/226514 and U.S. Pat. No. 9,181,588, each of which is incorporated herein by reference in its entirety. For use with Gene Pairs 1-8 and A-H, one of ordinary skill in the art can design appropriate NanoString probes based on the sequences of the corresponding gene products. Exemplary NanoString probes are identified in Table 6. In some embodiments, and particularly wherein different assay techniques are employed with different samples, an internal control can be employed to normalize and/or harmonize gene expression data. In some embodiments, an internal control comprises a housekeeping gene. Exemplary housekeeping genes include the CTSE, LYZ, MUC17, MYOIA, NR112, PIP5KIB, RPLPO, B2M, ACTB, RPL19, GAPDH, LDHA, PGK1, TUBB, SDHA, CLTC, HPRTI, ABCF1, GUSB, TBP, and ALASI, and exemplary NanoString probes that can be employed with these genes are disclosed in SEQ ID NOs: 75-102, respectively.

In some embodiments, a gene product is a protein gene product, and gene expression is determined by quantifying an amount of protein present in a sample. Methods for quantifying gene expression at the protein level are known, and include but are not limited to enzyme-linked immunosorbent assay (ELISA), immunoprecipitation (IP), radioimmunoassay (RIA), mass spectroscopy (MS), quantitative western blotting, protein and/or peptide microarrays, etc. See e.g., U.S. Pat. Nos. 7,595,159; 8,008,025; 8,293,489; and 10,060,912; each of which is incorporated by reference herein in its entirety. For those assays that require the use of antibodies, various commercial sources of antibodies, including monoclonal antibodies, exist, including but not limited to ProMab Biotechnologies, Inc. (Richmond, California, United States of America), abcam plc (Cambridge, United Kingdowm), Santa Cruz Biotchnology, Inc. (California, United States of America), etc.

In some embodiments, the determination of subtype of a pancreatic tumor sample, optionally a PDAC sample, can be employed in making a differential treatment decision with respect to the subject since basal-like and classical subtypes respond differently to different treatments. By way of example and not limitation, if the assigned subtype is a basal-like subtype, a differential treatment strategy for that subject/patient could be with gemcitabine (i.e., 4-amino-1-[(2R,4R,SR)-3,3-difluoro-4-hydroxy-5-(hydroxymethyl) oxolan-2-yl]pyrimidin-2-one, which is often administered as a hydrochloride; see U.S. Patent Application Publication No. 2008/0262215 and U.S. Pat. No. 8,299,239), optionally in combination with paclitaxel (i.e., [(1S,2S,3R,4S, 7R,9S, 10S, 12R, 15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo [11.3.1.03,10.04,7]heptadec-13-en-2-yl]benzoate; see U.S. Pat. No. 6,753,006) or nab-paclitaxel (i.e., ABRAXANE® brand nanoparticle albumin-bound paclitaxel; see U.S. Pat. No. 7,758,891). Methods for treating pancreatic cancer with gemcitabine and/or paclitaxel/nab-paclitaxel are known (see e.g., U.S. Patent Application Publication No. 2017/0020824, which is incorporated herein by reference in its entirety).

If the subtype of the pancreatic tumor sample is classical, then in some embodiments the subject/patient is treated with FOLFIRINOX (composed of folinic acid (leucovorin), fluorouracil, irinotecan, and oxaliplatin; Conroy et al., 2011). In some embodiments, FOLFIRINOX can be combined with other treatments, including but not limited to the CCR2 inhibitor PF-04136309 (see Nywening et al., 2016).

In some embodiments, additional anti-pancreatic cancer/tumor strategies can be employed, including but not limited to surgery, radiation, or administration of other chemotherapeutics. Exemplary chemotherapeutics that can be employed in the methods of the presently disclosed subject matter include, but are not limited to protein kinase inhibitors (PKIs). A listing of exemplary PKIs, their targets, and their associations with basal-like and classical tumor subtypes is presented in Table 28. In some embodiments, a PKI that is associated with overexpression in basal-like subtypes tumors is employed in a combination therapy for samples that are of a basal-like subtype. In some embodiments, a PKI that is associated with overexpression in classical subtype tumors is employed in a combination therapy for samples that are of the classical subtype.

In some embodiments, the presently disclosed subject matter also provides methods for treating patients diagnosed with PDAC. In some embodiments, the methods comprise determining a subtype of the patient's PDAC as being basal-like or classical, and treating the subject as disclosed herein. In some embodiments, basal-like subtype patients are treated with gemcitabine, optionally in combination with nab-paclitaxel, and classical subtype patients are treated with FOLFIRINOX, optionally in combination with a CCR2 inhibitor. The combination therapies discussed herein above can also be employed in the treatment methods of the presently disclosed subject matter.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

Public datasets. Archival data were obtained from public sources (see Moffitt et al., 2015; Aung et al., 2017; Aguirre et al., 2018; Bailey et al., 2016; Nywening et al., 2016; Connor et al., 2017; and Cancer Genome Atlas Research Network, 2017) and are summarized in Table 4. For the public datasets, expression was used “as-is” with respect to the original publication; that is, RNAseq data were not realigned and gene-level expression estimates were provided in terms of fragments per kilobase per million reads (FPKM) or transcripts per million (TPM), depending on the study.

Sample collection. Deidentified bulk and FNA samples (see Table 5) were collected from the Institutional Review Board (IRB)-approved University of North Carolina Lineberger Comprehensive Cancer Center Tissue Procurement Core Facility after IRB exemption in accordance with the U.S. Common Rule and were flash frozen in liquid nitrogen. FNA samples were collected ex vivo at the time of resection. The FNA technique used mirrors standard cytopathology procedures, where three passes were performed using a 22-gauge needle. Palpation was used to localize the tumor. Samples were frozen in either PBS or RNALATER® brand stabilizing reagent (Sigma-Aldrich Corp., St. Louis, Missouri, United States of America). FFPE samples were prepared, hematoxylin and eosin stained, and reviewed by a single pathologist who was blinded to the results as described herein. See below for data processing and analysis of Yeh_Seq samples RNAseq (GSE131050) and NanoString (GSE131051) data generated from these samples are deposited in Gene Expression Omnibus (GEO).

RNAseg. Samples for Yeh_Seq were sequenced on a NEXTSEQ® 500 brand sequencing system (Illumina, inc., San Diego, California, United States of America). We converted BCL files to FASTQ using bcl2fastq2 Conversion Software 2.20.0 (Illumina, Inc.). Individual lane files were combined into one FASTQ for each sample. FASTQ of PDX samples were split into human and mouse reads using bbmap v37.90 (Bushnell, 2014). The total expected read counts per gene were quantified by Salmon 0.9.1 (Patro et al., 2017) using arguments “--gcBias--seqBias”. For human samples, Genome Reference Consortium Human Build 38 (GRCh38) was used. For PDX samples, GRCh38 was involved in quantification for human reads, while the mouse reference genome GRCm38/mm10 (December 2011) available at the website of the University of California Santa Cruz (UCSC) Genomics Institute was used to quantify mouse reads. The expression of each gene was measured by the Transcripts per Million (TPM), which was subjected to downstream analysis.

Customized quality control guidelines were used for low-input (FNA) and degraded (FFPE) samples (Adiconis et al., 2013). Bulk or FNA samples were flagged if the proportion of bases mapped to coding regions fell below 30%. For FFPE samples, samples were flagged if the proportion fell below 10%. We also checked the total number of unique reads after deduplication. Bulk and FFPE samples were flagged if the total number of unique reads were below 1 million. FNA samples were flagged if the total number of unique reads were below half a million. We also checked the uniformity of transcript coverage by assessing 5′-to-3′ bias using gene body plots, and insert size distribution, so that any sample that clearly distinguished itself as an outlier was flagged.

For the Linehan dataset, total RNA was isolated from matched patient tumor biopsies collected at baseline and post-treatment cycle two as part of clinical study NCT01413022 testing the efficacy of PF-04136309 in combination with FOLFIRINOX as previously described (Nywening et al., 2016). RNA expression libraries were generated with TruSeq Stranded mRNA kits according to the manufacturer's instructions and sequencing was performed on the HiSeq 2500 Sequencing System (Illumina, Inc.). BCL files were converted to FASTQ with beltofastq software v2.19.0 (Illumina, Inc.). The total expected read counts per gene were quantified by Salmon 0.9.1 using arguments “--gcBias--seqBias” and reference genome GRCh38, which were normalized to TPM as described above.

CC based subtype calling. Unsupervised CC was applied for each of the subtyping schemas (Collisson, Bailey, and Moffitt) on all public datasets included in our study as previously described using the ConsensusClusterPlus package in R (Aguirre et al., 2018), subsequent to sample filtering. In brief, 62 genes identified by Collisson (Collisson et al., 2011), 613 differentially expressed genes from the multiclass SAM analysis by Bailey (Bailey et al., 2016), and 50 tumor specific genes from Moffitt (Moffitt et al., 2015) were utilized for subtyping analysis, seeking the presence of 3, 4, and 2 clusters respectively For the Bailey and Collisson schemas and using published calls as the gold standard (Bailey subtypes in the PACA_AU_array and PACA_AU_seq datasets, and Bailey and Collisson subtypes in the TCGA_PAAD dataset), we found a better concordance of the subtype calls by applying row-scaling than without row-scaling prior to consensus clustering (CC). Therefore, for the Bailey and Collisson schemas, each dataset was subjected to gene-wise (row) scaling across samples so that expressions were normalized to z-scores for each gene as the input for CC. Row-scaling was not applied to the Moffitt schema. For the COMPASS and Connor datasets, the 10 least variable signature genes were dropped in subtype calling for the Bailey schema since, in these two datasets, the CC found subsamples with 0 variance which led to termination of the function in R.

### PurIST Single Sample Classifier.

Data pre-processing. For each RNAseq dataset, we first removed genes in the bottom 20% percentile in expression on average in that dataset. This is to remove consistently low expressing genes that may be unhelpful for prediction later. For microarray data, due to probe-specific effects, it is more difficult to assume that measured expression is correlated with actual biological expression, so we do not apply this filtering here. We then further reduced the list of remaining genes in each dataset to those belonging to a list of 500 Moffitt tumor-specific genes determined previously (Moffitt et al., 2015). Finally, we retained only those genes that were in common across all nine datasets after these filtering steps. At the end of this process, we had 412 genes out of 500 tumor-specific genes remaining that were in common across all 9 data datasets.

Training Datasets and candidate gene ranking. Training labels and expression values from the genes in our tumor-specific gene list served as the basis for our building the PurIST model. Training labels for PurIST were a subset of the Moffitt CC in the Training Group datasets (Aguirre, Moffitt_GEO_array and TCGA_PAAD; Table 7) were utilized. These samples were further filtered to provide final training labels for the PurIST algorithm by dropping poorly clustered samples on the clustered dendrogram in each dataset based on visual inspection. We considered these filtered calls as “training labels”. Because not all genes may be consistent in their relationship with tumor subtype across training datasets or may be strongly discriminatory between subtypes, we ranked candidate genes in based on the consistency of their Differential Expression (DE) between subtypes in each individual Training Group dataset, as well as the consistency in the direction of their DE for utilization in subsequent steps (Lusa et al, 2007; Paquet & Hallett, 2015). We applied the Wilcoxon Rank Sum test to each gene in a given study to test for differences in mean expression between basal-like and classical subjects. We then obtained a cross-study DE consistency score by summing the −log₁₀ p-values for differential expression across studies. In general, genes that were consistently differentially expressed were most likely to have higher scores. Then, we ranked genes based on this score from largest to smallest. We then considered the top 10% of this list for model training. Lastly, we removed genes where the sign of the difference in mean subtype expression was not the same in all Training Group datasets. The remaining genes then formed our final candidate gene list for downstream steps in PurIST model training.

kTSP selection for prediction: overview. Let us define a gene pair (g_{dis}, g_{dit}), where gais is the raw expression of gene s for subject i in study d, and gait is defined similarly with respect to some gene t. A TSP is an indicator variable based on this gene pair, I (g_{dis}>g_{dit})−1/2, where its value represents which gene in the pair has higher expression in subject i from study d (1/2 if g_{dis}>g_{dit}, and −1/2 otherwise). In traditional applications (k=1), a single TSP is selected out of the set of all possible gene pairs such that if I (g_{dis}>g_{dit})−1/2>0, this implies subtype A with high probability in the training data, otherwise implying subtype B (Geman et al., 2004). Therefore, in a new subject, binary class prediction is performed by checking whether I (g_{dis.1}>g_{dit.1})−1/2>0 vs otherwise. We view such binary variables as “biological switches” indicating how pairs of genes are expressed relative to some clinical outcome. TSPs were originally proposed in the context of binary classification (Geman et al., 2004; Tan et al., 2005; Afsari et al., 2014) In the kTSP setting, class prediction reduces to verifying whether the sum across k selected TSPs is greater than 0:

$${{\sum }_{l = 1}}^{k} I ( {g}_{dis , l} > {g}_{dit , l} ) - \frac{1}{2} > 0$$

This reduces to a majority vote across the selected k TSPs, where the contribution of each of the k TSPs are equally weighted to select subtype A if the above sum is greater than 0, and subtype B otherwise.

We describe this approach to select TSPs in the next section. However, several studies have found that equal weighting of TSPs in majority voting may be suboptimal, as some TSPs may be more informative than others (Shi et al., 2011). Therefore, we utilized penalized logistic regression (Breheny & Huang, 2011) to jointly estimate the effect of each of the k selected TSPs in predicting binary subtype, and to further remove TSPs with weak or redundant effects. Predicted probabilities of basal-like subtype membership may then be obtained from the fitted model logistic regression model on our training samples, where values greater than 0.5 indicate predicted membership to the basal-like subtype and classical otherwise.

Horizontal data integration and kTSP selection via switchbox. To apply the top scoring pairs transformation, we utilized the switchBox R package (Afsari et al., 2015) to enumerate all possible gene pairs based on our final candidate gene list and training samples (function SWAP.KTSP. Train, with optimal parameters featureNo=1000, krange=50,FilterFunc=NULL). Given the large number of potential gene pairs based on this list, in addition to the strong correlation between gene pairs sharing the same gene, the switchBox package utilized a greedy algorithm to select from this list a subset of gene pairs that were helpful for prediction, given the set of training labels. We merged data from each Training Group dataset without normalization prior to applying switchBox, as the method only looked at the relative gene expression ranking within each sample from each study. The method then selected a subset of k TSPs, where k is determined through a greedy optimization procedure.

Model training based on selected kTSP list. To remove redundant TSPs and to jointly estimate their contribution in predicting subtype in our training samples, we utilized the ncvreg R package (Breheny & Huang, 20111) to fit a penalized logistic regression model based upon the selected TSPs from switchBox. Our design matrix was an Nx (k+1) matrix, where the first column pertained to the intercept and the remaining k columns pertained to the k selected TSPs from switchBox. Here N was the total number of training samples from each dataset employed for training. Each TSP in the design matrix was represented as a binary vector, taking on the value of 1 if gene A's expression was greater than gene B's expression. Our outcome variable here was binary subtype (1=Basal, 0 otherwise). We utilized optional parameters alpha=0.5 and nfolds=N. We allowed for correlation between TSPs by setting the nevreg alpha parameter to 0.5 in order to shrink the coefficients of highly correlated TSPs and also remove correlated uninformative TSPs from the model. We set nfolds=N to apply leave one out cross validation in order to choose the optimal MCP penalty tuning parameter for variable selection, where the optimal tuning parameter was the one that minimized the cross-validation error of the fitted model. Our final model then reported the set of coefficients estimated for each of the kTSPs, where each coefficient may be interpreted as the change in log odds of a patient being part of the basal-like subtype when the 1th TSP is equal to 1, given the others in the model. TSPs with coefficient of 0 were those that have been removed from the model for either weak effect or redundancy with other TSPs. Predicted probabilities of Basal subtype membership may be obtained by computing the inverse logit of the linear predictor X_{i,new}β (the Raw Score), where X_{i,new} was a 1×(k+1) TSP predictor vector from a new sample, and β was our estimated set of coefficients from the fitted penalized logistic regression model. Then, predicted probabilities of basal-like subtype membership for this new sample can be computed through the inverse logit function:

$${\hat{p}}_{i , new} = exp ( {X}_{i , new} \hat{β} ) / ( 1 + exp ( {X}_{i , new} \hat{β} ) )$$

P_{i,new} values greater than 0.5 indicated predicted membership basal-like subtype, and those less than 0.5 were those that were predicted those be of the classical subtype. This was equivalent to determining whether X_{i,new}>0 (basal-like subtype) vs X_{i,new}ß<0 (classical subtype), where X_{i,new}β may also be utilized as a continuous score for classification (“PurIST Score”). Therefore, prediction in new samples, such as from our validation datasets, reduced to simply checking the relative expression of each gene within the set of TSPs. Those TSPs with selected 0 coefficient can be ignored in this setting.

For all discussions regarding classifier performance, we obtained the predicted subtypes in the manner described above. The level of confidence in the prediction can be determined based upon the distance of P_{i,new} from 0.5, where values closer to P_{i,new} indicated lower confidence in the predicted subtype and higher confidence otherwise. Specifically, values of P_{i,new} between 0.5 and 0.6 indicated the lean basal-like prediction category, 0.6 and 0.9 represented the likely basal-like prediction category, and values greater than 0.9 indicated the strong basal-like prediction category. Values of P_{i,new} between 0.5 and 0.4 indicated the lean classical prediction category, 0.6 and 0.1 represented the likely classical prediction category, and values less than 0.1 indicated the strong classical prediction category.

NanoString and PurIST-n. We repeated the above procedure with a subset of genes using NanoString probes (PurIST-n; see Table 6). We then retrained our model in given our training datasets limiting to these genes, rebuilding candidate TSPs and applying our penalized logistic regression model to obtain our PurIST-n classifier. Matched samples from RNAseq were run on the NanoString nCounter platform as per manufacturers instruction. In brief, for each sample, RNA was combined with the NanoString master mix and the Capture Probe set. Hybridization of the RNA with the Capture Probe set took place overnight while incubating at 65° C. After hybridization completed, the samples were added to the NanoString nCounter cartridge and placed in the nCounter Prep Station using the high sensitivity setting. After the Prep Station run was complete, the cartridge was removed and placed in the NanoString Digital Analyzer for scanning.

Sample inclusion for consensus clustering analysis and PurIST training. For treatment response and survival analysis, samples with available clinical and RNAseq data were used. Specifically, for the pooled survival analysis, samples from the following datasets with RNAseq data and CC calls were utilized: Linehan, Moffitt_GEO_array, PACA_AU_seq, PACA_AU_array, and TCGA_PAAD (survival group; Table 7). Duplicated samples in PACA_AU_seq and PACA_AU_array datasets were only used once, with the subtypes called in PACA_AU_array used when mismatches of subtype calls were found between the two datasets. To train PurIST, Moffitt schema CC calls from the datasets in the training group (Aguirre, Moffitt_GEO_array, and TCGA_PAAD; Table 7) were utilized These samples were further filtered to provide final training labels for the PurIST algorithm by dropping poorly clustered samples on the clustered dendrogram in each dataset based on visual inspection. We considered these filtered calls as “training labels.” Model training for PurIST is described herein above.

Statistical Analysis. Overall survival estimates were calculated using the Kaplan-Meier method. Association between overall survival and individual covariates such as subtype were evaluated via the cox proportional hazards (coxph) models using the coxph function from the ‘survival’ R package, where a given subtyping schema was considered as a multi-level categorical predictor. The logrank p-value was utilized to evaluate overall association of a subtyping system with overall survival. In the pooled analyses, a stratified coxph model was utilized, where dataset of origin was used as a stratification factor to account for variation in baseline hazard across studies. To test for differences in survival between individual subtypes within a schema, linear contrasts were utilized in conjunction with the fitted stratified coxph model to construct a general linear hypothesis test. BIC pertaining to each fitted stratified coxph model was calculated for each schema using the “BIC” function in R, where smaller BIC values indicate better model fit. Agreement between subtype calls in patients within matched samples were performed using Cohen's Kappa via the “kappa2” function from the irr package in R. Hypothesis tests evaluating the null hypothesis that Kappa=0, indicating random agreement, was also performed using the kappa2 function. Kappa values of 1 indicate perfect agreement. Association between categorical response, defined by RECIST 1.1 criteria (PD, SD, PR, CR), and called subtypes from in a given clinical trial with treatment response was evaluated using the Generalized Cochran-Mantel-Haenszel test, with trial arm utilized as the stratification factor and assuming categorical treatment response as an ordinal variable. This is to correct for potential confounding due to differences between arms. This test was carried out using the “cmh_test” function from the coin R package. We determined an empirical null distribution for this test using permutation testing, assuming 5 million permutations to ensure robustness against any deviations from test assumptions. In modeling response as a continuous variable (% change in tumor volume from baseline) with respect to a given schema, two-way ANOVA was utilized, where schema subtype and arm were utilized as categorical factors, and BIC was calculated similar to before. When categorical response was utilized, a multinomial regression model utilizing schema subtypes as a categorical prediction was fit using the “polr” from the MASS R package, and BIC was calculated as mentioned previously. For the permutation test to compare correlation among various gene sets, we first evaluated the Spearman correlations between each of the PurIST TSP genes in FFPE vs. bulk, FFPE vs. FNA, and also bulk vs. FNA. This was also repeated for each of the Bailey ADEX genes and Bailey immunogenic genes. We then calculated paired Wilcoxon signed-rank statistic of to test if the 18 correlations among TSP genes was significantly higher than that of ADEX genes (or immunogenic genes). Since the 18 correlations were not independent observations, the null distribution was approximated using permutations. The permutation of the FFPE and FNA matches for the 6 bulk samples was done 10,000 times and the paired Wilcoxon statistic was likewise computed for each permutation. This generated the distribution of the statistic under the null hypothesis that the paired difference between correlations among TSP genes versus those among ADEX genes (or immunogenic genes) are centered around zero, which allowed us to derive a p-value for the observed statistic before permutation.

### Example 1

### The Moffitt Tumor-intrinsic Two-subtype Schema has Important Implications for Treatment Response

To evaluate the potential impact of molecular subtypes on treatment response, we utilized transcriptomic and treatment response data from two independent clinical trials, and performed a systematic analysis of treatment response with respect to CC calls from each of the three different subtyping schemas (described herein above)) for PDAC: Collisson, Bailey, and Moffitt (Collisson et al., 2011; Bailey et al., 2016; Moffitt et al., 2015). We first examined the association of the subtypes from each schema with treatment response using patient samples from a promising phase Ib trial by Nywening and colleagues (“Linehan,” Linehan_seq dataset; Tables 8-17) of FOLFIRINOX in combination with a CCR2 inhibitor (PF-04136309) in patients with locally advanced PDAC, where an objective response was seen in 49% of patients (Nywening et al., 2016). Enrolled patients had no prior treatment, and underwent core biopsies prior to the start of therapy. Twenty-eight patients with RNAseq and treatment data were available for analysis.

We found a significant overall association between categorical treatment response (based on RECIST 1.1 criteria) and pretreatment subtype classifications from the Moffitt schema (p=0.0117; Tables 18-21), where basal-like tumors showed no response to FOLFIRINOX alone or FOLFIRINOX plus PF-04136309 after stratifying by arm [overall response rate (ORR)=0%; disease control rate (DCR)=33%; Tables 18-21, generalized Cochran-Mantel-Haenszel test], whereas classical tumors showed a much stronger response overall (ORR=40%; DCR=100%). In contrast, we were unable to identify a relationship between subtype and treatment response under the Collisson (p=0.428) and Bailey (p=0.113) schemas (Tables 18-21). As the sample size in this phase Ib trial (n=28 patients) was small, we similarly reanalyzed the COMPASS trial results (n=40 patients) in the context of the three subtyping schemas.

Patients enrolled in COMPASS underwent core-needle biopsies and were treated with one of two standard first-line therapies, modified-FOLFIRINOX or gemcitabine plus nanoparticle albumin-bound paclitaxel (nab-paclitaxel). Collected patient samples in COMPASS underwent laser capture microdissection (LCM) followed by whole genome sequencing and RNAseq. Subtypes for each schema were determined as mentioned previously. Similar to our findings in the Linehan phase Ib trial, we found a significant association between the Moffitt two subtype schema with categorical treatment response stratifying by arm (P=0.00098, generalized Cochran-Mantel-Haenszel test), where the basal-like subtype had much lower response to either treatment (ORR=10%; DCR=50%) relative to the classical subtype (ORR=36.7%; DCR=100%). We also found significant associations between treatment response and the subtypes from the Collisson (p=0.0024) and Bailey (p=0.0067) schemas. However, we notably observe that the Bailey squamous subtype strongly overlaps with the Moffitt basal-like subtype, and the remaining nonsquamous Bailey subtypes appear to overlap strongly with the Moffitt classical subtype (Cohen Kappa=1.0, p=2.54×10⁻¹⁰). We similarly found that the Collisson QM-PDA and the remaining non-QM-PDA subtypes correspond strongly with the Moffitt basal-like and classical subtypes, respectively (Cohen Kappa=0.875, p=2.44×10⁻⁸), a fact also mirrored in the Linehan trial.

Given these observations, we formally evaluated the relative clinical utility of each subtyping system using non-nested model selection criteria such as Bayesian information criterion (BIC; Schwarz, 1978). Briefly, such criteria evaluate model fit relative to the complexity of the model, as models with more predictors (subtypes) may simply have better fit due to overfitting, and also may contain excess predictors (additional subtypes) that do not contribute meaningfully in differentiating clinical outcomes. The model with the lowest BIC in a series of competing candidate models is preferred in statistical applications, and is agnostic to the magnitude of the difference (Kass et al., 1995). Considering response as a continuous outcome (% change in tumor volume), we find that the Moffitt schema had the best (lowest) BIC score in both datasets (Linehan BIC=247.37, COMPASS BIC=378.75, two-way ANOVA model; Tables 18-21), compared with the Collisson (Linehan BIC=254.63, COMPASS BIC=382.8) and Bailey (Linehan BIC=250.75, COMPASS BIC=385.66) schemas. This result similarly held if we considered response as a categorical variable (ordinal regression model: Tables 18-21). This finding was also reflected among the non-QM-PDA and nonsquamous subtypes (Tables 18-21), where little difference in response can be seen between these subtypes. Our results using BIC suggested that the additional subtypes found in the Collisson and Bailey schemas do not demonstrate additional benefit in differentiating treatment response over the Moffitt two-subtype schema. Taken together, these results suggest that the Moffitt basal-like and classical subtypes strongly and parsimoniously explained treatment response relative to other schemas in both clinical trials.

The Linehan phase Ib trial captured both pre- and posttreatment biopsies, providing a unique opportunity to evaluate the stability of molecular subtypes after treatment. As pre- and post-treatment biopsies were unlikely to be obtained from the same location, these samples may also provide an opportunity to evaluate intrapatient tumor heterogeneity. Interestingly, we found strong stability in the Moffitt schema subtypes in pre- and post-treatment biopsies (Cohen Kappa=1.0; p=2.54 10-10), suggesting that not only may there be less tumor-intrinsic subtype heterogeneity within a tumor, but also that the Moffitt schema subtypes are not affected by treatment, either with FOLFIRINOX or with the addition of the CCR2 inhibitor In contrast, we found higher rates of switching in Collisson subtypes pre to posttreatment (Tables 23 and 24), where changes in the exocrine-like and classical subtypes were more common. Similarly, the nonsquamous Bailey subtypes appeared to show the highest rate of subtype switching pre- and posttreatment, with the ADEX subtype demonstrating the highest rate of switching among these subtypes (Tables 23 and 24).

It was unclear whether there is any clinical significance to such subtype transitions Prior studies had suggested that the Bailey ADEX, Bailey immunogenic, and Collisson exocrine-like subtypes are confounded by tumor purity in contrast to the Moffitt subtypes (Cancer Genome Atlas Research Network, 2017; Puleo et al., 2018; Maurer et al., 2019), which may explain some of the increased heterogeneity in subtypes pre- and posttreatment in these schemas. In contrast, the Collisson QM-PDA and Bailey squamous subtypes, which were shown to overlap strongly with the Moffitt basal-like subtype, were observed to be much more stable between the two time points.

### Example 2

### The Tumor-intrinsic Two-subtype Schema Strongly and Replicably Differentiates Patient Survival Across Multiple Studies

Given the paucity of available genomic data in the context of treatment response in PDAC, we also performed a meta-analysis of five independent patient cohorts with OS data available: Linehan_seq, Moffitt_GEO_array (GSE71729), ICGC PACA_AU_array, ICGC PACA_AU_seq, and TCGA_PAAD (survival group; Table 7). To determine the potential replicability of the different subtyping schemas (Collisson, Bailey, Moffitt) in differentiating clinical outcomes, we utilized CC subtype calls from each schema.

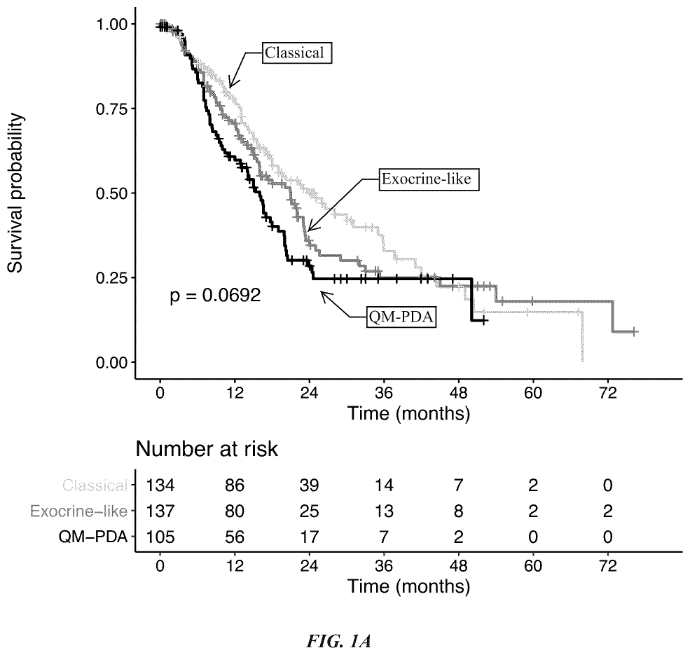

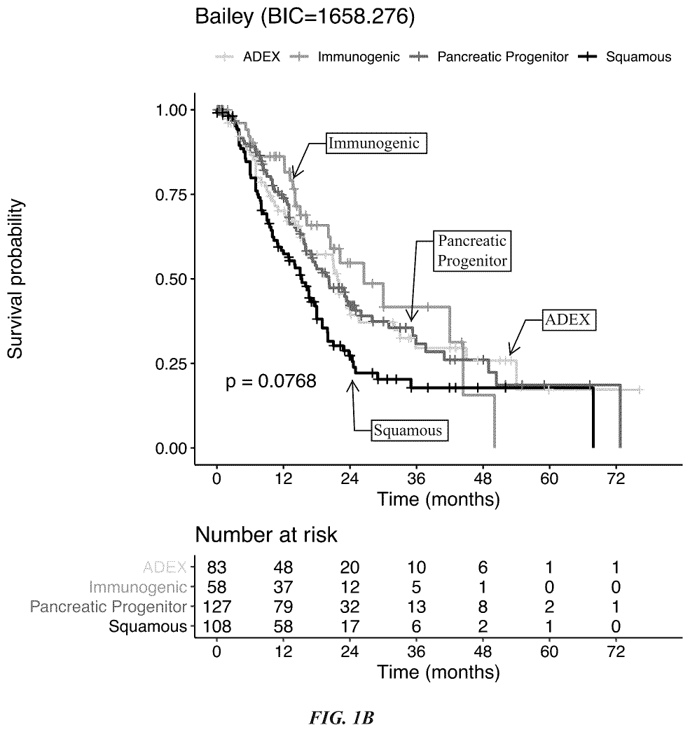

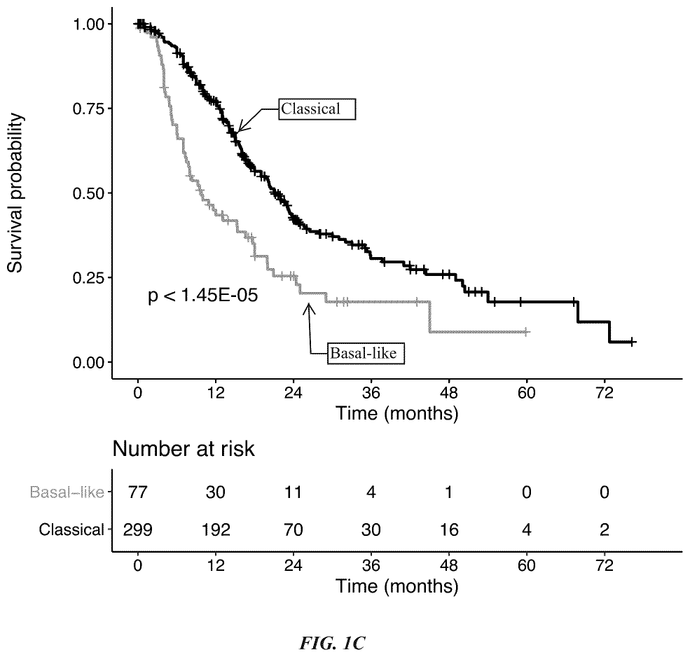

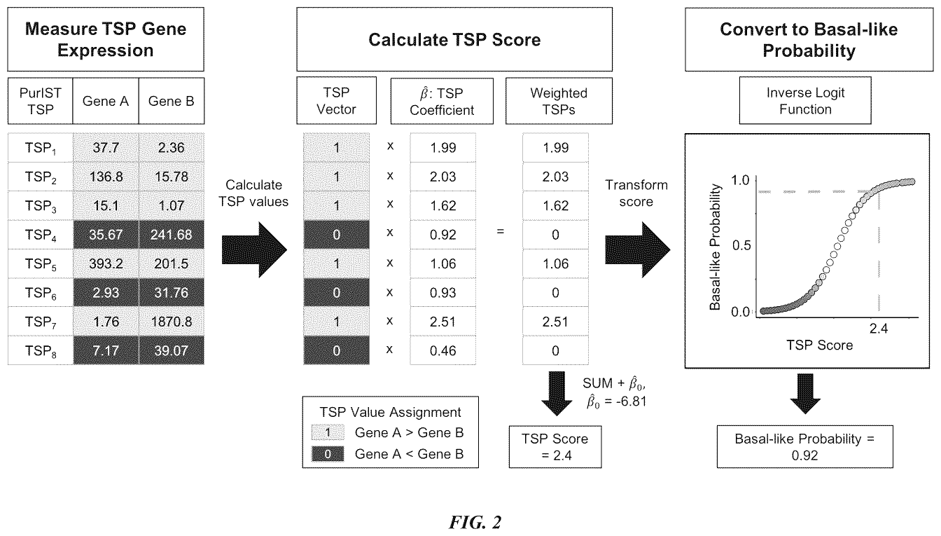

We found that the Moffitt tumor-intrinsic two-subtype schema reliably differentiated survival across individual datasets (Table 22), showing significant associations with OS in the majority of individual studies in contrast to other schemas. After pooling datasets, we found that patients with Moffitt basal-like subtype tumors had significantly worse prognosis compared with the Moffitt classical subtype (FIG. 1C, stratified HR=1.98, p<0.0001, stratified Cox proportional hazards model). We also observed similar trends in the Bailey squamous and Collisson QM-PDA subtypes relative to other subtypes in the same schemas (FIGS. 1A and 1B), mirroring our treatment response results described herein above. However, overall subtype-specific survival differences were most pronounced within the two-subtype schema across studies (Table 22), compared with the Collisson (p=0.069) and Bailey (p=0.076) schemas.

Moreover, we found that nonsquamous subtypes in the Bailey schema had very similar OS to one another (FIG. 1B), where a direct overall comparison of these subtypes showed no statistically significant differences in survival in our pooled dataset (immunogenic vs. ADEX stratified HR=1.07, pancreatic progenitor vs. ADEX HR=1.01, overall p=0.82). We found a similar result when comparing survival among patients from the non-QM-PDA subtypes in the Collisson schema in the pooled data (FIG. 1A; exocrine-like vs. classical stratified HR=1.17; p=0.344).

In our pooled dataset, strong correspondence was again found between the Bailey squamous, Collisson QM-PDA, and Moffitt basal-like subtypes, and between the Moffitt classical subtype and the remaining subtypes in the Bailey (Cohen Kappa=0.56, p=0) and Collisson (Cohen Kappa=0.4, p=0) schemas. In TCGA_PAAD, where estimates of tumor purity were available, Moffitt classical patients that were also classified as QM-PDA in the Collisson schema had much lower tumor purity than other samples (p=0.0016). The Bailey ADEX and immunogenic samples also had lower tumor purity, regardless of whether they were called Moffitt classical or basal-like. These findings were similar to other studies (Cancer Genome Atlas Research Network, 2017; Puleo et al., 2018; Maurer et al., 2019), and suggested that the discordance in subtype assignment between schemas may be driven by tumor purity.

To determine the best fitting model for OS, we calculated BIC with respect to the stratified Cox proportional hazards model pertaining to each schema. Similar to our analysis of treatment response, we found that the Moffitt two-subtype schema had the best (lowest) BIC and therefore had the best and most parsimonious fit to the pooled survival data (Figures JA-1C; Table 22). We also found this to be the case in the majority of individual studies, replicated across each of our validation datasets (Table 22). These results reflected our finding that no difference in OS can be observed among the Collisson non-QM-PDA and Bailey nonsquamous subtypes in our pooled analysis.

Taken together, these findings supported the conclusion that the Moffitt two-subtype schema strongly and parsimoniously explained differences in OS as compared to alternate subtyping schemas. Our results further suggested that the additional subtypes found in the Collisson and Bailey schemas did not demonstrate additional clinical benefit in terms of predicting OS relative to the simpler Moffitt two-subtype schema, based on BIC and direct statistical comparison of the Collisson non-QM-PDA and Bailey nonsquamous subtypes. Given the robustness and highly replicable clinical utility of the Moffitt schema, we next developed a SSC based on this tumor-intrinsic two-subtype schema to avoid reliance on CC-based analysis.

### Example 3

### PurIST SSC

The ability to resolve and assign subtypes via clustering is limited when applied to individual patients. Reclustering new samples with existing training samples may also change existing subtype assignments. Thus, we developed a robust SSC, PurIST, to predict subtype in individual patients, based on our three largest bulk gene expression datasets (TCGA_PAAD, Aguirre Biopsies, and Moffitt GSE71729, training group). A key element of our method includes the utilization of tumor-intrinsic genes previously identified (Moffitt et al., 2015) to avoid the possible confounding of tumor gene expression with those from other tissue types. For model training, we designated training labels as described herein above. We used rank-derived quantities as predictors in our final SSC model instead of the raw expression values, utilizing the k Top Scoring Pair (kTSP) approach to generate these predictors (described herein above). The motivation of this approach was that while the raw values of gene expression may be on different scales in different studies, their relative magnitudes can be preserved by ranks.

We found that this type of rank transformation of the raw expression data had several advantages. First, a single predictor (TSP) only depends on the ranks of raw gene expression of a gene pair in a sample. Hence, its value is robust to overall technical shifts in raw expression values (i.e., due to variation in sequencing depth), and, as a result, is less sensitive to common between-sample normalization procedures of data preprocessing (Leek, 2009; Afsari et al., 2014; Patil et al., 2015). Second, it simplifies data integration over different training studies as data are on the same scale. Finally, prediction in new patients is also simplified, as normalizing new patient data to the training set is no longer necessary, which may further affect the accuracy of model predictions (Patil et al., 2015).

### Example 4

### Development and External Validation of PurIST Classifier

We applied the systematic procedure described herein implementing the above approach to derive our PurIST model for prediction in the tumor-intrinsic two-subtype schema given the training labels and ranked transformed predictors for each training samples. The selected eight gene pairs (TSP), fitted model, and model coefficients are given in Tables 25 and 26. The validation that is performed in a hypothetical new patient comprises computing the values of each of the eight selected TSPs in that patient, where a value of 1 is assigned if the first gene in a TSP—gene A—has greater expression than the second gene—gene B—in that patient (and assigned 0 value otherwise). These values are then multiplied by the corresponding set of estimated TSP model coefficients, summing these values to get the patient “TSP Score” after correction for estimated baseline effects. This score is then converted to a predicted probability of belonging to the basal-like subtype, where values greater than 0.5 suggest basal-like subtype membership and the classical subtype otherwise.

To assess the quality of our prediction model, we evaluated the cross-validation error of the final model in our training group. We found that the internal leave-one-out cross-validation error for PurIST on the training group was low (3.1%).

To validate this model, we applied it to the validation group datasets and determined whether PurIST predictions recapitulated the CC subtypes in each study. We found that pooled validation samples strongly segregated by CC subtype when sorted by their predicted basal-like probability, despite diverse studies of origin. These suggested that our methodology avoided potential study-level batch effects. The relative expression of classifier genes within each classifier TSP (paired rows) strongly discriminated between subtypes in each sample, forming the basis of our robust TSP-oriented approach for subtype prediction. We also found that, visually, predicted subtypes from PurIST had strong correspondence with independently determined CC subtypes.

Overall, the PurIST classifier predicted subtypes with high levels of confidence with most basal-like subtype predictions having predicted basal-like probabilities >0.9 (strong basal-like) and most classical subtype predictions with predicted basal probabilities of <0.1 (strong classical). Among these high confidence predictions, the majority of these calls corresponded with subtypes obtained independently via CC. Lower confidence calls (likely/lean basal-like/classical categories of prediction) had higher rates of misclassification, although these less confident calls were more rare in our validation datasets.

To evaluate the overall classification performance of PurIST across studies, we applied a nonparametric meta-analysis approach to obtain a consensus ROC curve based on the individual ROC curves from each validation study (Martinez-Camblor, 2017). We found that the overall consensus AUC was high, with a value of 0.993. ROC curves from individual studies were also consistent. In addition, we found that the estimated interstudy variability of these ROC curves with respect to predicted basal-like probability threshold t was low overall, with relatively higher variance at low thresholds and almost no variability at our standard threshold of 0.5 or greater. These reflected the similarity of individual ROC curves that were observed.

We found that within our validation datasets, the prediction accuracy rates were in general 90% or higher, and individual study AUCs were 0.95 or greater (see Table 27). Furthermore, sensitivities and specificities were often high and in some cases equal to 1, reflecting near perfect classification accuracy. These results suggested that PurIST was robust across multiple datasets and platforms and recapitulated the subtypes independently obtained via CC, which we have shown to have high clinical utility.

### Example 5

### Replicability of PurIST in Archival Formalin-fixed and Paraffin-embedded and FNA Samples

Because frozen bulk tumor samples are not commonly available in routine clinical practice, we next looked at the replicability of PurIST predictions across sample types that are more widely collected in clinical practice. Notably, nearly all preoperative and metastatic biopsies are obtained using either FNA or core biopsy techniques. Prior studies have shown the feasibility of performing RNAseq on core biopsies (Aguirre et al., 2018) and endoscopic ultrasound guided FNAs, both of which are commonly utilized in the diagnosis of pancreatic cancer (Rodriguez et al., 2016). We therefore evaluated the performance of PurIST in both formalin-fixed and paraffin embedded (FFPE) and FNA samples.

Among 47 pairs of matched FNA and bulk samples that passed quality control (Yeh_Seq dataset), we found significant agreement between the PurIST subtype calls of the matched FNA and bulk samples (Cohen Kappa=0.544; p=2.8×10⁻⁵). Only three pairs of samples (6.4%) show disagreement in subtype calling results using PurIST. CC calls of the bulk samples are also shown as a comparison.

We performed a similar evaluation with tumors that we had matched FFPE, FNA, and bulk samples available We found complete agreement among PurIST subtype predictions among FFPE, FNA, and bulk samples in patients that had all three sample types available (five sets total), further supporting that PurIST was robust across different sample preparations.

We also found that the genes pertaining to PurIST TSPs are comparatively less variable than genes not designated as tumor-intrinsic. For example, PurIST TSP genes, originally selected from our tumor-intrinsic gene list, had significantly higher Spearman correlation between sample types than Bailey immunogenic (p=0.0149) or ADEX genes (p=0.0083) using a permutation test described herein above. The stability of TSP genes across sample types supported their robustness and their ability to identify tumor-intrinsic signals in samples that may be confounded by low-input or degradation.

### Example 6

### Replicability of PurIST Predictions on a NanoString Platform

RNAseq assays in Clinical Laboratory Improvement Amendments (CLIA)-certified laboratories are still in their infancy. Thus, we evaluated the performance of PurIST on samples using NCOUNTER® brand detection technology (NanoString Technologies, Inc, Seattle, Washington, United States of America), a gene expression quantification system that directly quantifies molecular barcodes. This platform has been widely used in cancer molecular subtyping (Veldman-Jones et al., 2015), and is more widely available in CLIA-certified laboratories.

In samples with both RNAseq and NanoString platform expression data available, we evaluated the consistency between subtype calls based on their RNAseq and NanoString expression data using PurIST-n. This updated classifier was trained in a manner similar to PurIST, with the exception that genes were limited to those in common between the two platforms, as a more limited set of genes were available for our NanoString probeset. We found that there was strong agreement between PurIST-n calls in 51 patients with matched RNAseq/NanoString samples (Cohen Kappa=0.879; p=2.25× 10-11), where only one sample showed disagreement in its PurIST-n call. This discrepancy may have been due to the relatively lower read count in the RNAseq sample for this patient. In addition, it is noteworthy that the PurIST-n call for this sample was a low confidence call (“lean classical”). These results supported the replicability of PurIST on the NanoString platform and suggested that NanoString may be more robust at overcoming the hurdles of low input or degraded samples.

### Example 7

### Applicability of PurIST to Treatment Decision Making

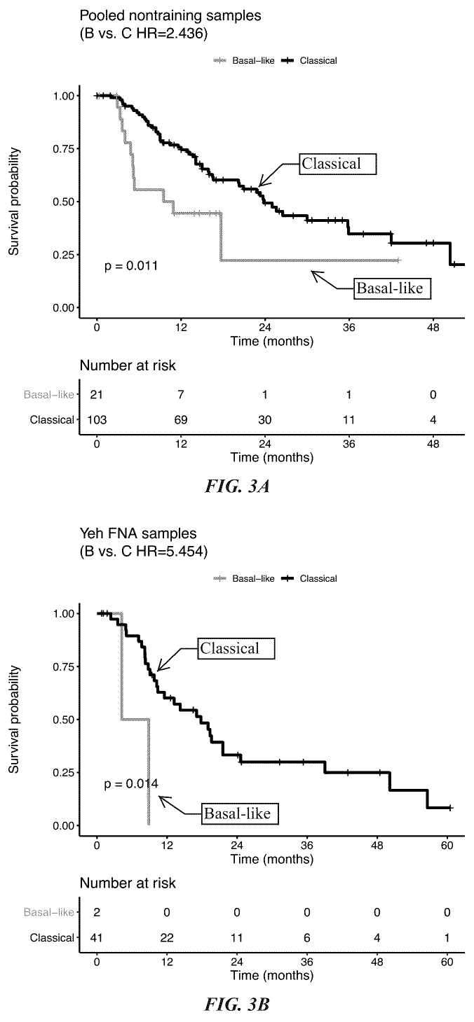

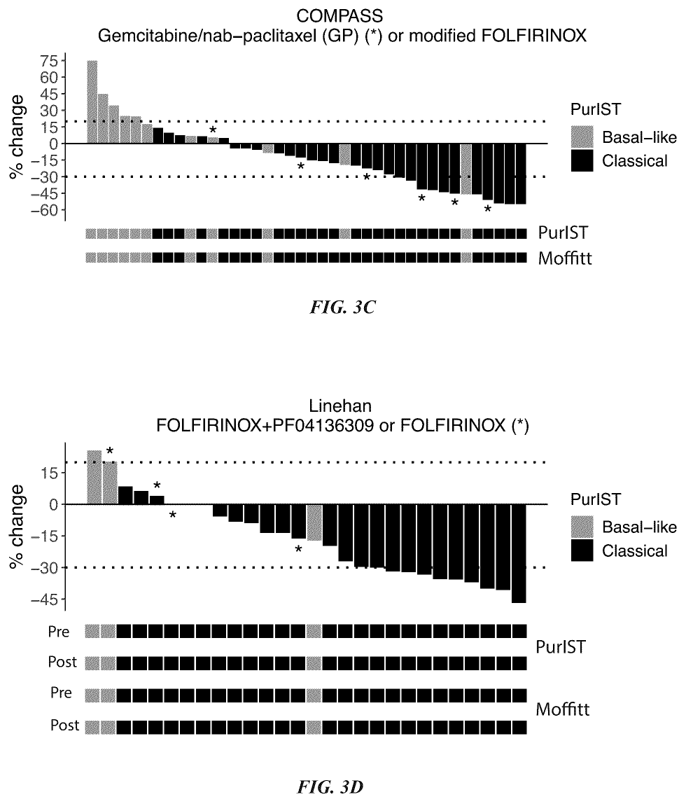

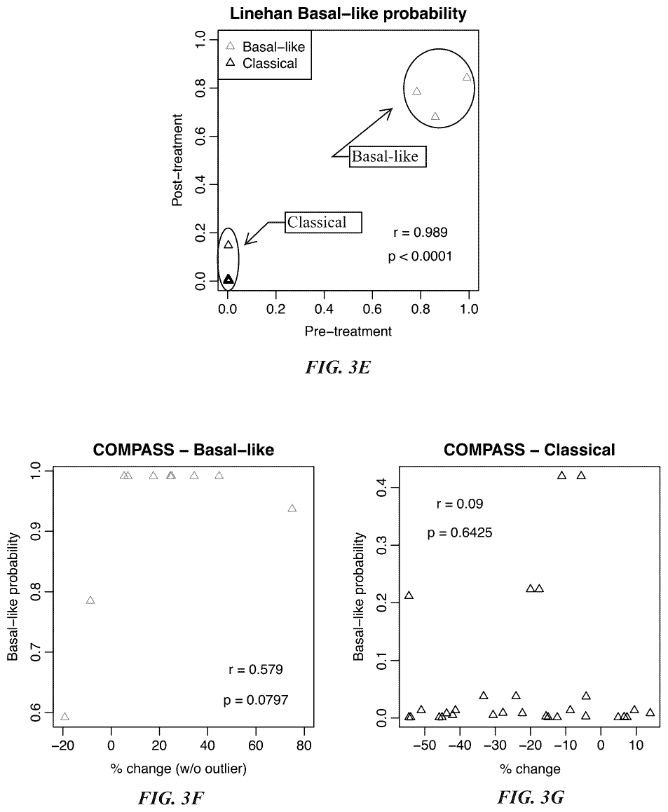

We next evaluated the potential utility of using PurIST for clinical decision making. In basal-like and classical samples that were classified by PurIST, we found significant survival differences in both the pooled public (with all training group samples removed) and the Yeh_Seq FNA datasets, with basal-like samples showing shorter OS (FIGS. 3A and 3B; Table 22).

We then looked at the relevance of PurIST to treatment response in the COMPASS and Linehan trials (FIGS. 3C and 3D). PurIST recapitulated 48 of 49 PDAC subtype calls compared with the previous CC-based calls in the COMPASS dataset, and 66 of 66 subtype calls in the Linehan dataset. Only one patient with a CC classical tumor was called basal-like by PurIST and had stable disease (SD, % change>−30% and <20%) in the COMPASS trial. Notably, the only PR seen in a PurIST basal-like tumor was in a patient with an unstable DNA subtype (Aung et al., 2018).

In agreement with our CC analysis, we found that PurIST-predicted subtype tumors had similar associations with treatment response (FIGS. 3C and 3D; Tables 18-21). We also found no change in PurIST subtype or the confidence of the call after treatment, suggesting that PurIST tumor subtypes were unchanged after treatment with FOLFIRINOX and PF-04136300 (FIGS. 3D and 3E). Finally, after excluding the sample with an unstable-DNA-subtype, we showed a positive correlation between PurIST basal-like predicted class probabilities and worse treatment response in basal-like tumors (FIG. 3F). No association of PurIST classical confidence and treatment response was seen (FIG. 3G).

### Discussion of the EXAMPLES

The availability of next-generation sequencing has facilitated a wealth of genomic studies in pancreatic cancer (Collisson et al., 2011; Moffitt et al., 2015; Bailey et al., 2016; Cancer Genome Atlas Research Network, 2017; Puleo et al., 2018; Maurer et al., 2019). Paired with the increasing availability of promising treatment options for patients with pancreatic ductal adenocarcinomas (PDAC), the ability to predict optimal treatment regimens for patients is becoming ever more critical. Treatments such as FOLFIRINOX have nearly doubled median overall survival (OS) from 6.8 to 11.1 months (Conroy et al., 2011), and gemcitabine plus nab-paclitaxel has increased median OS to 8.5 months (Von Hoff et al., 2013) in patients with metastatic disease. Determining the optimal choice of therapy given a patient's individual clinical or molecular characteristics, thereby enabling “precision medicine” approaches (Ashley, 2016) in PDAC, may improve these outcomes further.

The ongoing multi-center study of changes and characteristics of genes in patients with pancreatic cancer for better treatment selection (COMPASS) was the first study to show treatment ramifications with two molecular subtypes (Aung et al., 2018) first introduced by Moffitt and co-workers in 2015 (Moffitt et al., 2015). Patients enrolled in COMPASS underwent percutaneous core needle biopsies and were treated with one of two standard first-line therapies, modified-FOLFIRINOX or gemcitabine plus nab-paclitaxel according to physician choice. Collected patient samples in COMPASS underwent laser capture microdissection (LCM) followed by whole genome and RNA sequencing, providing an essential opportunity to evaluate genomic associations with treatment response. The findings from COMPASS demonstrated strong associations of molecular subtypes derived from consensus clustering (CC) with treatment response, and further support the need for a clinically usable subtyping system that can be integrated into future clinical studies.

While the development of subtype-based precision medicine approaches is advanced for some cancers (Parker, 2009; Hood, 2011; Vargas, 2016; Dienstmann, 2017;), consensus regarding such molecular subtypes for clinical decision-making in pancreatic ductal adenocarcinoma (PDAC) has been elusive. Multiple molecular subtyping systems for pancreatic cancer have been recently proposed in the literature with some studies isolated to PDAC and others that include additional histologies that fall under pancreatic cancer. For example, three molecular subtypes with potential clinical and therapeutic relevance (Collisson classical, quasi-mesenchymal and exocrine-like) were first described in Collisson et al., 2011, leveraging a combination of cell line, bulk, and microdissected patient samples. In contrast, a subsequent study of pancreatic cancer patients later found four molecular subtypes (Bailey et al., 2016) based upon the more diverse pancreatic cancer types: PDAC, adenosquamous, colloid, IPMN with invasive cancer, acinar cell and undifferentiated cancers (Bailey pancreatic progenitor, squamous, immunogenic, and aberrantly differentiated endocrine exocrine (ADEX)). More recently, Puleo et al., described five subtypes which are based on features specific to tumor cells and the local microenvironment (Puleo et al., 2018). Maurer et al. experimentally demonstrated the epithelial and stromal origin of many these transcripts with a cohort of microdissected samples (Maurer et al., 2019). Using non-negative matrix factorization to virtually microdissect tumor samples, we previously have shown two tumor-specific subtypes of PDAC (Moffitt et al., 2015) that we called basal-like, given the similarities with basal breast and basal bladder cancer, and classical, given the overlap with Collisson classical.

Comparative evaluations of these proposed subtyping systems have been limited, partially due to the difficulty in curating and applying these diverse subtyping approaches in new datasets. In one study, The Cancer Genome Atlas (TCGA) pancreatic cancer (PAAD) working group showed that the Collisson quasi-mesenchymal, Bailey immunogenic, and Bailey ADEX subtypes are enriched in low molecular purity PDAC samples (Cancer Genome Atlas Research Network, 2017). In samples of sufficient purity, Collisson classical/Moffitt classical/Bailey pancreatic progenitor and Collisson quasi-mesenchymal/Moffitt basal-like/Bailey squamous were most closely aligned. However, no other independent molecular or clinical evaluations of alternate subtyping systems have been proposed.

Through the careful curation of a large number of publicly available PDAC gene expression datasets, we perform, for the first time, a systematic interrogation of the aforementioned subtyping systems based upon their molecular fidelity and clinical utility across multiple validation datasets. We describe herein that the two-tumor subtype model developed by Moffitt et al. (Moffitt et al., 2015) is robust to confounders such as purity and best explains clinical outcomes across multiple validation datasets. Given the performance of this two-tumor subtype model, we have developed a single sample classifier that we call Purity Independent Subtyping of Tumors (PurIST) to perform subtype calling for clinical use. We showed that PurIST performs well on multiple gene expression platforms including microarray, RNA sequencing, and NanoString. In addition, we demonstrated its potential utility for small sample volumes such as fine needle aspirations (FNAs), given the preponderance of non-surgical biopsies in the neoadjuvant and metastatic settings. Lastly, we confirmed the stability of PurIST subtypes after treatment, and augmented the prior findings in COMPASS that subtypes are associated with treatment response. Particularly, we showed that PurIST basal-like subtype tumors were associated with treatment resistance to FOLFIRINOX, strongly supporting the need to incorporate subtyping into clinical trials of patients with PDAC.

Several subtyping systems for pancreatic cancer have now been proposed. Despite this, several limitations remain before they can be clinically usable. Here we leverage the wealth of transcriptomic studies that have been performed in pancreatic cancer to determine the molecular subtypes that may be most clinically useful and replicable across studies. Our results show that while multiple molecular subtypes may be used to characterize patient samples, the two tumor-intrinsic subtypes from the Moffitt schema: basal-like (overlaps with Bailey squamous/Collisson QM-PDA) and classical (overlaps with non-Bailey squamous/non-Collisson QMPDA) are the most concordant and clinically robust. The compelling findings of basal-like tumors showing resistance to FOLFIRINOX and the lack of objective studies comparing current first-line therapies FOLFIRINOX versus gemcitabine plus nab-paclitaxel strongly support the need to evaluate the role of molecular subtyping in treatment decision making for patients with PDAC. Therefore, we have developed a SSC based on the two tumor-intrinsic subtypes that avoids the instability associated with current strategies of clustering multiple samples and the low tumor purity issues in PDAC samples

Prior studies have shown that merging samples from multiple studies (horizontal data integration) can improve the performance of prediction models, relative to training on individual studies (Richardson et al., 2016). However, systematic differences in the scales of the expression values in each dataset are often observed, as some may have been separately normalized prior to their publication or were generated from a variety of expression platforms. Complicated cross-platform normalizations are often employed in such situations prior to model training. Furthermore, new samples must be normalized to the training dataset prior to prediction to obtain relevant predicted values. This often results in a “test-set bias” (Patil et al., 2015), where predictions may change due to the samples in the test set or the normalization approach used. In addition, prediction models may change with the addition of new training samples, as renormalizations may be warranted among training samples. In all, this leads to potential complications for data merging, stability of prediction, and model accuracy (Lusa et al., 2007; Paquet & Hallett, 2015).

These drawbacks are largely addressed by the presently disclosed PurIST approach, which is not dependent on cross-study normalization, and is robust to platform type and sample collection differences. We showed that the sensitivity and specificity of PurIST calls are high across multiple independent studies, demonstrating that the PurIST classifier recapitulated the tumor-intrinsic subtype calling obtained initially by CC. Given the significant clinical relevance of the two tumor-intrinsic subtypes for both prognosis and treatment response and the high accuracy of predicted subtype calls in our validation datasets, PurIST would appear to have tremendous clinical value. Specifically, PurIST worked for gene expression data assayed across multiple platforms, including microarrays, RNAseq, and NanoString. Furthermore, the algorithm provided replicable classification for matched samples from snap-frozen bulk tissue as well as FNA, core biopsies, and archival tissues.

Thus, PurIST may be flexibly used on low input and more degraded samples and may be performed with targeted gene expression platforms such as NanoString, avoiding the need for a CLIA RNAseq assay. Our enduring findings that basal-like subtype tumors were significantly less likely to respond to FOLFIRINOX-based regimens strongly supported the need for the incorporation of molecular subtyping in treatment decision making to determine the association of molecular subtypes with this and other therapies. In addition, the stability of PurIST subtypes after treatment is a noteworthy finding and may point to fundamental biological differences in the tumor subtypes. Our ability to subtype based on either core or FNA biopsies considerably increases the flexibility and practicality of integrating PDAC molecular subtypes into future clinical trials in the metastatic and neoadjuvant setting where bulk specimens are rarely available.

Summarily, several genomic studies in pancreatic cancer suggest clinically relevant expression-based subtypes. However, consensus subtypes remain unclear. Using the explosion of publicly available data, the relationships of the different subtypes were examined and it has been demonstrated that a two-tumor subtype schema was most robust and clinically relevant. A single-sample classifier (SSC) that is referred to herein as Purity Independent Subtyping of Tumors (PurIST) with robust and highly replicable performance on a wide range of platforms and sample types has been produced and is described herein. That PurIST subtypes have meaningful associations with patient prognosis and have significant implications for treatment response has been demonstrated. The flexibility and utility of PurIST on low-input samples such as tumor biopsies allows it to be used at the time of diagnosis to facilitate the choice of effective therapies for PDAC patients and should be considered in the context of future clinical trials.

### REFERENCES

All references cited in the instant disclosure, including but not limited to all patents, patent applications and publications thereof, scientific journal articles, and database entries (including but not limited to GENBANK® biosequence database entries and all annotations available therein) are incorporated herein by reference in their entireties to the extent that they supplement, explain, provide a background for, or teach methodology, techniques, and/or compositions employed herein.

Adiconis et al. (2013) Comparative analysis of RNA sequencing methods for degraded or low-input samples. Nat Methods 10 (7): 623-629.

Afsari et al. (2014) Rank Discriminants for Predicting Phenotypes from RNA Expression. Annals of Applied Statistics 8 (3): 1469-1491.

Afsari et al. (2015) switchBox: an R package for k-Top Scoring Pairs classifier development. Bioinformatics 31 (2): 273-274.

Aguirre et al. (2018) Real-time genomic characterization of advanced pancreatic cancer to enable precision medicine. Cancer Discovery 8 (9): 1096-1111.

Aung et al. (2018) Genomics-Driven Precision Medicine for Advanced Pancreatic Cancer-Early Results from the COMPASS Trial. Clin Cancer Res 24 (6): 1344-1354.

Bailey et al. (2016) Genomic analyses identify molecular subtypes of pancreatic cancer. Nature 531 (7592): 47-52.

Breheny & Huang (2011) Coordinate Descent Algorithms for Nonconvex Penalized Regression, with Applications to Biological Feature Selection. Ann Appl Stat 5 (1): 232-253.

Bushnell (2014) BBMap: A Fast, Accurate, Splice-Aware Aligner. United States: N. p., 2014. Web.

Cancer Genome Atlas Research Network. (2017) Integrated Genomic Characterization of Pancreatic Ductal Adenocarcinoma. Cancer Cell 32 (2): 185-203 e113.

Carter et al. (2012) Absolute quantification of somatic DNA alterations in human cancer. Nat Biotechnol 30 (5): 413-421.

Collisson et al. (2011) Subtypes of pancreatic ductal adenocarcinoma and their differing responses to therapy. Nat Med 17 (4): 500-503.

Connor et al. (2017) Association of Distinct Mutational Signatures With Correlates of Increased Immune Activity in Pancreatic Ductal Adenocarcinoma. JAMA Oncol 3 (6): 774-783.

Conroy et al. (2011) FOLFIRINOX versus gemcitabine for metastatic pancreatic cancer. New England Journal of Medicine 364 (19): 1817-1825.

Geman et al. (2004) Classifying gene expression profiles from pairwise mRNA comparisons. Statistical applications in genetics and molecular biology 3 (1): 1-19.

Kass et al. (1995) Bayes factors. J Am Statist Assoc 90:773-795.

Kindler et al. (2019) Olaparib as maintenance treatment following first-line platinum-based chemotherapy (PBC) in patients (pts) with a germline BRCA mutation and metastatic pancreatic cancer (mPC): Phase III POLO trial. J Clin Oncol 37:18_suppl.

Leek (2009) The tspair package for finding top scoring pair classifiers in R. Bioinformatics 25 (9): 1203-1204.

Lusa et al. (2007) Challenges in projecting clustering results across gene expression-profiling datasets. J Natl Cancer Inst 99 (22): 1715-1723.

Martinez-Camblor (2017) Fully non-parametric receiver operating characteristic curve estimation for random-effects meta-analysis. Stat Methods Med Res 26:5-20.

Maurer et al. (2019) Experimental microdissection enables functional harmonisation of pancreatic cancer subtypes. Gut 68:1034-1043.

Moffitt et al. (2015) Virtual microdissection identifies distinct tumor- and stroma-specific subtypes of pancreatic ductal adenocarcinoma. Nat Genet 47 (10): 1168-1178.

Nywening et al. (2016) Targeting tumour-associated macrophages with CCR2 inhibition in combination with FOLFIRINOX in patients with borderline resectable and locally advanced pancreatic cancer: a single-centre, open-label, dose-finding, non-randomised, phase 1b trial. Lancet Oncol 17 (5): 651-662.

Paquet & Hallett (2015) Absolute assignment of breast cancer intrinsic molecular subtype. J Natl Cancer Inst 107 (1): dju357.

Patil et al. (2015) Test set bias affects reproducibility of gene signatures. Bioinformatics 31 (14): 2318-2323.

Patro et al. (2017) Salmon provides fast and bias-aware quantification of transcript expression. Nat Methods 14 (4): 417-419.

PCT International Patent Application Publication No. WO 2019/226514.

Puleo et al. (2018) Stratification of Pancreatic Ductal Adenocarcinomas Based on Tumor and Microenvironment Features. Gastroenterology 155 (6): 1999-2013.e1993.

Rashid et al. (2020) Purity Independent Subtyping of Tumors (PurIST), A Clinically Robust, Single-sample Classifier for Tumor Subtyping in Pancreatic Cancer. Clinical Cancer Research 26 (1): 82-92.

Richardson et al. (2016) Statistical Methods in Integrative Genomics. Annual Review of Statistics and Its Application. 3:181-209

Rodriguez et al. (2016) RNA sequencing distinguishes benign from malignant pancreatic lesions sampled by EUS-guided FNA. Gastrointest Endosc 84 (2): 252-258.

Schwarz (1978) Estimating Dimension of a Model. Annals of Statistics 6 (2): 461-464.

Shi et al. (2011) Top scoring pairs for feature selection in machine learning and applications to cancer outcome prediction. BMC Bioinformatics 12: Article 375.

Tan et al. (2005) Simple decision rules for classifying human cancers from gene expression profiles. Bioinformatics 21 (20): 3896-3904.

U.S. Patent Application Publication Nos. 2008/0262215, 2010/0120097, 2011/0189679, 2014/0113333, 2015/0307874, 2017/0020824, 2017/0233827.

U.S. Pat. Nos. 5,800,992; 6,004,755; 6,013,449; 6,020, 135; 6,033,860; 6,040, 138; 6, 177,248; 6,251,601; 6,309,822; 7,595,159; 7,824,856; 8,008,025; 8,293,489; 8,299,239; 9,181,588; 9,920,367; 10,060,912; 10,227,584.

Veldman-Jones et al. (2015) Reproducible, Quantitative, and Flexible Molecular Subtyping of Clinical DLBCL Samples Using the NanoString nCounter System. Clin Cancer Res 21 (10): 2367-2378.

Von Hoff et al. (2013) Increased survival in pancreatic cancer with nab-paclitaxel plus gemcitabine. N Engl J Med 369 (18): 1691-1703.

Zhao et al. (2014) Comparison of RNA-Seq by poly (A) capture, ribosomal RNA depletion, and DNA microarray for expression profiling. BMC Genomics 15: Article 419.

It will be understood that various details of the presently disclosed subject matter can be changed without departing from the scope of the presently disclosed subject matter. Furthermore, the foregoing description is for the purpose of illustration only, and not for the purpose of limitation.

TABLE 1
Gene Pairs and Related Coefficients for PurIST and PurIST-n

| GENE PAIR | GENE A | GENE B | Coefficient |
|---|---|---|---|
| 1 | GPR87 | REG4 | 1.994 |
| 2 | KRT6A | ANXA10 | 2.031 |
| 3 | BCAR3 | GATA6 | 1.618 |
| 4 | PTGES | CLDN18 | 0.922 |
| 5 | ITGA3 | LGALS4 | 1.059 |
| 6 | C16orf74 | DDC | 0.929 |
| 7 | S100A2 | SLC40A1 | 2.505 |
| 8 | KRT5 | CLRN3 | 0.485 |
| A | GPR87 | REG4 | 3.413 |
| B | KRT6A | ANXA10 | 3.437 |
| C | KRT17 | LGALS4 | 2.078 |
| D | S100A2 | TFF1 | 2.651 |
| E | C16orf74 | DDC | 0.901 |
| F | KRT15 | PLA2G10 | 2.677 |
| G | PTGES | CDH17 | 2.911 |
| H | DCBLD2 | TSPAN8 | 1.903 |

TABLE 2
Exemplary NanoString Probes and SEQ ID NOs.

| GENE PAIR | GENE A | SEQ ID NO: | GENE B | SEQ ID NO: |
|---|---|---|---|---|
| 1 | GPR87 | 64 | REG4 | 71 |
| 2 | KRT6A | 65 | ANXA10 | 59 |
| 3 | BCAR3 | 81 | GATA6 | 82 |
| 4 | PTGES | 70 | CLDN18 | 84 |
| 5 | ITGA3 | 85 | LGALS4 | 68 |
| 6 | C16orf74 | 60 | DDC | 63 |
| 7 | S100A2 | 72 | SLC40A1 | 86 |
| 8 | KRT5 | 87 | CLRN3 | 83 |
| A | GPR87 | 64 | REG4 | 71 |
| B | KRT6A | 65 | ANXA10 | 59 |
| C | KRT17 | 67 | LGALS4 | 68 |
| D | S100A2 | 72 | TFF1 | 73 |
| E | C16orf74 | 60 | DDC | 63 |
| F | KRT15 | 66 | PLA2G10 | 69 |
| G | PTGES | 70 | CDH17 | 61 |
| H | DCBLD2 | 62 | TSPAN8 | 74 |

TABLE 3
Listing of Exemplary Nucleic acid and Amino acid Sequences
with GENBANK ® Accession Nos.

| Gene Name | Nucleic Acid and Amino Acid |
|---|---|
| (Coding Nucleotides*) | Accession Nos.** (SEQ ID NO:) |
| ANXA10 (165-1139) | NM_007193.5 (1); NP_009124.2 (2) |
| BCAR3 (359-2836) | NM_001261408.2 (3); |
| | NP_001248337.1 (4) |
| C16orf74 (190-420) | NM_206967.3 (5); NP_996850.1 (6) |
| CDH17 (94-2592) | NM_004063.4 (7); NP_004054.3 (8) |
| CLDNI8 (62-847) | NM_016369.4 (9); NP_057453.1 (10) |
| CLRN3 (158-838) | NM_152311.5 (11); NP_689524.1 (12) |
| CTSE (105-1295) | NM_001910.4 (13); NP_001901.1(14) |
| DCBLD2 (370-2697) | NM_080927.4 (15); NP_563615.3 (16) |
| DDC (87-1529) | NM_000790.4 (17); NP_000781.2 (18) |
| GATA6 (132-1919) | NM_005257.6 (19); NP_005248.2 (20) |
| GPR87 (334-1410) | NM_023915.4 (21); NP_076404.3 (22) |
| ITGA3 (331-3486) | NM_002204.4 (23); NP_002195.1 (24) |
| KRT5 (99-1871) | NM_000424.4 (25); NP_000415.2 (26) |
| KRT6A (70-1764) | NM_005554.4 (27); NP_005545.1 (28) |
| KRT15 (64-1434) | NM_002275.4 (29); NP_002266.3 (30) |
| KRT17 (67-1365) | NM_000422.3 (31); NP_000413.1 (32) |
| LGALS4 (60-1031) | NM_006149.4 (33); NP_006140.1 (34) |
| LYZ (29-475) | NM_000239.3 (35); NP_000230.1 (36) |
| MUC17 (56-13537) | NM_001040105.2 (37); |
| | NP_001035194.1 (38) |
| MYO1A (264-3395) | NM_005379.4 (39); NP_005370.1 (40) |
| NR1I2 (49-1470) | NM_022002.2 (41); NP_071285.1 (42) |
| PIP5K1B (766-2388) | NM_003558.4 (43); NP_003549.1 (44) |
| PLA2G10 (80-577) | NM_003561.3 (45); NP_003552.1 (46) |
| PTGES (31-489) | NM_004878.5 (47); NP_004869.1 (48) |
| REG4 (147-623) | NM_032044.4 (49); NP_114433.1 (50) |
| S100A2 (350-646) | NM_005978.4 (51); NP_005969.2 (52) |
| SLC40A1 (327-2042) | NM_014585.6 (53); NP_055400.1 (54) |
| TFF1 (41-295) | NM_003225.3 (55); NP_003216.1 (56) |
| TSPAN8 (180-893) | NM_004616.3 (57); NP_004607.1 (58) |

*nucleotide positions in the corresponding an Accession No.
**Accession Nos. in the GENBANK ® biosequence database.

TABLE 4
Summary of Public Datasets

| | | Sample | | | |
|---|---|---|---|---|---|
| Dataset | Platform | Collection | Sample Types | Samples | Reference |
| MoffittGEO | microarray | Bulk | Primary PDAC, PDAC | 357 | Moffitt et |
| (G5E71729) | | | metastases, normal | | al., 2015 |
| | | | tissues | | |
| COMPASS | RNAseq | Core | Primary PDAC, PDAC | 50 | Auna et |
| | | biopsies, | metastases | | al., 2017 |
| | | LCM | | | |
| Aguirre | RNAseq | Core | Primary PDAC, PDAC | 73 | Aguirre et |
| Biopsies | | biopsies, | metastases, acinar cell | | al., 2018 |
| | | FNA | carcinoma | | |
| ICGC | RNAseq | Bulk, | Primary pancreatic | 92 | Bailey et |
| PACA-AU | | >12% | cancers: PDAC, | | al., 2016 |
| seq | | celluarity | adenosquamous, colloid, | | |
| | | | IPMN with invasive | | |
| | | | cancer, acinar cell and | | |
| | | | undifferentiated | | |
| ICGC | microarray | Bulk, | Primary pancreatic | 131 | Bailey et |
| PACA-AU | | >12% | cancers: PDAC, | | al., 2016 |
| array | | celluarity | adenosquamous, colloid, | | |
| | | | IPMN with invasive | | |
| | | | cancer, acinar cell and | | |
| | | | undifferentiated, | | |
| | | | mucinous non-cystic | | |
| | | | carcinoma, and signet | | |
| | | | ring | | |
| Moffitt | RNAseq | Bulk | PDX, PDAC cell lines, | 61 | Moffitt et |
| | | | CAFS | | al., 2015 |
| Linehan seq | RNAseq | Core | Primary PDAC | 66 | Nywening |
| | | biopsies, | | | et al., |
| | | bulk | | | 2016 |
| Connor | RNAseq | LCM | Primary PDAC, PDAC | 74 | Connor et |
| | | | metastases | | al., 2017 |
| TCGA | RNAseq | Bulk | Primary PDAC | 181 | CGARN, |
| PAAD | | | | | 2017 |

* Cancer Genome Atlas Research Network

TABLE 5
Yeh_Seq Samples

| | | Platform | | | |
|---|---|---|---|---|---|
| | | RNA-seq | | NanoString | |
| Sample type | | Primary | PDX | Primary | PDX |
| Bulk | FF* | 47 | 18 | 16 | 18 |
| | FFPE | 5 | 7 | 1 | 7 |
| FNA | | 45 | 3 | 16 | 0 |

*FF: flash frozen

TABLE 6
Genes and Probes Analyzed by NanoString

| GENE PAIR | GENE A | SEQ ID NO: | GENE B | SEQ ID NO: |
|---|---|---|---|---|
| A | GPR87 | 64 | REG4 | 71 |
| B | KRT6A | 65 | ANXA10 | 59 |
| C | KRT17 | 67 | LGALS4 | 68 |
| D | S100A2 | 77 | TFF1 | 73 |
| E | C16orf74 | 60 | DDC | 63 |
| F | KRT15 | 66 | PLA2G10 | 69 |
| G | PTGES | 70 | CDH17 | 61 |
| H | DCRLD2 | 62 | TSPAN8 | 74 |

TABLE 7
Group Membership

| | Treatment | Survival | Training | Validation |
|---|---|---|---|---|
| Public Dataset | Group | Group | Group | Group |
| and Citation | (#) | (#) | (#) | (#) |
| Moffitt GEO (GSE71729); | N | Y | Y | N |
| Moffitt et al. 2015 | | (125) | (139) | |
| COMPASS | Y | N | N | Y |
| Aung et al., 2017 | (40) | | | (49) |
| Aguirre Biopsies; | N | N | Y | N |
| Aguirre et al., 2018 | | | (46) | |
| ICGC PACA-AU seq; | N | Y | N | Y |
| Bailey et al., 2016 | | (57) | | (65) |
| ICGC PACA-AU array; | N | Y | N | Y |
| Bailey et al., 2016 | | (71) | | (97) |
| Moffitt; | N | N | N | Y |
| Moffitt et al., 2015 | | | | (37) |
| Linehan seq; | Y | Y | N | Y |
| Nywening et al., 2016 | (28) | (28) | | (66) |
| Connor; | N | N | N | Y |
| Connor et al., 2017 | | | | (66) |
| TCGA PAAD; | N | Y | Y | N |
| CGARN*, 2017 | | (146) | (136) | |
| Pooled | | 376 | 321 | 378 |
| Group Notes | A | B | C | D |
| (see below) | | | | |

#: number of samples in Group
*: CGARN: Cancer Genome Atlas Research Network
A: Only samples with RNA-seq AND treatment response were considered.
B: duplicated samples between ICGC PACA-AU seq and ICGC PACA-AU array were removed when pooling.
C: Training Samples used here are a subset of the CC subtypes derived on each dataset.
D: Samples with CC labels were considered for validation.

TABLE 8
Aguirre_seq

| ID and Method | Collisson | Bailey | Moffitt | PurIST.training | PurIST | PurIST.basal.prob |
|---|---|---|---|---|---|---|
| 0400001_T1; resection | | | | FALSE | Classical | 0.032063 |
| 0400003_T1; resection | | | | FALSE | Basal | 0.991223 |
| 0400005_T1; resection | | | | FALSE | Classical | 0.0055 |
| 0400008_T1; biopsy | classical | Pancreatic | classical | TRUE | Classical | 0.001779 |
| | | Progenitor | | | | |
| 0400009_T1; resection | exocrine-like | Immunogenic | classical | TRUE | Classical | 0.002749 |
| 0400010_T1; biopsy | classical | Immunogenic | classical | TRUE | Classical | 0.013709 |
| 0400017_T1; resection | | | | FALSE | Classical | 0.146883 |
| 0400025_T1; resection | | | | FALSE | Classical | 0.001096 |
| 0400027_T1; biopsy | | | | FALSE | Classical | 0.012753 |
| 0400040_T1; resection | | | | FALSE | Classical | 0.023545 |
| 0400047_T1; biopsy | classical | Pancreatic | classical | TRUE | Classical | 0.001096 |
| | | Progenitor | | | | |
| 0400047_T2; resection | | | | FALSE | Classical | 0.001779 |
| 0400049_T1; resection | | | | FALSE | Basal | 0.785925 |
| 0400050_T1; resection | | | | FALSE | Classical | 0.008293 |
| 0400055_T1; biopsy | exocrine-like | Immunogenic | classical | TRUE | Classical | 0.019979 |
| 0400062_T1; biopsy | classical | Immunogenic | classical | TRUE | Classical | 0.001096 |
| 0400067_T1; biopsy | exocrine-like | Immunogenic | classical | TRUE | Classical | 0.001096 |
| 0400068_T1; biopsy | QM | Squamous | basal | TRUE | Basal | 0.991223 |
| 0400069_T1; biopsy | classical | Pancreatic | classical | TRUE | Classical | 0.001096 |
| | | Progenitor | | | | |
| 0400070_T1; resection | | | | FALSE | Classical | 0.001096 |
| 0400071_T1; resection | | | | FALSE | Classical | 0.019979 |
| 0400075_T1; biopsy | classical | Pancreatic | classical | TRUE | Classical | 0.001096 |
| | | Progenitor | | | | |
| 0400078_T1; biopsy | QM | Squamous | basal | TRUE | Basal | 0.991223 |
| 0400081_T1; resection | | | | FALSE | Classical | 0.013709 |
| 0400083_T1; biopsy | | | | FALSE | Classical | 0.013709 |
| 0400087_T1; biopsy | classical | Pancreatic | classical | TRUE | Classical | 0.00799 |
| | | Progenitor | | | | |
| 0400088_T1; resection | | | | FALSE | Classical | 0.003153 |
| 0400089_T1; biopsy | exocrine-like | Squamous | classical | TRUE | Classical | 0.00693 |
| 0400091_T1; biopsy | exocrine-like | Squamous | basal | TRUE | Basal | 0.902224 |
| 0400096_T1; biopsy | classical | Immunogenic | classical | TRUE | Classical | 0.002749 |
| 0400097_T1; biopsy | classical | Immunogenic | classical | TRUE | Classical | 0.001096 |
| 0400098_T1; biopsy | classical | Pancreatic | classical | TRUE | Classical | 0.002769 |
| | | Progenitor | | | | |
| 0400123_T1; biopsy | classical | Pancreatic | classical | TRUE | Classical | 0.001096 |
| | | Progenitor | | | | |
| 0400124_T1; biopsy | classical | Squamous | basal | TRUE | Basal | 0.784733 |
| 0400127_T1; biopsy | classical | Pancreatic | classical | TRUE | Classical | 0.280897 |
| | | Progenitor | | | | |
| 0400127_T2; resection | | | | FALSE | Classical | 0.020585 |
| 0400129_T1; biopsy | QM | Squamous | basal | TRUE | Basal | 0.991223 |
| 0400136_T1; biopsy | QM | Squamous | basal | TRUE | Basal | 0.991223 |
| 0400137_T1; biopsy | exocrine-like | ADEX | classical | TRUE | Classical | 0.236376 |
| 0400142_T1; biopsy | QM | Squamous | basal | TRUE | Basal | 0.991223 |
| 0400148_T1; biopsy | exocrine-like | ADEX | classical | TRUE | Classical | 0.001096 |
| 0400151_T2; biopsy | classical | Pancreatic | classical | TRUE | Classical | 0.002769 |
| | | Progenitor | | | | |
| 0400164_T1; biopsy | classical | ADEX | classical | TRUE | Classical | 0.001096 |
| 0400165_T1; biopsy | exocrine-like | ADEX | classical | TRUE | Classical | 0.001096 |
| 0400167_T1; biopsy | exocrine-like | ADEX | basal | TRUE | Basal | 0.784733 |
| 0400171_T1; biopsy | QM | Squamous | basal | TRUE | Basal | 0.991223 |
| 0400172_T1; biopsy | exocrine-like | ADEX | classical | TRUE | Classical | 0.426918 |
| 0400174_T1; biopsy | classical | Pancreatic | classical | TRUE | Classical | 0.001096 |
| | | Progenitor | | | | |
| 0400177_T1; biopsy | classical | Pancreatic | classical | TRUE | Classical | 0.032865 |
| | | Progenitor | | | | |
| 0400179_T1; biopsy | classical | Immunogenic | classical | TRUE | Classical | 0.001096 |
| 0400192_T1; biopsy | QM | ADEX | basal | TRUE | Basal | 0.975101 |
| 0400193_T1; biopsy | | | | FALSE | Classical | 0.001096 |
| 0400195_T1; biopsy | QM | Squamous | basal | TRUE | Basal | 0.985816 |
| 0400198_T1; biopsy | | | | FALSE | Classical | 0.002749 |
| 0400202_T1; biopsy | | | | FALSE | Classical | 0.280897 |
| 0400203_T1; biopsy | | | | FALSE | Classical | 0.002749 |
| 0400208_T1; biopsy | QM | Squamous | classical | TRUE | Classical | 0.032865 |
| 0400214_T1; biopsy | exocrine-like | ADEX | classical | TRUE | Classical | 0.001779 |
| 0400215_T1; biopsy | classical | Pancreatic | classical | TRUE | Classical | 0.002749 |
| | | Progenitor | | | | |
| 0400220_T1; biopsy | QM | Squamous | basal | TRUE | Basal | 0.850276 |
| 0400231_T1; biopsy | QM | Squamous | classical | FALSE | Classical | 0.211492 |
| 0400233_T1; biopsy | QM | Squamous | basal | TRUE | Basal | 0.991223 |
| 0400235_T1; biopsy | exocrine-like | ADEX | basal | FALSE | Basal | 0.96486 |
| 0400237_T1; biopsy | exocrine-like | ADEX | classical | TRUE | Classical | 0.001096 |
| 0400242_T1; biopsy | classical | Immunogenic | classical | TRUE | Classical | 0.437577 |
| 0400243_T1; biopsy | QM | ADEX | basal | TRUE | Basal | 0.991223 |
| 0400245_T1; biopsy | | | | FALSE | Classical | 0.092608 |
| 0400251_T1; biopsy | classical | Pancreatic | classical | TRUE | Classical | 0.002769 |
| | | Progenitor | | | | |
| 0400253_T1; biopsy | | | | FALSE | Classical | 0.193605 |
| 0400267_T1; biopsy | | | | FALSE | Classical | 0.002749 |
| 0400268_T1; biopsy | | | | FALSE | Classical | 0.092608 |
| 0400270_T1; biopsy | classical | Immunogenic | classical | TRUE | Classical | 0.00799 |
| 0400278_T1; biopsy | | | | FALSE | Classical | 0.205302 |

TABLE 9
COMPASS

| ID | Histology | Change | RECIST | Treatment | Collisson | Bailey | Moffitt | PurIST.training | PurIST | PurIST.basal.prob |
|---|---|---|---|---|---|---|---|---|---|---|
| COMP0014 | | −8.7 | SD | FFX | Classical | Pancreatic | Classical | FALSE | Classical | 0.013405 |
| | | | | | | Progenitor | | | | |
| COMP0001 | Adenoca. | −30.6 | PR | FFX | Classical | Immunogenic | Classical | FALSE | Classical | 0.005113 |
| COMP0002 | Adenoca. | −45.1 | PR | GP | Exocrine-like | ADEX | Classical | FALSE | Classical | 0.001096 |
| COMP0004 | Adenoca. | −15.6 | SD | FFX | Classical | Pancreatic | Classical | FALSE | Classical | 0.002749 |
| | | | | | | Progenitor | | | | |
| COMP0005 | Adenoca. | −4.2 | SD | FFX | Classical | Immunogenic | Classical | FALSE | Classical | 0.037269 |
| COMP0006 | Adenoca. | −54 | PR | FFX | Classical | Pancreatic | Classical | FALSE | Classical | 0.001096 |
| | | | | | | Progenitor | | | | |
| COMP0007 | Adenoca. | | | GP | QM-PDA | Squamous | Basal-like | FALSE | Basal-like | 0.691693 |
| COMP0008 | | −22.3 | SD | GP | Classical | Immunogenic | Classical | FALSE | Classical | 0.008293 |
| COMP0010 | Adenoca. | 5.4 | SD | GP | QM-PDA | Squamous | Basal-like | FALSE | Basal-like | 0.991223 |
| COMP0009 | | −27.8 | SD | FFX | Classical | ADEX | Classical | FALSE | Classical | 0.008906 |
| COMP0011 | Adenoca. | | | FFX | QM-PDA | Squamous | Basal-like | FALSE | Basal-like | 0.902224 |
| COMP0012 | Adenoca. | | | GP | Exocrine-like | Pancreatic | Classical | FALSE | Classical | 0.001096 |
| | | | | | | Progenitor | | | | |
| COMP0013 | Adenosq. | 75 | PD | FFX | QM-PDA | Squamous | Basal-like | FALSE | Basal-like | 0.936765 |
| COMP0015 | Adenoca. | 25 | PD | FFX | QM-PDA | Squamous | Basal-like | FALSE | Basal-like | 0.991223 |
| COMP0017 | Adenoca. | 9.5 | SD | FFX | Classical | Immunogenic | Classical | FALSE | Classical | 0.013405 |
| COMP0018 | | 44.7 | PD | FFX | QM-PDA | Squamous | Basal-like | FALSE | Basal-like | 0.991223 |
| COMP0019 | Adenoca. | −45.9 | PR | FFX | Classical | Immunogenic | Classical | FALSE | Classical | 0.001096 |
| COMP0020 | Adenoca. | 17.5 | SD | FFX | QM-PDA | Squamous | Basal-like | FALSE | Basal-like | 0.991223 |
| COMP0021 | Adenosq. | −45.8 | PR | FFX | QM-PDA | Squamous | Basal-like | FALSE | Basal-like | 0.854066 |
| COMP0023 | | −42.1 | PR | FFX | Classical | Immunogenic | Classical | FALSE | Classical | 0.005113 |
| COMP0025 | Adenoca. | | | FFX | Classical | Pancreatic | Classical | FALSE | Classical | 0.001096 |
| | | | | | | Progenitor | | | | |
| COMP0026 | Adenoca. | −8.6 | SD | FFX | QM-PDA | Squamous | Basal-like | FALSE | Basal-like | 0.784733 |
| COMP0028 | | 14 | SD | FFX | Exocrine-like | Pancreatic | Classical | FALSE | Classical | 0.008293 |
| | | | | | | Progenitor | | | | |
| COMP0030 | Adenoca. | −4.3 | SD | FFX | Classical | Immunogenic | Classical | FALSE | Classical | 0.002749 |
| COMP0029 | Adenoca. | −15 | SD | FFX | Classical | Pancreatic | Classical | FALSE | Classical | 0.001096 |
| | | | | | | Progenitor | | | | |
| COMP0032 | Adenoca. | 6.6 | SD | FFX | Classical | ADEX | Classical | FALSE | Classical | 0.001779 |
| COMP0033 | | | | GP | Classical | Pancreatic | Classical | FALSE | Classical | 0.008293 |
| | | | | | | Progenitor | | | | |
| COMP0034 | | 24.5 | PD | FFX | QM-PDA | Squamous | Basal-like | FALSE | Basal-like | 0.991223 |
| COMP0035 | | −33.3 | PR | FFX | Classical | Immunogenic | Classical | FALSE | Classical | 0.037703 |
| COMP0036 | Adenoca. | 4.9 | SD | FFX | Classical | Pancreatic | Classical | FALSE | Classical | 0.001096 |
| | | | | | | Progenitor | | | | |
| COMP0037 | | −43.8 | PR | FFX | Classical | Immunogenic | Classical | FALSE | Classical | 0.007887 |
| COMP0038 | Adenoca. | 7.4 | SD | FFX | Classical | Pancreatic | Classical | FALSE | Classical | 0.001096 |
| | | | | | | Progenitor | | | | |
| COMP0039 | Adenoca. | | | FFX | QM-PDA | Squamous | Basal-like | FALSE | Basal-like | 0.991223 |
| COMP0041 | | | | FFX | Exocrine-like | Pancreatic | Classical | FALSE | Classical | Classical |
| | | | | | | Progenitor | | | | |
| COMP0042 | Adenoca. | −17.5 | SD | FFX | Classical | Immunogenic | Classical | FALSE | Classical | 0.223407 |
| COMP0043 | | −20 | SD | FFX | Exocrine-like | ADEX | Classical | FALSE | Classical | 0.223407 |
| COMP0044 | Adenoca. | −24.1 | SD | FFX | Exocrine-like | Immunogenic | Classical | FALSE | Classical | 0.037703 |
| COMP0045 | | 34.4 | PD | FFX | QM-PDA | Squamous | Basal-like | FALSE | Basal-like | 0.991223 |
| COMP0046 | Adenoca. | −11.1 | SD | FFX | QM-PDA | ADEX | Classical | FALSE | Classical | 0.419634 |
| COMP0047 | | −54.5 | PR | FFX | Exocrine-like | Pancreatic | Classical | FALSE | Classical | 0.001096 |
| | | | | | | Progenitor | | | | |
| COMP0048 | | 6.9 | SD | FFX | QM-PDA | Squamous | Basal-like | FALSE | Basal-like | 0.991223 |
| COMP0050 | | −12.4 | SD | GP | Classical | Immunogenic | Classical | FALSE | Classical | 0.001096 |
| COMP0049 | Adenoca. | −19.2 | SD | FFX | Classical | ADEX | Classical | FALSE | Basal-like | 0.591897 |
| COMP0052 | Adenoca. | | | Classical | Immunogenic | Classical | FALSE | Classical | 0.00446 | 0.001096 |
| COMP0055 | Acinar | −8.1 | SD | FFX | Exocrine-like | ADEX | | FALSE | Classical | 0.005113 |
| COMP0056 | Adenoca. | −54.5 | PR | FFX | Exocrine-like | ADEX | Classical | FALSE | Classical | 0.211492 |
| COMP0057 | | −5.6 | SD | FFX | QM-PDA | ADEX | Classical | FALSE | Classical | 0.419634 |
| COMP0058 | Adenoca. | −51 | PR | GP | Exocrine-like | Immunogenic | Classical | FALSE | Classical | 0.013405 |
| COMP0059 | Adenoca. | −41.3 | PR | GP | Classical | Immunogenic | Classical | FALSE | Classical | 0.013405 |
| COMP0060 | Adenoca. | | GP + | Classical | Immunogenic | Classical | FALSE | Classical | 0.001096 | |
| | | | Medi + | | | | | | | |
| | | | Tremi | | | | | | | |

TABLE 10
Connor

| ID | SampleType | Collisson | Bailey | Moffitt | PurIST.training | PurIST | PurIST.basal.prob |
|---|---|---|---|---|---|---|---|
| PCSI_0083 | Primary | Exocrine-like | Squamous | Basal-like | FALSE | Basal-like | 0.936765 |
| PCSI_0103 | Primary | Classical | Pancreatic | Classical | FALSE | Classical | 0.001096 |
| | | | Progenitor | | | | |
| PCSI_0132 | Primary | QM-PDA | Pancreatic | Classical | FALSE | Classical | 0.005113 |
| | | | Progenitor | | | | |
| PCSI_0142 | Primary | QM-PDA | Pancreatic | Classical | FALSE | Classical | 0.001096 |
| | | | Progenitor | | | | |
| PCSI_0145 | Primary | Exocrine-like | Squamous | Basal-like | FALSE | Classical | 0.150416 |
| PCSI_0173 | Primary | QM-PDA | Squamous | Classical | FALSE | Classical | 0.325049 |
| PCSI_0226 | Primary | Exocrine-like | Squamous | Basal-like | FALSE | Basal-like | 0.978228 |
| PCSI_0233 | Primary | Exocrine-like | Squamous | Classical | FALSE | Classical | 0.005113 |
| PCSI_0235 | Primary | Exocrine-like | ADEX | Classical | FALSE | Classical | 0.001779 |
| PCSI_0240 | Primary | Exocrine-like | Squamous | Basal-like | FALSE | Basal-like | 0.978077 |
| PCSI_0261 | Primary | QM-PDA | Immunogenic | Classical | FALSE | Classical | 0.001096 |
| PCSI_0263 | Primary | QM-PDA | Immunogenic | Classical | FALSE | Classical | 0.020585 |
| PCSI_0264 | Primary | QM-PDA | Pancreatic | Classical | FALSE | Classical | 0.001096 |
| | | | Progenitor | | | | |
| PCSI_0268 | Primary | Classical | Pancreatic | Classical | FALSE | Classical | 0.008293 |
| | | | Progenitor | | | | |
| PCSI_0269 | Primary | QM-PDA | Immunogenic | Classical | FALSE | Classical | 0.001779 |
| PCSI_0274 | Primary | Classical | Pancreatic | Classical | FALSE | Classical | 0.001096 |
| | | | Progenitor | | | | |
| PCSI_0279 | Primary | Classical | Pancreatic | Classical | FALSE | Classical | 0.001096 |
| | | | Progenitor | | | | |
| PCSI_0280 | Primary | Classical | ADEX | Classical | FALSE | Classical | 0.001779 |
| PCSI_0283 | Primary | Classical | Immunogenic | Classical | FALSE | Classical | 0.001779 |
| PCSI_0284 | Primary | Exocrine-like | Squamous | Basal-like | FALSE | Classical | 0.089648 |
| PCSI_0285 | Primary | Classical | Immunogenic | Classical | FALSE | Classical | 0.001779 |
| PCSI_0286 | Primary | Exocrine-like | ADEX | Classical | FALSE | Classical | 0.001779 |
| PCSI_0287 | Primary | Classical | Pancreatic | Classical | FALSE | Classical | 0.001096 |
| | | | Progenitor | | | | |
| PCSI_0290 | Primary | QM-PDA | Pancreatic | Classical | FALSE | Classical | 0.001096 |
| | | | Progenitor | | | | |
| PCSI_0292 | Primary | Exocrine-like | Squamous | Basal-like | FALSE | Basal-like | 0.946668 |
| PCSI_0302 | Primary | QM-PDA | Pancreatic | Classical | FALSE | Classical | 0.005113 |
| | | | Progenitor | | | | |
| PCSI_0303 | Primary | Classical | Pancreatic | Classical | FALSE | Classical | 0.001096 |
| | | | Progenitor | | | | |
| PCSI_0305 | Primary | Classical | Pancreatic | Classical | FALSE | Classical | 0.001096 |
| | | | Progenitor | | | | |
| PCSI_0307 | Primary | QM-PDA | Squamous | Classical | FALSE | Basal-like | 0.556881 |
| PCSI_0309 | Primary | QM-PDA | Immunogenic | Classical | FALSE | Classical | 0.001096 |
| PCSI_0310 | Primary | QM-PDA | Squamous | Classical | FALSE | Classical | 0.001096 |
| PCSI_0311 | Primary | Classical | Pancreatic | Classical | FALSE | Classical | 0.001096 |
| | | | Progenitor | | | | |
| PCSI_0312 | Primary | QM-PDA | Immunogenic | Classical | FALSE | Classical | 0.001096 |
| PCSI_0324 | Primary | Classical | Pancreatic | Classical | FALSE | Classical | 0.001096 |
| | | | Progenitor | | | | |
| PCSI_0325 | Primary | Classical | Pancreatic | Classical | FALSE | Classical | 0.001096 |
| | | | Progenitor | | | | |
| PCSI_0326 | Primary | Classical | Pancreatic | Classical | FALSE | Classical | 0.001779 |
| | | | Progenitor | | | | |
| PCSI_0328 | Primary | Classical | Immunogenic | Classical | FALSE | Classical | 0.001096 |
| PCSI_0329 | Primary | Classical | Pancreatic | Classical | FALSE | Classical | 0.001096 |
| | | | Progenitor | | | | |
| PCSI_0330 | Primary | QM-PDA | Immunogenic | Classical | FALSE | Classical | 0.001096 |
| PCSI_0334 | Primary | Classical | Immunogenic | Classical | FALSE | Classical | 0.001096 |
| PCSI_0337 | Primary | Classical | ADEX | Classical | FALSE | Classical | 0.06411 |
| PCSI_0338 | Primary | Classical | Pancreatic | Classical | FALSE | Classical | 0.001096 |
| | | | Progenitor | | | | |
| PCSI_0340 | Primary | Classical | Pancreatic | Classical | FALSE | Classical | 0.001096 |
| | | | Progenitor | | | | |
| PCSI_0341 | Primary | Classical | Pancreatic | Classical | FALSE | Classical | 0.00799 |
| | | | Progenitor | | | | |
| PCSI_0345 | Primary | Exocrine-like | ADEX | Classical | FALSE | Classical | 0.013405 |
| PCSI_0350 | Primary | Classical | Pancreatic | Classical | FALSE | Classical | 0.001096 |
| | | | Progenitor | | | | |
| PCSI_0353 | Primary | QM-PDA | Immunogenic | Classical | FALSE | Classical | 0.023545 |
| PCSI_0355 | Primary | QM-PDA | Pancreatic | Classical | FALSE | Classical | 0.001096 |
| | | | Progenitor | | | | |
| PCSI_0403 | Primary | Classical | Pancreatic | Classical | FALSE | Classical | 0.001096 |
| | | | Progenitor | | | | |
| PCSI_0453 | Primary | QM-PDA | Immunogenic | Classical | FALSE | Classical | 0.012753 |
| PCSI_0456 | Primary | Classical | Pancreatic | Classical | FALSE | Classical | 0.001779 |
| | | | Progenitor | | | | |
| PCSI_0457 | Primary | Classical | Pancreatic | Classical | FALSE | Classical | 0.001779 |
| | | | Progenitor | | | | |
| PCSI_0458 | Primary | Classical | ADEX | Classical | FALSE | Classical | 0.033022 |
| PCSI_0477 | Primary | Exocrine-like | Squamous | Basal-like | FALSE | Classical | 0.090712 |
| PCSI_0489 | Liver | | | | FALSE | Classical | 0.001096 |
| | Metastasis | | | | | | |
| PCSI_0506 | Primary | Classical | Immunogenic | Classical | FALSE | Classical | 0.019841 |
| PCSI_0508 | Primary | QM-PDA | ADEX | Classical | FALSE | Classical | 0.001779 |
| PCSI_0509 | Primary | Exocrine-like | ADEX | Classical | FALSE | Classical | 0.001779 |
| PCSI_0511 | Primary | QM-PDA | Pancreatic | Classical | FALSE | Classical | 0.013405 |
| | | | Progenitor | | | | |
| PCSI_0528 | Primary | QM-PDA | Immunogenic | Classical | FALSE | Classical | 0.00799 |
| PCSI_0531 | Primary | Classical | Pancreatic | Classical | FALSE | Classical | 0.001096 |
| | | | Progenitor | | | | |
| PCSI_0537 | Primary | Classical | Pancreatic | Classical | FALSE | Classical | 0.001096 |
| | | | Progenitor | | | | |
| PCSI_0572 | Primary | Exocrine-like | Squamous | Basal-like | FALSE | Classical | 0.309466 |
| RAMP_0002 | Lymph Node | | | | FALSE | Classical | 0.146883 |
| | Metastasis | | | | | | |
| RAMP_0002 | Liver | | | | FALSE | Classical | 0.001096 |
| | Metastasis | | | | | | |
| RAMP_0004 | Lymph Node | | | | FALSE | Basal-like | 0.991223 |
| | Metastasis | | | | | | |
| RAMP_0004 | Liver | | | | FALSE | Basal-like | 0.978077 |
| | Metastasis | | | | | | |
| RAMP_0004 | Primary | Exocrine-like | Squamous | Basal-like | FALSE | Basal-like | 0.991223 |
| RAMP_0006 | Liver | | | | FALSE | Classical | 0.037703 |
| | Metastasis | | | | | | |
| RAMP_0006 | Primary | Exocrine-like | Squamous | Basal-like | FALSE | Classical | 0.419634 |
| RAMP_0007 | Primary | QM-PDA | ADEX | Basal-like | FALSE | Basal-like | 0.556881 |
| RAMP_0008 | Lymph Node | | | | FALSE | Classical | 0.013405 |
| | Metastasis | | | | | | |
| RAMP_0008 | Liver | | | | FALSE | Classical | 0.001096 |
| | Metastasis | | | | | | |
| RAMP_0008 | Primary | QM-PDA | Immunogenic | Classical | FALSE | Classical | 0.037703 |

TABLE 11
Linehan_seq

| ID | Treatment | Pre.Post | Change | RECIST | Collisson | Bailey | Moffitt | PurIST.training | PurIST | PurIST.basal.prob |
|---|---|---|---|---|---|---|---|---|---|---|
| S1124.02.01 | FOLF | Pre | | | Classical | Immunogenic | Classical | FALSE | Classical | 0.001096 |
| S1124.02.02 | FOLF | Post | | | Exocrine-like | ADEX | Classical | FALSE | Classical | 0.001096 |
| S1124.03.01 | FOLF | Pre | 4 | SD | Classical | Immunogenic | Classical | FALSE | Classical | 0.001096 |
| S1124.03.02 | FOLF | Post | 4 | SD | Exocrine-like | Immunogenic | Classical | FALSE | Classical | 0.00799 |
| S1124.04.01 | FOLF | Pre | | | Exocrine-like | Squamous | Classical | FALSE | Classical | 0.054078 |
| S1124.07.01 | FOLF | Pre | 20.40816 | PD | QM-PDA | Squamous | Basal-like | FALSE | Basal-like | 0.860249 |
| S1124.07.02 | FOLF | Post | 20.40816 | PD | QM-PDA | Squamous | Basal-like | FALSE | Basal-like | 0.679704 |
| S1124.08.01 | FOLF | Pre | 0 | SD | Classical | Immunogenic | Classical | FALSE | Classical | 0.00693 |
| S1124.08.02 | FOLF | Post | 0 | SD | Classical | Pancreatic | Classical | FALSE | Classical | 0.001096 |
| | | | | | | Progenitor | | | | |
| S1124.09.01 | FOLF | Pre | | | Exocrine-like | Squamous | Classical | FALSE | Classical | 0.052325 |
| S1124.11.01 | FOLF | Pre | | | Exocrine-like | Pancreatic | Classical | FALSE | Classical | 0.0055 |
| | | | | | | Progenitor | | | | |
| S1124.12.01 | FOLF | Pre | | | QM-PDA | Squamous | Classical | FALSE | Classical | 0.001779 |
| S1124.13.01 | FOLF | Pre | −16.2791 | SD | Classical | Immunogenic | Classical | FALSE | Classical | 0.001096 |
| S1124.13.02 | FOLF | Post | −16.2791 | SD | Exocrine-like | Pancreatic | Classical | FALSE | Classical | 0.001096 |
| | | | | | | Progenitor | | | | |
| S1124.14.01 | FOLF + | Pre | −31.8182 | PR | Exocrine-like | ADEX | Classical | FALSE | Classical | 0.0055 |
| | PF | | | | | | | | | |
| S1124.14.02 | FOLF + | Post | −31.8182 | PR | Exocrine-like | ADEX | Classical | FALSE | Classical | 0.0055 |
| | PF | | | | | | | | | |
| S1124.15.01 | FOLF + | Pre | −32.1429 | PR | Exocrine-like | Pancreatic | Classical | FALSE | Classical | 0.0055 |
| | PF | | | | | Progenitor | | | | |
| S1124.15.02 | FOLF + | Post | −32.1429 | PR | Classical | Pancreatic | Classical | FALSE | Classical | 0.0055 |
| | PF | | | | | Progenitor | | | | |
| S1124.16.01 | FOLF + | Pre | −8.82353 | SD | Exocrine-like | ADEX | Classical | FALSE | Classical | 0.001096 |
| | PF | | | | | | | | | |
| S1124.16.02 | FOLF + | Post | −8.82353 | SD | Classical | Pancreatic | Classical | FALSE | Classical | 0.001096 |
| | PF | | | | | Progenitor | | | | |
| S1124.17.01 | FOLF + | Pre | 0 | SD | Exocrine-like | ADEX | Classical | FALSE | Classical | 0.001096 |
| | PF | | | | | | | | | |
| S1124.17.02 | FOLF + | Post | 0 | SD | Classical | Immunogenic | Classical | FALSE | Classical | 0.001096 |
| | PF | | | | | | | | | |
| S1124.21.01 | FOLF + | Pre | | | Exocrine-like | ADEX | Classical | FALSE | Classical | 0.001096 |
| | PF | | | | | | | | | |
| S1124.23.01 | FOLF + | Pre | | | Classical | Immunogenic | Classical | FALSE | Classical | 0.002769 |
| | PF | | | | | | | | | |
| S1124.24.01 | FOLF + | Pre | −40.625 | PR | Classical | Pancreatic | Classical | FALSE | Classical | 0.001096 |
| | PF | | | | | Progenitor | | | | |
| S1124.24.02 | FOLF + | Post | −40.625 | PR | Classical | Immunogenic | Classical | FALSE | Classical | 0.001096 |
| | PF | | | | | | | | | |
| S1124.25.01 | FOLF + | Pre | −19.697 | SD | Classical | Immunogenic | Classical | FALSE | Classical | 0.001096 |
| | PF | | | | | | | | | |
| S1124.25.02 | FOLF + | Post | −19.697 | SD | QM-PDA | Squamous | Classical | FALSE | Classical | 0.001096 |
| | PF | | | | | | | | | |
| S1124.28.01 | FOLF + | Pre | −37.037 | PR | QM-PDA | Immunogenic | Classical | FALSE | Classical | 0.0055 |
| | PF | | | | | | | | | |
| S1124.28.02 | FOLF + | Post | −37.037 | PR | QM-PDA | Immunogenic | Classical | FALSE | Classical | 0.002769 |
| | PF | | | | | | | | | |
| S1124.30.01 | FOLF + | Pre | −46.875 | PR | QM-PDA | Squamous | Classical | FALSE | Classical | 0.002749 |
| | PF | | | | | | | | | |
| S1124.30.02 | FOLF + | Post | −46.875 | PR | QM-PDA | Squamous | Classical | FALSE | Classical | 0.147772 |
| | PF | | | | | | | | | |
| S1124.31.01 | FOLF + | Pre | 25.64103 | PD | QM-PDA | Squamous | Basal-like | FALSE | Basal-like | 0.783895 |
| | PF | | | | | | | | | |
| S1124.31.02 | FOLF + | Post | 25.64103 | PD | QM-PDA | Squamous | Basal-like | FALSE | Basal-like | 0.783895 |
| | PF | | | | | | | | | |
| S1124.32.01 | FOLF + | Pre | −17.1429 | SD | QM-PDA | Squamous | Basal-like | FALSE | Basal-like | 0.991223 |
| | PF | | | | | | | | | |
| S1124.32.02 | FOLF + | Post | −17.1429 | SD | QM-PDA | Squamous | Basal-like | FALSE | Basal-like | 0.842116 |
| | PF | | | | | | | | | |
| S1124.33.01 | FOLF + | Pre | −8.16327 | SD | Classical | Pancreatic | Classical | FALSE | Classical | 0.0055 |
| | PF | | | | | Progenitor | | | | |
| S1124.33.02 | FOLF + | Post | −8.16327 | SD | QM-PDA | Squamous | Classical | FALSE | Classical | 0.0055 |
| | PF | | | | | | | | | |
| S1124.34.01 | FOLF + | Pre | −40 | PR | Classical | Immunogenic | Classical | FALSE | Classical | 0.001096 |
| | PF | | | | | | | | | |
| S1124.34.02 | FOLF + | Post | −40 | PR | Classical | Immunogenic | Classical | FALSE | Classical | 0.002769 |
| | PF | | | | | | | | | |
| S1124.35.01 | FOLF + | Pre | −26.9841 | SD | QM-PDA | Immunogenic | Classical | FALSE | Classical | 0.002769 |
| | PF | | | | | | | | | |
| S1124.35.02 | FOLF + | Post | −26.9841 | SD | QM-PDA | Immunogenic | Classical | FALSE | Classical | 0.002769 |
| | PF | | | | | | | | | |
| S1124.37.01 | FOLF + | Pre | −30 | PR | Classical | Immunogenic | Classical | FALSE | Classical | 0.002769 |
| | PF | | | | | | | | | |
| S1124.37.02 | FOLF + | Post | −30 | PR | Classical | Immunogenic | Classical | FALSE | Classical | 0.004491 |
| | PF | | | | | | | | | |
| S1124.38.01 | FOLF + | Pre | 8.571429 | SD | Exocrine-like | Pancreatic | Classical | FALSE | Classical | 0.0055 |
| | PF | | | | | Progenitor | | | | |
| S1124.38.02 | FOLF + | Post | 8.571429 | SD | Classical | Immunogenic | Classical | FALSE | Classical | 0.001096 |
| | PF | | | | | | | | | |
| S1124.40.01 | FOLF + | Pre | | | Classical | Pancreatic | Classical | FALSE | Classical | 0.001096 |
| | PF | | | | | Progenitor | | | | |
| S1124.41.01 | FOLF + | Pre | 6.451613 | SD | Exocrine-like | Pancreatic | Classical | FALSE | Classical | 0.0055 |
| | PF | | | | | Progenitor | | | | |
| S1124.41.02 | FOLF + | Post | 6.451613 | SD | Exocrine-like | ADEX | Classical | FALSE | Classical | 0.001096 |
| | PF | | | | | | | | | |
| S1124.42.01 | FOLF + | Pre | −29.6296 | SD | QM-PDA | Immunogenic | Classical | FALSE | Classical | 0.001096 |
| | PF | | | | | | | | | |
| S1124.42.02 | FOLF + | Post | −29.6296 | SD | QM-PDA | Immunogenic | Classical | FALSE | Classical | 0.001096 |
| | PF | | | | | | | | | |
| S1124.43.01 | FOLF + | Pre | −35.7143 | PR | Classical | Immunogenic | Classical | FALSE | Classical | 0.001096 |
| | PF | | | | | | | | | |
| S1124.43.02 | FOLF + | Post | −35.7143 | PR | QM-PDA | Squamous | Classical | FALSE | Classical | 0.0055 |
| | PF | | | | | | | | | |
| S1124.46.01 | FOLF + | Pre | −35.5556 | PR | QM-PDA | Immunogenic | Classical | FALSE | Classical | 0.001096 |
| | PF | | | | | | | | | |
| S1124.46.02 | FOLF + | Post | −35.5556 | PR | Classical | Immunogenic | Classical | FALSE | Classical | 0.001096 |
| | PF | | | | | | | | | |
| S1124.48.01 | FOLF + | Pre | 0 | SD | Classical | Pancreatic | Classical | FALSE | Classical | 0.0055 |
| | PF | | | | | Progenitor | | | | |
| S1124.48.02 | FOLF + | Post | 0 | SD | Classical | Immunogenic | Classical | FALSE | Classical | 0.0055 |
| | PF | | | | | | | | | |
| S1124.51.01 | FOLF + | Pre | −13.5135 | SD | Classical | Pancreatic | Classical | FALSE | Classical | 0.0055 |
| | PF | | | | | Progenitor | | | | |
| S1124.51.02 | FOLF + | Post | −13.5135 | SD | Exocrine-like | Pancreatic | Classical | FALSE | Classical | 0.001096 |
| | PF | | | | | Progenitor | | | | |
| S1124.52.01 | FOLF + | Pre | | | QM-PDA | Squamous | Classical | FALSE | Classical | 0.002769 |
| | PF | | | | | | | | | |
| S1124.53.01 | FOLF + | Pre | −13.5135 | SD | Exocrine-like | Pancreatic | Classical | FALSE | Classical | 0.0055 |
| | PF | | | | | Progenitor | | | | |
| S1124.53.02 | FOLF + | Post | −13.5135 | SD | Exocrine-like | ADEX | Classical | FALSE | Classical | 0.001096 |
| | PF | | | | | | | | | |
| S1124.54.01 | FOLF + | Pre | −5.71429 | SD | QM-PDA | Squamous | Classical | FALSE | Classical | 0.002769 |
| | PF | | | | | | | | | |
| S1124.54.02 | FOLF + | Post | −5.71429 | SD | Classical | Immunogenic | Classical | FALSE | Classical | 0.001779 |
| | PF | | | | | | | | | |
| S1124.57.01 | FOLF + | Pre | −33.3333 | PR | Exocrine-like | ADEX | Classical | FALSE | Classical | 0.001096 |
| | PF | | | | | | | | | |
| S1124.57.02 | FOLF + | Post | −33.3333 | PR | Exocrine-like | ADEX | Classical | FALSE | Classical | 0.001096 |
| | PF | | | | | | | | | |

TABLE 12
Moffitt_GEO_array

| ID | SampleType | Collisson | Bailey | Moffitt | PurIST.training | PurIST | PurIST.basal.prob |
|---|---|---|---|---|---|---|---|
| 53862-Primary-Pancreas | Primary | Exocrine-like | Squamous | Classical | TRUE | Classical | 0.007943 |
| 49360-Primary-Pancreas | Primary | QM-PDA | Squamous | Basal-like | TRUE | Basal-like | 0.766498 |
| 54249-Primary-Pancreas | Primary | QM-PDA | Pancreatic Progenitor | Classical | TRUE | Classical | 0.013247 |
| 48661-Primary-Pancreas | Primary | Exocrine-like | Pancreatic Progenitor | Classical | TRUE | Classical | 0.001096 |
| 49071-Primary-Pancreas | Primary | Exocrine-like | ADEX | Classical | TRUE | Classical | 0.001779 |
| 53838-Primary-Pancreas | Primary | Exocrine-like | ADEX | Classical | TRUE | Classical | 0.002749 |
| 49073-Primary-Pancreas | Primary | QM-PDA | Squamous | Classical | TRUE | Classical | 0.050513 |
| 48556-Primary-Pancreas | Primary | QM-PDA | Squamous | Basal-like | TRUE | Basal-like | 0.936765 |
| 48558-Primary-Pancreas | Primary | Classical | Squamous | Classical | TRUE | Classical | 0.146264 |
| 52042-Primary-Pancreas | Primary | QM-PDA | Squamous | Classical | TRUE | Classical | 0.002769 |
| 52043-Primary-Pancreas | Primary | Classical | Pancreatic Progenitor | Classical | TRUE | Classical | 0.001096 |
| 48562-Primary-Pancreas | Primary | Classical | Pancreatic Progenitor | Classical | TRUE | Classical | 0.001096 |
| 48564-Primary-Pancreas | Primary | QM-PDA | Squamous | Basal-like | FALSE | Classical | 0.429034 |
| 48567-Primary-Pancreas | Primary | Classical | Immunogenic | Classical | TRUE | Classical | 0.019979 |
| 48568-Primary-Pancreas | Primary | Exocrine-like | Squamous | Classical | TRUE | Basal-like | 0.755533 |
| 49388-Primary-Pancreas | Primary | Exocrine-like | ADEX | Classical | TRUE | Classical | 0.001096 |
| 46648-Primary-Pancreas | Primary | Classical | Pancreatic Progenitor | Classical | TRUE | Classical | 0.001096 |
| 46649-Primary-Pancreas | Primary | QM-PDA | Squamous | Classical | TRUE | Classical | 0.001096 |
| 46650-Primary-Pancreas | Primary | Exocrine-like | Pancreatic Progenitor | Classical | TRUE | Classical | 0.001096 |
| 46651-Primary-Pancreas | Primary | Exocrine-like | Pancreatic Progenitor | Classical | TRUE | Classical | 0.019841 |
| 47702-Primary-Pancreas | Primary | Classical | Squamous | Classical | TRUE | Classical | 0.001096 |
| 46652-Primary-Pancreas | Primary | Classical | Pancreatic Progenitor | Classical | TRUE | Classical | 0.001096 |
| 46987-Primary-Pancreas | Primary | Exocrine-like | Immunogenic | Classical | TRUE | Classical | 0.002769 |
| 46653-Primary-Pancreas | Primary | Classical | Squamous | Basal-like | TRUE | Basal-like | 0.827009 |
| 46832-Primary-Pancreas | Primary | Exocrine-like | Squamous | Basal-like | TRUE | Basal-like | 0.975101 |
| 46831-Primary-Pancreas | Primary | QM-PDA | Squamous | Basal-like | TRUE | Basal-like | 0.991223 |
| 46985-Primary-Pancreas | Primary | Classical | Squamous | Classical | TRUE | Classical | 0.013247 |
| 46828-Primary-Pancreas | Primary | Classical | Pancreatic Progenitor | Classical | TRUE | Classical | 0.089725 |
| 47692-Primary-Pancreas | Primary | QM-PDA | Squamous | Basal-like | TRUE | Basal-like | 0.991223 |
| 46986-Primary-Pancreas | Primary | Classical | Squamous | Classical | TRUE | Classical | 0.001096 |
| 47590-Primary-Pancreas | Primary | Exocrine-like | Pancreatic Progenitor | Classical | TRUE | Classical | 0.001096 |
| 47969-Primary-Pancreas | Primary | Exocrine-like | ADEX | Classical | TRUE | Classical | 0.002769 |
| 47989-Primary-Pancreas | Primary | Classical | Pancreatic Progenitor | Classical | TRUE | Classical | 0.013247 |
| 46581-Primary-Pancreas | Primary | Exocrine-like | ADEX | Classical | TRUE | Classical | 0.001779 |
| 46582-Primary-Pancreas | Primary | Classical | Pancreatic Progenitor | Classical | TRUE | Classical | 0.001096 |
| 46830-Primary-Pancreas | Primary | QM-PDA | Squamous | Classical | TRUE | Classical | 0.001096 |
| 46584-Primary-Pancreas | Primary | Classical | Pancreatic Progenitor | Classical | TRUE | Classical | 0.001096 |
| 47703-Primary-Pancreas | Primary | QM-PDA | Squamous | Basal-like | TRUE | Basal-like | 0.991223 |
| 47708-Primary-Pancreas | Primary | Exocrine-like | ADEX | Classical | TRUE | Classical | 0.001096 |
| 46450-Primary-Pancreas | Primary | Classical | Pancreatic Progenitor | Classical | TRUE | Classical | 0.001096 |
| 47695-Primary-Pancreas | Primary | Classical | Pancreatic Progenitor | Classical | TRUE | Classical | 0.001096 |
| 48550-Primary-Pancreas | Primary | Exocrine-like | Immunogenic | Classical | TRUE | Classical | 0.001096 |
| 46339-Primary-Pancreas | Primary | Classical | Squamous | Basal-like | TRUE | Basal-like | 0.975101 |
| 46578-Primary-Pancreas | Primary | Classical | Pancreatic Progenitor | Classical | TRUE | Classical | 0.001096 |
| 46585-Primary-Pancreas | Primary | Exocrine-like | Squamous | Classical | TRUE | Classical | 0.052325 |
| 46337-Primary-Pancreas | Primary | QM-PDA | Squamous | Classical | TRUE | Classical | 0.048743 |
| 46587-Primary-Pancreas | Primary | Classical | Pancreatic Progenitor | Classical | TRUE | Classical | 0.013247 |
| 47700-Primary-Pancreas | Primary | Exocrine-like | Squamous | Classical | TRUE | Classical | 0.002749 |
| 46826-Primary-Pancreas | Primary | Classical | Pancreatic Progenitor | Classical | TRUE | Classical | 0.001096 |
| 46592-Primary-Pancreas | Primary | Classical | Pancreatic Progenitor | Classical | TRUE | Classical | 0.001096 |
| 46452-Primary-Pancreas | Primary | Exocrine-like | ADEX | Classical | TRUE | Classical | 0.001096 |
| 46460-Primary-Pancreas | Primary | QM-PDA | Squamous | Basal-like | FALSE | Classical | 0.288464 |
| 47983-Primary-Pancreas | Primary | Classical | Pancreatic Progenitor | Classical | TRUE | Classical | 0.00799 |
| 46642-Primary-Pancreas | Primary | Classical | Pancreatic Progenitor | Classical | TRUE | Classical | 0.001096 |
| 47701-Primary-Pancreas | Primary | QM-PDA | Squamous | Classical | TRUE | Classical | 0.199683 |
| 46643-Primary-Pancreas | Primary | Classical | Pancreatic Progenitor | Classical | TRUE | Classical | 0.013247 |
| 47965-Primary-Pancreas | Primary | Exocrine-like | Squamous | Classical | TRUE | Classical | 0.00693 |
| 46644-Primary-Pancreas | Primary | Classical | Pancreatic Progenitor | Classical | TRUE | Classical | 0.002769 |
| 46645-Primary-Pancreas | Primary | Exocrine-like | Pancreatic Progenitor | Classical | TRUE | Classical | 0.019841 |
| 46646-Primary-Pancreas | Primary | Classical | Pancreatic Progenitor | Classical | TRUE | Classical | 0.001096 |
| 49390-Primary-Pancreas | Primary | Exocrine-like | ADEX | Classical | TRUE | Classical | 0.001096 |
| 49392-Primary-Pancreas | Primary | QM-PDA | Squamous | Basal-like | TRUE | Basal-like | 0.837062 |
| 64482-Primary-Pancreas | Primary | Exocrine-like | ADEX | Classical | TRUE | Classical | 0.00799 |
| 64500-Primary-Pancreas | Primary | Exocrine-like | ADEX | Classical | TRUE | Classical | 0.001096 |
| 72613-Primary-Pancreas | Primary | Classical | Pancreatic Progenitor | Classical | TRUE | Classical | 0.001096 |
| 64501-Primary-Pancreas | Primary | Classical | Pancreatic Progenitor | Classical | TRUE | Classical | 0.001096 |
| 72616-Primary-Pancreas | Primary | Exocrine-like | ADEX | Classical | TRUE | Classical | 0.002749 |
| 64502-Primary-Pancreas | Primary | Exocrine-like | ADEX | Classical | TRUE | Classical | 0.13805 |
| 64503-Primary-Pancreas | Primary | Exocrine-like | Pancreatic Progenitor | Classical | TRUE | Classical | 0.021347 |
| 64504-Primary-Pancreas | Primary | Exocrine-like | ADEX | Classical | TRUE | Classical | 0.001096 |
| 64505-Primary-Pancreas | Primary | Exocrine-like | ADEX | Classical | TRUE | Classical | 0.001096 |
| 64507-Primary-Pancreas | Primary | Exocrine-like | ADEX | Classical | TRUE | Classical | 0.204595 |
| 64508-Primary-Pancreas | Primary | Exocrine-like | ADEX | Classical | TRUE | Classical | 0.001096 |
| 64509-Primary-Pancreas | Primary | Exocrine-like | ADEX | Basal-like | TRUE | Basal-like | 0.898468 |
| 64510-Primary-Pancreas | Primary | Exocrine-like | Pancreatic Progenitor | Classical | TRUE | Classical | 0.013247 |
| 46647-Primary-Pancreas | Primary | QM-PDA | Squamous | Basal-like | TRUE | Basal-like | 0.991223 |
| 48569-Primary-Pancreas | Primary | Classical | Pancreatic Progenitor | Classical | TRUE | Classical | 0.013247 |
| 64498-Primary-Pancreas | Primary | QM-PDA | Squamous | Basal-like | TRUE | Basal-like | 0.957264 |
| 64490-Primary-Pancreas | Primary | QM-PDA | Squamous | Classical | FALSE | Classical | 0.020585 |
| 64491-Primary-Pancreas | Primary | Exocrine-like | ADEX | Classical | TRUE | Classical | 0.001779 |
| 64492-Primary-Pancreas | Primary | Exocrine-like | ADEX | Classical | TRUE | Classical | 0.001096 |
| 64494-Primary-Pancreas | Primary | QM-PDA | Squamous | Basal-like | TRUE | Basal-like | 0.957264 |
| 64495-Primary-Pancreas | Primary | QM-PDA | Squamous | Classical | TRUE | Classical | 0.001096 |
| 56525-Primary-Pancreas | Primary | QM-PDA | Squamous | Basal-like | TRUE | Basal-like | 0.671478 |
| 56527-Primary-Pancreas | Primary | Exocrine-like | Pancreatic Progenitor | Classical | TRUE | Classical | 0.013247 |
| 56536-Primary-Pancreas | Primary | QM-PDA | Squamous | Basal-like | TRUE | Basal-like | 0.978228 |
| 56537-Primary-Pancreas | Primary | Exocrine-like | ADEX | Classical | TRUE | Classical | 0.001096 |
| 56538-Primary-Pancreas | Primary | Classical | Pancreatic Progenitor | Classical | TRUE | Classical | 0.001096 |
| 56539-Primary-Pancreas | Primary | Classical | Pancreatic Progenitor | Classical | TRUE | Classical | 0.013247 |
| 56367-Primary-Pancreas | Primary | QM-PDA | Pancreatic Progenitor | Classical | TRUE | Classical | 0.019841 |
| 56369-Primary-Pancreas | Primary | Classical | Pancreatic Progenitor | Classical | TRUE | Classical | 0.001096 |
| 56528-Primary-Pancreas | Primary | Classical | Pancreatic Progenitor | Classical | TRUE | Classical | 0.001096 |
| 56529-Primary-Pancreas | Primary | Exocrine-like | Squamous | Classical | TRUE | Classical | 0.001096 |
| 56530-Primary-Pancreas | Primary | Exocrine-like | Squamous | Classical | TRUE | Classical | 0.001096 |
| 56540-Primary-Pancreas | Primary | Exocrine-like | Squamous | Classical | TRUE | Classical | 0.001779 |
| 56377-Primary-Pancreas | Primary | Exocrine-like | ADEX | Basal-like | FALSE | Classical | 0.012917 |
| 56373-Primary-Pancreas | Primary | Exocrine-like | Pancreatic Progenitor | Classical | TRUE | Classical | 0.001096 |
| 56374-Primary-Pancreas | Primary | Exocrine-like | ADEX | Classical | TRUE | Classical | 0.00799 |
| 56541-Primary-Pancreas | Primary | Exocrine-like | Squamous | Basal-like | FALSE | Classical | 0.005113 |
| 56542-Primary-Pancreas | Primary | Classical | Pancreatic Progenitor | Classical | TRUE | Classical | 0.013247 |
| 56375-Primary-Pancreas | Primary | Exocrine-like | ADEX | Classical | TRUE | Classical | 0.001096 |
| 56535-Primary-Pancreas | Primary | Classical | Pancreatic Progenitor | Classical | TRUE | Classical | 0.013247 |
| 54175-Primary-Pancreas | Primary | QM-PDA | Squamous | Classical | TRUE | Classical | 0.001096 |
| 54301-Primary-Pancreas | Primary | Exocrine-like | ADEX | Classical | TRUE | Classical | 0.001779 |
| 54291-Primary-Pancreas | Primary | QM-PDA | Squamous | Classical | TRUE | Classical | 0.089725 |
| 54302-Primary-Pancreas | Primary | QM-PDA | Squamous | Basal-like | TRUE | Basal-like | 0.991223 |
| 54303-Primary-Pancreas | Primary | Classical | Pancreatic Progenitor | Classical | TRUE | Classical | 0.001096 |
| 54172-Primary-Pancreas | Primary | QM-PDA | Squamous | Classical | TRUE | Classical | 0.001096 |
| 54304-Primary-Pancreas | Primary | Classical | Pancreatic Progenitor | Classical | TRUE | Classical | 0.001096 |
| 54305-Primary-Pancreas | Primary | QM-PDA | Squamous | Classical | TRUE | Classical | 0.013247 |
| 54309-Primary-Pancreas | Primary | Exocrine-like | ADEX | Classical | TRUE | Classical | 0.001096 |
| 54306-Primary-Pancreas | Primary | Exocrine-like | Squamous | Basal-like | TRUE | Basal-like | 0.754224 |
| 54307-Primary-Pancreas | Primary | QM-PDA | Squamous | Classical | TRUE | Classical | 0.00799 |
| 54292-Primary-Pancreas | Primary | Exocrine-like | ADEX | Classical | TRUE | Classical | 0.001096 |
| 54243-Primary-Pancreas | Primary | Classical | Pancreatic Progenitor | Classical | TRUE | Classical | 0.001096 |
| 54308-Primary-Pancreas | Primary | QM-PDA | Squamous | Classical | TRUE | Classical | 0.019979 |
| 54293-Primary-Pancreas | Primary | QM-PDA | Squamous | Classical | TRUE | Classical | 0.002769 |
| 54310-Primary-Pancreas | Primary | QM-PDA | Squamous | Classical | TRUE | Classical | 0.001096 |
| 54315-Primary-Pancreas | Primary | QM-PDA | Squamous | Basal-like | TRUE | Basal-like | 0.936765 |
| 54311-Primary-Pancreas | Primary | Classical | Pancreatic Progenitor | Classical | TRUE | Classical | 0.0055 |
| 54312-Primary-Pancreas | Primary | Exocrine-like | Pancreatic Progenitor | Classical | TRUE | Classical | 0.001096 |
| 54299-Primary-Pancreas | Primary | Exocrine-like | ADEX | Classical | TRUE | Classical | 0.001096 |
| 54313-Primary-Pancreas | Primary | Exocrine-like | Squamous | Basal-like | TRUE | Basal-like | 0.978077 |
| 54314-Primary-Pancreas | Primary | QM-PDA | Pancreatic Progenitor | Classical | TRUE | Classical | 0.013247 |
| 54294-Primary-Pancreas | Primary | Exocrine-like | ADEX | Classical | TRUE | Classical | 0.001096 |
| 54295-Primary-Pancreas | Primary | Classical | Pancreatic Progenitor | Classical | TRUE | Classical | 0.013247 |
| 54173-Primary-Pancreas | Primary | Exocrine-like | Pancreatic Progenitor | Classical | TRUE | Classical | 0.001096 |
| 54316-Primary-Pancreas | Primary | QM-PDA | Squamous | Basal-like | TRUE | Basal-like | 0.859538 |
| 54317-Primary-Pancreas | Primary | Classical | Pancreatic Progenitor | Classical | TRUE | Classical | 0.001096 |
| 54297-Primary-Pancreas | Primary | Exocrine-like | ADEX | Classical | TRUE | Classical | 0.001096 |
| 54300-Primary-Pancreas | Primary | QM-PDA | Squamous | Classical | TRUE | Classical | 0.089725 |
| 54318-Primary-Pancreas | Primary | Classical | Immunogenic | Classical | TRUE | Classical | 0.013247 |
| 54296-Primary-Pancreas | Primary | Classical | Pancreatic Progenitor | Classical | TRUE | Classical | 0.001096 |
| 54174-Primary-Pancreas | Primary | Classical | Pancreatic Progenitor | Classical | TRUE | Classical | 0.001096 |
| 54298-Primary-Pancreas | Primary | Exocrine-like | ADEX | Classical | TRUE | Classical | 0.00446 |
| 54171-Primary-Pancreas | Primary | QM-PDA | Squamous | Classical | TRUE | Classical | 0.004491 |
| 64496-Primary-Pancreas | Primary | QM-PDA | Squamous | Basal-like | FALSE | Classical | 0.031663 |
| 56322-Primary-Pancreas | Primary | Exocrine-like | Immunogenic | Classical | TRUE | Classical | 0.013247 |
| 56326-Primary-Pancreas | Primary | QM-PDA | Squamous | Basal-like | TRUE | Basal-like | 0.790185 |
| 56534-Primary-Pancreas | Primary | QM-PDA | Pancreatic Progenitor | Classical | TRUE | Classical | 0.013247 |
| 56531-Primary-Pancreas | Primary | QM-PDA | Squamous | Classical | TRUE | Classical | 0.001096 |
| 56523-Primary-Pancreas | Primary | QM-PDA | Squamous | Classical | TRUE | Classical | 0.001096 |
| 56316-Primary-Pancreas | Primary | QM-PDA | ADEX | Classical | TRUE | Classical | 0.001096 |
| 56320-Primary-Pancreas | Primary | QM-PDA | Squamous | Classical | TRUE | Classical | 0.052325 |
| 64497-Primary-Pancreas | Primary | Exocrine-like | ADEX | Classical | TRUE | Classical | 0.002749 |

TABLE 13
Moffitt_S2

| ID | SampleType | Collisson | Bailey | Moffitt | PurIST.training | PurIST | PurIST.basal.prob |
|---|---|---|---|---|---|---|---|
| PDX-1 | PDX | | | Classical | FALSE | Classical | 0.001096 |
| PDX-2 | PDX | | | Classical | FALSE | Classical | 0.001096 |
| PDX-3 | PDX | | | Classical | FALSE | Basal-like | 0.514077 |
| PDX-4 | PDX | | | Classical | FALSE | Classical | 0.001096 |
| PDX-5 | PDX | | | Basal-like | FALSE | Basal-like | 0.991223 |
| PDX-6 | PDX | | | Classical | FALSE | Classical | 0.078689 |
| PDX-7 | PDX | | | Classical | FALSE | Classical | 0.001096 |
| PDX-8 | PDX | | | Classical | FALSE | Classical | 0.001096 |
| PDX-9 | PDX | | | Classical | FALSE | Classical | 0.001096 |
| PDX-10 | PDX | | | Classical | FALSE | Classical | 0.001096 |
| PDX-11 | PDX | | | Classical | FALSE | Classical | 0.002769 |
| PDX-12 | PDX | | | Classical | FALSE | Classical | 0.001096 |
| PDX-13 | PDX | | | Classical | FALSE | Classical | 0.001096 |
| PDX-14 | PDX | | | Classical | FALSE | Classical | 0.001096 |
| PDX-15 | PDX | | | Classical | FALSE | Classical | 0.020585 |
| PDX-16 | PDX | | | Classical | FALSE | Classical | 0.001096 |
| PDX-17 | PDX | | | Classical | FALSE | Classical | 0.001096 |
| PDX-18 | PDX | | | Classical | FALSE | Classical | 0.013805 |
| PDX-19 | PDX | | | Classical | FALSE | Classical | 0.013247 |
| PDX-20 | PDX | | | Classical | FALSE | Classical | 0.013805 |
| PDX-21 | PDX | | | Classical | FALSE | Classical | 0.001096 |
| PDX-22 | PDX | | | Classical | FALSE | Classical | 0.0055 |
| PDX-23 | PDX | | | Classical | FALSE | Classical | 0.02224 |
| PDX-24 | PDX | | | Classical | FALSE | Classical | 0.001096 |
| PDX-25 | PDX | | | Classical | FALSE | Classical | 0.002749 |
| PDX-26 | PDX | | | Basal-like | FALSE | Basal-like | 0.691693 |
| PDX-27 | PDX | | | Classical | FALSE | Classical | 0.001096 |
| PDX-28 | PDX | | | Classical | FALSE | Classical | 0.001096 |
| PDX-29 | PDX | | | Classical | FALSE | Classical | 0.013247 |
| PDX-30 | PDX | | | Basal-like | FALSE | Basal-like | 0.960163 |
| PDX-31 | PDX | | | Classical | FALSE | Classical | 0.001096 |
| PDX-32 | PDX | | | Classical | FALSE | Classical | 0.001096 |
| PDX-33 | PDX | | | Classical | FALSE | Classical | 0.001096 |
| PDX-34 | PDX | | | Basal-like | FALSE | Basal-like | 0.991223 |
| PDX-35 | PDX | | | Classical | FALSE | Classical | 0.001096 |
| PDX-36 | PDX | | | Classical | FALSE | Classical | 0.001096 |
| PDX-37 | PDX | | | Basal-like | FALSE | Basal-like | 0.991223 |

TABLE 14
PACA_AU_array

| ID | SampleType | Collisson | Bailey_original | Bailey | Moffitt | PurIST.training | PurIST | PurIST.basal.prob |
|---|---|---|---|---|---|---|---|---|
| SA407779 | Primary tumour | Exocrine-like | | ADEX | | FALSE | Classical | 0.014 |
| SA407918 | Primary tumour | Exocrine-like | Immunogenic | ADEX | Classical | FALSE | Classical | 0.014 |
| SA407946 | Cell line | Exocrine-like | | ADEX | | FALSE | Classical | 0.005 |
| SA408003 | Primary tumour | Exocrine-like | Squamous | Squamous | | FALSE | Classical | 0.427 |
| SA408106 | Primary tumour | Classical | | Pancreatic Progenitor | Classical | FALSE | Classical | 0.005 |
| SA408266 | Cell line | Exocrine-like | | ADEX | | FALSE | Classical | 0.014 |
| SA408314 | Primary tumour | Exocrine-like | | ADEX | Classical | FALSE | Classical | 0.005 |
| SA408414 | Primary tumour | QM-PDA | Squamous | ADEX | Classical | FALSE | Classical | 0.005 |
| SA408530 | Primary tumour | Exocrine-like | Squamous | Squamous | Basal-like | FALSE | Classical | 0.211 |
| SA408570 | Primary tumour | Exocrine-like | ADEX | ADEX | Classical | FALSE | Classical | 0.014 |
| SA408650 | Metastatic tumour | QM-PDA | | ADEX | | FALSE | Classical | 0.014 |
| SA408706 | Primary tumour | Classical | Pancreatic Progenitor | Pancreatic Progenitor | Classical | FALSE | Classical | 0.014 |
| SA408726 | Cell line | QM-PDA | | Immunogenic | | FALSE | Classical | 0.014 |
| SA408758 | Primary tumour | Exocrine-like | ADEX | ADEX | Classical | FALSE | Classical | 0.014 |
| SA408774 | Cell line | QM-PDA | | ADEX | | FALSE | Classical | 0.014 |
| SA408806 | Primary tumour | QM-PDA | | Immunogenic | | FALSE | Classical | 0.005 |
| SA408843 | Primary tumour | QM-PDA | | Immunogenic | Classical | FALSE | Classical | 0.093 |
| SA408867 | Primary tumour | QM-PDA | Squamous | Squamous | Basal-like | FALSE | Classical | 0.427 |
| SA408891 | Primary tumour | QM-PDA | Squamous | Squamous | Basal-like | FALSE | Basal-like | 0.975 |
| SA408946 | Primary tumour | Exocrine-like | | Squamous | Classical | FALSE | Classical | 0.412 |
| SA408963 | Cell line | Classical | | Pancreatic Progenitor | | FALSE | Classical | 0.005 |
| SA409186 | Primary tumour | Exocrine-like | ADEX | ADEX | Classical | FALSE | Classical | 0.005 |
| SA409258 | Primary tumour | QM-PDA | Pancreatic Progenitor | Pancreatic Progenitor | Classical | FALSE | Classical | 0.005 |
| SA409310 | Primary tumour | Exocrine-like | Squamous | Squamous | Basal-like | FALSE | Basal-like | 0.975 |
| SA409342 | Primary tumour | QM-PDA | Pancreatic Progenitor | ADEX | | FALSE | Classical | 0.034 |
| SA409398 | Primary tumour | Exocrine-like | | ADEX | Classical | FALSE | Classical | 0.022 |
| SA409446 | Primary tumour | Classical | Pancreatic Progenitor | Pancreatic Progenitor | Classical | FALSE | Classical | 0.005 |
| SA409498 | Primary tumour | QM-PDA | Immunogenic | ADEX | | FALSE | Classical | 0.034 |
| SA409527 | Cell line | Exocrine-like | | ADEX | | FALSE | Classical | 0.022 |
| SA409543 | Primary tumour | Classical | Immunogenic | Pancreatic Progenitor | | FALSE | Classical | 0.014 |
| SA409590 | Primary tumour | Exocrine-like | | Squamous | Classical | FALSE | Classical | 0.014 |
| SA409622 | Primary tumour | QM-PDA | Pancreatic Progenitor | Immunogenic | Classical | FALSE | Classical | 0.014 |
| SA409662 | Primary tumour | QM-PDA | Squamous | Immunogenic | Classical | FALSE | Classical | 0.005 |
| SA409678 | Cell line | Exocrine-like | | Squamous | | FALSE | Classical | 0.054 |
| SA409711 | Primary tumour | Exocrine-like | ADEX | Squamous | Classical | FALSE | Classical | 0.438 |
| SA409775 | Primary tumour | QM-PDA | Squamous | Immunogenic | Classical | FALSE | Classical | 0.034 |
| SA409818 | Cell line | Exocrine-like | | ADEX | | FALSE | Classical | 0.005 |
| SA409838 | Primary tumour | Classical | | Pancreatic Progenitor | Classical | FALSE | Classical | 0.005 |
| SA409891 | Primary tumour | Exocrine-like | ADEX | ADEX | Classical | FALSE | Classical | 0.005 |
| SA409923 | Primary tumour | Classical | Pancreatic Progenitor | Pancreatic Progenitor | | FALSE | Classical | 0.014 |
| SA410030 | Primary tumour | QM-PDA | | Immunogenic | Classical | FALSE | Classical | 0.005 |
| SA410054 | Primary tumour | QM-PDA | | ADEX | Classical | FALSE | Classical | 0.205 |
| SA410103 | Primary tumour | QM-PDA | Immunogenic | ADEX | Classical | FALSE | Classical | 0.039 |
| SA410118 | Primary tumour | Exocrine-like | Immunogenic | Immunogenic | Classical | FALSE | Classical | 0.014 |
| SA410207 | Primary tumour | Classical | ADEX | Pancreatic Progenitor | Classical | FALSE | Classical | 0.005 |
| SA410234 | Primary tumour | Exocrine-like | Pancreatic Progenitor | Pancreatic Progenitor | | FALSE | Classical | 0.005 |
| SA410263 | Primary tumour | QM-PDA | Immunogenic | Squamous | Classical | FALSE | Basal-like | 0.548 |
| SA410286 | Primary tumour | QM-PDA | | Immunogenic | Classical | FALSE | Classical | 0.014 |
| SA410310 | Primary tumour | Classical | Immunogenic | Pancreatic Progenitor | Classical | FALSE | Classical | 0.005 |
| SA410382 | Primary tumour | Exocrine-like | | Pancreatic Progenitor | Classical | FALSE | Classical | 0.005 |
| SA410383 | Primary tumour | QM-PDA | Squamous | Squamous | Basal-like | FALSE | Basal-like | 0.986 |
| SA410410 | Primary tumour | Exocrine-like | ADEX | ADEX | Classical | FALSE | Classical | 0.009 |
| SA410503 | Primary tumour | Exocrine-like | Pancreatic Progenitor | ADEX | Classical | FALSE | Classical | 0.014 |
| SA410535 | Primary tumour | Classical | Immunogenic | Pancreatic Progenitor | Classical | FALSE | Classical | 0.005 |
| SA410559 | Cell line | Exocrine-like | | ADEX | | FALSE | Classical | 0.005 |
| SA410566 | Primary tumour | Classical | | Pancreatic Progenitor | Classical | FALSE | Classical | 0.005 |
| SA410582 | Primary tumour | QM-PDA | Immunogenic | Pancreatic Progenitor | Classical | FALSE | Classical | 0.005 |
| SA410606 | | QM-PDA | | Squamous | | FALSE | Classical | 0.205 |
| SA410687 | Primary tumour | QM-PDA | Pancreatic Progenitor | Immunogenic | | FALSE | Classical | 0.211 |
| SA410742 | Primary tumour | Classical | Pancreatic Progenitor | Pancreatic Progenitor | Classical | FALSE | Classical | 0.014 |
| SA410750 | Primary tumour | Classical | Immunogenic | Pancreatic Progenitor | Classical | FALSE | Classical | 0.005 |
| SA410758 | Primary tumour | Classical | Pancreatic Progenitor | Pancreatic Progenitor | Classical | FALSE | Classical | 0.005 |
| SA410763 | Primary tumour | Classical | Immunogenic | Pancreatic Progenitor | Classical | FALSE | Classical | 0.005 |
| SA410859 | Primary tumour | QM-PDA | Squamous | Squamous | Basal-like | FALSE | Basal-like | 0.837 |
| SA410883 | Primary tumour | Exocrine-like | ADEX | ADEX | Classical | FALSE | Classical | 0.014 |
| SA410899 | Primary tumour | Classical | Pancreatic Progenitor | Pancreatic Progenitor | Classical | FALSE | Classical | 0.005 |
| SA410911 | Primary tumour | Exocrine-like | Squamous | Squamous | Basal-like | FALSE | Basal-like | 0.991 |
| SA410933 | Primary tumour | Classical | Pancreatic Progenitor | Pancreatic Progenitor | | FALSE | Classical | 0.005 |
| SA411001 | Primary tumour | Classical | Pancreatic Progenitor | Pancreatic Progenitor | | FALSE | Classical | 0.205 |
| SA411029 | Primary tumour | Classical | Pancreatic Progenitor | Pancreatic Progenitor | | FALSE | Classical | 0.005 |
| SA411042 | Primary tumour | QM-PDA | | Immunogenic | Classical | FALSE | Classical | 0.211 |
| SA411189 | Primary tumour | QM-PDA | Squamous | Immunogenic | Classical | FALSE | Classical | 0.054 |
| SA411209 | Primary tumour | Exocrine-like | ADEX | ADEX | Classical | FALSE | Classical | 0.093 |
| SA411241 | Primary tumour | Classical | Pancreatic Progenitor | Pancreatic Progenitor | Classical | FALSE | Classical | 0.014 |
| SA411261 | Primary tumour | QM-PDA | | Immunogenic | Classical | FALSE | Classical | 0.205 |
| SA411305 | Primary tumour | QM-PDA | Squamous | Immunogenic | Classical | FALSE | Classical | 0.034 |
| SA411360 | Cell line | QM-PDA | | Immunogenic | | FALSE | Classical | 0.014 |
| SA411397 | Primary tumour | Classical | Squamous | Immunogenic | Classical | FALSE | Classical | 0.014 |
| SA411406 | Primary tumour | Classical | Immunogenic | Pancreatic Progenitor | Classical | FALSE | Classical | 0.005 |
| SA411430 | Primary tumour | Classical | Immunogenic | Pancreatic Progenitor | | FALSE | Classical | 0.005 |
| SA411454 | Primary tumour | Exocrine-like | | Pancreatic Progenitor | Classical | FALSE | Classical | 0.005 |
| SA411557 | Primary tumour | Classical | Immunogenic | Pancreatic Progenitor | | FALSE | Classical | 0.005 |
| SA411578 | Primary tumour | QM-PDA | Squamous | Immunogenic | Classical | FALSE | Classical | 0.296 |
| SA411721 | Primary tumour | Classical | Pancreatic Progenitor | Pancreatic Progenitor | Classical | FALSE | Classical | 0.005 |
| SA411745 | Primary tumour | Classical | Immunogenic | Pancreatic Progenitor | Classical | FALSE | Classical | 0.014 |
| SA411769 | Primary tumour | Classical | Pancreatic Progenitor | Pancreatic Progenitor | Classical | FALSE | Classical | 0.005 |
| SA411797 | Primary tumour | Classical | Pancreatic Progenitor | Pancreatic Progenitor | | FALSE | Classical | 0.005 |
| SA411833 | Primary tumour | Classical | Immunogenic | Pancreatic Progenitor | Classical | FALSE | Classical | 0.005 |
| SA411907 | Cell line | QM-PDA | | Pancreatic Progenitor | | FALSE | Classical | 0.014 |
| SA411923 | Primary tumour | Exocrine-like | Squamous | Immunogenic | Classical | FALSE | Classical | 0.301 |
| SA412003 | Primary tumour | QM-PDA | | Immunogenic | Classical | FALSE | Classical | 0.034 |
| SA412076 | Primary tumour | QM-PDA | Squamous | Immunogenic | Classical | FALSE | Classical | 0.093 |
| SA412212 | Primary tumour | QM-PDA | Pancreatic Progenitor | Immunogenic | Classical | FALSE | Classical | 0.014 |
| SA412299 | Primary tumour | Exocrine-like | ADEX | ADEX | | FALSE | Classical | 0.039 |
| SA412367 | Primary tumour | Exocrine-like | | ADEX | Classical | FALSE | Classical | 0.034 |
| SA412455 | Primary tumour | Exocrine-like | | Squamous | Classical | FALSE | Classical | 0.412 |
| SA518603 | Primary tumour | Classical | Pancreatic Progenitor | Pancreatic Progenitor | Classical | FALSE | Classical | 0.014 |
| SA518614 | Primary tumour | Classical | Immunogenic | Pancreatic Progenitor | | FALSE | Classical | 0.014 |
| SA518615 | Primary tumour | Classical | Immunogenic | Pancreatic Progenitor | Classical | FALSE | Classical | 0.005 |
| SA518624 | | Classical | | Pancreatic Progenitor | | FALSE | Classical | 0.005 |
| SA518630 | Primary tumour | Exocrine-like | ADEX | ADEX | | FALSE | Classical | 0.005 |
| SA518633 | Primary tumour | QM-PDA | Squamous | Squamous | Basal-like | FALSE | Basal-like | 0.991 |
| SA518637 | Primary tumour | QM-PDA | Squamous | Squamous | Basal-like | FALSE | Basal-like | 0.975 |
| SA518665 | Primary tumour | Exocrine-like | ADEX | Pancreatic Progenitor | Classical | FALSE | Classical | 0.005 |
| SA518689 | Primary tumour | Classical | Immunogenic | Pancreatic Progenitor | Classical | FALSE | Classical | 0.005 |
| SA518695 | Primary tumour | Classical | Pancreatic Progenitor | Pancreatic Progenitor | Classical | FALSE | Classical | 0.005 |
| SA518701 | Primary tumour | Classical | Pancreatic Progenitor | Pancreatic Progenitor | | FALSE | Classical | 0.005 |
| SA518704 | Primary tumour | Exocrine-like | ADEX | Immunogenic | Classical | FALSE | Classical | 0.014 |
| SA518709 | Primary tumour | Exocrine-like | | Immunogenic | Classical | FALSE | Classical | 0.034 |
| SA518712 | Primary tumour | Exocrine-like | | Immunogenic | Classical | FALSE | Classical | 0.014 |
| SA518716 | Primary tumour | Exocrinc-like | Immunogenic | Pancreatic Progenitor | Classical | FALSE | Classical | 0.005 |
| SA518724 | Primary tumour | Classical | Pancreatic Progenitor | Pancreatic Progenitor | Classical | FALSE | Classical | 0.005 |
| SA518765 | Primary tumour | Classical | Pancreatic Progenitor | Pancreatic Progenitor | Classical | FALSE | Classical | 0.014 |
| SA518806 | Primary tumour | QM-PDA | Squamous | Immunogenic | Classical | FALSE | Classical | 0.205 |
| SA518817 | Primary tumour | Classical | Immunogenic | Pancreatic Progenitor | Classical | FALSE | Classical | 0.005 |
| SA518851 | Primary tumour | QM-PDA | Squamous | Squamous | Basal-like | FALSE | Basal-like | 0.991 |
| SA518854 | Primary tumour | Exocrine-like | | Squamous | Classical | FALSE | Classical | 0.034 |
| SA518868 | Primary tumour | QM-PDA | Immunogenic | Immunogenic | Classical | FALSE | Classical | 0.093 |
| SA518878 | Primary tumour | QM-PDA | Squamous | Immunogenic | Classical | FALSE | Classical | 0.039 |
| SA528670 | Primary tumour | Exocrine-like | | Squamous | Basal-like | FALSE | Basal-like | 0.975 |
| SA528675 | Primary tumour | Classical | Immunogenic | Pancreatic Progenitor | | FALSE | Classical | 0.014 |
| SA528676 | Primary tumour | QM-PDA | | Immunogenic | Classical | FALSE | Classical | 0.034 |
| SA528687 | Primary tumour | Classical | Immunogenic | Pancreatic Progenitor | Classical | FALSE | Classical | 0.005 |
| SA528693 | Primary tumour | QM-PDA | ADEX | Squamous | Basal-like | FALSE | Basal-like | 0.991 |
| SA528695 | Primary tumour | Classical | Pancreatic Progenitor | Pancreatic Progenitor | Classical | FALSE | Classical | 0.005 |
| SA528697 | Primary tumour | Exocrine-like | | Immunogenic | Classical | FALSE | Classical | 0.014 |
| SA528709 | Primary tumour | QM-PDA | Squamous | Squamous | Basal-like | FALSE | Basal-like | 0.991 |
| SA528712 | Cell line | QM-PDA | | Immunogenic | | FALSE | Classical | 0.022 |
| SA528713 | Metastatic tumour | Exocrine-like | | Squamous | | FALSE | Classical | 0.034 |
| SA528755 | Primary tumour | Classical | Immunogenic | Pancreatic Progenitor | Classical | FALSE | Classical | 0.005 |
| SA528761 | Primary tumour | QM-PDA | Squamous | Squamous | Basal-like | FALSE | Basal-like | 0.558 |

TABLE 15
PACA_AU_seq

| ID | SampleType | Collisson | Balley_original | Bailey | Moffitt | PurIST.training | PurIST | PurIST.basal.prob |
|---|---|---|---|---|---|---|---|---|
| SA407858 | Primary tumour | Classical | | Pancreatic Progenitor | | FALSE | Classical | 0.001 |
| SA408414 | Primary tumour | QM-PDA | Squamous | Immunogenic | Classical | FALSE | Classical | 0.002 |
| SA408530 | Primary tumour | Exocrine-like | Squamous | Squamous | Basal-like | FALSE | Basal-like | 0.991 |
| SA408570 | Primary tumour | Exocrine-like | ADEX | ADEX | Classical | FALSE | Classical | 0.096 |
| SA408758 | Primary tumour | Exocrine-like | ADEX | Immunogenic | Classical | FALSE | Classical | 0.001 |
| SA408867 | Primary tumour | QM-PDA | Squamous | Squamous | Basal-like | FALSE | Classical | 0.427 |
| SA409775 | Primary tumour | QM-PDA | Squamous | ADEX | Classical | FALSE | Basal-like | 0.850 |
| SA409923 | Primary tumour | Classical | Pancreatic Progenitor | Pancreatic Progenitor | | FALSE | Classical | 0.005 |
| SA409990 | Cell line | QM-PDA | | Squamous | | FALSE | Basal-like | 0.991 |
| SA410103 | Primary tumour | QM-PDA | Immunogenic | Immunogenic | Classical | FALSE | Classical | 0.138 |
| SA410118 | Primary tumour | Exocrine-like | Immunogenic | ADEX | Classical | FALSE | Classical | 0.014 |
| SA410263 | Primary tumour | QM-PDA | Immunogenic | Squamous | Basal-like | FALSE | Basal-like | 0.991 |
| SA410311 | Primary tumour | Classical | Pancreatic Progenitor | Pancreatic Progenitor | Classical | FALSE | Classical | 0.001 |
| SA410566 | Primary tumour | Classical | | Pancreatic Progenitor | Classical | FALSE | Classical | 0.005 |
| SA410742 | Primary tumour | Classical | Pancreatic Progenitor | Immunogenic | Classical | FALSE | Classical | 0.008 |
| SA410750 | Primary tumour | Classical | Immunogenic | Pancreatic Progenitor | Classical | FALSE | Classical | 0.005 |
| SA410758 | Primary tumour | Classical | Pancreatic Progenitor | Pancreatic Progenitor | Classical | FALSE | Classical | 0.001 |
| SA410763 | Primary tumour | Classical | Immunogenic | Pancreatic Progenitor | Classical | FALSE | Classical | 0.001 |
| SA410859 | Primary tumour | QM-PDA | Squamous | Squamous | Basal-like | FALSE | Basal-like | 0.991 |
| SA410883 | Primary tumour | Exocrine-like | ADEX | ADEX | Classical | FALSE | Classical | 0.014 |
| SA410899 | Primary tumour | Classical | Pancreatic Progenitor | Pancreatic Progenitor | Classical | FALSE | Classical | 0.008 |
| SA410911 | Primary tumour | QM-PDA | Squamous | Squamous | Basal-like | FALSE | Basal-like | 0.991 |
| SA410933 | Primary tumour | Classical | Pancreatic Progenitor | Pancreatic Progenitor | | FALSE | Classical | 0.001 |
| SA410977 | Cell line | QM-PDA | | Squamous | | FALSE | Basal-like | 0.937 |
| SA411001 | Primary tumour | Classical | Pancreatic Progenitor | Pancreatic Progenitor | | FALSE | Classical | 0.014 |
| SA411025 | Cell line | QM-PDA | | Squamous | | FALSE | Basal-like | 0.991 |
| SA411029 | Primary tumour | Classical | Pancreatic Progenitor | Pancreatic Progenitor | | FALSE | Classical | 0.005 |
| SA411189 | Primary tumour | QM-PDA | Squamous | Squamous | Classical | FALSE | Basal-like | 0.860 |
| SA411209 | Primary tumour | Exocrine-like | ADEX | ADEX | Classical | FALSE | Classical | 0.093 |
| SA411241 | Primary tumour | Classical | Pancreatic Progenitor | Pancreatic Progenitor | Classical | FALSE | Classical | 0.005 |
| SA411305 | Primary tumour | QM-PDA | Squamous | ADEX | Classical | FALSE | Basal-like | 0.680 |
| SA411397 | Primary tumour | Classical | Squamous | Immunogenic | Classical | FALSE | Classical | 0.099 |
| SA411406 | Primary tumour | Classical | Immunogenic | Pancreatic Progenitor | Classical | FALSE | Classical | 0.001 |
| SA411430 | Primary tumour | Classical | Immunogenic | Pancreatic Progenitor | | FALSE | Classical | 0.001 |
| SA411557 | Primary tumour | Classical | Immunogenic | Pancreatic Progenitor | | FALSE | Classical | 0.001 |
| SA411578 | Primary tumour | QM-PDA | Squamous | ADEX | Classical | FALSE | Basal-like | 0.902 |
| SA411682 | Cell line | QM-PDA | | Squamous | | FALSE | Basal-like | 0.937 |
| SA411709 | Cell line | QM-PDA | | Squamous | | FALSE | Basal-like | 0.937 |
| SA411721 | Primary tumour | Classical | Pancreatic Progenitor | Pancreatic Progenitor | Classical | FALSE | Classical | 0.001 |
| SA411745 | Primary tumour | Classical | Immunogenic | Pancreatic Progenitor | Classical | FALSE | Classical | 0.001 |
| SA411769 | Primary tumour | Classical | Pancreatic Progenitor | Pancreatic Progenitor | Classical | FALSE | Classical | 0.001 |
| SA411797 | Primary tumour | Classical | Pancreatic Progenitor | Pancreatic Progenitor | | FALSE | Classical | 0.001 |
| SA411833 | Primary tumour | Classical | Immunogenic | Pancreatic Progenitor | Classical | FALSE | Classical | 0.001 |
| SA411841 | Cell line | QM-PDA | | Squamous | | FALSE | Basal-like | 0.937 |
| SA411923 | Primary tumour | Exocrine-like | Squamous | Squamous | Classical | FALSE | Classical | 0.301 |
| SA412003 | Primary tumour | QM-PDA | | ADEX | Classical | FALSE | Classical | 0.092 |
| SA412060 | Cell line | QM-PDA | | Squamous | | FALSE | Basal-like | 0.991 |
| SA412076 | Primary tumour | QM-PDA | Squamous | ADEX | Classical | FALSE | Classical | 0.020 |
| SA412268 | Metastatic tumour | QM-PDA | | Squamous | | FALSE | Basal-like | 0.991 |
| SA412299 | Primary tumour | Exocrine-like | ADEX | ADEX | | FALSE | Classical | 0.020 |
| SA518492 | Cell line | QM-PDA | | Squamous | | FALSE | Basal-like | 0.901 |
| SA518603 | Primary tumour | Classical | Pancreatic Progenitor | Pancreatic Progenitor | Classical | FALSE | Classical | 0.001 |
| SA518614 | Primary tumour | Classical | Immunogenic | Pancreatic Progenitor | | FALSE | Classical | 0.001 |
| SA518615 | Primary tumour | Classical | Immunogenic | Pancreatic Progenitor | Classical | FALSE | Classical | 0.001 |
| SA518624 | | Classical | | Pancreatic Progenitor | | FALSE | Classical | 0.001 |
| SA518630 | Primary tumour | Exocrine-like | ADEX | ADEX | | FALSE | Classical | 0.001 |
| SA518633 | Primary tumour | QM-PDA | Squamous | Squamous | Basal-like | FALSE | Basal-like | 0.991 |
| SA518637 | Primary tumour | QM-PDA | Squamous | Squamous | Basal-like | FALSE | Basal-like | 0.991 |
| SA518665 | Primary tumour | Exocrine-like | ADEX | Pancreatic Progenitor | Classical | FALSE | Classical | 0.001 |
| SA518689 | Primary tumour | Classical | Immunogenic | Pancreatic Progenitor | Classical | FALSE | Classical | 0.001 |
| SA518695 | Primary tumour | Classical | Pancreatic Progenitor | Pancreatic Progenitor | Classical | FALSE | Classical | 0.005 |
| SA518701 | Primary tumour | Exocrine-like | Pancreatic Progenitor | Pancreatic Progenitor | | FALSE | Classical | 0.001 |
| SA518704 | Primary tumour | Exocrine-like | ADEX | ADEX | Classical | FALSE | Classical | 0.013 |
| SA518712 | Primary tumour | Exocrine-like | | ADEX | Classical | FALSE | Classical | 0.013 |
| SA518716 | Primary tumour | Exocrine-like | Immunogenic | Pancreatic Progenitor | Classical | FALSE | Classical | 0.001 |
| SA518724 | Primary tumour | Classical | Pancreatic Progenitor | Pancreatic Progenitor | Classical | FALSE | Classical | 0.001 |
| SA518750 | Primary tumour | Classical | Pancreatic Progenitor | Pancreatic Progenitor | Classical | FALSE | Classical | 0.001 |
| SA518765 | Primary tumour | Classical | Pancreatic Progenitor | Immunogenic | Classical | FALSE | Classical | 0.001 |
| SA518806 | Primary tumour | QM-PDA | Squamous | ADEX | Classical | FALSE | Classical | 0.142 |
| SA518817 | Primary tumour | Classical | Immunogenic | Pancreatic Progenitor | Classical | FALSE | Classical | 0.001 |
| SA518851 | Primary tumour | QM-PDA | Squamous | Squamous | Basal-like | FALSE | Basal-like | 0.991 |
| SA518854 | Primary tumour | Exocrine-like | | ADEX | Classical | FALSE | Classical | 0.092 |
| SA518868 | Primary tumour | QM-PDA | Immunogenic | ADEX | Classical | FALSE | Classical | 0.014 |
| SA518873 | | QM-PDA | | Squamous | | FALSE | Basal-like | 0.991 |
| SA518878 | Primary tumour | QM-PDA | Squamous | ADEX | Classical | FALSE | Classical | 0.001 |
| SA528675 | Primary tumour | Classical | Immunogenic | Pancreatic Progenitor | | FALSE | Classical | 0.001 |
| SA528676 | Primary tumour | QM-PDA | | ADEX | Basal-like | FALSE | Classical | 0.025 |
| SA528677 | Primary tumour | Exocrine-like | ADEX | ADEX | Classical | FALSE | Classical | 0.003 |
| SA528679 | Primary tumour | QM-PDA | Immunogenic | Squamous | Basal-like | FALSE | Basal-like | 0.991 |
| SA528687 | Primary tumour | Classical | Immunogenic | Pancreatic Progenitor | Classical | FALSE | Classical | 0.001 |
| SA528695 | Primary tumour | Classical | Pancreatic Progenitor | Pancreatic Progenitor | Classical | FALSE | Classical | 0.001 |
| SA528701 | Metastatic tumour | Classical | | Pancreatic Progenitor | | FALSE | Classical | 0.014 |
| SA528709 | Primary tumour | QM-PDA | Squamous | Squamous | Basal-like | FALSE | Basal-like | 0.991 |
| SA528711 | Primary tumour | QM-PDA | Pancreatic Progenitor | ADEX | Classical | FALSE | Classical | 0.014 |
| SA528755 | Primary tumour | Classical | Immunogenic | Pancreatic Progenitor | Classical | FALSE | Classical | 0.001 |
| SA528761 | Primary tumour | QM-PDA | Squamous | Squamous | Basal-like | FALSE | Basal-like | 0.762 |
| SA528763 | Primary tumour | QM-PDA | | ADEX | | FALSE | Classical | 0.036 |
| SA528766 | Primary tumour | Exocrine-like | | ADEX | Classical | FALSE | Basal-like | 0.548 |
| SA528767 | Primary tumour | QM-PDA | Squamous | ADEX | Classical | FALSE | Classical | 0.211 |
| SA528768 | Primary tumour | Classical | Pancreatic Progenitor | Pancreatic Progenitor | | FALSE | Classical | 0.002 |
| SA528769 | Primary tumour | Classical | | ADEX | Classical | FALSE | Classical | 0.001 |
| SA528771 | Primary tumour | Classical | | ADEX | Classical | FALSE | Classical | 0.005 |

TABLE 16
TCGA_PAAD

| | | | | | | PurIST.train- | | PurIST.bas- |
|---|---|---|---|---|---|---|---|---|
| ID | Collisson_original | Collisson | Bailey_original | Bailey | Moffitt | ing | PurIST | al.prob |
| TCGA-2L-AAQE-01A | Classical | Classical | Immunogenic | Immunogenic | Classical | FALSE | Classical | 0.436 |
| TCGA-XD-AAUL-01A | Classical | Classical | Immunogenic | Immunogenic | Classical | TRUE | Classical | 0.148 |
| TCGA-2L-AAQJ-01A | Classical | Classical | Progenitor | Pancreatic Progenitor | Classical | TRUE | Classical | 0.001 |
| TCGA-2L-AAQI-01A | Exocrine-like | Exocrine-like | Progenitor | Pancreatic Progenitor | Classical | TRUE | Classical | 0.038 |
| TCGA-3A-A9IB-01A | Exocrine-like | Exocrine-like | ADEX | Squamous | Basal-like | TRUE | Basal-like | 0.548 |
| TCGA-3A-A9IU-01A | Exocrine-like | Exocrine-like | ADEX | Squamous | Basal-like | TRUE | Basal-like | 0.548 |
| TCGA-FB-AAPS-01A | Exocrine-like | Exocrine-like | ADEX | Squamous | Classical | TRUE | Classical | 0.032 |
| TCGA-HV-AA8X-01A | Classical | Classical | Progenitor | Pancreatic Progenitor | Classical | TRUE | Classical | 0.001 |
| TCGA-LB-A9Q5-01A | Exocrine-like | Exocrine-like | Squamous | ADEX | Classical | TRUE | Classical | 0.165 |
| TCGA-HZ-A9TJ-01A | Classical | Classical | Progenitor | Pancreatic Progenitor | Classical | TRUE | Classical | 0.002 |
| TCGA-3A-A9IH-01A | Classical | Classical | Immunogenic | Immunogenic | Classical | TRUE | Basal-like | 0.785 |
| TCGA-RB-AA9M-01A | Classical | Classical | Immunogenic | Immunogenic | Basal-like | FALSE | Classical | 0.308 |
| TCGA-IB-AAUQ-01A | QM-PDA | QM-PDA | ADEX | Squamous | Basal-like | TRUE | Basal-like | 0.939 |
| TCGA-3A-A9J0-01A | Classical | Classical | Immunogenic | Immunogenic | Classical | TRUE | Classical | 0.008 |
| TCGA-FB-AAQ3-01A | Exocrine-like | Exocrine-like | Progenitor | Pancreatic Progenitor | Classical | TRUE | Classical | 0.001 |
| TCGA-FB-AAQ1-01A | Exocrine-like | Exocrine-like | Squamous | ADEX | Basal-like | TRUE | Basal-like | 0.991 |
| TCGA-2J-AAB9-01A | Exocrine-like | Exocrine-like | Squamous | ADEX | Basal-like | FALSE | Classical | 0.090 |
| TCGA-2J-AABA-01A | Classical | Classical | Immunogenic | Immunogenic | Classical | TRUE | Classical | 0.034 |
| TCGA-2J-AABR-01A | Exocrine-like | Exocrine-like | Squamous | ADEX | Classical | FALSE | Basal-like | 0.957 |
| TCGA-FB-AAQ6-01A | Classical | Classical | Progenitor | Pancreatic Progenitor | Classical | TRUE | Classical | 0.001 |
| TCGA-2J-AABE-01A | Classical | Classical | Progenitor | Pancreatic Progenitor | Classical | TRUE | Classical | 0.013 |
| TCGA-2J-AABT-01A | QM-PDA | QM-PDA | Immunogenic | Immunogenic | Classical | TRUE | Classical | 0.003 |
| TCGA-FB-AAPQ-01A | Classical | Classical | Progenitor | Pancreatic Progenitor | Classical | TRUE | Classical | 0.001 |
| TCGA-HV-AA8V-01A | Classical | Classical | ADEX | Squamous | Basal-like | FALSE | Classical | 0.087 |
| TCGA-2J-AABV-01A | Exocrine-like | Exocrine-like | Squamous | ADEX | Classical | TRUE | Classical | 0.001 |
| TCGA-2J-AABF-01A | Exocrine-like | Exocrine-like | Progenitor | Pancreatic Progenitor | Classical | TRUE | Classical | 0.001 |
| TCGA-2J-AABU-01A | QM-PDA | QM-PDA | ADEX | Squamous | Basal-like | TRUE | Basal-like | 0.939 |
| TCGA-FB-AAPU-01A | Classical | Classical | Progenitor | Pancreatic Progenitor | Classical | TRUE | Classical | 0.001 |
| TCGA-2J-AABH-01A | Exocrine-like | Exocrine-like | Progenitor | Pancreatic Progenitor | Classical | TRUE | Classical | 0.005 |
| TCGA-FB-AAPY-01A | Exocrine-like | Exoctine-like | Progenitor | Pancreatic Progenitor | Classical | TRUE | Classical | 0.002 |
| TCGA-2J-AAB1-01A | Classical | Classical | Progenitor | Pancreatic Progenitor | Classical | TRUE | Classical | 0.001 |
| TCGA-XD-AAUG-01A | Exocrine-like | Exocrine-like | Squamous | ADEX | Classical | TRUE | Classical | 0.002 |
| TCGA-2J-AAB4-01A | Classical | Classical | Progenitor | Pancreatic Progenitor | Classical | TRUE | Classical | 0.001 |
| TCGA-2J-AABI-01A | QM-PDA | QM-PDA | ADEX | Squamous | Basal-like | TRUE | Basal-like | 0.991 |
| TCGA-FB-AAPZ-01A | Exocrine-like | Exocrine-like | ADEX | Squamous | Classical | TRUE | Classical | 0.142 |
| TCGA-XD-AAUH-01A | QM-PDA | QM-PDA | ADEX | Squamous | Classical | TRUE | Classical | 0.003 |
| TCGA-3A-A9IX-01A | Exocrine-like | Exocrine-like | Squamous | ADEX | Classical | TRUE | Classical | 0.013 |
| TCGA-2J-AABK-01A | Exocrine-like | Exocrine-like | Progenitor | Pancreatic Progenitor | Classical | TRUE | Classical | 0.002 |
| TCGA-2J-AAB6-01A | QM-PDA | QM-PDA | ADEX | Squamous | Basal-like | TRUE | Basal-like | 0.991 |
| TCGA-FB-AAQ0-01A | Classical | Classical | Progenitor | Pancreatic Progenitor | Classical | TRUE | Classical | 0.001 |
| TCGA-XD-AAUI-01A | Exocrine-like | Exocrine-like | ADEX | Squamous | Classical | TRUE | Classical | 0.096 |
| TCGA-2J-AAB8-01A | Classical | Classical | Immunogenic | Immunogenic | Classical | TRUE | Classical | 0.001 |
| TCGA-3A-A9IZ-01A | QM-PDA | QM-PDA | Immunogenic | Immunogenic | Classical | TRUE | Classical | 0.054 |
| TCGA-2J-AABO-01A | Exocrine-like | Exocrine-like | ADEX | Squamous | Classical | FALSE | Classical | 0.064 |
| TCGA-Z5-AAPL-01A | QM-PDA | QM-PDA | ADEX | Squamous | Classical | TRUE | Classical | 0.004 |
| TCGA-FB-AAQ2-01A | QM-PDA | QM-PDA | ADEX | Squamous | Basal-like | TRUE | Basal-like | 0.991 |
| TCGA-F2-6879-01A | Classical | Classical | Progenitor | Pancreatic Progenitor | Classical | TRUE | Classical | 0.002 |
| TCGA-HZ-7925-01A | QM-PDA | QM-PDA | Immunogenic | Immunogenic | Classical | TRUE | Classical | 0.234 |
| TCGA-IB-7651-01A | Exocrine-like | Exocrine-like | Squamous | ADEX | Classical | TRUE | Classical | 0.008 |
| TCGA-HZ-7926-01A | Exocrine-like | Exocrine-like | Immunogenic | Immunogenic | Classical | TRUE | Classical | 0.008 |
| TCGA-IB-7885-01A | QM-PDA | QM-PDA | ADEX | Squamous | Classical | FALSE | Classical | 0.325 |
| TCGA-IB-7652-01A | Exocrine-like | Exocrine-like | Squamous | ADEX | Classical | TRUE | Classical | 0.002 |
| TCGA-IB-7644-01A | Classical | Classical | Progenitor | Pancreatic Progenitor | Classical | TRUE | Classical | 0.001 |
| TCGA-IB-7887-01A | Classical | Classical | Immunogenic | Immunogenic | Basal-like | FALSE | Classical | 0.087 |
| TCGA-IB-7889-01A | Exocrine-like | Exocrine-like | Squamous | ADEX | Classical | TRUE | Classical | 0.016 |
| TCGA-IB-7646-01A | QM-PDA | QM-PDA | ADEX | Squamous | Basal-like | TRUE | Basal-like | 0.991 |
| TCGA-IB-7886-01A | Classical | Classical | Immunogenic | Immunogenic | Classical | TRUE | Classical | 0.005 |
| TCGA-IB-7893-01A | QM-PDA | QM-PDA | ADEX | Squamous | Basal-like | TRUE | Basal-like | 0.855 |
| TCGA-HZ-7919-01A | Classical | Classical | Progenitor | Pancreatic Progenitor | Classical | TRUE | Classical | 0.005 |
| TCGA-HZ-8001-01A | Exocrine-like | Exocrine-like | ADEX | Squamous | Basal-like | TRUE | Basal-like | 0.978 |
| TCGA-IB-7647-01A | Classical | Classical | Immunogenic | Immunogenic | Classical | TRUE | Classical | 0.001 |
| TCGA-IB-7897-01A | QM-PDA | QM-PDA | Squamous | ADEX | Classical | TRUE | Classical | 0.036 |
| TCGA-IB-7888-01A | Classical | Classical | Immunogenic | Immunogenic | Classical | TRUE | Classical | 0.001 |
| TCGA-HZ-8002-01A | Exocrine-like | Exocrine-like | Squamous | ADEX | Classical | TRUE | Classical | 0.200 |
| TCGA-HZ-7922-01A | Exocrine-like | Exocrine-like | Squamous | ADEX | Classical | TRUE | Classical | 0.014 |
| TCGA-HZ-8003-01A | Exocrine-like | Exocrine-like | Squamous | ADEX | Classical | TRUE | Classical | 0.001 |
| TCGA-IB-7649-01A | Exocrine-like | Exocrine-like | Squamous | ADEX | Classical | TRUE | Classical | 0.002 |
| TCGA-IB-7645-01A | QM-PDA | QM-PDA | Immunogenic | Immunogenic | Classical | TRUE | Classical | 0.001 |
| TCGA-IB-7890-01A | QM-PDA | QM-PDA | ADEX | Squamous | Basal-like | TRUE | Basal-like | 0.978 |
| TCGA-IB-7891-01A | Exocrine-like | Exocrine-like | Squamous | ADEX | Classical | TRUE | Classical | 0.005 |
| TCGA-H6-8124-01A | QM-PDA | QM-PDA | ADEX | Squamous | Basal-like | TRUE | Basal-like | 0.937 |
| TCGA-HZ-8315-01A | Exocrine-like | Exocrine-like | Squamous | ADEX | Classical | TRUE | Classical | 0.034 |
| TCGA-HZ-8317-01A | Exocrine-like | Exocrine-like | Squamous | ADEX | Classical | TRUE | Classical | 0.038 |
| TCGA-HZ-8519-01A | Exocrine-like | Exocrine-like | Squamous | ADEX | Classical | TRUE | Classical | 0.008 |
| TCGA-HZ-8636-01A | QM-PDA | QM-PDA | Immunogenic | Immunogenic | Classical | TRUE | Classical | 0.005 |
| TCGA-HZ-8637-01A | QM-PDA | QM-PDA | Immunogenic | Immunogenic | Classical | TRUE | Classical | 0.001 |
| TCGA-IB-8127-01A | Exocrine-like | Exocrine-like | Immunogenic | Immunogenic | Classical | TRUE | Classical | 0.165 |
| TCGA-IB-8126-01A | Exocrine-like | Exocrine-like | Squamous | ADEX | Classical | TRUE | Classical | 0.001 |
| TCGA-F2-A44H-01A | Exocrine-like | Exocrine-like | Squamous | ADEX | Classical | TRUE | Classical | 0.005 |
| TCGA-FB-A4P6-01A | Exocrine-like | Exocrine-like | Squamous | ADEX | Classical | TRUE | Classical | 0.013 |
| TCGA-FB-A4P5-01A | Exocrine-like | Exocrine-like | Squamous | ADEX | Classical | TRUE | Classical | 0.038 |
| TCGA-H6-A45N-01A | Exocrine-like | Exocrine-like | Squamous | ADEX | Classical | TRUE | Classical | 0.002 |
| TCGA-HV-A5A3-01A | Classical | Classical | Immunogenic | Immunogenic | Basal-like | TRUE | Basal-like | 0.902 |
| TCGA-HV-A5A5-01A | Exocrine-like | Exocrine-like | Squamous | ADEX | Classical | TRUE | Classical | 0.002 |
| TCGA-HV-A5A4-01A | Classical | Classical | Progenitor | Pancreatic Progenitor | Classical | TRUE | Classical | 0.002 |
| TCGA-HZ-A49H-01A | Exocrine-like | Exocrine-like | Squamous | ADEX | Classical | TRUE | Classical | 0.002 |
| TCGA-HV-A5A6-01A | Classical | Classical | Immunogenic | Immunogenic | Classical | TRUE | Classical | 0.021 |
| TCGA-HZ-A49G-01A | Exocrine-like | Exocrine-like | Squamous | ADEX | Classical | TRUE | Classical | 0.038 |
| TCGA-HZ-A4BH-01A | Classical | Classical | Immunogenic | Immunogenic | Classical | TRUE | Classical | 0.013 |
| TCGA-HZ-A49I-01A | Classical | Classical | Progenitor | Pancreatic Progenitor | Classical | TRUE | Classical | 0.003 |
| TCGA-HZ-A4BK-01A | Exocrine-like | Exocrine-like | Progenitor | Pancreatic Progenitor | Classical | TRUE | Classical | 0.001 |
| TCGA-M8-A5N4-01A | Classical | Classical | Immunogenic | Immunogenic | Classical | TRUE | Classical | 0.229 |
| TCGA-F2-A44G-01A | Classical | Classical | Immunogenic | Immunogenic | Classical | TRUE | Classical | 0.003 |
| TCGA-HZ-8005-01A | QM-PDA | QM-PDA | ADEX | Squamous | Basal-like | TRUE | Basal-like | 0.902 |
| TCGA-PZ-A5RE-01A | Classical | Classical | Progenitor | Pancreatic Progenitor | Classical | TRUE | Classical | 0.005 |
| TCGA-FB-A78T-01A | Exocrine-like | Exocrine-like | Progenitor | Pancreatic Progenitor | Classical | TRUE | Classical | 0.001 |
| TCGA-FB-A5VM-01A | QM-PDA | QM-PDA | ADEX | Squamous | Basal-like | TRUE | Basal-like | 0.991 |
| TCGA-US-A774-01A | Exocrine-like | Exocrine-like | Squamous | ADEX | Classical | TRUE | Classical | 0.003 |
| TCGA-OE-A75W-01A | Classical | Classical | Progenitor | Pancreatic Progenitor | Classical | TRUE | Classical | 0.002 |
| TCGA-US-A779-01A | Classical | Classical | Progenitor | Pancreatic Progenitor | Classical | TRUE | Classical | 0.001 |
| TCGA-IB-A5SP-01A | Classical | Classical | Progenitor | Pancreatic Progenitor | Classical | TRUE | Classical | 0.001 |
| TCGA-IB-A5SQ-01A | QM-PDA | QM-PDA | ADEX | Squamous | Basal-like | TRUE | Basal-like | 0.965 |
| TCGA-US-A77G-01A | Exocrine-like | Exocrine-like | Progenitor | Pancreatic Progenitor | Classical | TRUE | Classical | 0.001 |
| TCGA-US-A77E-01A | Classical | Classical | Progenitor | Pancreatic Progenitor | Classical | TRUE | Classical | 0.002 |
| TCGA-Q3-A5QY-01A | QM-PDA | QM-PDA | ADEX | Squamous | Basal-like | FALSE | Basal-like | 0.557 |
| TCGA-IB-A5ST-01A | QM-PDA | QM-PDA | ADEX | Squamous | Classical | TRUE | Classical | 0.096 |
| TCGA-IB-A5SO-01A | Classical | Classical | Immunogenic | Immunogenic | Classical | TRUE | Classical | 0.005 |
| TCGA-IB-A5SS-01A | QM-PDA | QM-PDA | ADEX | Squamous | Basal-like | TRUE | Basal-like | 0.902 |
| TCGA-IB-A6UF-01A | Exocrine-like | Exocrine-like | ADEX | Squamous | Basal-like | TRUE | Basal-like | 0.991 |
| TCGA-HV-A7OL-01A | Classical | Classical | Progenitor | Pancreatic Progenitor | Classical | TRUE | Classical | 0.002 |
| TCGA-IB-A6UG-01A | Exocrine-like | Exocrine-like | Squamous | ADEX | Basal-like | TRUE | Basal-like | 0.557 |
| TCGA-HZ-A77P-01A | Exocrine-like | Exocrine-like | Squamous | ADEX | Classical | TRUE | Classical | 0.002 |
| TCGA-HZ-A77O-01A | Exocrine-like | Exocrine-like | Squamous | ADEX | Basal-like | FALSE | Basal-like | 0.548 |
| TCGA-LB-A7SX-01A | Classical | Classical | Immunogenic | Immunogenic | Basal-like | TRUE | Classical | 0.388 |
| TCGA-RB-A7B8-01A | Classical | Classical | Progenitor | Pancreatic Progenitor | Classical | TRUE | Classical | 0.001 |
| TCGA-US-A776-01A | Classical | Classical | Progenitor | Pancreatic Progenitor | Classical | TRUE | Classical | 0.005 |
| TCGA-HZ-A8P0-01A | Exocrine-like | Exocrine-like | Immunogenic | Immunogenic | Classical | TRUE | Classical | 0.004 |
| TCGA-IB-A7LX-01A | QM-PDA | QM-PDA | ADEX | Squamous | Basal-like | TRUE | Basal-like | 0.991 |
| TCGA-HZ-A77Q-01A | QM-PDA | QM-PDA | ADEX | Squamous | Classical | TRUE | Classical | 0.051 |
| TCGA-IB-A7M4-01A | QM-PDA | QM-PDA | ADEX | Squamous | Classical | FALSE | Basal-like | 0.860 |
| TCGA-XN-A8T5-01A | QM-PDA | QM-PDA | ADEX | Squamous | Basal-like | FALSE | Classical | 0.055 |
| TCGA-LB-A8F3-01A | Exocrine-like | Exocrine-like | Squamous | ADEX | Classical | TRUE | Classical | 0.024 |
| TCGA-YB-A89D-01A | Classical | Classical | Progenitor | Pancreatic Progenitor | Classical | TRUE | Classical | 0.002 |
| TCGA-YY-A8LH-01A | Classical | Classical | Progenitor | Pancreatic Progenitor | Classical | TRUE | Classical | 0.001 |
| TCGA-S4-A8RP-01A | Exocrine-like | Exocrine-like | Progenitor | Pancreatic Progenitor | Classical | TRUE | Classical | 0.001 |
| TCGA-XN-A8T3-01A | Exocrine-like | Exocrine-like | Squamous | ADEX | Basal-like | TRUE | Basal-like | 0.902 |
| TCGA-F2-A8YN-01A | Classical | Classical | Immunogenic | Immunogenic | Classical | TRUE | Classical | 0.022 |
| TCGA-S4-A8RO-01A | Classical | Classical | Immunogenic | Immunogenic | Basal-like | FALSE | Basal-like | 0.707 |
| TCGA-HZ-A8P1-01A | Classical | Classical | Progenitor | Pancreatic Progenitor | Classical | TRUE | Classical | 0.001 |
| TCGA-IB-AAUM-01A | Exocrine-like | Exocrine-like | Squamous | ADEX | Classical | TRUE | Classical | 0.001 |
| TCGA-IB-AAUP-01A | QM-PDA | QM-PDA | ADEX | Squamous | Classical | TRUE | Classical | 0.004 |
| TCGA-IB-AAUT-01A | Exocrine-like | Exocrine-like | Squamous | ADEX | Classical | TRUE | Classical | 0.002 |
| TCGA-YH-A8SY-01A | QM-PDA | QM-PDA | ADEX | Squamous | Basal-like | TRUE | Basal-like | 0.779 |
| TCGA-IB-AAUU-01A | Classical | Classical | Progenitor | Pancreatic Progenitor | Classical | TRUE | Classical | 0.001 |
| TCGA-IB-AAUS-01A | Exocrine-like | Exocrine-like | ADEX | Squamous | Classical | TRUE | Classical | 0.090 |
| TCGA-Q3-AA2A-01A | Classical | Classical | Progenitor | Pancreatic Progenitor | Classical | TRUE | Classical | 0.001 |
| TCGA-S4-A8RM-01A | Classical | Classical | Progenitor | Pancreatic Progenitor | Classical | TRUE | Classical | 0.001 |
| TCGA-2L-AAQA-01A | Classical | Classical | Progenitor | Pancreatic Progenitor | Classical | TRUE | Classical | 0.002 |
| TCGA-2L-AAQL-01A | Exocrine-like | Exocrine-like | Progenitor | Pancreatic Progenitor | Classical | TRUE | Classical | 0.001 |
| TCGA-3A-A9I5-01A | Exocrine-like | Exocrine-like | Squamous | ADEX | Basal-like | TRUE | Classical | 0.008 |
| TCGA-3A-A9I9-01A | Exocrine-like | Exocrine-like | Squamous | ADEX | Classical | TRUE | Classical | 0.003 |
| TCGA-3A-A9I7-01A | Exocrine-like | Exocrine-like | Immunogenic | Immunogenic | Classical | TRUE | Classical | 0.064 |
| TCGA-3E-AAAY-01A | Exocrine-like | Exocrine-like | Squamous | ADEX | Classical | TRUE | Classical | 0.002 |
| TCGA-3E-AAAZ-01A | Exocrine-like | Exocrine-like | Immunogenic | Immunogenic | Classical | TRUE | Classical | 0.003 |
| TCGA-F2-A7TX-01A | Exocrine-like | Exocrine-like | Squamous | ADEX | Basal-like | TRUE | Basal-like | 0.902 |
| TCGA-IB-AAUN-01A | Exocrine-like | Exocrine-like | Squamous | ADEX | Basal-like | FALSE | Basal-like | 0.786 |
| TCGA-M-AAUO-01A | QM-PDA | QM-PDA | ADEX | Squamous | Basal-like | TRUE | Basal-like | 0.899 |
| TCGA-3A-A9IC-01A | QM-PDA | QM-PDA | Immunogenic | Immunogenic | Classical | TRUE | Classical | 0.009 |
| TCGA-IB-AAUR-01A | QM-PDA | QM-PDA | ADEX | Squamous | Classical | TRUE | Classical | 0.013 |
| TCGA-FB-A545-01A | QM-PDA | QM-PDA | ADEX | Squamous | Basal-like | TRUE | Basal-like | 0.855 |

TABLE 17
Yeh_seq

| | | | | | | | | | | | | Neoadj. |
|---|---|---|---|---|---|---|---|---|---|---|---|---|
| | | | Sam- | | | PurIST. | Survival | | | | | Tx. |
| | Path- | Tis- | ple | | | basal. | Anal- | Clini- | Adj. | Adj.Tx. | Neoadj. | Regi- |
| ID | ology | sueType | Type | Moffitt | PurIST | prob | ysis | calType | Tx | Regimen | Tx | men |
| S001.FNA.Pi.0422T1 | adeno | Primary | FNA | Basal- | Basal- | 0.762 | TRUE | Panc | yes | gemcitabine | no | |
| | | PDAC | | like | like | | | | | | | |
| S002.FNA.Pi.0825T1 | adeno | Primary | FNA | Classi- | Classi- | 0.001 | TRUE | Panc | yes | gemcitabine | no | |
| | | PDAC | | cal | cal | | | Tumor | | | | |
| S003.FNA.Pi.1119T1 | adeno | Primary | FNA | Basal- | Basal- | 0.991 | TRUE | | yes | (gem w compl | no | |
| | | PDAC | | like | like | | | | | and difficulty | | |
| | | | | | | | | | | tol) | | |
| S004.FNA.Pi.0517T1 | adeno | Primary | FNA | Classi- | Classi- | 0.002 | TRUE | | yes | gem + erlotinib | no | |
| | | PDAC | | cal | cal | | | | | | | |
| S005.FNA.Pi.0818T1 | adeno | Primary | FNA | Classi- | Classi- | 0.001 | TRUE | | yes | 5FU/RT | no | |
| | | PDAC | | cal | cal | | | | | | | |
| S006.FNA.Pi.1012T1 | adeno | Primary | FNA | Classi- | Classi- | 0.001 | TRUE | | LTFO | | no | |
| | | PDAC | | cal | cal | | | | | | | |
| S007.FNA.Pi.1118T1 | adeno | Primary | FNA | Classi- | Classi- | 0.039 | TRUE | | yes | gem | no | |
| | | PDAC | | cal | cal | | | | | | | |
| S008.FNA.Pi.0105T1 | adeno | Primary | FNA | Classi- | Classi- | 0.013 | TRUE | | yes | gem | no | |
| | | PDAC | | cal | cal | | | | | | | |
| S009.FNA.Pi.0119T1 | adeno | Primary | FNA | Basal- | Classi- | 0.064 | TRUE | | yes | unknown | no | |
| | | PDAC | | like | cal | | | | | systemic | | |
| S010.FNA.Pi.0417T1 | adeno | Primary | FNA | Classi- | Classi- | 0.013 | TRUE | | yes | gem | no | |
| | | PDAC | | cal | cal | | | | | | | |
| S011.FNA.Pi.0503T1 | adeno | Primary | FNA | Classi- | Classi- | 0.001 | TRUE | | yes | gem + 5fu/rt | no | |
| | | PDAC | | cal | cal | | | | | | | |
| S012.FNA.Pi.0921T1 | adeno | Primary | FNA | Classi- | Classi- | 0.002 | TRUE | | yes | 5FU/RT | no | |
| | | PDAC | | cal | cal | | | | | | | |
| S013.FNA.Pi.1109T1 | adeno | Primary | FNA | Classi- | Classi- | 0.001 | TRUE | Panc | yes | gem | no | |
| | | PDAC | | cal | cal | | | Tumor | | | | |
| S014.FNA.Pi.1129T1 | adeno | Primary | FNA | Classi- | Classi- | 0.002 | TRUE | Panc | yes | gem + 5FU/RT | no | |
| | | PDAC | | cal | cal | | | Tumor | | | | |
| S015.FNA.Pi.1206T1 | adeno | Primary | FNA | Classi- | Classi- | 0.003 | TRUE | Panc | yes | Folfirinox | yes | folfiri- |
| | | PDAC | | cal | cal | | | Tumor | | | | nox |
| S016.FNA.Pi.1214T1 | adeno | Primary | FNA | Classi- | Classi- | 0.008 | TRUE | Panc | DOO | | no | |
| | | PDAC | | cal | cal | | | Tumor | | | | |
| S017.FNA.Pi.0124T1 | adeno | Primary | FNA | Classi- | Classi- | 0.001 | TRUE | Panc | yes | gem + 5FU/RT | no | |
| | | PDAC | | cal | cal | | | Tumor | | | | |
| S018.FNA.Pi.0221T1 | adeno | Primary | FNA | Classi- | Classi- | 0.001 | TRUE | Panc | yes | gem | no | |
| | | PDAC | | cal | cal | | | Tumor | | | | |
| S019.FNA.Pi.0222T1 | adeno | Primary | FNA | Classi- | Classi- | 0.001 | TRUE | Panc | yes | gem | no | |
| | | PDAC | | cal | cal | | | Tumor | | | | |
| S020.FNA.Pi.0327T1 | adeno | Primary | FNA | Classi- | Classi- | 0.002 | TRUE | Panc | yes | gem | yes | 5FU/ |
| | | PDAC | | cal | cal | | | Tumor | | | | RT |
| S021.FNA.Pi.0328T1 | adeno | Primary | FNA | Classi- | Classi- | 0.001 | TRUE | | yes | RT/gem + gem | no | |
| | | PDAC | | cal | cal | | | | | | | |
| S022.FNA.Pi.0411T1 | adeno | Primary | FNA | Classi- | Classi- | 0.001 | TRUE | | yes | gem | yes | 5FU/ |
| | | PDAC | | cal | cal | | | | | | | RT |
| S023.FNA.Pi.0417T1 | adeno | Primary | FNA | Classi- | Classi- | 0.001 | TRUE | | yes | gem + 5FU/RT | no | |
| | | PDAC | | cal | cal | | | | | | | |
| S024.FNA.Pi.0425T1 | adeno | Primary | FNA | Classi- | Classi- | 0.003 | TRUE | Panc | yes | gem + 5FU/RT | no | |
| | | PDAC | | cal | cal | | | Tumor | | | | |
| S025.FNA.Pi.0502T2 | adeno | Primary | FNA | Classi- | Classi- | 0.001 | TRUE | | yes | gem + 5FU/RT | no | |
| | | PDAC | | cal | cal | | | | | | | |
| S026.FNA.Pi.0508T1 | adeno | Primary | FNA | Classi- | Classi- | 0.040 | TRUE | | yes | gem + 5FU/RT | no | |
| | | PDAC | | cal | cal | | | | | | | |
| S027.FNA.Pi.0523T1 | adeno | Primary | FNA | Classi- | Classi- | 0.002 | TRUE | Panc | no | | no | |
| | | PDAC | | cal | cal | | | Tumor | | | | |
| S028.FNA.Pi.0524T1 | ampul- | Primary | FNA | Classi- | Classi- | 0.001 | FALSE | | | gem + 5FU | | |
| | lary | PDAC | | cal | cal | | | | | | | |
| S029.FNA.Pi.0605T1 | adeno | Primary | FNA | Classi- | Classi- | 0.001 | TRUE | | yes | gem | no | |
| | | PDAC | | cal | cal | | | | | | | |
| S030.FNA.Pi.0607T1 | adeno | Primary | FNA | Classi- | Classi- | 0.002 | TRUE | | yes | 5FU/RT | no | |
| | | PDAC | | cal | cal | | | | | | | |
| S031.FNA.Pi.0614T1 | adeno | Primary | FNA | Classi- | Classi- | 0.008 | TRUE | | yes | gem + 5FU/RT | no | |
| | | PDAC | | cal | cal | | | | | | | |
| S032.FNA.Pi.0710T2 | adeno | Primary | FNA | Classi- | Classi- | 0.001 | FALSE | Panc | yes | gem + 5fu/RT | no | |
| | | PDAC | | cal | cal | | | Normal, | | | | |
| | | | | | | | | IPMN | | | | |
| | | | | | | | | patient | | | | |
| S033.FNA.Pi.0711T1 | adeno | Primary | FNA | Classi- | Classi- | 0.013 | TRUE | | LTFO | | no | |
| | | PDAC | | cal | cal | | | | | | | |
| S034.FNA.Pi.0904T1 | adeno | Primary | FNA | Classi- | Classi- | 0.001 | TRUE | | yes | gem + RT/5FU | no | |
| | | PDAC | | cal | cal | | | | | | | |
| S035.FNA.Pi.1009T1 | adeno | Primary | FNA | Classi- | Classi- | 0.001 | TRUE | | no | | yes | 5FU/ |
| | | PDAC | | cal | cal | | | | | | | RT |
| S036.FNA.Pi.1119T1 | adeno | Primary | FNA | Basal- | Classi- | 0.165 | TRUE | | yes | Folfirinox | no | |
| | | PDAC | | like | cal | | | | | | | |
| S037.FNA.Pi.1204T1 | adeno | Primary | FNA | Classi- | Classi- | 0.001 | TRUE | | yes | gem | no | |
| | | PDAC | | cal | cal | | | | | | | |
| S038.FNA.Pi.1205T1 | adeno | Primary | FNA | Classi- | Classi- | 0.090 | TRUE | | yes | gem + 5FU/RT | yes | gem |
| | | PDAC | | cal | cal | | | | | | | |
| S039.FNA.Pi.0129T1 | adeno | Primary | FNA | Classi- | Classi- | 0.024 | TRUE | | yes | gem + RT | no | |
| | | PDAC | | cal | cal | | | | | | | |
| S040.FNA.Pi.0417T1 | adeno | Primary | FNA | Classi- | Classi- | 0.008 | TRUE | | yes | Gem + 5FU/RT | no | |
| | | PDAC | | cal | cal | | | | | | | |
| S041.FNA.Pi.0424T1 | adeno | Primary | FNA | Basal- | Classi- | 0.002 | TRUE | | yes | gem + 5FU/RT | no | |
| | | PDAC | | like | cal | | | | | | | |
| S042.FNA.Pi.0806T1 | adeno | Primary | FNA | Classi- | Classi- | 0.001 | TRUE | | yes | gem + 5FU/RT | no | |
| | | PDAC | | cal | cal | | | | | | | |
| S043.FNA.Pi.0121T1 | adeno | Primary | FNA | Classi- | Classi- | 0.001 | TRUE | | yes | gem + 5FU/RT | no | |
| | | PDAC | | cal | cal | | | | | | | |
| S044.FNA.Pi.0608T1 | adeno | Primary | FNA | Classi- | Classi- | 0.001 | TRUE | | yes | gem + 5fu/rt | no | |
| | | PDAC | | cal | cal | | | | | | | |
| S045.FNA.Pi.1207T1 | adeno | Primary | FNA | Classi- | Classi- | 0.002 | TRUE | | yes | gem + 5FU/RT | no | |
| | | PDAC | | cal | cal | | | | | | | |
| S046.FNA.PDX.0616T1 | adeno | PDX | FNA | Classi- | Classi- | 0.001 | FALSE | | | | | |
| | | | | cal | cal | | | | | | | |
| S047.FNA.PDX.0508T1 | adeno | PDX | FNA | Classi- | Classi- | 0.013 | FALSE | | | | | |
| | | | | cal | cal | | | | | | | |
| S048.FNA.PDX.0902T1B | | PDX | FNA | Classi- | Classi- | 0.001 | FALSE | | | | | |
| | | | | cal | cal | | | | | | | |
| S049.FFPE.PDX.1222T1 | adeno | PDX | FFPE | Classi- | Classi- | 0.001 | FALSE | | | | | |
| | | | | cal | cal | | | | | | | |
| S050.FFPE.PDX.0113T1 | | PDX | FFPE | Basal- | Classi- | 0.223 | FALSE | | | | | |
| | | | | like | cal | | | | | | | |
| S051.FFPE.PDX.1108T1 | adeno | PDX | FFPE | Classi- | Classi- | 0.005 | FALSE | | | | | |
| | | | | cal | cal | | | | | | | |
| S052.FFPE.PDX.1109T1 | adeno | PDX | FFPE | Classi- | Classi- | 0.002 | FALSE | | | | | |
| | | | | cal | cal | | | | | | | |
| S053.FFPE.PDX.1109T1 | adeno | PDX | FFPE | Classi- | Classi- | 0.001 | FALSE | | | | | |
| | | | | cal | cal | | | | | | | |
| S054.FFPE.PDX.0417T1 | adeno | PDX | FFPE | Classi- | Classi- | 0.003 | FALSE | | | | | |
| | | | | cal | cal | | | | | | | |
| S055.FFPE.PDX.0910T1 | adeno | PDX | FFPE | Classi- | Classi- | 0.001 | FALSE | | | | | |
| | | | | cal | cal | | | | | | | |
| S056.FF.PDX.1222T1 | adeno | PDX | FF | Classi- | Classi- | 0.003 | FALSE | | | | | |
| | | | | cal | cal | | | | | | | |
| S057.FF.PDX.0113T1 | | PDX | FF | Classi- | Classi- | 0.021 | FALSE | | | | | |
| | | | | cal | cal | | | | | | | |
| S058.FF.PDX.1108T1 | adeno | PDX | FF | Classi- | Classi- | 0.014 | FALSE | | | | | |
| | | | | cal | cal | | | | | | | |
| S059.FF.PDX.1108T1 | adeno | PDX | FF | Classi- | Classi- | 0.014 | FALSE | | | | | |
| | | | | cal | cal | | | | | | | |
| S060.FF.PDX.0411T1 | adeno | PDX | FF | Classi- | Classi- | 0.001 | FALSE | | | | | |
| | | | | cal | cal | | | | | | | |
| S061.FF.PDX.0523T1 | adeno | PDX | FF | Basal- | Classi- | 0.093 | FALSE | | | | | |
| | | | | like | cal | | | | | | | |
| S062.FF.PDX.0319T1 | | PDX | FF | Classi- | Classi- | 0.005 | FALSE | | | | | |
| | | | | cal | cal | | | | | | | |
| S063.FF.PDX.0119T1 | | PDX | FF | Classi- | Classi- | 0.001 | FALSE | | | | | |
| | | | | cal | cal | | | | | | | |
| S064.FF.PDX.0218T2 | | PDX | FF | Classi- | Classi- | 0.001 | FALSE | | | | | |
| | | | | cal | cal | | | | | | | |
| S065.FF.PDX.0225T1 | adeno- | PDX | FF | Basal- | Basal- | 0.991 | FALSE | | | | | |
| | squa- | | | like | like | | | | | | | |
| | mous | | | | | | | | | | | |
| S066.FF.PDX.0616T1 | adeno | PDX | FF | Classi- | Classi- | 0.001 | FALSE | | | | | |
| | | | | cal | cal | | | | | | | |
| S067.FF.PDX.1109T1 | adeno | PDX | FF | Classi- | Classi- | 0.001 | FALSE | | | | | |
| | | | | cal | cal | | | | | | | |
| S068.FF.PDX.0806T1 | adeno | PDX | FF | Classi- | Classi- | 0.003 | FALSE | | | | | |
| | | | | cal | cal | | | | | | | |
| S069.FF.PDX.0508T1 | adeno | PDX | FF | Classi- | Classi- | 0.013 | FALSE | | | | | |
| | | | | cal | cal | | | | | | | |
| S070.FF.PDX.0902T1B | | PDX | FF | Classi- | Classi- | 0.001 | FALSE | | | | | |
| | | | | cal | cal | | | | | | | |
| S071.FF.PDX.1112T1 | | PDX | FF | Classi- | Classi- | 0.001 | FALSE | | | | | |
| | | | | cal | cal | | | | | | | |
| S072.FF.PDX.1125T2 | | PDX | FF | Basal- | Basal- | 0.902 | FALSE | | | | | |
| | | | | like | like | | | | | | | |
| S073.FF.PDX.PancT6 | | PDX | FF | Basal- | Basal- | 0.991 | FALSE | | | | | |
| | | | | like | like | | | | | | | |
| S074.FFPE.Pi.0517T1 | adeno | Primary | FFPE | Classi- | Classi- | 0.024 | FALSE | | | | | |
| | | PDAC | | cal | cal | | | | | | | |
| S075.FFPE.Pi.0503T1 | adeno | Primary | FFPE | Classi- | Classi- | 0.038 | FALSE | | | | | |
| | | PDAC | | cal | cal | | | | | | | |
| S076.FFPE.Pi.0417T1 | adeno | Primary | FFPE | Classi- | Classi- | 0.001 | FALSE | | | | | |
| | | PDAC | | cal | cal | | | | | | | |
| S077.FFPE.Pi.0523T1 | adeno | Primary | FFPE | Classi- | Classi- | 0.002 | FALSE | | | | | |
| | | PDAC | | cal | cal | | | | | | | |
| S078.FFPE.Pi.0806T1 | adeno | Primary | FFPE | Classi- | Classi- | 0.013 | FALSE | | | | | |
| | | PDAC | | cal | cal | | | | | | | |
| S079.FF.Pi.0422T1 | adeno | Primary | FF | Basal- | Basal- | 0.991 | FALSE | | | | | |
| | | PDAC | | like | like | | | | | | | |
| S080.FF.Pi.0825T1 | adeno | Primary | FF | Classi- | Classi- | 0.003 | FALSE | | | | | |
| | | PDAC | | cal | cal | | | | | | | |
| S081.FF.Pi.1119T1 | adeno | Primary | FF | Basal- | Basal- | 0.991 | FALSE | | | | | |
| | | PDAC | | like | like | | | | | | | |
| S082.FF.Pi.0517T1 | adeno | Primary | FF | Classi- | Classi- | 0.002 | FALSE | | | | | |
| | | PDAC | | cal | cal | | | | | | | |
| S083.FF.Pi.0818T1 | adeno | Primary | FF | Classi- | Classi- | 0.001 | FALSE | | | | | |
| | | PDAC | | cal | cal | | | | | | | |
| S084.FF.Pi.1012T1 | adeno | Primary | FF | Classi- | Classi- | 0.002 | FALSE | | | | | |
| | | PDAC | | cal | cal | | | | | | | |
| S085.FF.Pi.1118T1 | adeno | Primary | FF | Classi- | Classi- | 0.093 | FALSE | | | | | |
| | | PDAC | | cal | cal | | | | | | | |
| S086.FF.Pi.0105T1 | adeno | Primary | FF | Classi- | Classi- | 0.002 | FALSE | | | | | |
| | | PDAC | | cal | cal | | | | | | | |
| S087.FF.Pi.0119T1 | adeno | Primary | FF | Classi- | Classi- | 0.001 | FALSE | | | | | |
| | | PDAC | | cal | cal | | | | | | | |
| S088.FF.Pi.0417T1 | adeno | Primary | FF | Classi- | Classi- | 0.001 | FALSE | | | | | |
| | | PDAC | | cal | cal | | | | | | | |
| S089.FF.Pi.0503T1 | adeno | Primary | FF | Classi- | Classi- | 0.004 | FALSE | | | | | |
| | | PDAC | | cal | cal | | | | | | | |
| S090.FF.Pi.1109T1 | adeno | Primary | FF | Classi- | Classi- | 0.014 | FALSE | | | | | |
| | | PDAC | | cal | cal | | | | | | | |
| S091.FF.Pi.1129T1 | adeno | Primary | FF | Classi- | Classi- | 0.002 | FALSE | | | | | |
| | | PDAC | | cal | cal | | | | | | | |
| S092.FF.Pi.1206T1 | adeno | Primary | FF | Classi- | Classi- | 0.001 | FALSE | | | | | |
| | | PDAC | | cal | cal | | | | | | | |
| S093.FF.Pi.1214T1 | adeno | Primary | FF | Classi- | Classi- | 0.001 | FALSE | | | | | |
| | | PDAC | | cal | cal | | | | | | | |
| S094.FF.Pi.0124T1 | adeno | Primary | FF | Classi- | Classi- | 0.001 | FALSE | | | | | |
| | | PDAC | | cal | cal | | | | | | | |
| S095.FF.Pi.0221T1 | adeno | Primary | FF | Classi- | Classi- | 0.003 | FALSE | | | | | |
| | | PDAC | | cal | cal | | | | | | | |
| S096.FF.Pi.0222T1 | adeno | Primary | FF | Classi- | Classi- | 0.001 | FALSE | | | | | |
| | | PDAC | | cal | cal | | | | | | | |
| S097.FF.Pi.0327T1 | adeno | Primary | FF | Classi- | Classi- | 0.004 | FALSE | | | | | |
| | | PDAC | | cal | cal | | | | | | | |
| S098.FF.Pi.0328T1 | adeno | Primary | FF | Classi- | Classi- | 0.001 | FALSE | | | | | |
| | | PDAC | | cal | cal | | | | | | | |
| S099.FF.Pi.0411T1 | adeno | Primary | FF | Classi- | Classi- | 0.001 | FALSE | | | | | |
| | | PDAC | | cal | cal | | | | | | | |
| S100.FF.Pi.0417T1 | adeno | Primary | FF | Classi- | Classi- | 0.001 | FALSE | | | | | |
| | | PDAC | | cal | cal | | | | | | | |
| S101.FF.Pi.0425T1 | adeno | Primary | FF | Classi- | Classi- | 0.002 | FALSE | | | | | |
| | | PDAC | | cal | cal | | | | | | | |
| S102.FF.Pi.0502T2 | adeno | Primary | FF | Classi- | Classi- | 0.003 | FALSE | | | | | |
| | | PDAC | | cal | cal | | | | | | | |
| S103.FF.Pi.0508T1 | adeno | Primary | FF | Classi- | Basal- | 0.557 | FALSE | | | | | |
| | | PDAC | | cal | like | | | | | | | |
| S104.FF.Pi.0523T1 | adeno | Primary | FF | Classi- | Classi- | 0.002 | FALSE | | | | | |
| | | PDAC | | cal | cal | | | | | | | |
| S105.FF.Pi.0523T1 | adeno | Primary | FF | Classi- | Classi- | 0.002 | FALSE | | | | | |
| | | PDAC | | cal | cal | | | | | | | |
| S106.FF.Pi.0524T1 | ampul- | Primary | FF | Classi- | Classi- | 0.001 | FALSE | | | | | |
| | lary | PDAC | | cal | cal | | | | | | | |
| S107.FF.Pi.0605T1 | adeno | Primary | FF | Classi- | Classi- | 0.001 | FALSE | | | | | |
| | | PDAC | | cal | cal | | | | | | | |
| S108.FF.Pi.0607T1 | adeno | Primary | FF | Classi- | Classi- | 0.002 | FALSE | | | | | |
| | | PDAC | | cal | cal | | | | | | | |
| S109.FF.Pi.0614T1 | adeno | Primary | FF | Classi- | Classi- | 0.008 | FALSE | | | | | |
| | | PDAC | | cal | cal | | | | | | | |
| S110.FF.Pi.0710T2 | adeno | Primary | FF | Classi- | Classi- | 0.005 | FALSE | | | | | |
| | | PDAC | | cal | cal | | | | | | | |
| S111.FF.Pi.0711T1 | adeno | Primary | FF | Classi- | Basal- | 0.860 | FALSE | | | | | |
| | | PDAC | | cal | like | | | | | | | |
| S112.FF.Pi.0904T1 | adeno | Primary | FF | Classi- | Classi- | 0.034 | FALSE | | | | | |
| | | PDAC | | cal | cal | | | | | | | |
| S113.FF.Pi.1009T1 | adeno | Primary | FF | Classi- | Classi- | 0.033 | FALSE | | | | | |
| | | PDAC | | cal | cal | | | | | | | |
| S114.FF.Pi.1119T1 | adeno | Primary | FF | Classi- | Classi- | 0.021 | FALSE | | | | | |
| | | PDAC | | cal | cal | | | | | | | |
| S115.FF.Pi.1204T1 | adeno | Primary | FF | Classi- | Classi- | 0.001 | FALSE | | | | | |
| | | PDAC | | cal | cal | | | | | | | |
| S116.FF.Pi.1205T1 | adeno | Primary | FF | Classi- | Classi- | 0.013 | FALSE | | | | | |
| | | PDAC | | cal | cal | | | | | | | |
| S117.FF.Pi.0129T1 | adeno | Primary | FF | Classi- | Classi- | 0.002 | FALSE | | | | | |
| | | PDAC | | cal | cal | | | | | | | |
| S118.FF.Pi.0417T1 | adeno | Primary | FF | Classi- | Classi- | 0.013 | FALSE | | | | | |
| | | PDAC | | cal | cal | | | | | | | |
| S119.FF.Pi.0424T1 | adeno | Primary | FF | Classi- | Classi- | 0.001 | FALSE | | | | | |
| | | PDAC | | cal | cal | | | | | | | |
| S120.FF.Pi.0806T1 | adeno | Primary | FF | Classi- | Classi- | 0.005 | FALSE | | | | | |
| | | PDAC | | cal | cal | | | | | | | |
| S121.FF.Pi.0806T1 | adeno | Primary | FF | Classi- | Classi- | 0.001 | FALSE | | | | | |
| | | PDAC | | cal | cal | | | | | | | |
| S122.FF.Pi.0121T1 | adeno | Primary | FF | Classi- | Classi- | 0.002 | FALSE | | | | | |
| | | PDAC | | cal | cal | | | | | | | |
| S123.FF.Pi.0608T1 | adeno | Primary | FF | Classi- | Classi- | 0.005 | FALSE | | | | | |
| | | PDAC | | cal | cal | | | | | | | |
| S124.FF.Pi.1207T1 | adeno | Primary | FF | Classi- | Basal- | 0.671 | FALSE | | | | | |
| | | PDAC | | cal | like | | | | | | | |
| S125.FF.Pi.PancT6 | | Primary | FF | Basal- | Basal- | 0.991 | FALSE | | | | | |
| | | PDAC | | like | like | | | | | | | |

TABLE 18
Collisson

| | | | | | | | Cochran- | Two-Way | Ordinal |
|---|---|---|---|---|---|---|---|---|---|
| | | | | | | | Mantel- | ANOVA | Regression |
| | | | | | SD | | Haenszel test | Model BIC | Model BIC |
| | | | | PD | (>−30% | PR | stratified by | (smaller is | (smaller is |
| Dataset | Treatment | # of samples | Collisson | (>=20%) | & <20%) | (<=−30%) | treatment | better) | better) |
| COMPASS | FFX | 34 | Classical | 0 | 12 | 6 | 0.0024 | 382.8 | 75.77 |
| | | | Exocrine-like | 0 | 3 | 2 | | | |
| | | | QM-PDA | 5 | 5 | 1 | | | |
| | GP | 6 | Classical | 0 | 2 | 1 | | | |
| | | | Exocrine-like | 0 | 0 | 2 | | | |
| | | | QM-PDA | 0 | 1 | 0 | | | |
| Linehan | FOLFIRINOX + | 24 | Classical | 0 | 4 | 4 | 0.4278 | 254.63 | 61.57 |
| | PF-04136309 | | Exocrine-like | 0 | 5 | 3 | | | |
| | | | QM-PDA | 1 | 4 | 3 | | | |
| | FOLFIRINOX | 4 | Classical | 0 | 3 | 0 | | | |
| | | | Exocrine-like | 0 | 0 | 0 | | | |
| | | | QM-PDA | 1 | 0 | 0 | | | |

TABLE 19
Bailey

| | | | | | | | Cochran- | Two-Way | Ordinal |
|---|---|---|---|---|---|---|---|---|---|
| | | | | | | | Mantel- | ANOVA | Regression |
| | | | | | SD | | Haenszel test | Model BIC | Model BIC |
| | | | | PD | (>−30% | PR | stratified by | (smaller is | (smaller is |
| Dataset | Treatment | # of samples | Bailey | (>=20%) | & <20%) | (<=−30%) | treatment | better) | better) |
| COMPASS | FFX | 34 | Squamous | 5 | 3 | 1 | 0.0067 | 385.66 | 78.68 |
| | | | Immunogenic | 0 | 5 | 5 | | | |
| | | | Pancreatic | 0 | 6 | 2 | | | |
| | | | Progenitor | | | | | | |
| | | | ADEX | 0 | 6 | 1 | | | |
| | GP | 6 | Squamous | 0 | 1 | 0 | | | |
| | | | Immunogenic | 0 | 2 | 2 | | | |
| | | | Pancreatic | 0 | 0 | 0 | | | |
| | | | Progenitor | | | | | | |
| | | | ADEX | 0 | 0 | 1 | | | |
| Linehan | FOLFIRINOX + | 24 | Squamous | 1 | 2 | 1 | 0.1126 | 250.75 | 60.64 |
| | PF-04136309 | | Immunogenic | 0 | 3 | 5 | | | |
| | | | Pancreatic | 0 | 6 | 2 | | | |
| | | | Progenitor | | | | | | |
| | | | ADEX | 0 | 2 | 2 | | | |
| | FOLFIRINOX | 4 | Squamous | 1 | 0 | 0 | | | |
| | | | Immunogenic | 0 | 3 | 0 | | | |
| | | | Pancreatic | 0 | 0 | 0 | | | |
| | | | Progenitor | | | | | | |
| | | | ADEX | 0 | 0 | 0 | | | |

TABLE 20
Moffitt

| | | | | | | | Cochran- | Two-Way | Ordinal |
|---|---|---|---|---|---|---|---|---|---|
| | | | | | | | Mantel- | ANOVA | Regression |
| | | | | | SD | | Haenszel test | Model BIC | Model BIC |
| | | | | PD | (>−30% | PR | stratified by | (smaller is | (smaller is |
| Dataset | Treatment | # of samples | Moffitt | (>=20%) | & <20%) | (<=−30%) | treatment | better) | better) |
| COMPASS | FFX | 34 | Basal-like | 5 | 3 | 1 | 0.00098 | 378.75 | 73.07 |
| | | | Classical | 0 | 17 | 8 | | | |
| | GP | 6 | Basal-like | 0 | 1 | 0 | | | |
| | | | Classical | 0 | 2 | 3 | | | |
| Linehan | FOLFIRINOX + | 24 | Basal-like | 1 | 1 | 0 | 0.01183 | 247.37 | 47.47 |
| | PF-04136309 | | Classical | 0 | 12 | 10 | | | |
| | FOLFIRINOX | 4 | Basal-like | 1 | 0 | 0 | | | |
| | | | Classical | 0 | 3 | 0 | | | |

TABLE 21
SSC

| | | | | | SD | | Cochran-Mantel-Haenszel |
|---|---|---|---|---|---|---|---|
| | | | | PD | (>−30% | PR | test stratified |
| Dataset | Treatment | # of samples | PurIST | (>=20%) | & <20%) | (<=−30%) | by treatment |
| COMPASS | FFX | 34 | Basal-like | 5 | 4 | 1 | 1.20E−03 |
| | | | Classical | 0 | 16 | 8 | |
| | GP | 6 | Basal-like | 0 | 1 | 0 | |
| | | | Classical | 0 | 2 | 3 | |
| Linehan | FOLFIRINOX + | 24 | Basal-like | 1 | 1 | 0 | 0.0118 |
| | PF-04136309 | | Classical | 0 | 12 | 10 | |
| | FOLFIRINOX | 4 | Basal-like | 1 | 0 | 0 | |
| | | | Classical | 0 | 3 | 0 | |

TABLE 22
Summary of Subtype Calls by Schema

| | | | Median | | | | | | | | | | | |
|---|---|---|---|---|---|---|---|---|---|---|---|---|---|---|
| | | | Follow- | | | | | | | | | | | |
| | | | up time (m) | | | | | | | | | | | |
| | | | | Cen- | Overall | | Subtypes | | | | | | | |
| | # | | All | sored | Survival (m) | | | | # of | % of | | | HR | |
| | Ana- | # | pa- | pa- | | 95% | | | sam- | sam- | Log- | | 95% | |
| Dataset | lyzed | Events | tients | tients | Median | CI | Subtype | | ples | ples | rank | HR | CI | BIC |
| Linehan_Seq | 28 | 7 | 16.5 | 18 | NA | [25, | Colli- | Classical | 10 | 35.7% | 0.67 | | | 44.503 |
| (FOLFIRINOX + | | | | | | NA] | sion | Exocrine- | 9 | 32.1% | | | | |
| PF-04136309) | | | | | | | | like | | | | | | |
| | | | | | | | | QM-PDA | 9 | 32.1% | | | | |
| | | | | | | | Bailey | ADEX | 5 | 17.9% | 0.35 | | | 44.321 |
| | | | | | | | | Immuno- | 9 | 32.1% | | | | |
| | | | | | | | | genic | | | | | | |
| | | | | | | | | Pancreatic | 9 | 32.1% | | | | |
| | | | | | | | | Progenitor | | | | | | |
| | | | | | | | | Squamous | 5 | 17.9% | | | | |
| | | | | | | | Moffitt | Basal-like | 2 | 7.1% | 0.05 | 6.937 | [0.707, | 41.442 |
| | | | | | | | | Classical | 26 | 92.9% | | | 68.027] | |
| | | | | | | | SSC | Basal-like | 2 | 7.1% | 0.05 | 6.937 | [0.707, | 41.442 |
| | | | | | | | | Classical | 26 | 92.9% | | | 68.027] | |
| Moffitt_GEO_array | 125 | 84 | 13 | 18 | 17 | [13, | Colli- | Classical | 43 | 34.4% | 0.79 | | | 683.915 |
| | | | | | | 20] | sion | Exocrine- | 48 | 38.4% | | | | |
| | | | | | | | | like | | | | | | |
| | | | | | | | | QM-PDA | 34 | 27.2% | | | | |
| | | | | | | | Bailey | ADEX | 27 | 21.6% | <0.0001 | | | 677.403 |
| | | | | | | | | Immuno- | 3 | 2.4% | | | | |
| | | | | | | | | genic | | | | | | |
| | | | | | | | | Pancreatic | 47 | 37.6% | | | | |
| | | | | | | | | Progenitor | | | | | | |
| | | | | | | | | Squamous | 48 | 38.4% | | | | |
| | | | | | | | | | | % | | | | |
| | | | | | | | Moffitt | Basal-like | 24 | 19.2% | 0.034 | 1.737 | [1.038, | 675.985 |
| | | | | | | | | Classical | 101 | 80.8% | | | 2.906] | |
| | | | | | | | SSC | Basal-like | 20 | 16.0% | 0.14 | 1.502 | [0.870, | 678.021 |
| | | | | | | | | Classical | 105 | 84.0% | | | 2.595] | |
| PACA_AU_array | 71 | 43 | 14 | 21.5 | 16.6 | [13.7, | Colli- | Classical | 27 | 38.0% | 0.019 | | | 305.231 |
| | | | | | | 30.0] | sion | Exocrine- | 18 | 25.4% | | | | |
| | | | | | | | | like | | | | | | |
| | | | | | | | | QM-PDA | 26 | 36.6% | | | | |
| | | | | | | | Bailey | ADEX | 12 | 16.9% | 0.12 | | | 311.789 |
| | | | | | | | | Immuno- | 19 | 26.8% | | | | |
| | | | | | | | | genic | | | | | | |
| | | | | | | | | Pancreatic | 17 | 23.9% | | | | |
| | | | | | | | | Progenitor | | | | | | |
| | | | | | | | | Squamous | 23 | 32.4% | | | | |
| | | | | | | | Moffitt | Basal-like | 13 | 18.3% | 0.009 | 2.516 | [1.228, | 304.408 |
| | | | | | | | | Classical | 58 | 81.7% | | | 5.155] | |
| | | | | | | | SSC | Basal-like | 12 | 16.9% | 0.038 | 2.218 | [1.022, | 306.343 |
| | | | | | | | | Classical | 59 | 83.1% | | | 4.815] | |
| PACA_AU_seq | 57 | 33 | 13.2 | 17.5 | 15 | [13.2, | Colli- | Classical | 24 | 42.1% | 0.006 | | | 211.127 |
| | | | | | | NA] | sion | Exocrine- | 11 | 19.3% | | | | |
| | | | | | | | | like | | | | | | |
| | | | | | | | | QM-PDA | 22 | 38.6% | | | | |
| | | | | | | | Bailey | ADEX | 7 | 12.3% | 0.47 | | | 222.647 |
| | | | | | | | | Immuno- | 16 | 28.1% | | | | |
| | | | | | | | | genic | | | | | | |
| | | | | | | | | Pancreatic | 14 | 24.6% | | | | |
| | | | | | | | | Progenitor | | | | | | |
| | | | | | | | | Squamous | 20 | 35.1% | | | | |
| | | | | | | | Moffitt | Basal-like | 11 | 19.3% | 0.014 | 2.835 | [1.188, | 213.574 |
| | | | | | | | | Classical | 46 | 80.7% | | | 6.766] | |
| | | | | | | | SSC | Basal-like | 14 | 24.6% | 0.072 | 2.016 | [0.921, | 215.414 |
| | | | | | | | | Classical | 43 | 75.4% | | | 4.417] | |
| TCGA_PAAD | 146 | 75 | 14.2 | 15.1 | 20.2 | [16.6, | Colli- | Classical | 52 | 35.6% | 0.41 | | | 623.054 |
| | | | | | | 23.4] | sion | Exocrine- | 61 | 41.8% | | | | |
| | | | | | | | | like | | | | | | |
| | | | | | | | | QM-PDA | 33 | 22.6% | | | | |
| | | | | | | | Bailey | ADEX | 38 | 26.0% | 0.54 | | | 626.808 |
| | | | | | | | | Immuno- | 26 | 17.8% | | | | |
| | | | | | | | | genic | | | | | | |
| | | | | | | | | Pancreatic | 51 | 34.9% | | | | |
| | | | | | | | | Progenitor | | | | | | |
| | | | | | | | | Squamous | 31 | 21.2% | | | | |
| | | | | | | | Moffitt | Basal-like | 37 | 25.3% | 0.0064 | 1.941 | [1.194, | 613.865 |
| | | | | | | | | Classical | 109 | 74.7% | | | 3.156] | |
| | | | | | | | SSC | Basal-like | 33 | 22.6% | 0.0031 | 2.113 | [1.271, | 612.969 |
| | | | | | | | | Classical | 113 | 77.4% | | | 3.512] | |
| Pooled public | 376 | 214 | 14.1 | 17 | 19 | [16.6, | Colli- | Classical | 134 | 35.6% | 0.0692 | | | 1654.238 |
| datasets | | | | | | 22.0] | sion | Exocrine- | 137 | 36.4% | | | | |
| of primary | | | | | | | | like | | | | | | |
| samples | | | | | | | | QM-PDA | 105 | 27.9% | | | | |
| | | | | | | | Bailey | ADEX | 83 | 22.1% | 0.0768 | | | 1658.276 |
| | | | | | | | | Immuno- | 58 | 15.4% | | | | |
| | | | | | | | | genic | | | | | | |
| | | | | | | | | Pancreatic | 127 | 33.8% | | | | |
| | | | | | | | | Progenitor | | | | | | |
| | | | | | | | | Squamous | 108 | 28.7% | | | | |
| | | | | | | | Moffitt | Basal-like | 77 | 20.5% | 1.43E−05 | 1.982 | [1.447, | 1637.78 |
| | | | | | | | | Classical | 299 | 79.5% | | | 2.715] | |
| | | | | | | | SSC | Basal-like | 68 | 18.1% | 0.0001 | 1.896 | [1.361, | 1641.295 |
| | | | | | | | | Classical | 308 | 81.9% | | | 2.640] | |
| | 124 | 63 | 15 | 17.5 | 23.3 | [16.6, | SSC | Basal-like | 21 | 16.9% | 0.0107 | 2.436 | [1.2086, | 384.622 |
| | | | | | | 35.8] | (exclud- | Classical | 103 | 83.1% | | | 4.9116] | |
| | | | | | | | ing | | | | | | | |
| | | | | | | | training | | | | | | | |
| | | | | | | | sam- | | | | | | | |
| | | | | | | | ples) | | | | | | | |
| Aguirre_seq | 48 | 35 | 10 | 15 | 11.5 | [9.73, | SSC | Basal-like | 15 | 31.3% | 0.14 | 1.688 | [0.835, | 219.125 |
| | | | | | | 19.80] | | Classical | 33 | 68.8% | | | 3.407] | |
| Yeh_seq_FNA | 42 | 30 | 12.9 | 24.4 | 17.1 | [10.2, | SSC | Basal-like | 2 | 4.8% | 0.017 | 5.289 | [1.151, | 178.179 |
| | | | | | | 24.6] | | Classical | 40 | 95.2% | | | 24.31] | |

TABLE 23
Collisson Transition Rates

| | | | | |
|---|---|---|---|---|
| Pre-treatment | QM | 0.22 | 0 | 0.78 |
| | Exocrine | 0.5 | 0.5 | 0 |
| | Classical | 0.45 | 0.27 | 0.27 |
| | | Classical | Exocrine | QM |

Post-treatment

TABLE 24
Bailey Transition Rates

| | | | | | |
|---|---|---|---|---|---|
| Pre-treatment | Squamous | 0 | 0.2 | 0 | 0.8 |
| | PP | 0.25 | 0.38 | 0.25 | 0.12 |
| | Immuno. | 0 | 0.64 | 0.18 | 0.18 |
| | ADEX | 0.5 | 0.25 | 0.25 | 0 |
| | | Squamous | PP | Immuno. | ADEX |

Post-treatment

TABLE 25
PurIST Coefficients

| | | Intercept: −6.815 |
|---|---|---|
| Gene A | Gene B | Coefficient |
| GPR87 | REG4 | 1.994 |
| KRT6A | ANXA10 | 2.031 |
| BCAR3 | GATA6 | 1.618 |
| PTGES | CLDN18 | 0.922 |
| ITGA3 | LGALS4 | 1.059 |
| C16orf74 | DDC | 0.929 |
| S100A2 | SLC40A1 | 2.505 |
| KRT5 | CLRN3 | 0.485 |

TABLE 26
PurIST-n Coefficients

| | | Intercept: −12.414 |
|---|---|---|
| Gene A | Gene B | Coefficients |
| GPR87 | REG4 | 3.413 |
| KRT6A | ANXA10 | 3.437 |
| KRT17 | LGALS4 | 2.078 |
| S100A2 | TFF1 | 2.651 |
| C16orf74 | DDC | 0.901 |
| KRT15 | PLA2G10 | 2.677 |
| PTGES | CDH17 | 2.911 |
| DCBLD2 | TSPAN8 | 1.903 |
| PIP5K1B | MUC17 | 0.036 |
| NR1I2 | MYO1A | −0.638 |
| CTSE | LYZ | 0.977 |

TABLE 27
Validation Dataset Individual Study Areas Under the Curves

| Dataset | N | Basal-like | Accuracy | Sensitivity | Specificity | AUC |
|---|---|---|---|---|---|---|
| PACA_AU_seq | 65 | 12 | 0.892 | 0.833 | 0.906 | 0.965 |
| PACA_AU_array | 95 | 14 | 0.958 | 0.929 | 0.963 | 0.973 |
| Moffitt | 37 | 56 | 0.973 | 11 | 0.969 | 11 |
| Linehan_Seq | 66 | 11 | 1 | 0.545 | 1 | 0.984 |
| Connor | 66 | 13 | 0.909 | 1 | 0.982 | 1 |
| COMPASS | 49 | 12 | 0.98 | 0.833 | 0.972 | 0.965 |

TABLE 28
Exemplary PKIs and Their Targets

| Gene | | | Overexpressed |
|---|---|---|---|
| Name | Aliases | Compounds | in Subtype |
| AAK1 | KIAA1048, DKF2p686K16132 | GSK3236425A; LP-935509; UNC-AA-1-0013 | Basal-like |
| | | (SGC-AAK1-1); UNC-AA-1-0017 | |
| ABL1 | JTK7, c-ABL, p150 | asciminib; canertinib, CI-1033; erlotinib, OSI-744; | Basal-like |
| | | GNF-5; imatinib; LDN-214117; masitinib AB1010; | |
| | | XMD-17-51 | |
| CDK1 | CDC28A, CDC2, P34CDC2 | GW276655; GW300657X; GW300660X; | Basal-like |
| | | GW416981X | |
| CDK16 | PCTAIRE, PCTAIRE1, | CAF-204; SNS-032 | Basal-like |
| | PCTGAIRE, FLJ16665, | | |
| | PCTK1 | | |
| CDK17 | PCTAIRE2, PCTK2 | YL-206; SNS-032 | Basal-like |
| CDK4 | CMM3; PSK-J3 | abemaciclib; LY2857785; palbociclib; ribociclib; | Basal-like |
| | | PFE-PKIS 32; PFE-PKIS 44; SIHR CDK4/6 | |
| | | compound 83; SIHR CDK4/6 compound 91 | |
| CDK7 | CAK1, CDKN7, MO15, STK1, | BMS-387032/SNS-032; BS-181; THZ1 | Basal-like |
| | CAK, HCAK, p39MO15 | | |
| CSNK2A2 | CSNK2A1, CK2α, CK2A2, | G59973, entospletinib; GO289; CX-4945, | Basal-like |
| | CK2a2, CK2α2 | silmasertib; AZ-G | |
| DDR1 | RTK6, CD167, CAK, CD167, | AC220, quizartinib; DDR1 compound 7ae; | Basal-like |
| | DDR, EDDR1, HGK2, MCK10, | DDR1-1N-1; imatinib; LY2801653; masitinib | |
| | NEP, NTRK4, PTK3, PTK3A, | AB1010; PD173074; RAF-265, CHIR-265; | |
| | TRKE | DDR-TRK-1; GW832467; TPKI-39 | |
| EPHA2 | ARCC2, CTPA, CTPP1, | LY3009120; MLN8237/Alisertib; GW693917A; | Basal-like |
| | CTRCT6, ECK, EphA2 | ALW-II-41-27 | |
| FER | PPP1R74, TYK3, p94-Fer | GSK1838705A; PF-06463922, Lorlatinib; | Basal-like |
| FRK | RAK, GTK, PTK5 | GSK1904529 Abbott Compound 530; PF-06463922, | Classical |
| | | Lorlatinib; XMD8-87; GSK1904529; TPKI-113 | |
| GSK3A/G | | BAY-61-3606; Carna compound 13; CHIR-99021; | Basal-like |
| SK3B | | EHT5372; GW784752X; SB-742609; TPKI-91; | |
| | | ARA014418; LY-317615, enzastaurin; | |
| | | GW513184X; GW810372X; SB-725317; TPKI-85 | |
| INSR | CD220, HHF5, IR | OSI-906, linsitinib; GSK1392956A; GSK1904529; | Basal-like |
| | | GSK2219385 | |
| LIMK1 | LIMK, LIMK-1 | CRT0105446; LIMKi compound 3; Amakem | Basal-like |
| | | tetrahydropyrimido-indole compound 3; Scripps FL | |
| | | 18b; LX7101; TH-257; R10015 | |
| LYN | JTK8, p53Lyn, p56Lyn | masitinib AB1010; saturated ibrutinib; Maly LYN | Classical |
| | | compound 19 | |
| MAP2K2 | CFC4, MAPKK2, MEK2, | Trametinib (GSK1120212); cobimetinib/GDC0973; | |
| | MKK2, PRKMK2 | binimetinib; refametinib; ESD0001937 | |
| MAP3K11 | SPRK, MEKK11, MLK-3, | PFE-PKIS18; SGK1 Sanofi 14n | Classical |
| | PTK1 | | |
| MAP3K2 | MEKK2B, MEKK2 | MRKI-19; GSK2656157; AKI00000018a; | Basal-like |
| | | AK100000021a | |
| MAP3K5 | MAPKKK5, ASK1, | Compound 10.HCl; MSC 2032964A; PF3644022; | Basal-like |
| | MEKK5 | TPKI-58 | |
| MAP4K5 | KHSqqq1, GCKR, KHS, | FRAX1036; G-5555 | Basal-like |
| | MAPKKKK5 | | |
| MAPK1 | ERK, ERK2, p41mapk, | Carna compound 13; SCH772984; Vertex 11e; AZ | Basal-like |
| | MAPK2, PRKM2, PRKM1, | compound 35 | |
| | ERT1, ERK-2, P42MAPK, | | |
| | PRKM1, p38, p40, p41, | | |
| | p42-MAPK, Erk2 | | |
| MAPK3 | ERK1, p44mapk, p44erk1, | SCH+E:E772984; | Classical |
| | PRKM3, ERK-1, ERT2, | GAN-305074X (aka GW5074) | |
| | HS44KDAP, HUMKER1A, | | |
| | P44ERK1, P44MAPK, | | |
| | p44-ERK1, p44-MAPK, Erk1 | | |
| PAK4 | | GenentechPAK compound 13; | Basal-like |
| | | Novartis compound 11 | |
| PIP4K2C | PIP5K2C | G1T28 | Basal-like |
| PKM | | TLN-232 (aka CAP-232) | |
| PRKCD | ALPS3, CVID9, MAY1, PKCD, | LY-317615; enzastaurin; uprosertib, GSK2141795 | Classical |
| | nPKC-delta PKCd | | |
| PTK2B | CAKB, PYK2, RAFTK, PTK, | PF-06463922; Lorlatinib; GSK1392956A | Basal-like |
| | CADTK, FADK2, FAK2, PKB | | |
| PIK6 | BtK, p21cdc42Hs, | PLX-4720; Vemurafenib; saturated ibrutinib; XMD8-87; | Classical |
| | | 21a; PF-6698840 | |
| RIPK2 | RICK, RIP2, CARDIAK, | LDN-214117; Novartis Compound 2; OD36; OD38; | Basal-like |
| | CARD3, CCK, GIG30 | saturated ibrutinib; SB-203580; SB-590885; WEHI- | |
| | | 345; GSK583; GSK RIPK2 inhibitor 7 | |
| ROCK2 | ROCK-II | GSK269962A; GSK429286; SB-747651A; | Classical |
| | | Scripps compound 35; Netarsudil; netarsudil hydrolysis | |
| | | product; Abbvie ROCK compound 16; Abbvie | |
| | | ROCK compound 58 | |
| SRC | ASV, c-src, ASV1, THC6, | many inhibitors, usually src family | Classical |
| | c-SRC, p60-Src | | |
| sTK10 | LOK, PRO2729 | erlotinib, OSI-744; GSK461364A; RAF-265, | Basal-like |
| | | CHIR-265; GSK204607; SB-633825 | |
| TBK1 | NAK, FTDALS4; T2K | WEHI-112; GSK8612 | Basal-like |
| YES1 | Yes, c-yes, HsT441, P61-YES | PF-477736; saturated ibrutinib; GW621970X | Basal-like |

1. A method for determining a subtype of a pancreatic tumor in a biological sample comprising, consisting essentially of, or consisting of pancreatic tumor cells obtained from a subject, the method comprising:
(a) obtaining gene expression levels for each of the following genes in the biological sample: GPR87, KRT6A, BCAR3, PTGES, ITGA3, C16orf74, S100A2, KRT5, REG4, ANXA10, GATA6, CLDN18, LGALS4, DDC, SLC40A1, CLRN3;
(b) performing a pair-wise comparison of the gene expression levels for each of Gene Pairs 1-8 or for each of Gene Pairs A-H, wherein Gene Pairs 1-8 and Gene Pairs A-H are as follows:

| GENE | GENE | GENE | |
|---|---|---|---|
| PAIR | A | B | Coefficient |
| 1 | GPR87 | REG4 | 1.994 |
| 2 | KRT6A | ANXA10 | 2.031 |
| 3 | BCAR3 | GATA6 | 1.618 |
| 4 | PTGES | CLDN18 | 0.922 |
| 5 | ITGA3 | LGALS4 | 1.059 |
| 6 | C16orf74 | DDC | 0.929 |
| 7 | S100A2 | SLC40A1 | 2.505 |
| 8 | KRT5 | CLRN3 | 0.485 |
| A | GPR87 | REG4 | 3.413 |
| B | KRT6A | ANXA10 | 3.437 |
| C | KRT17 | LGALS4 | 2.078 |
| D | S100A2 | TFF1 | 2.651 |
| E | C16orf74 | DDC | 0.901 |
| F | KRT15 | PLA2G10 | 2.677 |
| G | PTGES | CDH17 | 2.911 |
| H | DCBLD2 | TSPAN8 | 1.903 |

and
(c) calculating a Raw Score for the biological sample, wherein the calculating comprises:
(i) assigning a value of 1 for each Gene Pair for which Gene A of the Gene Pair has a higher expression level than Gene B of the Gene Pair, and a value of 0 for each Gene Pair for which Gene A of the Gene Pair has a lower expression level than Gene B of the Gene Pair;
(ii) multiplying each assigned value by the coefficient listed above for the corresponding Gene Pair to calculate eight individual Gene Pair scores; and
(iii) adding the eight individual Gene Pair scores together along with a baseline effect to calculate a Raw Score for the biological sample, wherein the baseline effect is −6.815 for Gene Pairs 1-8 and −12.414 for Gene Pairs A-H,
wherein if the calculated Raw Score is greater than or equal to 0, the tumor subtype is determined to be a basal-like subtype, and if the calculated Raw Score if less than 0, the tumor subtype is determined to be a classical subtype.

2. The method of claim 1, further comprising converting the Raw Score to a predicted basal-like probability (PBP) using the inverse-logit transformation
$$P B P = {e}^{Raw Score} / ( 1 + {e}^{Raw Score} ) ,$$
wherein if the PBP is greater than 0.5, the tumor subtype is determined to be a basal-like subtype and if the PBP if less than or equal to 0.5, the tumor subtype is determined to be a classical subtype.

3. The method of claim 1, wherein the pancreatic tumor is a pancreatic ductal adenocarcinoma (PDAC).

4. The method of claim 1, wherein the biological sample comprises a biopsy sample, optionally a fine needle biopsy aspiration or a percutaneous core needle biopsy, or comprises a frozen or archival sample derived therefrom.

5. The method of claim 1, wherein the obtaining employs a technique selected from the group consisting of microarray analysis, RNAseq, quantitative RT-PCR, NanoString, or any combination thereof.

6. The method of claim 5, wherein the technique comprises NanoString and employs probes comprising the following SEQ ID NOs:

| GENE | GENE | SEQ ID | GENE | SEQ ID |
|---|---|---|---|---|
| PAIR | A | NO: | B | NO: |
| 1 | GPR87 | 64 | REG4 | 71 |
| 2 | KRT6A | 65 | ANXA10 | 59 |
| 3 | BCAR3 | 81 | GATA6 | 82 |
| 4 | PTGES | 70 | CLDN18 | 84 |
| 5 | ITGA3 | 85 | LGALS4 | 68 |
| 6 | C16orf74 | 60 | DDC | 63 |
| 7 | S100A2 | 72 | SLC40A1 | 86 |
| 8 | KRT5 | 87 | CLRN3 | 83 |
| A | GPR87 | 64 | REG4 | 71 |
| B | KRT6A | 65 | ANXA10 | 59 |
| C | KRT17 | 67 | LGALS4 | 68 |
| D | S100A2 | 72 | TFF1 | 73 |
| E | C16orf74 | 60 | DDC | 63 |
| F | KRT15 | 66 | PLA2G10 | 69 |
| G | PTGES | 70 | CDH17 | 61 |
| H | DCBLD2 | 62 | TSPAN8 | 74. |

7. The method of claim 1, wherein the subject is a human.

8-12. (canceled)

13. A method for treating a patient diagnosed with pancreatic ductal adenocarcinoma (PDAC), the method comprising:
(a) identifying a subtype of the patient's PDAC via the method of claim 1; and
(b) treating the patient with gemcitabine, optionally in combination with nab-paclitaxel, if the assigned subtype is a basal-like subtype and treating the patient with FOLFIRINOX if the assigned subtype is classical.

14. The method of claim 13, where the treating comprises at least one additional anti-PDAC treatment.

15. The method of claim 14, where the at least one additional anti-PDAC treatment is surgery, radiation, administration of an additional chemotherapeutic agent, administration of a protein kinase inhibitor (PKI), and any combination thereof.

16. The method of claim 15, wherein the additional chemotherapeutic agent is a CCR2 inhibitor, a checkpoint inhibitor, or any combination thereof.

17. The method of claim 13, wherein the patient is a human.

18-23. (canceled)


SEQUENCE LISTING (102 sequences)

SEQ ID NO: 1 - DNA, 1441 bp, Homo sapiens
  source 1..1441: mol_type = other DNA
  CDS 165..1139
atccagatttgcttttacattttcttgcctgagtctgaggtgaacagtgaacatatttacatttgatttaacagtgaaccttaattctttctggcttcacagtgaaacaagtttatgcaatcgatcaaatattttcatccctgaggttaacaattaccatcaaaatgttttgtggagactatgtgcaaggaaccatcttcccagctcccaatttcaatcccataatggatgcccaaatgctaggaggagcactccaaggatttgactgtgacaaagacatgctgatcaacattctgactcagcgctgcaatgcacaaaggatgatgattgcagaggcataccagagcatgtatggccgggacctgattggggatatgagggagcagctttcggatcacttcaaagatgtgatggctggcctcatgtacccaccaccactgtatgatgctcatgagctctggcatgccatgaagggagtaggcactgatgagaattgcctcattgaaatactagcttcaagaacaaatggagaaattttccagatgcgagaagcctactgcttgcaatacagcaataacctccaagaggacatttattcagagacctcaggacacttcagagatactctcatgaacttggtccaggggaccagagaggaaggatatacagaccctgcgatggctgctcaggatgcaatggtcctatgggaagcctgtcagcagaagacgggggagcacaaaaccatgctgcaaatgatcctgtgcaacaagagctaccagcagctgcggctggttttccaggaatttcaaaatatttctgggcaagatatggtagatgccattaatgaatgttatgatggatactttcaggagctgctggttgcaattgttctctgtgttcgagacaaaccagcctattttgcttatagattatatagtgcaattcatgactttggtttccataataaaactgtaatcaggattctcattgccagaagtgaaatagacctgctgaccataaggaaacgatacaaagagcgatatggaaaatccctatttcatgatatcagaaattttgcttcagggcattataagaaagcactgcttgccatctgtgctggtgatgctgaggactactaaaatgaagaggacttggagtactgtgcactcctctttctagacacttccaaatagagattttctcacaaatttgtactgttcatggcactattaacaaaactatacaatcatattttctcttctatctttgaaattattctaagccaaagaaaactatgaatgaaagtatatgatactgaatttgcctactatcctgaatttgcctactatctaatcagcaattaaataaattgtgcatgatggaataatagaaaaattgcattggaatagattttatttaaatgtgaaccatcaacaaccta

SEQ ID NO: 2 - protein, 324 aa, Homo sapiens
MFCGDYVQGTIFPAPNFNPIMDAQMLGGALQGFDCDKDMLINILTQRCNAQRMMIAEAYQSMYGRDLIGDMREQLSDHFKDVMAGLMYPPPLYDAHELWHAMKGVGTDENCLIEILASRTNGEIFQMREAYCLQYSNNLQEDIYSETSGHFRDTLMNLVQGTREEGYTDPAMAAQDAMVLWEACQQKTGEHKTMLQMILCNKSYQQLRLVFQEFQNISGQDMVDAINECYDGYFQELLVAIVLCVRDKPAYFAYRLYSAIHDFGFHNKTVIRILIARSEIDLLTIRKRYKERYGKSLFHDIRNFASGHYKKALLAICAGDAEDY

SEQ ID NO: 3 - DNA, 3336 bp, Homo sapiens
  source 1..3336: mol_type = other DNA
  CDS 359..2836
ctctccggcggccgctccagcccggccgggaccccagagttgcaggcccaggccagccgttgcaccgttaagccgggccggtgacgccgggcctttacgtcgcgcctgtgagcggccgaggccgagcggcctcccctcgggctgcgagacggagacattacctcatccctcaagattgctcgctgcaaacacaacgctgagaaatggcccaagtaaagcgtgatcagggtcttgcattcttggcacatctaggaagaacgtgcaaggatgcgaattgtgaatcgcttctccaaacagagctgcttcatctgacccaagctggcctggagaaaggaggaagaggcaagagttgagaattatggctgcaggaaaatttgcaagccttcccagaaacatgccggtgaatcaccagttccccctggcctcatccatggaccttctgagcagcaggtcccctctcgctgagcatcgcccagatgcctatcaagatgtgtctatacatggcacccttccacggaagaaaaaaggtcctcctcccataaggtcctgtgatgacttcagtcacatgggcaccctcccccactccaaatccccacggcagaactcgcctgtgacccaggatggcatccaggagagcccatggcaggaccggcacggcgaaaccttcaccttcagggatccacatcttctggacccaactgtggaatatgtgaagttctccaaggagaggcacatcatggacaggacccccgagaaactgaagaaggagctggaggaggagctgctcctgagcagcgaggacctgcgcagccatgcctggtaccacggccgcatcccccgacaggtgtctgaaaaccttgtgcagcgagatggtgacttcctagttcgtgactctctgtccagccctgggaactttgtcctgacctgtcagtggaagaacctcgctcagcacttcaaaatcaaccggacagttctgcgactcagcgaggcctacagccgcgtgcagtaccagttcgagatggagagcttcgactccatccccggcctggtgcgctgctacgtgggcaaccgccggcccatctcccagcagagtggcgccatcatcttccagcccatcaacaggacggtgcctctgcggtgcctggaggagcattatggcacctccccaggccaggcccgggagggcagcctcaccaagggaaggccggatgtggccaagaggctgagcctcaccatgggtggcgtccaggcccgagagcagaatttgcccaggggaaacctcctcagaaacaaagaaaagagtggtagccagcccgcctgcctggatcacatgcaggacagaagagccttgtccctcaaagcccaccagtcagagagctacctgccgattggctgcaagctgccacctcagtcctcgggtgtggacacaagcccctgcccaaactcacctgtgttcaggacgggaagcgagcctgccctgagcccagcagtggttcggagggtctcctcagacgccagggctggggaggcgctgaggggatcagacagtcaactgtgccctaagcccccgcctaagccctgcaaggtgccgttcctcaaggttccctcgtctccctctgcctggctcaactcagaggccaactactgtgaactgaacccagcgtttgccacaggctgcggcaggggagcaaagctaccctcatgtgcccagggaagccacacagaactgctcacagccaagcagaatgaggcgccaggtccccggaactctggcgtcaactacttgatccttgatgatgatgacagggaaagaccttgggaacctgcggcagctcagatggagaaggggcagtgggacaagggcgagtttgtgacgcccctcctggagactgtctcctccttcaggcccaacgagtttgagtcaaagttccttccccctgagaataagcccctggaaacagcaatgttgaaacgtgcaaaagaactgttcaccaacaacgaccccaaggtcatcgcccagcacgtactgagcatggactgcagggttgctaggatacttggagtctctgaagagatgaggaggaacatgggggtgagctcaggcctggaactcattaccttgcctcacggacaccagctgcgcctggacataattgaaagacacaacacaatggccatcggcattgcagtggacattctgggatgcacgggcactttggaggaccgagcggccactctgagtaagatcatccaggtggcggtggaactgaaggattccatgggggacctctattccttctcagctctcatgaaagccctggaaatgccacagatcacaaggttagaaaagacgtggactgctctgcggcaccagtacacccaaactgccattctctatgagaaacagctgaagcccttcagcaaactcctgcatgaaggcagagagtccacatgtgttcccccaaacaatgtatcagtcccactgctgatgccgcttgtgacgttaatggagcgccaggctgtgacttttgaaggaaccgacatgtgggaaaaaaacgaccagagctgtgaaatcatgctgaaccatttggcaacagcgcgattcatggccgaggctgcagacagctaccggatgaatgctgagaggatcctggcaggttttcaaccagatgaagaaatgaatgaaatctgcaagactgaatttcaaatgcgattgctatggggcagcaaaggtgcacaagtcaatcagacagagagatatgagaaattcaaccagattttaactgccctctcgcgtaaattggaacctcctcctgtaaagcaggcagagctttgataactctccagagaacctttagaatatcttttcaagtttccccagcttcatctttgggaaagcttactgtttttgataaagtaataatgtgcaaatctgacaatatacaagcttttagtatccacaggatattaaacgtgtaaattgcacagagcacacttatttatgaattgtctaaagttactactgattttaaaatgaataatttattattaaggtaactactgctaatgttgatcagcaaatttaagagaagacctagctatgttggctggttgctttctattatcatggtatttgaccattttagttttaattccatgtcagataagtgtaaatagaagagtttaaaagcatgaaacatttcagaaggtatcagttatatgatattctttaaacaaatatgaaaaatgtaaatactcatgaatgaaaatacatctttttgtgaaacagttgtatccagtctctttcatattaaacaactcatcttggtacaataa

SEQ ID NO: 4 - protein, 825 aa, Homo sapiens
MAAGKFASLPRNMPVNHQFPLASSMDLLSSRSPLAEHRPDAYQDVSIHGTLPRKKKGPPPIRSCDDFSHMGTLPHSKSPRQNSPVTQDGIQESPWQDRHGETFTFRDPHLLDPTVEYVKFSKERHIMDRTPEKLKKELEEELLLSSEDLRSHAWYHGRIPRQVSENLVQRDGDFLVRDSLSSPGNFVLTCQWKNLAQHFKINRTVLRLSEAYSRVQYQFEMESFDSIPGLVRCYVGNRRPISQQSGAIIFQPINRTVPLRCLEEHYGTSPGQAREGSLTKGRPDVAKRLSLTMGGVQAREQNLPRGNLLRNKEKSGSQPACLDHMQDRRALSLKAHQSESYLPIGCKLPPQSSGVDTSPCPNSPVFRTGSEPALSPAVVRRVSSDARAGEALRGSDSQLCPKPPPKPCKVPFLKVPSSPSAWLNSEANYCELNPAFATGCGRGAKLPSCAQGSHTELLTAKQNEAPGPRNSGVNYLILDDDDRERPWEPAAAQMEKGQWDKGEFVTPLLETVSSFRPNEFESKFLPPENKPLETAMLKRAKELFTNNDPKVIAQHVLSMDCRVARILGVSEEMRRNMGVSSGLELITLPHGHQLRLDIIERHNTMAIGIAVDILGCTGTLEDRAATLSKIIQVAVELKDSMGDLYSFSALMKALEMPQITRLEKTWTALRHQYTQTAILYEKQLKPFSKLLHEGRESTCVPPNNVSVPLLMPLVTLMERQAVTFEGTDMWEKNDQSCEIMLNHLATARFMAEAADSYRMNAERILAGFQPDEEMNEICKTEFQMRLLWGSKGAQVNQTERYEKFNQILTALSRKLEPPPVKQAEL

SEQ ID NO: 5 - DNA, 911 bp, Homo sapiens
  source 1..911: mol_type = other DNA
  CDS 190..420
aggcgcgcggctgcccgagcgccggccgggccatgacccccgctgctctgtcttgcaggctcgtcgccgcggccccccgagcccgaccgccgccgccaccaccaccagcgcccgggcgggcctcgcgcgcctcgggcgcggctccgcagtgagcccaccaagaaggaagcggcctgcagaggtgccgacatggggcttaagatgtcctgcctgaaaggctttcaaatgtgtgtcagcagcagcagcagcagccacgacgaggcccccgtcctgaacgacaagcacctggacgtgcccgacatcatcatcacgccccccacccccacgggcatgatgctgccgagggacttggggagcacagtctggctggatgagacagggtcgtgcccagatgatggagaaatcgacccagaagcctgaggaggtgtcctgggtttggctggctggctcctgctccagcggcccggcttcaggtgtccgggggcgtggctgcctggagcaggtgtgctgaataccctggatgggaactgagcgaacccgggcctccgctcagagagacgtggcaggaccagcgaggaatccagcctgtccacttccagaacagtgtttcccaggccccgctgagtggaccggacctctgacacctccaggttcttgctgactccggcctggtgaaagggagcgccatggtcctggctgttggggtcccagggagaggctctcttctggacaaacacaccctcccagcccccagggctgtgcaaacacatgcccctgccataagcaccaacaagaacttcttgcaggtggagtggctgttttttataagttgttttacagatacggaaacagtccaaaatgggatttataatttcttttttgcattataaataaagatcctctgtaacaaa

SEQ ID NO: 6 - protein, 76 aa, Homo sapiens
MGLKMSCLKGFQMCVSSSSSSHDEAPVLNDKHLDVPDIIITPPTPTGMMLPRDLGSTVWLDETGSCPDDGEIDPEA

SEQ ID NO: 7 - DNA, 3670 bp, Homo sapiens
  source 1..3670: mol_type = other DNA
  CDS 94..2592
agtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccataaaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttgttga

SEQ ID NO: 8 - protein, 832 aa, Homo sapiens
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 9 - DNA, 3355 bp, Homo sapiens
  source 1..3355: mol_type = other DNA
  CDS 62..847
acaccttcggcagcaggagggcggcagcttctcgcaggcggcagggcgggcggccaggatcatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctaca

SEQ ID NO: 10 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 11 - DNA, 1146 bp, Homo sapiens
  source 1..1146: mol_type = other DNA
  CDS 158..838
cattagcataacccttcctcaggaagagtgagattttatatttgacaataaagtgttagactccatttctaaataccagacttcaaaagataaggttcaaaagtgttataagaagatattcctttttttgtcctagagaacttattttcctgtgaaaatgcctaccacaaagaagacattgatgttcttatcaagctttttcaccagccttgggtccttcattgtaatttgctctattcttgggacacaagcatggatcaccagtacaattgctgttagagactctgcttcaaatgggagcattttcatcacttacggactttttcgtggggagagtagtgaagaattgagtcacggacttgcagaaccaaagaaaaagtttgcagttttagagatactgaataattcttcccaaaaaactctgcattcggtgactatcctgttcctggtcctgagtttgatcacgtcgctgctgagctctgggtttaccttctacaacagcatcagcaacccttaccagacattcctggggccgacgggggtgtacacctggaacgggctcggtgcatccttcgtttttgtgaccatgatactgtttgtggcgaacacgcagtccaaccaactctccgaagagttgttccaaatgctttacccggcaaccaccagtaaaggaacgacccacagttacggatactcgttctggctcatactgctcgtcattcttctaaatatagtcactgtaaccatcatcattttctaccagaaggccagataccagcggaagcaggagcagagaaagccaatggaatatgctccaagggacggaattttattctgaattctctttcatctcattttggcgttgcatctattgtacatcagccctgagtagtaactggttagcttctctggacaattcagcatggtaacgtgactgtcatctgtgacagcatttgtgtttcatgacactgtgttcttcattgatgctgtactcctgaaaatttttcccacaaggttggggaaatgaatgggaaatgtcgctggtctgtgtggtattcaaagcagtagtatcatgatgagcgtaacgacccttctgacctggtctcacgatctgaaataataaaaggctgtgtcatgtttcttttc

SEQ ID NO: 12 - protein, 226 aa, Homo sapiens
MPTTKKTLMFLSSFFTSLGSFIVICSILGTQAWITSTIAVRDSASNGSIFITYGLFRGESSEELSHGLAEPKKKFAVLEILNNSSQKTLHSVTILFLVLSLITSLLSSGFTFYNSISNPYQTFLGPTGVYTWNGLGASFVFVTMILFVANTQSNQLSEELFQMLYPATTSKGTTHSYGYSFWLILLVILLNIVTVTIIIFYQKARYQRKQEQRKPMEYAPRDGILF

SEQ ID NO: 13 - DNA, 2214 bp, Homo sapiens
  source 1..2214: mol_type = other DNA
  CDS 105..1295
agactgggctgggcaggtctgagagttagggaaagtccgttcccactgccctcggggagagaagaaaggagggggcaagggagaagctgctggtcggactcacaatgaaaacgctccttcttttgctgctggtgctcctggagctgggagaggcccaaggatcccttcacagggtgcccctcaggaggcatccgtccctcaagaagaagctgcgggcacggagccagctctctgagttctggaaatcccataatttggacatgatccagttcaccgagtcctgctcaatggaccagagtgccaaggaacccctcatcaactacttggatatggaatacttcggcactatctccattggctccccaccacagaacttcactgtcatcttcgacactggctcctccaacctctgggtcccctctgtgtactgcactagcccagcctgcaagacgcacagcaggttccagccttcccagtccagcacatacagccagccaggtcaatctttctccattcagtatggaaccgggagcttgtccgggatcattggagccgaccaagtctctgtggaaggactaaccgtggttggccagcagtttggagaaagtgtcacagagccaggccagacctttgtggatgcagagtttgatggaattctgggcctgggatacccctccttggctgtgggaggagtgactccagtatttgacaacatgatggctcagaacctggtggacttgccgatgttttctgtctacatgagcagtaacccagaaggtggtgcggggagcgagctgatttttggaggctacgaccactcccatttctctgggagcctgaattgggtcccagtcaccaagcaagcttactggcagattgcactggataacatccaggtgggaggcactgttatgttctgctccgagggctgccaggccattgtggacacagggacttccctcatcactggcccttccgacaagattaagcagctgcaaaacgccattggggcagcccccgtggatggagaatatgctgtggagtgtgccaaccttaacgtcatgccggatgtcaccttcaccattaacggagtcccctataccctcagcccaactgcctacaccctactggacttcgtggatggaatgcagttctgcagcagtggctttcaaggacttgacatccaccctccagctgggcccctctggatcctgggggatgtcttcattcgacagttttactcagtctttgaccgtgggaataaccgtgtgggactggccccagcagtcccctaaggaggggccttgtgtctgtgcctgcctgtctgacagaccttgaatatgttaggctggggcattctttacacctacaaaaagttattttccagagaatgtagctgtttccagggttgcaacttgaattaagaccaaacagaacatgagaatacacacacacacacacatatacacacacacacacttcacacatacacaccactcccaccaccgtcatgatggaggaattacgttatacattcatattttgtattgatttttgattatgaaaatcaaaaattttcacatttgattatgaaaatctccaaacatatgcacaagcagagatcatggtataataaatccctttgcaactccactcagccctgacaacccatccacacacggccaggcctgtttatctacactgctgcccactcctctctccagctccacatgctgtacctggatcattctgaagcaaattccgagcattacatcattttgtccataaatatttctaacatccttaaatatacaatcggaattcaagcatctcccattgtcccacaaatgtttggctgtttttgtagttggattgtttgtattaggattcaagcaaggcccatatattgcatttatttgaaatgtctgtaagtctctttccatctacagagtttagcacatttgaacgttgctggttgaaatcccgaggtgtcatttgacatggttctctgaacttatctttcctataaaatggtagttagatctggaggtctgattttgtggcaaaaatacttcctaggtggtgctgggtacttcttgttgcatcctgtcaggaggcagataatgctggtgcctctctattggtaatgttaagactgctgggtgggtttggagttcttggctttaatcattcattacaaagttcagcatttta

SEQ ID NO: 14 - protein, 396 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 15 - DNA, 6128 bp, Homo sapiens
  source 1..6128: mol_type = other DNA
  CDS 370..2697
gggcctgcctgccagctagccggagccgcgggtgagcgcggcgagcggcgaccctggtgaggagcgcggcgcgggaggcacgttccttagctccgccgcggccgtcctccgcggctcgaggactccgcttccttccctcccctcccctgcgctccggcctggggtctcggcgcggggagcggagggaagggacgaaggaggagtaggtgaaagcggggtgaggggcggaagggtcccggcgcggggtgaggcgagggctgcctcttgttctcccgccgctgccgccgtctcctggtcgggtgccgcggccagaggcgcgcggggctgccgaggcacccgcactatgcaggcagactgccggccgccgcgatggcgagccgggcggtggtgagagccaggcgctgcccgcagtgtccccaagtccgggccgcggccgccgcccccgcctgggccgcgctccccctctcccgctccctccctccctgctccaactcctcctccttctccatgcctctgttcctcctgctcttacttgtcctgctcctgctgctcgaggacgctggagcccagcaaggtgatggatgtggacacactgtactaggccctgagagtggaacccttacatccataaactacccacagacctatcccaacagcactgtttgtgaatgggagatccgtgtaaagatgggagagagagttcgcatcaaatttggtgactttgacattgaagattctgattcttgtcactttaattacttgagaatttataatggaattggagtcagcagaactgaaataggcaaatactgtggtctggggttgcaaatgaaccattcaattgaatcaaaaggcaatgaaatcacattgctgttcatgagtggaatccatgtttctggacgcggatttttggcctcatactctgttatagataaacaagatctaattacttgtttggacactgcatccaattttttggaacctgagttcagtaagtactgcccagctggttgtctgcttccttttgctgagatatctggaacaattcctcatggatatagagattcctcgccattgtgcatggctggtgtgcatgcaggagtagtgtcaaacacgttgggcggccaaatcagtgttgtaattagtaaaggtatcccctattatgaaagttctttggctaacaacgtcacatctgtggtgggacacttatctacaagtctttttacatttaagacaagtggatgttatggaacactggggatggagtctggtgtgatcgcggatcctcaaataacagcatcatctgtgctggagtggactgaccacacagggcaagagaacagttggaaacccaaaaaagccaggctgaaaaaacctggaccgccttgggctgcttttgccactgatgaataccagtggttacaaatagatttgaataaggaaaagaaaataacaggcattataaccactggatccaccatggtggagcacaattactatgtgtctgcctacagaatcctgtacagtgatgatgggcagaaatggactgtgtacagagagcctggtgtggagcaagataagatatttcaaggaaacaaagattatcaccaggatgtgcgtaataactttttgccaccaattattgcacgttttattagagtgaatcctacccaatggcagcagaaaattgccatgaaaatggagctgctcggatgtcagtttattcctaaaggtcgtcctccaaaacttactcaacctccacctcctcggaacagcaatgacctcaaaaacactacagcccctccaaaaatagccaaaggtcgtgccccaaaatttacgcaaccactacaacctcgcagtagcaatgaatttcctgcacagacagaacaaacaactgccagtcctgatatcagaaatactaccgtaactccaaatgtaaccaaagatgtagcgctggctgcagttcttgtccctgtgctggtcatggtcctcactactctcattctcatattagtgtgtgcttggcactggagaaacagaaagaaaaaaactgaaggcacctatgacttaccttactgggaccgggcaggttggtggaaaggaatgaagcagtttcttcctgcaaaagcagtggaccatgaggaaaccccagttcgctatagcagcagcgaagttaatcacctgagtccaagagaagtcaccacagtgctgcaggctgactctgcagagtatgctcagccactggtaggaggaattgttggtacacttcatcaaagatctacctttaaaccagaagaaggaaaagaagcaggctatgcagacctagatccttacaactcaccagggcaggaagtttatcatgcctatgctgaaccactcccaattacggggcctgagtatgcaaccccaatcatcatggacatgtcagggcaccccacaacttcagttggtcagccctccacatccactttcaaggctacggggaaccaacctcccccactagtgggaacttacaatacacttctctccaggactgacagctgctcctcagcccaggcccagtatgataccccgaaagctgggaagccaggtctacctgccccagacgaattggtgtaccaggtgccacagagcacacaagaagtatcaggagcaggaagggatggggaatgtgatgtttttaaagaaatcctttgaagatgatgctgctttttacaaagcatcgttttaaagcacatggccttttttttttaattattagtggtagtaatatatagaatgtattacataactgtcactgaagtggttggggaaaatgtggtgactgaggtacaggaaactactaatcttgccatcttgctttaaggtgttatggtggcacagttactgctcgcctgttaaatttcaaatgtcctgtttgatactactgtagaacactatttttaatacagaaaaagctccctataatgcacttcagagaaattaaaaatcacagagtatttattaccaatgctgcaggtacattaatgaactcgagatggctctgtaagcctgactggcaataacgcacggtactgttcttgaaatacctaatggcttgaaattctagtctgtttgtgaaagatgggtactatcatgatttcctcttctattcctatattcttttctggattttttttaataattagtgatataagcattgtttttattgcagccatatccacttatccatcttaagatctgtagctgggattttctgacttgtaatgagcagggggattgctttttcactttgtgacactctttagagctttaatgcttcacagtatatggcctggtctcatccttgcgtgttccacttgaggccctttggtgtcttgccccattcttgtgtttataaaatgtttgagtatttctgatgagtgatgcttgccttagtctcatgaattcagatcccttcatgtcctttaagtatgctcctcaatgtgtaaacaggaacaactttatgatttgaaagctttaaaggagattcttctcccacccccaactttatttgcaatgggatttttcctaggagagttatgaaaagttgaaggcttctaagggaatactgtaaacatgacccacttatatttatcacagtgaaaggcaaaattattcactcagaagtaatataaattacctctttaaaaagtaaccagaatttgtcctttttggttttatacattcacaaacatatacatttttcttgagtctcaaggtattttatatttttagtcagaaaaaataatttttcatttcagttttccataaactgttacacaaaatataaacctaacgtgtatttttcaggactgcgtgatcgtgcactttgtgtggtaagaggtttgagtagtcctatatgtcacctagggaacagacattatagcttactagcaaatgaatattcatgccttgtttttgatacctcctggcagcttccatgtcaccacttgttcatacctgcccagagctagttttagacatggcaaaatagaaatcatctgtaatttattagctaacaatgtaaaaccatcttttaaagccttcagactgtcaagacgacatgagcagctcaccatatgataaaaatacataaatttgacattccctcttccataaacctttgtttgtagatttaatgttgaacagtacttttccataaagttctagtcacttctgttggcctgagccaccagattatgatgttgccagaattcactcaatttgaataaagatgaacagtatttgttttcttgtttccatgaattatatcagtattctaaaacatcgcttcagaaagagaactgtttatttctgcaggcttcctgtccttttgtggtatggttttttggccttattttcactggcttttccttctccaaactttgaggcgtgatttcattcattgaagaatcaatacatattttgtttcaaaatgtttgaaacaaaagacatagatggtagacttttattaaaacatatatggatgtggaaagcacatatattaatgcagtcatcccttttcaggtgggaagagagcaaaccagttgattttttaattcatccttagtacacagagaatatacttttcctcaagtaatatacctgtttgaagctttaagagagatgtttttggtaactatttcattttcccaaagaagtttgctattcttgtgttaattgtgtatacctgattgttttttcctggaggtttttgttgttgttgtttagttttgggtttttttttttttaagaggggcaagtgttttctgaaatgatgcatattttaagactcgattcatattgccactgtgctatccttgaactaccaataatttttataaaatatctagtttttactacttttatataaactttactttccagatgaagagctgagcctgattcaaatggtttttctgctttatacttctttttagttcattggtttttatagtagaggttttctattttttttttttttttttttactacatttatatgtctgatacatatacggctttggagacaatcaagtaacaactgaaaatgtgaaagtaaccatatctgacaaaattcccttgaatttttatcctttgcttgcaacatttaagactcaaagtcactggtatattggattaagttttttcctgttaatgcaattatagaaatacatcggagacacaacaaatgtggccattacaggtttcataaaattacactgacttggctgttacttgatcttaggaaacagcacagtttaagatattgtgaattctgacttatactttattaaatgctgtaaatctaaatagatcctgttggatgtgatgggtctagtccagtttatttaagttcatgtttcactgtttgcactttgcattgaacaatgggtttattcgctgatgtaaacggttcgagtgaagaattaatgcagtaagtatgacaacacatacacacttgcctctccccatctccagaagaggggagcagagtccgagcttatctaaatatgaatgtggccacaaagctgtggaaggtgacaaagcttaaacacctttgccctggctctgcattgtcacctagagagcaagaggtctatagaaacatcatgtcacatgaaacgattctctgctttttggttctgaacttgaagtccctaaactgcaaaatctaagagttgggtggttattaaaatgcttttaaagtcaactgtggcaccaattctaatgtaatccaacttgtgactgtttttttttgttttgttttgtttttgtgtgtgtgtgtgtggcactgggaaaagtggaaacaaacatgtattgaaatacatattggaaataaaaatggtttgagcgtcagtgatattctcccagaatgtacttatcttacctcggcatgtactgtagtcactcagtatttgtatatgttgctagaatttagattgtaaaatagtgaaattttaatgtgttcatttgtttttaatgtatatatgtcttgctcagattatttggtttaaataaaacaaccttgaggtttgtagcttttccttatactataaaatgccagattcttctattttaacctca

SEQ ID NO: 16 - protein, 775 aa, Homo sapiens
MASRAVVRARRCPQCPQVRAAAAAPAWAALPLSRSLPPCSNSSSFSMPLFLLLLLVLLLLLEDAGAQQGDGCGHTVLGPESGTLTSINYPQTYPNSTVCEWEIRVKMGERVRIKFGDFDIEDSDSCHFNYLRIYNGIGVSRTEIGKYCGLGLQMNHSIESKGNEITLLFMSGIHVSGRGFLASYSVIDKQDLITCLDTASNFLEPEFSKYCPAGCLLPFAEISGTIPHGYRDSSPLCMAGVHAGVVSNTLGGQISVVISKGIPYYESSLANNVTSVVGHLSTSLFTFKTSGCYGTLGMESGVIADPQITASSVLEWTDHTGQENSWKPKKARLKKPGPPWAAFATDEYQWLQIDLNKEKKITGIITTGSTMVEHNYYVSAYRILYSDDGQKWTVYREPGVEQDKIFQGNKDYHQDVRNNFLPPIIARFIRVNPTQWQQKIAMKMELLGCQFIPKGRPPKLTQPPPPRNSNDLKNTTAPPKIAKGRAPKFTQPLQPRSSNEFPAQTEQTTASPDIRNTTVTPNVTKDVALAAVLVPVLVMVLTTLILILVCAWHWRNRKKKTEGTYDLPYWDRAGWWKGMKQFLPAKAVDHEETPVRYSSSEVNHLSPREVTTVLQADSAEYAQPLVGGIVGTLHQRSTFKPEEGKEAGYADLDPYNSPGQEVYHAYAEPLPITGPEYATPIIMDMSGHPTTSVGQPSTSTFKATGNQPPPLVGTYNTLLSRTDSCSSAQAQYDTPKAGKPGLPAPDELVYQVPQSTQEVSGAGRDGECDVFKEIL

SEQ ID NO: 17 - DNA, 1949 bp, Homo sapiens
  source 1..1949: mol_type = other DNA
  CDS 87..1529
aactgtcactgtggagaggagagagagaggacagagagcaagtcactcccggctgcctttttcacctctgacagagcccagacaccatgaacgcaagtgaattccgaaggagagggaaggagatggtggattacatggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaacaattcaagtgaaa

SEQ ID NO: 18 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 19 - DNA, 3624 bp, Homo sapiens
  source 1..3624: mol_type = other DNA
  CDS 132..1919
ggactcgcgctcgcccgctggcgccctcggcttctctccgcgcctgggagcaccctccgccgcggccgttctccatgcgcagcgcccgcccgaggagctagacgtcagcttggagcggcgccggaccgtggatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccagagttttaaaaataaaaagggtattgttttgtcttctgtacagtgagttccttcccttttcaaagctttctttttatgctgtatgtgactatagatattcatataaaacaagtgcacgtgaagtttgcaaaatgctttaaggccttcctttcaaagcatagtccttttggagccgttttgtaccttttataccttggcttatttgaagttgacacatggggttagttactactctccatgtgcattggggacagtttttataagtgggaaggactcagtattattatatttgagatgataagcattttgtttgggaacaatgcttaaaaatattccagaaagttcagattttttttctttgtgaatgaaatatattctggcccacgaacagggcgatttcctttcagttttttccttttgcaacgtgccttgaagtctcaaagctcacctgaggttgcagacgttacccccaacagaagataggtagaaatgattccagtggcctctttgtattttcttcattgttgagtagatttcaggaaatcaggaggtgtttcacaatacagaatgatggcctttaactgtg

SEQ ID NO: 20 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 21 - DNA, 1493 bp, Homo sapiens
  source 1..1493: mol_type = other DNA
  CDS 334..1410
agtagtcgcctggcaggagaatttgaaagggtgccccaaaggacaatctctaaaggggtaagggagatacctaccttgtctggtaggggagatgtttcgttttcatgctttaccagaaaatccacttccctgccgaccttagtttcaaagcttattcttaattagagacaagaaacctgtttcaacttgaagacaccgtatgaggtgaatggacagccagccaccacaatgaaagaaatcaaaccaggaataacctatgctgaacccacgcctcaatcgtccccaagtgtttcctgacacgcatctttgcttacagtgcatcacaactgaagaatggggttcaacttgacgcttgcaaaattaccaaataacgagctgcacggccaagagagtcacaattcaggcaacaggagcgacgggccaggaaagaacaccacccttcacaatgaatttgacacaattgtcttgccggtgctttatctcattatatttgtggcaagcatcttgctgaatggtttagcagtgtggatcttcttccacattaggaataaaaccagcttcatattctatctcaaaaacatagtggttgcagacctcataatgacgctgacatttccatttcgaatagtccatgatgcaggatttggaccttggtacttcaagtttattctctgcagatacacttcagttttgttttatgcaaacatgtatacttccatcgtgttccttgggctgataagcattgatcgctatctgaaggtggtcaagccatttggggactctcggatgtacagcataaccttcacgaaggttttatctgtttgtgtttgggtgatcatggctgttttgtctttgccaaacatcatcctaacaaatggtcagccaacagaggacaatatccatgactgctcaaaacttaaaagtcctttgggggtcaaatggcatacggcagtcacctatgtgaacagctgcttgtttgtggccgtgctggtgattctgatcggatgttacatagccatatccaggtacatccacaaatccagcaggcaattcataagtcagtcaagccgaaagcgaaaacataaccagagcatcagggttgttgtggctgtgttttttacctgctttctaccatatcacttgtgcagaattccttttacttttagtcacttagacaggcttttagatgaatctgcacaaaaaatcctatattactgcaaagaaattacacttttcttgtctgcgtgtaatgtttgcctggatccaataatttactttttcatgtgtaggtcattttcaagaaggctgttcaaaaaatcaaatatcagaaccaggagtgaaagcatcagatcactgcaaagtgtgagaagatcggaagttcgcatatattatgattacactgatgtgtaggccttttattgtttgttggaatcgatatgtacaaagtgtaaataaatgtttcttttcattatccttgcttgagcccatcaaaa

SEQ ID NO: 22 - protein, 358 aa, Homo sapiens
MGFNLTLAKLPNNELHGQESHNSGNRSDGPGKNTTLHNEFDTIVLPVLYLIIFVASILLNGLAVWIFFHIRNKTSFIFYLKNIVVADLIMTLTFPFRIVHDAGFGPWYFKFILCRYTSVLFYANMYTSIVFLGLISIDRYLKVVKPFGDSRMYSITFTKVLSVCVWVIMAVLSLPNIILTNGQPTEDNIHDCSKLKSPLGVKWHTAVTYVNSCLFVAVLVILIGCYIAISRYIHKSSRQFISQSSRKRKHNQSIRVVVAVFFTCFLPYHLCRIPFTFSHLDRLLDESAQKILYYCKEITLFLSACNVCLDPIIYFFMCRSFSRRLFKKSNIRTRSESIRSLQSVRRSEVRIYYDYTDV

SEQ ID NO: 23 - DNA, 4889 bp, Homo sapiens
  source 1..4889: mol_type = other DNA
  CDS 331..3486
agatccgccgcgaagccgggatcgaaggcgacagcgcggccaagggggcgcggccgggacaagctgggggccggttgcccggggcagggacggcggcgacccggccgctggggaggcaggaagatagacccacggatcttaggaagggatccgagagcgcagctgtgaaactggctggggctgggggcacgaaaccgatcagcgctacggagcgcagcggccggcgggttccagtgtcctccggcggcgcggggagcaggtgaacaggtcctcacgcccagctccgcgccctcacgcgctctcgccgggaccccgcttccgctggcagccatgggccccggccccagccgcgcgccccgcgccccacgcctgatgctctgtgcgctcgccttgatggtggcggccggcggctgcgtcgtctccgccttcaacctggatacccgattcctggtagtgaaggaggccgggaacccgggcagcctcttcggctactcggtcgccctccatcggcagacagagcggcagcagcgctacctgctcctggctggtgccccccgggagctcgctgtgcccgatggctacaccaaccggactggtgctgtgtacctgtgcccactcactgcccacaaggatgactgtgagcggatgaacatcacagtgaaaaatgaccctggccatcacattattgaggacatgtggcttggagtgactgtggccagccagggccctgcaggcagagttctggtctgtgcccaccgctacacccaggtgctgtggtcagggtcagaagaccagcggcgcatggtgggcaagtgctacgtgcgaggcaatgacctagagctggactccagtgatgactggcagacctaccacaacgagatgtgcaatagcaacacagactacctggagacgggcatgtgccagctgggcaccagcggtggcttcacccagaacactgtgtacttcggcgcccccggtgcctacaactggaaaggaaacagctacatgattcagcgcaaggagtgggacttatctgagtatagttacaaggacccagaggaccaaggaaacctctatattgggtacacgatgcaggtaggcagcttcatcctgcaccccaaaaacatcaccattgtgacaggtgccccacggcaccgacatatgggcgcggtgttcttgctgagccaggaggcaggcggagacctgcggaggaggcaggtgctggagggctcgcaggtgggcgcctattttggcagcgccattgccctggcagacctgaacaatgatgggtggcaggacctcctggtgggcgccccctactacttcgagaggaaagaggaagtagggggtgccatctatgtcttcatgaaccaggcgggaacctccttccctgctcacccctcactccttcttcatggccccagtggctctgcctttggtttatctgtggccagcattggtgacatcaaccaggatggatttcaggatattgctgtgggagctccgtttgaaggcttgggcaaagtgtacatctatcacagtagctctaaggggctccttagacagccccagcaggtaatccatggagagaagctgggactgcctgggttggccaccttcggctattccctcagtgggcagatggatgtggatgagaacttctacccagaccttctagtgggaagcctgtcagaccacattgtgctgctgcgggcccggcccgtcatcaacatcgtccacaagaccttggtgcccaggccagctgtgctggaccctgcactttgcacggccacctcttgtgtgcaagtggagctgtgctttgcttacaaccagagtgccgggaaccccaactacaggcgaaacatcaccctggcctacactctggaggctgacagggaccgccggccgccccggctccgctttgccggcagtgagtccgctgtcttccacggcttcttctccatgcccgagatgcgctgccagaagctggagctgctcctgatggacaacctccgtgacaaactccgccccatcatcatctccatgaactactctttacctttgcggatgcccgatcgcccccggctggggctgcggtccctggacgcctacccgatcctcaaccaggcacaggctctggagaaccacactgaggtccagttccagaaggagtgcgggcctgacaacaagtgtgagagcaacttgcagatgcgggcagccttcgtgtcagagcagcagcagaagctgagcaggctccagtacagcagagacgtccggaaattgctcctgagcatcaacgtgacgaacacccggacctcggagcgctccggggaggacgcccacgaggcgctgctcaccctggtggtgcctcccgccctgctgctgtcctcagtgcgcccccccggggcctgccaagctaatgagaccatcttttgcgagctggggaaccccttcaaacggaaccagaggatggagctgctcatcgcctttgaggtcatcggggtgaccctgcacacaagggaccttcaggtgcagctgcagctctccacgtcgagtcaccaggacaacctgtggcccatgatcctcactctgctggtggactatacactccagacctcgcttagcatggtaaatcaccggctacaaagcttctttggggggacagtgatgggtgagtctggcatgaaaactgtggaggatgtaggaagccccctcaagtatgaattccaggtgggcccaatgggggaggggctggtgggcctggggaccctggtcctaggtctggagtggccctacgaagtcagcaatggcaagtggctgctgtatcccacggagatcaccgtccatggcaatgggtcctggccctgccgaccacctggagaccttatcaaccctctcaacctcactctttctgaccctggggacaggccatcatccccacagcgcaggcggcgacagctggatccagggggaggccagggccccccacctgtcactctggctgctgccaaaaaagccaagtctgagactgtgctgacctgtgccacagggcgtgcccactgtgtgtggctagagtgccccatccctgatgcccccgttgtcaccaacgtgactgtgaaggcacgagtgtggaacagcaccttcatcgaggattacagagactttgaccgagtccgggtaaatggctgggctaccctattcctccgaaccagcatccccaccatcaacatggagaacaagaccacgtggttctctgtggacattgactcggagctggtggaggagctgccggccgaaatcgagctgtggctggtgctggtggccgtgggtgcagggctgctgctgctggggctgatcatcctcctgctgtggaagtgcggcttcttcaagcgagcccgcactcgcgccctgtatgaagctaagaggcagaaggcggagatgaagagccagccgtcagagacagagaggctgaccgacgactactgagggggcagccccccgcccccggcccacctggtgtgacttctttaagcggacccgctattatcagatcatgcccaagtaccacgcagtgcggatccgggaggaggagcgctacccacctccagggagcaccctgcccaccaagaagcactgggtgaccagctggcagactcgggaccaatactactgacgtcctccctgatcccaccccctcctcccccagtgtcccctttcttcctatttatcataagttatgcctctgacagtccacaggggccaccacctttggctggtagcagcaggctcaggcacatacacctcgtcaagagcatgcacatgctgtctggccctggggatcttcccacaggagggccagcgctgtggaccttacaacgccgagtgcactgcattcctgtgccctagatgcacgtggggcccactgctcgtggactgtgctggtgcatcacggatggtgcatgggctcgccgtgtctcagcctctgccagcgccaaaacaagccaaagagcctcccaccagagccgggaggaaaaggcccctgcaatgtggtgacacctccccctttcacactggatccatcttgagccacagtcactggattgactttgctgtcaaaactactgacagggagcagcccccgggccgctggctggtgggcccccaatgacacccatgccagagaggtggggatcctgcctaaggttgtctacgggggcacttggaggacctggcgtgctcagacccaacagcaaaggaactagaaagaaggacccagaacggcttgctttcctgcatctctgtgaagcctctctccttggccacagactgaactcgcagggaatgcagcaggaaggaacaaagacaggcaaacggcaacgtagcctgggctcactgtgctggggcacggcgggatcctccacagagaggaggggaccaattctggacagacagatgttgggaggatacagaggagatgccacttctcactcaccactaccagccagcctcagaaggccccagagagaccctgcaagaccacggagggagcgacacttgaatgtagaataggcagggggccctgccccaccccatccagccagaccccacgctgaccatgcgtcaggggcctagaggtggagttcttagctatccttggctttcagagccagcctggctctgccccctcccccatgggctgtgtcctaaggcccatttgagaagctgaggctagttccagaaaacctctcctgacccctgcctgttggcaggcccactccccagccccagccccttccatggtactgtagcaggggaattccctccccctccttgtgccttctttgtatataggcttctcacggcgaccaataaacagctcccagtttgta

SEQ ID NO: 24 - protein, 1051 aa, Homo sapiens
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCGFFKRARTRALYEAKRQKAEMKSQPSETERLTDDY

SEQ ID NO: 25 - DNA, 2238 bp, Homo sapiens
  source 1..2238: mol_type = other DNA
  CDS 99..1871
aacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatataa

SEQ ID NO: 26 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 27 - DNA, 2308 bp, Homo sapiens
  source 1..2308: mol_type = other DNA
  CDS 70..1764
agtcctgcttctcttccctctctcctccagcctctcacactctcctcagctctctcatctcctggaaccatggccagcacatccaccaccatcaggagccacagcagcagccgccggggtttcagtgccaactcagccaggctccctggggtcagccgctctggcttcagcagcgtctccgtgtcccgctccaggggcagtggtggcctgggtggtgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcaccgtcaaccagagtctcctgactcccctcaacctgcaaatcgatcccaccatccagcgggtgcgggctgaggagcgtgaacagatcaagaccctcaacaacaagtttgcctccttcatcgacaaggtgcggttcctggagcagcagaacaaggttctggaaacaaagtggaccctgctgcaggagcagggcaccaagactgtgaggcagaacctggagccgttgttcgagcagtacatcaacaacctcaggaggcagctggacagcattgtcggggaacggggccgcctggactcagagctcagaggcatgcaggacctggtggaggacttcaagaacaaatatgaggatgaaatcaacaagcgcacagcagcagagaatgaatttgtgactctgaagaaggatgtggatgctgcctacatgaacaaggttgaactgcaagccaaggcagacactctcacagacgagatcaacttcctgagagccttgtatgatgcagagctgtcccagatgcagacccacatctcagacacatctgtggtgctgtccatggacaacaaccgcaacctggacctggacagcatcatcgctgaggtcaaggcccaatatgaggagattgctcagagaagccgggctgaggctgagtcctggtaccagaccaagtacgaggagctgcaggtcacagcaggcagacatggggacgacctgcgcaacaccaagcaggagattgctgagatcaaccgcatgatccagaggctgagatctgagatcgaccacgtcaagaagcagtgcgccaacctgcaggccgccattgctgatgctgagcagcgtggggagatggccctcaaggatgccaagaacaagctggaagggctggaggatgccctgcagaaggccaagcaggacctggcccggctgctgaaggagtaccaggagctgatgaatgtcaagctggccctggacgtggagatcgccacctaccgcaagctgctggagggtgaggagtgcaggctgaatggcgaaggcgttggacaagtcaacatctctgtggtgcagtccaccgtctccagtggctatggcggtgccagtggtgtcggcagtggcttaggcctgggtggaggaagcagctactcctatggcagtggtcttggcgttggaggtggcttcagttccagcagtggcagagccattgggggtggcctcagctctgttggaggcggcagttccaccatcaagtacaccaccacctcctcctccagcaggaagagctataagcactaaagtgcgtctgctagctctcggtcccacagtcctcaggcccctctctggctgcagagccctctcctcaggttgcctttcctctcctggcctccagtctcccctgctgtcccaggtagagctgggtatggatgcttagtgccctcacttcttctctctctctctataccatctgagcacccattgctcaccatcagatcaacctctgattttacatcatgatgtaatcaccactggagcttcactgttactaaattattaatttcttgcctccagtgttctatctctgaggctgagcattataagaaaatgacctctgctccttttcattgcagaaaattgccaggggcttatttcagaacaacttccacttactttccactggctctcaaactctctaacttataagtgttgtgaacccccacccaggcagtatccatgaaagcacaagtgactagtcctatgatgtacaaagcctgtatctctgtgatgatttctgtgctcttcgctgtttgcaattgctaaataaagcagatttataatacaa

SEQ ID NO: 28 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 29 - DNA, 1712 bp, Homo sapiens
  source 1..1712: mol_type = other DNA
  CDS 64..1434
ctggtacctcctgccagcatctcttgggtttgctgagaactcacgggctccagctacctggccatgaccaccacatttctgcaaacttcttcctccacctttgggggtggctcaacccgagggggttccctcctggctgggggaggtggctttggtggggggagtctctctgggggaggtggaagccgaagtatctcagcttcttctgctaggtttgtctcttcagggtcaggaggaggatatgggggtggcatgagggtctgtggctttggtggaggggctggtagtgttttcggtggaggctttggagggggcgttggtgggggttttggtggtggctttggtggtggcgatggtggtctcctctctggcaatgagaaaattaccatgcagaacctcaatgaccgcctggcctcctacctggacaaggtacgtgccctggaggaggccaatgctgacctggaggtgaagatccatgactggtaccagaagcagaccccaaccagcccagaatgcgactacagccaatacttcaagaccattgaagagctccgggacaagatcatggccaccaccatcgacaactcccgggtcatcctggagatcgacaatgccaggctggctgcggacgacttcaggctcaagtatgagaatgagctggccctgcgccagggcgttgaggctgacatcaacggcttgcgccgagtcctggatgagctgaccctggccaggactgacctggagatgcagatcgagggcctgaatgaggagctagcctacctgaagaagaaccacgaagaggagatgaaggagttcagcagccagctggccggccaggtcaatgtggagatggacgcagcaccgggtgtggacctgacccgtgtgctggcagagatgagggagcagtacgaggccatggcggagaagaaccgccgggatgtcgaggcctggttcttcagcaagactgaggagctgaacaaagaggtggcctccaacacagaaatgatccagaccagcaagacggagatcacagacctgagacgcacgatgcaggagctggagatcgagctgcagtcccagctcagcatgaaagctgggctggagaactcactggccgagacagagtgccgctatgccacgcagctgcagcagatccaggggctcattggtggcctggaggcccagctgagtgagctccgatgcgagatggaggctcagaaccaggagtacaagatgctgcttgacataaagacacggctggagcaggagatcgctacttaccgcagcctgctcgagggccaggatgccaagatggctggcattgccatcagggaagcctcttcaggaggtggtggtagcagcagcaatttccacatcaatgtagaagagtcagtggatggacaggtggtttcttcccacaagagagaaatctaagtgtctattgcaggagaaacgtcccttgccactccccactctcatcaggccaagtggaggactggccagagggcctgcacatgcaaactccagtccctgccttcagagagctgaaaagggtccctcggtcttttatttcagggctttgcatgcgctctattccccctctgcctctccccaccttctttggagcaaggagatgcagctgtattgtgtaacaagctcatttgtacagtgtctgttcatgtaataaagaattacttttccttttgcaaata

SEQ ID NO: 30 - protein, 456 aa, Homo sapiens
MTTTFLQTSSSTFGGGSTRGGSLLAGGGGFGGGSLSGGGGSRSISASSARFVSSGSGGGYGGGMRVCGFGGGAGSVFGGGFGGGVGGGFGGGFGGGDGGLLSGNEKITMQNLNDRLASYLDKVRALEEANADLEVKIHDWYQKQTPTSPECDYSQYFKTIEELRDKIMATTIDNSRVILEIDNARLAADDFRLKYENELALRQGVEADINGLRRVLDELTLARTDLEMQIEGLNEELAYLKKNHEEEMKEFSSQLAGQVNVEMDAAPGVDLTRVLAEMREQYEAMAEKNRRDVEAWFFSKTEELNKEVASNTEMIQTSKTEITDLRRTMQELEIELQSQLSMKAGLENSLAETECRYATQLQQIQGLIGGLEAQLSELRCEMEAQNQEYKMLLDIKTRLEQEIATYRSLLEGQDAKMAGIAIREASSGGGGSSSNFHINVEESVDGQVVSSHKREI

SEQ ID NO: 31 - DNA, 1517 bp, Homo sapiens
  source 1..1517: mol_type = other DNA
  CDS 67..1365
acacaacttggggcccctctcctctccagcccttctcctgtgtgcctgcctcctgccgccgccaccatgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctgaggactcagctaccccggccggccacccaggaggcagggaggcagccgccccatctgccccacagtctccggcctctccagcctcagccccctgcttcagtcccttccccatgcttccttgcctgatgacaataaagcttgttgactcagcta

SEQ ID NO: 32 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

SEQ ID NO: 33 - DNA, 1121 bp, Homo sapiens
  source 1..1121: mol_type = other DNA
  CDS 60..1031
accatctcccactcctgcagctcttctcacaggaccagccactagcgcagcctcgagcgatggcctatgtccccgcaccgggctaccagcccacctacaacccgacgctgccttactaccagcccatcccgggcgggctcaacgtgggaatgtctgtttacatccaaggagtggccagcgagcacatgaagcggttcttcgtgaactttgtggttgggcaggatccgggctcagacgtcgccttccacttcaatccgcggtttgacggctgggacaaggtggtcttcaacacgttgcagggcgggaagtggggcagcgaggagaggaagaggagcatgcccttcaaaaagggtgccgcctttgagctggtcttcatagtcctggctgagcactacaaggtggtggtaaatggaaatcccttctatgagtacgggcaccggcttcccctacagatggtcacccacctgcaagtggatggggatctgcaacttcaatcaatcaacttcatcggaggccagcccctccggccccagggacccccgatgatgccaccttaccctggtcccggacattgccatcaacagctgaacagcctgcccaccatggaaggacccccaaccttcaacccgcctgtgccatatttcgggaggctgcaaggagggctcacagctcgaagaaccatcatcatcaagggctatgtgcctcccacaggcaagagctttgctatcaacttcaaggtgggctcctcaggggacatagctctgcacattaatccccgcatgggcaacggtaccgtggtccggaacagccttctgaatggctcgtggggatccgaggagaagaagatcacccacaacccatttggtcccggacagttctttgatctgtccattcgctgtggcttggatcgcttcaaggtttacgccaatggccagcacctctttgactttgcccatcgcctctcggccttccagagggtggacacattggaaatccagggtgatgtcaccttgtcctatgtccagatctaatctattcctggggccataactcatgggaaaacagaattatcccctaggactcctttctaagcccctaataaaatgtctgagggtgtctca

SEQ ID NO: 34 - protein, 323 aa, Homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 35 - DNA, 1490 bp, Homo sapiens
  source 1..1490: mol_type = other DNA
  CDS 29..475
agcctagcactctgacctagcagtcaacatgaaggctctcattgttctggggcttgtcctcctttctgttacggtccagggcaaggtctttgaaaggtgtgagttggccagaactctgaaaagattgggaatggatggctacaggggaatcagcctagcaaactggatgtgtttggccaaatgggagagtggttacaacacacgagctacaaactacaatgctggagacagaagcactgattatgggatatttcagatcaatagccgctactggtgtaatgatggcaaaaccccaggagcagttaatgcctgtcatttatcctgcagtgctttgctgcaagataacatcgctgatgctgtagcttgtgcaaagagggttgtccgtgatccacaaggcattagagcatgggtggcatggagaaatcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttctttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatacgattatgcccaatattaagtaaaaaatataagaaaaggttatcttaaatagatcttaggcaaaataccagctgatgaaggcatctgatgccttcatctgttcagtcatctccaaaaacagtaaaaataaccactttttgttgggcaatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaattaaaggctttgcaacaca

SEQ ID NO: 36 - protein, 148 aa, Homo sapiens
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 37 - DNA, 14352 bp, Homo sapiens
  source 1..14352: mol_type = other DNA
  CDS 56..13537
catttcgccagctcctctgggggtgacaggcaagtgagacgtgctcagagctccgatgccaaggccagggaccatggcgctgtgtctgctgaccttggtcctctcgctcttgcccccacaagctgctgcagaacaggacctcagtgtgaacagggctgtgtgggatggaggagggtgcatctcccaaggggacgtcttgaaccgtcagtgccagcagctgtctcagcacgttaggacaggttctgcggcaaacaccgccacaggtacaacatctacaaatgtcgtggagccaagaatgtatttgagttgcagcaccaaccctgagatgacctcgattgagtccagtgtgacttcagacactcctggtgtctccagtaccaggatgacaccaacagaatccagaacaacttcagaatctaccagtgacagcaccacacttttccccagttctactgaagacacttcatctcctacaactcctgaaggcaccgacgtgcccatgtcaacaccaagtgaagaaagcatttcatcaacaatggcttttgtcagcactgcacctcttcccagttttgaggcctacacatctttaacatataaggttgatatgagcacacctctgaccacttctactcaggcaagttcatctcctactactcctgaaagcaccaccatacccaaatcaactaacagtgaaggaagcactccattaacaagtatgcctgccagcaccatgaaggtggccagttcagaggctatcacccttttgacaactcctgttgaaatcagcacacctgtgaccatttctgctcaagccagttcatctcctacaactgctgaaggtcccagcctgtcaaactcagctcctagtggaggaagcactccattaacaagaatgcctctcagcgtgatgctggtggtcagttctgaggctagcaccctttcaacaactcctgctgccaccaacattcctgtgatcacttctactgaagccagttcatctcctacaacggctgaaggcaccagcataccaacctcaacttatactgaaggaagcactccattaacaagtacgcctgccagcaccatgccggttgccacttctgaaatgagcacactttcaataactcctgttgacaccagcacacttgtgaccacttctactgaacccagttcacttcctacaactgctgaagctaccagcatgctaacctcaactcttagtgaaggaagcactccattaacaaatatgcctgtcagcaccatattggtggccagttctgaggctagcaccacttcaacaattcctgttgactccaaaacttttgtgaccactgctagtgaagccagctcatctcccacaactgctgaagataccagcattgcaacctcaactcctagtgaaggaagcactccattaacaagtatgcctgtcagcaccactccagtggccagttctgaggctagcaacctttcaacaactcctgttgactccaaaactcaggtgaccacttctactgaagccagttcatctcctccaactgctgaagttaacagcatgccaacctcaactcctagtgaaggaagcactccattaacaagtatgtctgtcagcaccatgccggtggccagttctgaggctagcaccctttcaacaactcctgttgacaccagcacacctgtgaccacttctagtgaagccagttcatcttctacaactcctgaaggtaccagcataccaacctcaactcctagtgaaggaagcactccattaacaaacatgcctgtcagcaccaggctggtggtcagttctgaggctagcaccacttcaacaactcctgctgactccaacacttttgtgaccacttctagtgaagctagttcatcttctacaactgctgaaggtaccagcatgccaacctcaacttacagtgaaagaggcactacaataacaagtatgtctgtcagcaccacactggtggccagttctgaggctagcaccctttcaacaactcctgttgactccaacactcctgtgaccacttcaactgaagccacttcatcttctacaactgcggaaggtaccagcatgccaacctcaacttatactgaaggaagcactccattaacaagtatgcctgtcaacaccacactggtggccagttctgaggctagcaccctttcaacaactcctgttgacaccagcacacctgtgaccacttcaactgaagccagttcctctcctacaactgctgatggtgccagtatgccaacctcaactcctagtgaaggaagcactccattaacaagtatgcctgtcagcaaaacgctgttgaccagttctgaggctagcaccctttcaacaactcctcttgacacaagcacacatatcaccacttctactgaagccagttgctctcctacaaccactgaaggtaccagcatgccaatctcaactcctagtgaaggaagtcctttattaacaagtatacctgtcagcatcacaccggtgaccagtcctgaggctagcaccctttcaacaactcctgttgactccaacagtcctgtgaccacttctactgaagtcagttcatctcctacacctgctgaaggtaccagcatgccaacctcaacttatagtgaaggaagaactcctttaacaagtatgcctgtcagcaccacactggtggccacttctgcaatcagcaccctttcaacaactcctgttgacaccagcacacctgtgaccaattctactgaagcccgttcgtctcctacaacttctgaaggtaccagcatgccaacctcaactcctggggaaggaagcactccattaacaagtatgcctgacagcaccacgccggtagtcagttctgaggctagaacactttcagcaactcctgttgacaccagcacacctgtgaccacttctactgaagccacttcatctcctacaactgctgaaggtaccagcataccaacctcgactcctagtgaaggaacgactccattaacaagcacacctgtcagccacacgctggtggccaattctgaggctagcaccctttcaacaactcctgttgactccaacactcctttgaccacttctactgaagccagttcacctcctcccactgctgaaggtaccagcatgccaacctcaactcctagtgaaggaagcactccattaacacgtatgcctgtcagcaccacaatggtggccagttctgaaacgagcacactttcaacaactcctgctgacaccagcacacctgtgaccacttattctcaagccagttcatcttctacaactgctgacggtaccagcatgccaacctcaacttatagtgaaggaagcactccactaacaagtgtgcctgtcagcaccaggctggtggtcagttctgaggctagcaccctttccacaactcctgtcgacaccagcatacctgtcaccacttctactgaagccagttcatctcctacaactgctgaaggtaccagcataccaacctcacctcccagtgaaggaaccactccgttagcaagtatgcctgtcagcaccacgctggtggtcagttctgaggctaacaccctttcaacaactcctgtggactccaaaactcaggtggccacttctactgaagccagttcacctcctccaactgctgaagttaccagcatgccaacctcaactcctggagaaagaagcactccattaacaagtatgcctgtcagacacacgccagtggccagttctgaggctagcaccctttcaacatctcccgttgacaccagcacacctgtgaccacttctgctgaaaccagttcctctcctacaaccgctgaaggtaccagcttgccaacctcaactactagtgaaggaagtactctattaacaagtatacctgtcagcaccacgctggtgaccagtcctgaggctagcacccttttaacaactcctgttgacactaaaggtcctgtggtcacttctaatgaagtcagttcatctcctacacctgctgaaggtaccagcatgccaacctcaacttatagtgaaggaagaactcctttaacaagtatacctgtcaacaccacactggtggccagttctgcaatcagcatcctttcaacaactcctgttgacaacagcacacctgtgaccacttctactgaagcctgttcatctcctacaacttctgaaggtaccagcatgccaaactcaaatcctagtgaaggaaccactccgttaacaagtatacctgtcagcaccacgccggtagtcagttctgaggctagcaccctttcagcaactcctgttgacaccagcacccctgggaccacttctgctgaagccacttcatctcctacaactgctgaaggtatcagcataccaacctcaactcctagtgaaggaaagactccattaaaaagtatacctgtcagcaacacgccggtggccaattctgaggctagcaccctttcaacaactcctgttgactctaacagtcctgtggtcacttctacagcagtcagttcatctcctacacctgctgaaggtaccagcatagcaatctcaacgcctagtgaaggaagcactgcattaacaagtatacctgtcagcaccacaacagtggccagttctgaaatcaacagcctttcaacaactcctgctgtcaccagcacacctgtgaccacttattctcaagccagttcatctcctacaactgctgacggtaccagcatgcaaacctcaacttatagtgaaggaagcactccactaacaagtttgcctgtcagcaccatgctggtggtcagttctgaggctaacaccctttcaacaacccctattgactccaaaactcaggtgaccgcttctactgaagccagttcatctacaaccgctgaaggtagcagcatgacaatctcaactcctagtgaaggaagtcctctattaacaagtatacctgtcagcaccacgccggtggccagtcctgaggctagcaccctttcaacaactcctgttgactccaacagtcctgtgatcacttctactgaagtcagttcatctcctacacctgctgaaggtaccagcatgccaacctcaacttatactgaaggaagaactcctttaacaagtataactgtcagaacaacaccggtggccagctctgcaatcagcaccctttcaacaactcccgttgacaacagcacacctgtgaccacttctactgaagcccgttcatctcctacaacttctgaaggtaccagcatgccaaactcaactcctagtgaaggaaccactccattaacaagtatacctgtcagcaccacgccggtactcagttctgaggctagcaccctttcagcaactcctattgacaccagcacccctgtgaccacttctactgaagccacttcgtctcctacaactgctgaaggtaccagcataccaacctcgactcttagtgaaggaatgactccattaacaagcacacctgtcagccacacgctggtggccaattctgaggctagcaccctttcaacaactcctgttgactctaacagtcctgtggtcacttctacagcagtcagttcatctcctacacctgctgaaggtaccagcatagcaacctcaacgcctagtgaaggaagcactgcattaacaagtatacctgtcagcaccacaacagtggccagttctgaaaccaacaccctttcaacaactcccgctgtcaccagcacacctgtgaccacttatgctcaagtcagttcatctcctacaactgctgacggtagcagcatgccaacctcaactcctagggaaggaaggcctccattaacaagtatacctgtcagcaccacaacagtggccagttctgaaatcaacaccctttcaacaactcttgctgacaccaggacacctgtgaccacttattctcaagccagttcatctcctacaactgctgatggtaccagcatgccaaccccagcttatagtgaaggaagcactccactaacaagtatgcctctcagcaccacgctggtggtcagttctgaggctagcactctttccacaactcctgttgacaccagcactcctgccaccacttctactgaaggcagttcatctcctacaactgcaggaggtaccagcatacaaacctcaactcctagtgaacggaccactccattagcaggtatgcctgtcagcactacgcttgtggtcagttctgagggtaacaccctttcaacaactcctgttgactccaaaactcaggtgaccaattctactgaagccagttcatctgcaaccgctgaaggtagcagcatgacaatctcagctcctagtgaaggaagtcctctactaacaagtatacctctcagcaccacgccggtggccagtcctgaggctagcaccctttcaacaactcctgttgactccaacagtcctgtgatcacttctactgaagtcagttcatctcctatacctactgaaggtaccagcatgcaaacctcaacttatagtgacagaagaactcctttaacaagtatgcctgtcagcaccacagtggtggccagttctgcaatcagcaccctttcaacaactcctgttgacaccagcacacctgtgaccaattctactgaagcccgttcatctcctacaacttctgaaggtaccagcatgccaacctcaactcctagtgaaggaagcactccattcacaagtatgcctgtcagcaccatgccggtagttacttctgaggctagcaccctttcagcaactcctgttgacaccagcacacctgtgaccacttctactgaagccacttcatctcctacaactgctgaaggtaccagcataccaacttcaactcttagtgaaggaacgactccattaacaagtatacctgtcagccacacgctggtggccaattctgaggttagcaccctttcaacaactcctgttgactccaacactcctttcactacttctactgaagccagttcacctcctcccactgctgaaggtaccagcatgccaacctcaacttctagtgaaggaaacactccattaacacgtatgcctgtcagcaccacaatggtggccagttttgaaacaagcacactttctacaactcctgctgacaccagcacacctgtgactacttattctcaagccggttcatctcctacaactgctgacgatactagcatgccaacctcaacttatagtgaaggaagcactccactaacaagtgtgcctgtcagcaccatgccggtggtcagttctgaggctagcacccattccacaactcctgttgacaccagcacacctgtcaccacttctactgaagccagttcatctcctacaactgctgaaggtaccagcataccaacctcacctcctagtgaaggaaccactccgttagcaagtatgcctgtcagcaccacgccggtggtcagttctgaggctggcaccctttccacaactcctgttgacaccagcacacctatgaccacttctactgaagccagttcatctcctacaactgctgaagatatcgtcgtgccaatctcaactgctagtgaaggaagtactctattaacaagtatacctgtcagcaccacgccagtggccagtcctgaggctagcaccctttcaacaactcctgttgactccaacagtcctgtggtcacttctactgaaatcagttcatctgctacatccgctgaaggtaccagcatgcctacctcaacttatagtgaaggaagcactccattaagaagtatgcctgtcagcaccaagccgttggccagttctgaggctagcactctttcaacaactcctgttgacaccagcatacctgtcaccacttctactgaaaccagttcatctcctacaactgcaaaagataccagcatgccaatctcaactcctagtgaagtaagtacttcattaacaagtatacttgtcagcaccatgccagtggccagttctgaggctagcaccctttcaacaactcctgttgacaccaggacacttgtgaccacttccactggaaccagttcatctcctacaactgctgaaggtagcagcatgccaacctcaactcctggtgaaagaagcactccattaacaaatatacttgtcagcaccacgctgttggccaattctgaggctagcaccctttcaacaactcctgttgacaccagcacacctgtcaccacttctgctgaagccagttcttctcctacaactgctgaaggtaccagcatgcgaatctcaactcctagtgatggaagtactccattaacaagtatacttgtcagcaccctgccagtggccagttctgaggctagcaccgtttcaacaactgctgttgacaccagcatacctgtcaccacttctactgaagccagttcctctcctacaactgctgaagttaccagcatgccaacctcaactcctagtgaaacaagtactccattaactagtatgcctgtcaaccacacgccagtggccagttctgaggctggcaccctttcaacaactcctgttgacaccagcacacctgtgaccacttctactaaagccagttcatctcctacaactgctgaaggtatcgtcgtgccaatctcaactgctagtgaaggaagtactctattaacaagtatacctgtcagcaccacgccggtggccagttctgaggctagcaccctttcaacaactcctgttgataccagcatacctgtcaccacttctactgaaggcagttcttctcctacaactgctgaaggtaccagcatgccaatctcaactcctagtgaagtaagtactccattaacaagtatacttgtcagcaccgtgccagtggccggttctgaggctagcaccctttcaacaactcctgttgacaccaggacacctgtcaccacttctgctgaagctagttcttctcctacaactgctgaaggtaccagcatgccaatctcaactcctggcgaaagaagaactccattaacaagtatgtctgtcagcaccatgccggtggccagttctgaggctagcaccctttcaagaactcctgctgacaccagcacacctgtgaccacttctactgaagccagttcctctcctacaactgctgaaggtaccggcataccaatctcaactcctagtgaaggaagtactccattaacaagtatacctgtcagcaccacgccagtggccattcctgaggctagcaccctttcaacaactcctgttgactccaacagtcctgtggtcacttctactgaagtcagttcatctcctacacctgctgaaggtaccagcatgccaatctcaacttatagtgaaggaagcactccattaacaggtgtgcctgtcagcaccacaccggtgaccagttctgcaatcagcaccctttcaacaactcctgttgacaccagcacacctgtgaccacttctactgaagcccattcatctcctacaacttctgaaggtaccagcatgccaacctcaactcctagtgaaggaagtactccattaacatatatgcctgtcagcaccatgctggtagtcagttctgaggatagcaccctttcagcaactcctgttgacaccagcacacctgtgaccacttctactgaagccacttcatctacaactgctgaaggtaccagcattccaacctcaactcctagtgaaggaatgactccattaactagtgtacctgtcagcaacacgccggtggccagttctgaggctagcatcctttcaacaactcctgttgactccaacactcctttgaccacttctactgaagccagttcatctcctcccactgctgaaggtaccagcatgccaacctcaactcctagtgaaggaagcactccattaacaagtatgcctgtcagcaccacaacggtggccagttctgaaacgagcaccctttcaacaactcctgctgacaccagcacacctgtgaccacttattctcaagccagttcatctcctccaattgctgacggtactagcatgccaacctcaacttatagtgaaggaagcactccactaacaaatatgtctttcagcaccacgccagtggtcagttctgaggctagcaccctttccacaactcctgttgacaccagcacacctgtcaccacttctactgaagccagtttatctcctacaactgctgaaggtaccagcataccaacctcaagtcctagtgaaggaaccactccattagcaagtatgcctgtcagcaccacgccggtggtcagttctgaggttaacaccctttcaacaactcctgtggactccaacactctggtgaccacttctactgaagccagttcatctcctacaatcgctgaaggtaccagcttgccaacctcaactactagtgaaggaagcactccattatcaattatgcctctcagtaccacgccggtggccagttctgaggctagcaccctttcaacaactcctgttgacaccagcacacctgtgaccacttcttctccaaccaattcatctcctacaactgctgaagttaccagcatgccaacatcaactgctggtgaaggaagcactccattaacaaatatgcctgtcagcaccacaccggtggccagttctgaggctagcaccctttcaacaactcctgttgactccaacacttttgttaccagttctagtcaagccagttcatctccagcaactcttcaggtcaccactatgcgtatgtctactccaagtgaaggaagctcttcattaacaactatgctcctcagcagcacatatgtgaccagttctgaggctagcacaccttccactccttctgttgacagaagcacacctgtgaccacttctactcagagcaattctactcctacacctcctgaagttatcaccctgccaatgtcaactcctagtgaagtaagcactccattaaccattatgcctgtcagcaccacatcggtgaccatttctgaggctggcacagcttcaacacttcctgttgacaccagcacacctgtgatcacttctacccaagtcagttcatctcctgtgactcctgaaggtaccaccatgccaatctggacgcctagtgaaggaagcactccattaacaactatgcctgtcagcaccacacgtgtgaccagctctgagggtagcaccctttcaacaccttctgttgtcaccagcacacctgtgaccacttctactgaagccatttcatcttctgcaactcttgacagcaccaccatgtctgtgtcaatgcccatggaaataagcacccttgggaccactattcttgtcagtaccacacctgttacgaggtttcctgagagtagcaccccttccataccatctgtttacaccagcatgtctatgaccactgcctctgaaggcagttcatctcctacaactcttgaaggcaccaccaccatgcctatgtcaactacgagtgaaagaagcactttattgacaactgtcctcatcagccctatatctgtgatgagtccttctgaggccagcacactttcaacacctcctggtgataccagcacacctttgctcacctctaccaaagccggttcattctccatacctgctgaagtcactaccatacgtatttcaattaccagtgaaagaagcactccattaacaactctccttgtcagcaccacacttccaactagctttcctggggccagcatagcttcgacacctcctcttgacacaagcacaacttttaccccttctactgacactgcctcaactcccacaattcctgtagccaccaccatatctgtatcagtgatcacagaaggaagcacacctgggacaaccatttttattcccagcactcctgtcaccagttctactgctgatgtctttcctgcaacaactggtgctgtatctacccctgtgataacttccactgaactaaacacaccatcaacctccagtagtagtaccaccacatctttttcaactactaaggaatttacaacacccgcaatgactactgcagctcccctcacatatgtgaccatgtctactgcccccagcacacccagaacaaccagcagaggctgcactacttctgcatcaacgctttctgcaaccagtacacctcacacctctacttctgtcaccacccgtcctgtgaccccttcatcagaatccagcaggccgtcaacaattacttctcacaccatcccacctacatttcctcctgctcactccagtacacctccaacaacctctgcctcctccacgactgtgaaccctgaggctgtcaccaccatgaccaccaggacaaaacccagcacacggaccacttccttccccacggtgaccaccaccgctgtccccacgaatactacaattaagagcaaccccacctcaactcctactgtgccaagaaccacaacatgctttggagatgggtgccagaatacggcctctcgctgcaagaatggaggcacctgggatgggctcaagtgccagtgtcccaacctctattatggggagttgtgtgaggaggtggtcagcagcattgacatagggccaccggagactatctctgcccaaatggaactgactgtgacagtgaccagtgtgaagttcaccgaagagctaaaaaaccactcttcccaggaattccaggagttcaaacagacattcacggaacagatgaatattgtgtattccgggatccctgagtatgtcggggtgaacatcacaaagctacgtcttggcagtgtggtggtggagcatgacgtcctcctaagaaccaagtacacaccagaatacaagacagtattggacaatgccaccgaagtagtgaaagagaaaatcacaaaagtgaccacacagcaaataatgattaatgatatttgctcagacatgatgtgtttcaacaccactggcacccaagtgcaaaacattacggtgacccagtacgaccctgaagaggactgccggaagatggccaaggaatatggagactacttcgtagtggagtaccgggaccagaagccatactgcatcagcccctgtgagcctggcttcagtgtctccaagaactgtaacctcggcaagtgccagatgtctctaagtggacctcagtgcctctgcgtgaccacggaaactcactggtacagtggggagacctgtaaccagggcacccagaagagtctggtgtacggcctcgtgggggcaggggtcgtgctgatgctgatcatcctggtagctctcctgatgctcgttttccgctccaagagagaggtgaaacggcaaaagtacagattgtctcagttatacaagtggcaagaagaggacagtggaccagctcctgggaccttccaaaacattggctttgacatctgccaagatgatgattccatccacctggagtccatctatagtaatttccagccctccttgagacacatagaccctgaaacaaagatccgaattcagaggcctcaggtaatgacgacatcattttaaggcatggagctgagaagtctgggagtgaggagatcccagtccggctaagcttggtggagcattttcccattgagagccttccatgggaactcaatgttcccattgtaagtacaggaaacaagccctgtacttaccaaggagaaagaggagagacagcagtgctgggagattctcaaatagaaacccgtggacgctccaatgggcttgtcatgatatcaggctaggctttcctgctcatttttcaaagacgctccagatttgagggtactctgactgcaacatctttcaccccattgatcgccaggattgatttggttgatctggctgagcaggcgggtgtccccgtcctccctcactgccccatatgtgtccctcctaaagctgcatgctcagttgaagaggacgagaggacgaccttctctgatagaggaggaccacgcttcagtcaaaggcatacaagtatctatctggacttccctgctagcacttccaaacaagctcagagatgttcctcccctcatctgcccgggttcagtaccatggacagcgccctcgacccgctgtttacaaccatgaccccttggacactggactgcatgcactttacatatcacaaaatgctctcataagaattattgcataccatcttcatgaaaaacacctgtatttaaatatagagcatttaccttttggtatataagattgtgggtattttttaagttcttattgttatgagttctgattttttccttagtaaatattataatatatatttgtagtaactaaaaataataaagcaattttattacaatttt

SEQ ID NO: 38 - protein, 4493 aa, Homo sapiens
MPRPGTMALCLLTLVLSLLPPQAAAEQDLSVNRAVWDGGGCISQGDVLNRQCQQLSQHVRTGSAANTATGTTSTNVVEPRMYLSCSTNPEMTSIESSVTSDTPGVSSTRMTPTESRTTSESTSDSTTLFPSSTEDTSSPTTPEGTDVPMSTPSEESISSTMAFVSTAPLPSFEAYTSLTYKVDMSTPLTTSTQASSSPTTPESTTIPKSTNSEGSTPLTSMPASTMKVASSEAITLLTTPVEISTPVTISAQASSSPTTAEGPSLSNSAPSGGSTPLTRMPLSVMLVVSSEASTLSTTPAATNIPVITSTEASSSPTTAEGTSIPTSTYTEGSTPLTSTPASTMPVATSEMSTLSITPVDTSTLVTTSTEPSSLPTTAEATSMLTSTLSEGSTPLTNMPVSTILVASSEASTTSTIPVDSKTFVTTASEASSSPTTAEDTSIATSTPSEGSTPLTSMPVSTTPVASSEASNLSTTPVDSKTQVTTSTEASSSPPTAEVNSMPTSTPSEGSTPLTSMSVSTMPVASSEASTLSTTPVDTSTPVTTSSEASSSSTTPEGTSIPTSTPSEGSTPLTNMPVSTRLVVSSEASTTSTTPADSNTFVTTSSEASSSSTTAEGTSMPTSTYSERGTTITSMSVSTTLVASSEASTLSTTPVDSNTPVTTSTEATSSSTTAEGTSMPTSTYTEGSTPLTSMPVNTTLVASSEASTLSTTPVDTSTPVTTSTEASSSPTTADGASMPTSTPSEGSTPLTSMPVSKTLLTSSEASTLSTTPLDTSTHITTSTEASCSPTTTEGTSMPISTPSEGSPLLTSIPVSITPVTSPEASTLSTTPVDSNSPVTTSTEVSSSPTPAEGTSMPTSTYSEGRTPLTSMPVSTTLVATSAISTLSTTPVDTSTPVTNSTEARSSPTTSEGTSMPTSTPGEGSTPLTSMPDSTTPVVSSEARTLSATPVDTSTPVTTSTEATSSPTTAEGTSIPTSTPSEGTTPLTSTPVSHTLVANSEASTLSTTPVDSNTPLTTSTEASSPPPTAEGTSMPTSTPSEGSTPLTRMPVSTTMVASSETSTLSTTPADTSTPVTTYSQASSSSTTADGTSMPTSTYSEGSTPLTSVPVSTRLVVSSEASTLSTTPVDTSIPVTTSTEASSSPTTAEGTSIPTSPPSEGTTPLASMPVSTTLVVSSEANTLSTTPVDSKTQVATSTEASSPPPTAEVTSMPTSTPGERSTPLTSMPVRHTPVASSEASTLSTSPVDTSTPVTTSAETSSSPTTAEGTSLPTSTTSEGSTLLTSIPVSTTLVTSPEASTLLTTPVDTKGPVVTSNEVSSSPTPAEGTSMPTSTYSEGRTPLTSIPVNTTLVASSAISILSTTPVDNSTPVTTSTEACSSPTTSEGTSMPNSNPSEGTTPLTSIPVSTTPVVSSEASTLSATPVDTSTPGTTSAEATSSPTTAEGISIPTSTPSEGKTPLKSIPVSNTPVANSEASTLSTTPVDSNSPVVTSTAVSSSPTPAEGTSIAISTPSEGSTALTSIPVSTTTVASSEINSLSTTPAVTSTPVTTYSQASSSPTTADGTSMQTSTYSEGSTPLTSLPVSTMLVVSSEANTLSTTPIDSKTQVTASTEASSSTTAEGSSMTISTPSEGSPLLTSIPVSTTPVASPEASTLSTTPVDSNSPVITSTEVSSSPTPAEGTSMPTSTYTEGRTPLTSITVRTTPVASSAISTLSTTPVDNSTPVTTSTEARSSPTTSEGTSMPNSTPSEGTTPLTSIPVSTTPVLSSEASTLSATPIDTSTPVTTSTEATSSPTTAEGTSIPTSTLSEGMTPLTSTPVSHTLVANSEASTLSTTPVDSNSPVVTSTAVSSSPTPAEGTSIATSTPSEGSTALTSIPVSTTTVASSETNTLSTTPAVTSTPVTTYAQVSSSPTTADGSSMPTSTPREGRPPLTSIPVSTTTVASSEINTLSTTLADTRTPVTTYSQASSSPTTADGTSMPTPAYSEGSTPLTSMPLSTTLVVSSEASTLSTTPVDTSTPATTSTEGSSSPTTAGGTSIQTSTPSERTTPLAGMPVSTTLVVSSEGNTLSTTPVDSKTQVTNSTEASSSATAEGSSMTISAPSEGSPLLTSIPLSTTPVASPEASTLSTTPVDSNSPVITSTEVSSSPIPTEGTSMQTSTYSDRRTPLTSMPVSTTVVASSAISTLSTTPVDTSTPVTNSTEARSSPTTSEGTSMPTSTPSEGSTPFTSMPVSTMPVVTSEASTLSATPVDTSTPVTTSTEATSSPTTAEGTSIPTSTLSEGTTPLTSIPVSHTLVANSEVSTLSTTPVDSNTPFTTSTEASSPPPTAEGTSMPTSTSSEGNTPLTRMPVSTTMVASFETSTLSTTPADTSTPVTTYSQAGSSPTTADDTSMPTSTYSEGSTPLTSVPVSTMPVVSSEASTHSTTPVDTSTPVTTSTEASSSPTTAEGTSIPTSPPSEGTTPLASMPVSTTPVVSSEAGTLSTTPVDTSTPMTTSTEASSSPTTAEDIVVPISTASEGSTLLTSIPVSTTPVASPEASTLSTTPVDSNSPVVTSTEISSSATSAEGTSMPTSTYSEGSTPLRSMPVSTKPLASSEASTLSTTPVDTSIPVTTSTETSSSPTTAKDTSMPISTPSEVSTSLTSILVSTMPVASSEASTLSTTPVDTRTLVTTSTGTSSSPTTAEGSSMPTSTPGERSTPLTNILVSTTLLANSEASTLSTTPVDTSTPVTTSAEASSSPTTAEGTSMRISTPSDGSTPLTSILVSTLPVASSEASTVSTTAVDTSIPVTTSTEASSSPTTAEVTSMPTSTPSETSTPLTSMPVNHTPVASSEAGTLSTTPVDTSTPVTTSTKASSSPTTAEGIVVPISTASEGSTLLTSIPVSTTPVASSEASTLSTTPVDTSIPVTTSTEGSSSPTTAEGTSMPISTPSEVSTPLTSILVSTVPVAGSEASTLSTTPVDTRTPVTTSAEASSSPTTAEGTSMPISTPGERRTPLTSMSVSTMPVASSEASTLSRTPADTSTPVTTSTEASSSPTTAEGTGIPISTPSEGSTPLTSIPVSTTPVAIPEASTLSTTPVDSNSPVVTSTEVSSSPTPAEGTSMPISTYSEGSTPLTGVPVSTTPVTSSAISTLSTTPVDTSTPVTTSTEAHSSPTTSEGTSMPTSTPSEGSTPLTYMPVSTMLVVSSEDSTLSATPVDTSTPVTTSTEATSSTTAEGTSIPTSTPSEGMTPLTSVPVSNTPVASSEASILSTTPVDSNTPLTTSTEASSSPPTAEGTSMPTSTPSEGSTPLTSMPVSTTTVASSETSTLSTTPADTSTPVTTYSQASSSPPIADGTSMPTSTYSEGSTPLTNMSFSTTPVVSSEASTLSTTPVDTSTPVTTSTEASLSPTTAEGTSIPTSSPSEGTTPLASMPVSTTPVVSSEVNTLSTTPVDSNTLVTTSTEASSSPTIAEGTSLPTSTTSEGSTPLSIMPLSTTPVASSEASTLSTTPVDTSTPVTTSSPTNSSPTTAEVTSMPTSTAGEGSTPLTNMPVSTTPVASSEASTLSTTPVDSNTFVTSSSQASSSPATLQVTTMRMSTPSEGSSSLTTMLLSSTYVTSSEASTPSTPSVDRSTPVTTSTQSNSTPTPPEVITLPMSTPSEVSTPLTIMPVSTTSVTISEAGTASTLPVDTSTPVITSTQVSSSPVTPEGTTMPIWTPSEGSTPLTTMPVSTTRVTSSEGSTLSTPSVVTSTPVTTSTEAISSSATLDSTTMSVSMPMEISTLGTTILVSTTPVTRFPESSTPSIPSVYTSMSMTTASEGSSSPTTLEGTTTMPMSTTSERSTLLTTVLISPISVMSPSEASTLSTPPGDTSTPLLTSTKAGSFSIPAEVTTIRISITSERSTPLTTLLVSTTLPTSFPGASIASTPPLDTSTTFTPSTDTASTPTIPVATTISVSVITEGSTPGTTIFIPSTPVTSSTADVFPATTGAVSTPVITSTELNTPSTSSSSTTTSFSTTKEFTTPAMTTAAPLTYVTMSTAPSTPRTTSRGCTTSASTLSATSTPHTSTSVTTRPVTPSSESSRPSTITSHTIPPTFPPAHSSTPPTTSASSTTVNPEAVTTMTTRTKPSTRTTSFPTVTTTAVPTNTTIKSNPTSTPTVPRTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRLGSVVVEHDVLLRTKYTPEYKTVLDNATEVVKEKITKVTTQQIMINDICSDMMCFNTTGTQVQNITVTQYDPEEDCRKMAKEYGDYFVVEYRDQKPYCISPCEPGFSVSKNCNLGKCQMSLSGPQCLCVTTETHWYSGETCNQGTQKSLVYGLVGAGVVLMLIILVALLMLVFRSKREVKRQKYRLSQLYKWQEEDSGPAPGTFQNIGFDICQDDDSIHLESIYSNFQPSLRHIDPETKIRIQRPQVMTTSF

SEQ ID NO: 39 - DNA, 3633 bp, Homo sapiens
  source 1..3633: mol_type = other DNA
  CDS 264..3395
ctctcctggtttgactggcacagggagcctgggctggaagaggcagcaaaagggaaaatcagaagagtggacactggcaagaggagggcagcctttttcccagcttccttgcaccatggacagctcccattaagccacctctccatcctggggccaggactcttatgccccattcctgtcaaattgagatttcatccaccattctccaaggacagtgaagttataccctagttccagtgttgggatcagtggcccctctggacatgcctctcctggaaggttctgtgggggtggaggatcttgtcctcctggaacccttggtggaggagtcactgctcaagaatcttcagcttcgctatgaaaacaaggagatttatacctacattgggaatgtggtgatctcagtgaatccctatcaacagcttcccatctatgggccagagttcattgccaaatatcaagactatactttctatgagctgaagccccatatctacgcattggcaaatgtggcgtaccagtcactgagggacagggaccgagaccagtgtatcctcatcacaggcgagagtggatcagggaagactgaggccagcaagctggtgatgtcttatgtggctgccgtctgtgggaaaggagagcaggtgaactctgtgaaggagcagctgctacagtctaacccagtgctggaggcttttggcaatgccaagaccattcgcaacaacaattcctcccgatttggaaaatacatggatattgaatttgacttcaagggatcccccctcggtggtgtcatcacaaactatctgcttgagaaatcccgattagtgaagcagctcaaaggagaaaggaacttccacatcttctatcagctgctggctggagcagatgaacagctgctgaaggccctgaagcttgagcgggatacaactggctatgcctatctgaatcatgaagtatccagagtggatggcatggacgacgcctccagcttcagggctgtacagagtgcaatggcagtgattgggttctcggaggaggagattcgacaagtgctagaggtgacatccatggtgctaaagctggggaacgtgttggtggctgatgagttccaggccagtgggataccagcaagtggcatccgtgatgggagaggtgttcgggagattggggagatggtgggcttgaattcagaagaagtagagagagctttgtgctcgaggaccatggaaacagccaaggaaaaggtggtcactgcactgaatgttatgcaggctcagtatgctcgggacgccctggctaagaacatctacagccgcctctttgactggatagtgaatcgaatcaatgagagcatcaaggtgggcatcggggaaaagaagaaggtaatgggagtccttgatatctacggttttgagatattagaggataatagctttgagcaatttgtgatcaactactgcaatgagaagctgcagcaggtgttcatagagatgaccctgaaagaagagcaagaggaatataagagagaaggcataccgtggacaaaggtggactactttgataatggcatcatttgtaagctcattgagcataatcagcgaggtatcctggccatgttggatgaggagtgcctgcggcctggggtggtcagtgactccactttcctagcaaagctgaaccagctcttctccaagcatggccactacgagagcaaagtcacccagaatgcccagcgtcagtatgaccacaccatgggcctcagctgcttccgcatctgccactatgcgggcaaggtgacatacaacgtgaccagctttattgacaagaataatgacctactcttccgagacctgttgcaggccatgtggaaggcccagcaccccctccttcggtccttgtttcctgagggcaatcctaagcaggcatctctcaaacgccccccgactgctggggcccagttcaagagttctgtggccatcctcatgaagaatctgtattccaagagccccaactacatcaggtgcataaagcccaatgagcatcagcagcgaggtcagttctcttcagacctggtggcaacccaggctcggtacctgggactgctggagaacgtacgggtgcgacgggcaggctatgcccaccgccagggttatgggcccttcctggaaaggtaccgattgctgagccggagcacctggcctcactggaatgggggagaccgggaaggtgttgagaaggtcctgggggagctgagcatgtcctcgggggagctggcctttggcaagacaaagatcttcattagaagccccaagactcttttctacctcgaagaacagaggcgcctgagactccagcagctggccacactcatacagaagatttaccgaggctggcgctgccgcacccactaccaactgatgcgaaagagtcagatcctcatctcctcttggtttcggggaaacatgcaaaagaaatgctatgggaagataaaggcatccgtgttattgatccaggcttttgtgagagggtggaaggcccgaaagaattatcgcaaatatttccggtcagaggctgccctcaccttggcagatttcatctacaagagcatggtacagaaattcctactggggctgaagaacaatttgccatccacaaacgtcttagacaagacatggccagccgccccctacaagtgcctcagcacagcaaatcaggagctgcagcagctcttctaccagtggaagtgcaagaggttccgggatcagctgtccccgaagcaggtagagatcctgagggaaaagctctgtgccagtgaactgttcaagggcaagaaggcttcatatccccagagtgtccccattccattctgtggtgactacattgggctgcaagggaaccccaagctgcagaagctgaaaggcggggaggaggggcctgttctgatggcagaggccgtgaagaaggtcaatcgtggcaatggcaagacttcttctcggattctcctcctgaccaagggccatgtgattctcacagacaccaagaagtcccaggccaaaattgtcattgggctagacaatgtggctggggtgtcagtcaccagcctcaaggatgggctctttagcttgcatctgagtgagatgtcatcggtgggctccaagggggacttcctgctggtcagcgagcatgtgattgaactgctgaccaaaatgtaccgggctgtgctggatgccacgcagaggcagcttacagtcaccgtgactgagaagttctcagtgaggttcaaggagaacagtgtggctgtcaaggtcgtccagggccctgcaggtggtgacaacagcaagctacgctacaaaaaaaaggggagtcattgcttggaggtgactgtgcagtgaggagggggcaccatgcagagatggcagttgcttcctcctgaaccagcactaatccccctctgccctcctgtgtgggaggatctctaacccctctgatcgtggcgcatggcttggggattaaactacccttgaagaggacccttgtcccaaacccttcttgttctctcctccaaaagtagcttcctccaacccgcagcctctctgcacactaataaaacatgtggcttggaaaggttca

SEQ ID NO: 40 - protein, 1043 aa, Homo sapiens
MPLLEGSVGVEDLVLLEPLVEESLLKNLQLRYENKEIYTYIGNVVISVNPYQQLPIYGPEFIAKYQDYTFYELKPHIYALANVAYQSLRDRDRDQCILITGESGSGKTEASKLVMSYVAAVCGKGEQVNSVKEQLLQSNPVLEAFGNAKTIRNNNSSRFGKYMDIEFDFKGSPLGGVITNYLLEKSRLVKQLKGERNFHIFYQLLAGADEQLLKALKLERDTTGYAYLNHEVSRVDGMDDASSFRAVQSAMAVIGFSEEEIRQVLEVTSMVLKLGNVLVADEFQASGIPASGIRDGRGVREIGEMVGLNSEEVERALCSRTMETAKEKVVTALNVMQAQYARDALAKNIYSRLFDWIVNRINESIKVGIGEKKKVMGVLDIYGFEILEDNSFEQFVINYCNEKLQQVFIEMTLKEEQEEYKREGIPWTKVDYFDNGIICKLIEHNQRGILAMLDEECLRPGVVSDSTFLAKLNQLFSKHGHYESKVTQNAQRQYDHTMGLSCFRICHYAGKVTYNVTSFIDKNNDLLFRDLLQAMWKAQHPLLRSLFPEGNPKQASLKRPPTAGAQFKSSVAILMKNLYSKSPNYIRCIKPNEHQQRGQFSSDLVATQARYLGLLENVRVRRAGYAHRQGYGPFLERYRLLSRSTWPHWNGGDREGVEKVLGELSMSSGELAFGKTKIFIRSPKTLFYLEEQRRLRLQQLATLIQKIYRGWRCRTHYQLMRKSQILISSWFRGNMQKKCYGKIKASVLLIQAFVRGWKARKNYRKYFRSEAALTLADFIYKSMVQKFLLGLKNNLPSTNVLDKTWPAAPYKCLSTANQELQQLFYQWKCKRFRDQLSPKQVEILREKLCASELFKGKKASYPQSVPIPFCGDYIGLQGNPKLQKLKGGEEGPVLMAEAVKKVNRGNGKTSSRILLLTKGHVILTDTKKSQAKIVIGLDNVAGVSVTSLKDGLFSLHLSEMSSVGSKGDFLLVSEHVIELLTKMYRAVLDATQRQLTVTVTEKFSVRFKENSVAVKVVQGPAGGDNSKLRYKKKGSHCLEVTVQ

SEQ ID NO: 41 - DNA, 2772 bp, Homo sapiens
  source 1..2772: mol_type = other DNA
  CDS 49..1470
ggcctgctgggttagtgctggcagccccctgaggccaaggacagcagcatgacagtcaccaggactcaccacttcaaggaggggtccctcagagcacctgccatacccctgcacagtgctgcggctgagttggcttcaaaccatccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagactcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggatgggccatctggggtctatgcccacatacccacgtttgttcgcttcctgagtcttttcattgctacctctaatagtcctgtctcccacttcccactcgttcccctcctcttccgagctgctttgtgggctccaggcctgtactcatcggcaggcgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctaaaaaaaaaaaaaaaaaaaaaaaaaaaaaa

SEQ ID NO: 42 - protein, 473 aa, Homo sapiens
MTVTRTHHFKEGSLRAPAIPLHSAAAELASNHPRGPEANLEVRPKESWNHADFVHCEDTESVPGKPSVNADEEVGGPQICRVCGDKATGYHFNVMTCEGCKGFFRRAMKRNARLRCPFRKGACEITRKTRRQCQACRLRKCLESGMKKEMIMSDEAVEERRALIKRKKSERTGTQPLGVQGLTEEQRMMIRELMDAQMKTFDTTFSHFKNFRLPGVLSSGCELPESLQAPSREEAAKWSQVRKDLCSLKVSLQLRGEDGSVWNYKPPADSGGKEIFSLLPHMADMSTYMFKGIISFAKVISYFRDLPIEDQISLLKGAAFELCQLRFNTVFNAETGTWECGRLSYCLEDTAGGFQQLLLEPMLKFHYMLKKLQLHEEEYVLMQAISLFSPDRPGVLQHRVVDQLQEQFAITLKSYIECNRPQPAHRFLFLKIMAMLTELRSINAQHTQRLLRIQDIHPFATPLMQELFGITGS

SEQ ID NO: 43 - DNA, 3115 bp, Homo sapiens
  source 1..3115: mol_type = other DNA
  CDS 766..2388
ctctctgcgcctcgctccgactccggcgcgcaccaagcgggaacccgcgcccgagcccggcgggcgccgctgctgctcctctcggtccccggttcccggtccccgaacgcgccgcggcgaggctgcgcccggcagtggagccgctcggagctgctgggctttggcggccgctcgctgctccgtctggccggaggaccggcggggaaggaaggggaaagcggggacacacacactcgccgcggcgcgcgcgcactgcacactcgtagccgcgcgcccccgccaaggcgcgtccggagcgagtttggccccgcggctccagccccggcacctgcccgccctcagcgttgcccccggccccggccccgcccgccgccccctccgccctcccgcccctcccgcccctcccgcccctcgcctgcactgctctcccttcgctgtggggaagcgacaacgtcccgataacttgcagactgtggcgcaactggtcttggtagcggaggcacccgaatgctgcccgggtgagatgaggaagccaaggcccagcagagctgagatgtgactgcagagccgtccaaccccagtcctgtgacctttctctggtgcctgatacctctcagcatttgagggccttttctcttcctgcttcatctctaaaggtccttctaggagagaggtgaaagaaacctggcaaagaaaacggtctcgacaatgagtaggccacccatcactactaactacagatgacttgccatttcatttacaaagatgtcttctgctgctgaaaatggagaggcagcacctggaaaacaaaatgaagaaaaaacctataaaaagactgcatcatctgctattaaaggtgctattcagctgggaataggatacacagtgggtaatctcacttccaagccagaacgagatgttcttatgcaagacttttatgtggtggaaagtgtgttcctacccagcgaagggagcaatctgaccccagcacatcactacccagactttagatttaagacatacgctccattagcattccgatatttcagagaactttttggtatcaagcctgatgattacttgtattccatctgcagtgaacctctaatagaactgtctaaccctggagccagtggatccttgttttttgtgaccagtgatgatgaatttatcatcaaaacagttcagcacaaagaagctgagtttcttcagaagctactgccaggctattacatgaatttaaaccagaatccaaggactcttttgccaaaattttacggactgtattgtatgcaatcaggaggcattaatatcaggattgtggtgatgaacaacgttttgccacgctccatgagaatgcactttacatatgacttgaaaggctcaacgtataagcgaagagcatcccgtaaagagagagagaaatccaaccccacatttaaggacttagatttcctgcaagacatgcacgaagggttgtattttgatacggaaacatacaacgcgcttatgaaaacacttcagagagactgccgggtgctagaaagcttcaagatcatggattatagccttctgttgggaattcatttcctggaccattccctcaaagagaaagaggaggagaccccacaaaatgtgcctgatgctaagcggactgggatgcagaaggttctctactcaacagccatggaatctatccagggtccagggaaatctggagatgggataatcacagagaacccagacacaatgggaggcattccagctaaaagccataggggagaaaaactacttttatttatgggcattattgacattctgcaatcatataggttaatgaagaagttagaacattcctggaaagctcttgtttatgatggggacactgtttctgttcatagaccaagcttttatgcagacagatttcttaagttcatgaattccagagttttcaagaaaattcaagctttgaaggcttcaccgtctaagaaacggtgcaattcaatcgccgccctaaaggccacttcacaggagattgtgtcctcaattagccaggaatggaaggatgagaagcgggatttgctgactgaaggacaaagttttagcagccttgatgaagaagccctgggatcccgacacaggccagacctggtccctagcactccatcactgtttgaagctgcttccttggcaaccacaatttcatcttcttccttatacgtcaatgagcactatccacacgacaggcctacactctattcaaacagcaaagggttaccttccagttcaacatttaccttggaagaggggaccatctacttgaccgctgagcccaacactctggaagtgcaggatgacaatgcttctgtgcttgacgtctatttataagtgaaaatggtgatcacctaagcacatggatgagacgtgagcacagttatggcagagaagtttctccgcaccagaattatccacagcaacttggctgagccccactacacacagagaaatcatcaacctgacttaagagttttcaagatgtcaacttcaggctgatcagcagatgggatgtgaaaaatactaccctattctatcatttgctgttgcttgctgaactgtgaagaactgcatgaactatatttaagctgctttctgtaccattgccaatcacctttttggagttggaagtgctattttcctatggacttttgcattatttcattgtgcatgcatccagtgattatacataagcaacatatgtaatctgcttatatatttttaaaaatccatccacacacatggtaaattaagtataaattcttttgcaaaattatagttcatgtcattgaaagtttaaattggtttcatttaaagatcaatatactaggtctgccttcactttatagaaaactagcttctataaagattttttcactgtttactagtgaaatgagaaaagcaaagctatttataaaaggccttatgtcgtgtacatacattgtctttgaaatatttgtgatctagtttattgcttgtaaaagagaaattatataatttatttagtaaatactactgtaaactatagttttgtgagagaaataaaatattttgttctcaa

SEQ ID NO: 44 - protein, 540 aa, Homo sapiens
MSSAAENGEAAPGKQNEEKTYKKTASSAIKGAIQLGIGYTVGNLTSKPERDVLMQDFYVVESVFLPSEGSNLTPAHHYPDFRFKTYAPLAFRYFRELFGIKPDDYLYSICSEPLIELSNPGASGSLFFVTSDDEFIIKTVQHKEAEFLQKLLPGYYMNLNQNPRTLLPKFYGLYCMQSGGINIRIVVMNNVLPRSMRMHFTYDLKGSTYKRRASRKEREKSNPTFKDLDFLQDMHEGLYFDTETYNALMKTLQRDCRVLESFKIMDYSLLLGIHFLDHSLKEKEEETPQNVPDAKRTGMQKVLYSTAMESIQGPGKSGDGIITENPDTMGGIPAKSHRGEKLLLFMGIIDILQSYRLMKKLEHSWKALVYDGDTVSVHRPSFYADRFLKFMNSRVFKKIQALKASPSKKRCNSIAALKATSQEIVSSISQEWKDEKRDLLTEGQSFSSLDEEALGSRHRPDLVPSTPSLFEAASLATTISSSSLYVNEHYPHDRPTLYSNSKGLPSSSTFTLEEGTIYLTAEPNTLEVQDDNASVLDVYL

SEQ ID NO: 45 - DNA, 636 bp, Homo sapiens
  source 1..636: mol_type = other DNA
  CDS 80..577
gtgacagccggggtgtgtgtccgccgggcttggtgcctccggtggccctgcagcaccgtcccacctctgccaccctccgatggggccgctacctgtgtgcctgccaatcatgctgctcctgctactgccgtcgctgctgctgctgctgcttctacctggccccgggtccggcgaggcctccaggatattacgtgtgcaccggcgtgggatcctggaactggcaggaactgtgggttgtgttggtccccgaacccccatcgcctatatgaaatatggttgcttttgtggcttgggaggccatggccagccccgcgatgccattgactggtgctgccatggccacgactgttgttacactcgagctgaggaggccggctgcagccccaagacagagcgctactcctggcagtgcgtcaatcagagcgtcctgtgcggaccggcagagaacaaatgccaagaactgttgtgcaagtgtgaccaggagattgctaactgcttagcccaaactgagtacaacttaaagtacctcttctacccccagttcctatgtgagccggactcgcccaagtgtgactgactaccttgacttgaaatgctcttttgcacaaggaaataaagcgtcctctcagtaatgaa

SEQ ID NO: 46 - protein, 165 aa, Homo sapiens
MGPLPVCLPIMLLLLLPSLLLLLLLPGPGSGEASRILRVHRRGILELAGTVGCVGPRTPIAYMKYGCFCGLGGHGQPRDAIDWCCHGHDCCYTRAEEAGCSPKTERYSWQCVNQSVLCGPAENKCQELLCKCDQEIANCLAQTEYNLKYLFYPQFLCEPDSPKCD

SEQ ID NO: 47 - DNA, 1751 bp, Homo sapiens
  source 1..1751: mol_type = other DNA
  CDS 31..489
acacccacagttgagctgcgctggccagagatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaccagcagctgatgcctccttggccaccagaccatgggccaagagccgccgtggctatacctggggacttgatgttccttccagattgtggtgggccctgagtcctggtttcctggcagcctgctgcgcgtgtgggtctctgggcacagtgggcctgtgtgtgtgcccgtgtgtgtgtatgtgtgtgtgtatgtttcttagccccttggattcctgcacgaagtggctgatgggaaccatttcaagacagattgtgaagattgatagaaaatccttcagctaaagtaacagagcatcaaaaacatcactccctctccctccctaacagtgaaaagagagaagggagactctatttaagattcccaaacctaatgatcatctgaatcccgggctaagaatgcagacttttcagactgaccccagaaattctggcccagccaatctagaggcaagcctggccatctgtatttttttttttccaagacagagtcttgctctgttgcccaagctggagtgaagtggtacaatctggctcactgcagcctccgcctcccgggttcaagcgattctcccgcctcagcctcctgagtagctgggattacaggcgcgtatcaccatacccagctaatttttgtatttttagtagagacgggttcaccatgttgcccaggagggtctcgaactcctggcctcaagtgatccaccggcctcggcctcccaaagtgctgggatgacaggcatgaatcactgtgctcagccaccatctggagttttaaaaggctcccatgtgagtccctgtgatggccaggccaggggacccctgccagttctctgtggaagcaaggctggggtcttgggttcctgtatggtggaagctgggtgagccaaggacagggctggctcctctgcccccgctgacgcttcccttgccgttggctttggatgtctttgctgcagtcttctctctggctcaggtgtgggtgggaggggcccacaggaagctcagccttctcctcccaaggtttgagtccctccaaagggcagtgggtggaggaccgggagctttgggtgaccagccactcaaaggaactttctggtcccttcagtatcttcaaggtttggaaactgcaaatgtccccttgatggggaatccgtgtgtgtgtgtgtgtgtgtgtgtgtgtgtgtgtgtgtgtgtgtgtgttttctcctagacccgtgacctgagatgtgtgatttttagtcattaaatggaagtgtctgcca

SEQ ID NO: 48 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 49 - DNA, 1224 bp, Homo sapiens
  source 1..1224: mol_type = other DNA
  CDS 147..623
actggagacactgaagaaggcaggggcccttagagtcttggttgccaaacagatttgcagatcaaggagaacccaggagtttcaaagaagcgctagtaaggtctctgagatccttgcactagctacatcctcagggtaggaggaagatggcttccagaagcatgcggctgctcctattgctgagctgcctggccaaaacaggagtcctgggtgatatcatcatgagacccagctgtgctcctggatggttttaccacaagtccaattgctatggttacttcaggaagctgaggaactggtctgatgccgagctcgagtgtcagtcttacggaaacggagcccacctggcatctatcctgagtttaaaggaagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctagaagagtgcatctccagcctatgaaacagctgggtctttggccataagaagtaaagatttgaagacagaaggaagaaactcaggagtaagcttctagaccccttcagcttctacacccttctgccctctctccattgcctgcaccccaccccagccactcaactcctgcttgtttttcctttggccatgggaaggtttaccagtagaatccttgctaggttgatgtgggccatacattcctttaataaaccattgtgtacataagaggttgctgtgttccagttcagtaatggtgaatgtggaaaagtgaaataagaccaagaaatacacccaa

SEQ ID NO: 50 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 51 - DNA, 977 bp, Homo sapiens
  source 1..977: mol_type = other DNA
  CDS 350..646
agagcaggggttgcctcccctcaccccggtccaggatgcccagtccccacgacacctcccacttcccactgtggcctgggtgggctcaggggctgcccttgacctggcctagagccctcccccagctggtggtggagctggcactctctgggagggagggggctgggagggaatgagtgggaatggcaagaggccagggtttggtgggatcaggttgaggcaggtttggtttccttaaaatgccaagttgggggccagtggggcccacatataaatcctcaccctgggagcctggctgccttgctctccttcctgggtctgtctctgccacctggtctgccacagatccatgatgtgcagttctctggagcaggcgctggctgtgctggtcactaccttccacaagtactcctgccaagagggcgacaagttcaagctgagtaagggggaaatgaaggaacttctgcacaaggagctgcccagctttgtgggggagaaagtggatgaggaggggctgaagaagctgatgggcagcctggatgagaacagtgaccagcaggtggacttccaggagtatgctgttttcctggcactcatcactgtcatgtgcaatgacttcttccagggctgcccagaccgaccctgaagcagaactcttgacttcctgccatggatctcttgggcccaggactgttgatgcctttgagttttgtattcaataaactttttttgtctgttgataatattttaattgctcagtgatgttccataacccggctggctcagctggagtgctgggagatgagggcctcctggatcctgctcccttctgggctctgactctcctggaaatctctccaaggccagagctatgctttaggtctcaattttggaatttcaaacaccagcaaaaaattggaaatcgagataggttgctgacttttattttgtcaaataaagatattaaaaaaggcaaa

SEQ ID NO: 52 - protein, 98 aa, Homo sapiens
MMCSSLEQALAVLVTTFHKYSCQEGDKFKLSKGEMKELLHKELPSFVGEKVDEEGLKKLMGSLDENSDQQVDFQEYAVFLALITVMCNDFFQGCPDRP

SEQ ID NO: 53 - DNA, 3330 bp, Homo sapiens
  source 1..3330: mol_type = other DNA
  CDS 327..2042
ggcggcggcggcggcggagagagctggctcagggcgtccgctaggctcggacgacctgctgagcctcccaaaccgcttccataaggctttgcctttccaacttcagctacagtgttagctaagtttggaaagaaggaaaaaagaaaatccctgggccccttttcttttgttctttgccaaagtcgtcgttgtagtctttttgcccaaggctgttgtgtttttagaggtgctatctccagttccttgcactcctgttaacaagcacctcagcgagagcagcagcagcgatagcagccgcagaagagccagcggggtcgcctagtgtcatgaccagggcgggagatcacaaccgccagagaggatgctgtggatccttggccgactacctgacctctgcaaaattccttctctaccttggtcattctctctctacttggggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaagtggcccagacctcgctggtggtacagaatgtttcagtcatcctgtgtggaatcatcctgatgatggttttcttacataaacatgagcttctgaccatgtaccatggatgggttctcacttcctgctatatcctgatcatcactattgcaaatattgcaaatttggccagtactgctactgcaatcacaatccaaagggattggattgttgttgttgcaggagaagacagaagcaaactagcaaatatgaatgccacaatacgaaggattgaccagttaaccaacatcttagcccccatggctgttggccagattatgacatttggctccccagtcatcggctgtggctttatttcgggatggaacttggtatccatgtgcgtggagtacgttctgctctggaaggtttaccagaaaaccccagctctagctgtgaaagctggtcttaaagaagaggaaactgaattgaaacagctgaatttacacaaagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtctttggtcctttgatttaactgtgacacagttgctgcaagaaaatgtaattgaatctgaaagaggcattataaatggtgtacagaactccatgaactatcttcttgatcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaccacagctgtgccttgagaactaaaagctgtttaggaaacctaagtcagcagaaattaactgattaatttcccttatgttgaggcatggaaaaaaaattggaaaagaaaaactcagtttaaatacggagactataatgataacactgaattcccctatttctcatgagtagatacaatcttacgtaaaagagtggttagtcacgtgaattcagttatcatttgacagattcttatctgtactagaattcagatatgtcagttttctgcaaaactcactcttgttcaagactagctaatttatttttttgcatcttagttatttttaaaaacaaattcttcaagtatgaagactaaattttgataactaatattatccttattgatcctattgatcttaaggtatttacatgtatgtggaaaaacaaaacacttaactagaattctctaataaggtttatggtttagcttaaagagcacctttgtatttttattatcagatggggcaacatattgtatgaagcatatgtagcacttcacagcatggttatcatgtaagctgcaggtagaagcaaagctgtaaagtagatttatcacacaatgactgcatacagacttcaaatatgtcaatagtttggtcatagaacctagaagccaaaagccacacagaagggcaagaatcccaatttaactcatgttatcatcattagtgatctgtgttgtagaacatgagggtgtaagccttcagcctggcaagttacatgtagaaagcccacacttgtgaaggttttgttttacaaatcacttgatttaacacactcaggtagaatatttttatttttactgttttatacccagaagttatttctacattgttctacagcaagaatattcataaaagtatccctttcaaatgcctttgagaagaatagaagaaaaaaagtttgtatatattttaaaaaattgttttaaaagtcagtttgcaacatgtctgtaccaagatggtactttgccttaaccgtttatatgcactttcatggagactgcaatacgttgctatgagcactttctttatccttggagtttaatcctttgcttcatctttctacagtatgacataatgatttgctatgttgtaaaatctttgtaaaaaatttctatataaaaatattttgaaaatcttaa

SEQ ID NO: 54 - protein, 571 aa, Homo sapiens
MTRAGDHNRQRGCCGSLADYLTSAKFLLYLGHSLSTWGDRMWHFAVSVFLVELYGNSLLLTAVYGLVVAGSVLVLGAIIGDWVDKNARLKVAQTSLVVQNVSVILCGIILMMVFLHKHELLTMYHGWVLTSCYILIITIANIANLASTATAITIQRDWIVVVAGEDRSKLANMNATIRRIDQLTNILAPMAVGQIMTFGSPVIGCGFISGWNLVSMCVEYVLLWKVYQKTPALAVKAGLKEEETELKQLNLHKDTEPKPLEGTHLMGVKDSNIHELEHEQEPTCASQMAEPFRTFRDGWVSYYNQPVFLAGMGLAFLYMTVLGFDCITTGYAYTQGLSGSILSILMGASAITGIMGTVAFTWLRRKCGLVRTGLISGLAQLSCLILCVISVFMPGSPLDLSVSPFEDIRSRFIQGESITPTKIPEITTEIYMSNGSNSANIVPETSPESVPIISVSLLFAGVIAARIGLWSFDLTVTQLLQENVIESERGIINGVQNSMNYLLDLLHFIMVILAPNPEAFGLLVLISVSFVAMGHIMYFRFAQNTLGNKLFACGPDAKEVRKENQANTSVV

SEQ ID NO: 55 - DNA, 492 bp, Homo sapiens
  source 1..492: mol_type = other DNA
  CDS 41..295
atccctgactcggggtcgcctttggagcagagaggaggcaatggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttagacacttctgcagggatctgcctgcatcctgacgcggtgccgtccccagcacggtgattagtcccagagctcggctgccacctccaccggacacctcagacacgcttctgcagctgtgcctcggctcacaacacagattgactgctctgactttgactactcaaaattggcctaaaaattaaaagagatcgatattaa

SEQ ID NO: 56 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 57 - DNA, 1131 bp, Homo sapiens
  source 1..1131: mol_type = other DNA
  CDS 180..893
agtgccccaggagctatgacaagcaaaggaacatacttgcctggagatagcctttgcgatatttaaatgtccgtggatacagaaatctctgcaggcaagttgctccagagcatattgcaggacaagcctgtaacgaatagttaaattcacggcatctggattcctaatccttttccgaaatggcaggtgtgagtgcctgtataaaatattctatgtttaccttcaacttcttgttctggctatgtggtatcttgatcctagcattagcaatatgggtacgagtaagcaatgactctcaagcaatttttggttctgaagatgtaggctctagctcctacgttgctgtggacatattgattgctgtaggtgccatcatcatgattctgggcttcctgggatgctgcggtgctataaaagaaagtcgctgcatgcttctgttgtttttcataggcttgcttctgatcctgctcctgcaggtggcgacaggtatcctaggagctgttttcaaatctaagtctgatcgcattgtgaatgaaactctctatgaaaacacaaagcttttgagcgccacaggggaaagtgaaaaacaattccaggaagccataattgtgtttcaagaagagtttaaatgctgcggtttggtcaatggagctgctgattggggaaataattttcaacactatcctgaattatgtgcctgtctagataagcagagaccatgccaaagctataatggaaaacaagtttacaaagagacctgtatttctttcataaaagacttcttggcaaaaaatttgattatagttattggaatatcatttggactggcagttattgagatactgggtttggtgttttctatggtcctgtattgccagatcgggaacaaatgaatctgtggatgcatcaacctatcgtcagtcaaacccctttaaaatgttgctttggctttgtaaatttaaatatgtaagtgctatataagtcaggagcagctgtctttttaaaatgtctcggctagctagaccacagatatcttctagacatattgaacacatttaagatttgagggatataagggaaaatgatatgaatgtgtatttttactcaaaataaaagtaactgtttacgttg

SEQ ID NO: 58 - protein, 237 aa, Homo sapiens
MAGVSACIKYSMFTFNFLFWLCGILILALAIWVRVSNDSQAIFGSEDVGSSSYVAVDILIAVGAIIMILGFLGCCGAIKESRCMLLLFFIGLLLILLLQVATGILGAVFKSKSDRIVNETLYENTKLLSATGESEKQFQEAIIVFQEEFKCCGLVNGAADWGNNFQHYPELCACLDKQRPCQSYNGKQVYKETCISFIKDFLAKNLIIVIGISFGLAVIEILGLVFSMVLYCQIGNK

SEQ ID NO: 59 - DNA, 100 bp, Homo sapiens
  source 1..100: mol_type = other DNA
ttgtggagactatgtgcaaggaaccatcttcccagctcccaatttcaatcccataatggatgcccaaatgctaggaggagcactccaaggatttgactgt

SEQ ID NO: 60 - DNA, 100 bp, Homo sapiens
  source 1..100: mol_type = other DNA
ctcagagagacgtggcaggaccagcgaggaatccagcctgtccacttccagaacagtgtttcccaggccccgctgagtggaccggacctctgacacctcc

SEQ ID NO: 61 - DNA, 100 bp, Homo sapiens
  source 1..100: mol_type = other DNA
gacttttgaactaactggggagacagacaacatatttgtgatagaacgggagggacttctgtattacaacagagccttggacagggaaacaagatctact

SEQ ID NO: 62 - DNA, 100 bp, Homo sapiens
  source 1..100: mol_type = other DNA
tttcaggactgcgtgatcgtgcactttgtgtggtaagaggtttgagtagtcctatatgtcacctagggaacagacattatagcttactagcaaatgaata

SEQ ID NO: 63 - DNA, 100 bp, Homo sapiens
  source 1..100: mol_type = other DNA
gttgccaccctggggaccacaacatgctgctcctttgacaatctcttagaagtcggtcctatctgcaacaaggaagacatatggctgcacgttgatgcag

SEQ ID NO: 64 - DNA, 100 bp, Homo sapiens
  source 1..100: mol_type = other DNA
accaaataacgagctgcacggccaagagagtcacaattcaggcaacaggagcgacgggccaggaaagaacaccacccttcacaatgaatttgacacaatt

SEQ ID NO: 65 - DNA, 100 bp, Homo sapiens
  source 1..100: mol_type = other DNA
ctgctgccccgagcctgattcctagtcctgcttctcttccctctctcctccagcctctcacactctcctcagctctctcatctcctggaaccatggccag

SEQ ID NO: 66 - DNA, 100 bp, Homo sapiens
  source 1..100: mol_type = other DNA
tggtagcagcagcaatttccacatcaatgtagaagagtcagtggatggacaggtggtttcttcccacaagagagaaatctaagtgtctattgcaggagaa

SEQ ID NO: 67 - DNA, 100 bp, Homo sapiens
  source 1..100: mol_type = other DNA
agccagtactacaggacaattgaggagctgcagaacaagatcctcacagccaccgtggacaatgccaacatcctgctacagattgacaatgcccgtctgg

SEQ ID NO: 68 - DNA, 100 bp, Homo sapiens
  source 1..100: mol_type = other DNA
agaagatcacccacaacccatttggtcccggacagttctttgatctgtccattcgctgtggcttggatcgcttcaaggtttacgccaatggccagcacct

SEQ ID NO: 69 - DNA, 100 bp, Homo sapiens
  source 1..100: mol_type = other DNA
cggcagagaacaaatgccaagaactgttgtgcaagtgtgaccaggagattgctaactgcttagcccaaactgagtacaacttaaagtacctcttctaccc

SEQ ID NO: 70 - DNA, 100 bp, Homo sapiens
  source 1..100: mol_type = other DNA
tcctgggcttcgtctactcctttctgggtcctaacccttttgtcgcctggatgcacttcctggtcttcctcgtgggccgtgtggcacacaccgtggccta

SEQ ID NO: 71 - DNA, 100 bp, Homo sapiens
  source 1..100: mol_type = other DNA
aacccaggagtttcaaagaagcgctagtaaggtctctgagatccttgcactagctacatcctcagggtaggaggaagatggcttccagaagcatgcggct

SEQ ID NO: 72 - DNA, 100 bp, Homo sapiens
  source 1..100: mol_type = other DNA
ttttcctggcactcatcactgtcatgtgcaatgacttcttccagggctgcccagaccgaccctgaagcagaactcttgacttcctgccatggatctcttg

SEQ ID NO: 73 - DNA, 100 bp, Homo sapiens
  source 1..100: mol_type = other DNA
cggtgccgtccccagcacggtgattagtcccagagctcggctgccacctccaccggacacctcagacacgcttctgcagctgtgcctcggctcacaacac

SEQ ID NO: 74 - DNA, 100 bp, Homo sapiens
  source 1..100: mol_type = other DNA
ctggctatgtggtatcttgatcctagcattagcaatatgggtacgagtaagcaatgactctcaagcaatttttggttctgaagatgtaggctctagctcc

SEQ ID NO: 75 - DNA, 100 bp, Homo sapiens
  source 1..100: mol_type = other DNA
tttgtggcaaaaatacttcctaggtggtgctgggtacttcttgttgcatcctgtcaggaggcagataatgctggtgcctctctattggtaatgttaagac

SEQ ID NO: 76 - DNA, 100 bp, Homo sapiens
  source 1..100: mol_type = other DNA
atgatggcaaaaccccaggagcagttaatgcctgtcatttatcctgcagtgctttgctgcaagataacatcgctgatgctgtagcttgtgcaaagagggt

SEQ ID NO: 77 - DNA, 100 bp, Homo sapiens
  source 1..100: mol_type = other DNA
cagttctgcaatcagcaccctttcaacaactcctgttgacaccagcacacctgtgaccaattctactgaagcccgttcatctcctacaacttctgaaggt

SEQ ID NO: 78 - DNA, 100 bp, Homo sapiens
  source 1..100: mol_type = other DNA
catctcctcttggtttcggggaaacatgcaaaagaaatgctatgggaagataaaggcatccgtgttattgatccaggcttttgtgagagggtggaaggcc

SEQ ID NO: 79 - DNA, 100 bp, Homo sapiens
  source 1..100: mol_type = other DNA
gcaattcgccattactctgaagtcctacattgaatgcaatcggccccagcctgctcataggttcttgttcctgaagatcatggctatgctcaccgagctc

SEQ ID NO: 80 - DNA, 100 bp, Homo sapiens
  source 1..100: mol_type = other DNA
cttgctgaactgtgaagaactgcatgaactatatttaagctgctttctgtaccattgccaatcacctttttggagttggaagtgctattttcctatggac

SEQ ID NO: 81 - DNA, 100 bp, Homo sapiens
  source 1..100: mol_type = other DNA
gcaaaggtgcacaagtcaatcagacagagagatatgagaaattcaaccagattttaactgccctctcgcgtaaattggaacctcctcctgtaaagcaggc

SEQ ID NO: 82 - DNA, 100 bp, Homo sapiens
  source 1..100: mol_type = other DNA
gacagtggcgactgcgctgacagaacgtgattctcgtgcctttattttgaaagagatgtttttcccaagaggcttgctgaaagagtgagagaagatggaa

SEQ ID NO: 83 - DNA, 100 bp, Homo sapiens
  source 1..100: mol_type = other DNA
aactctccgaagagttgttccaaatgctttacccggcaaccaccagtaaaggaacgacccacagttacggatactcgttctggctcatactgctcgtcat

SEQ ID NO: 84 - DNA, 100 bp, Homo sapiens
  source 1..100: mol_type = other DNA
gaaagtcctcacatcaatagtacatatgaaagtgacctccaaggggattggtgaatactcataaggatcttcaggctgaacagactatgtctggggaaag

SEQ ID NO: 85 - DNA, 100 bp, Homo sapiens
  source 1..100: mol_type = other DNA
tccacagagaggaggggaccaattctggacagacagatgttgggaggatacagaggagatgccacttctcactcaccactaccagccagcctcagaaggc

SEQ ID NO: 86 - DNA, 100 bp, Homo sapiens
  source 1..100: mol_type = other DNA
tgtggcaatgggccacattatgtatttccgatttgcccaaaatactctgggaaacaagctctttgcttgcggtcctgatgcaaaagaagttaggaaggaa

SEQ ID NO: 87 - DNA, 100 bp, Homo sapiens
  source 1..100: mol_type = other DNA
ctggttctcttgctccaccaggaacaagccaccatgtctcgccagtcaagtgtgtccttccggagcgggggcagtcgtagcttcagcaccgcctctgcca

SEQ ID NO: 88 - DNA, 100 bp, Homo sapiens
  source 1..100: mol_type = other DNA
ggaggagtcggacgaggatatgggatttggtctctttgactaatcaccaaaaagcaaccaacttagccagttttatttgcaaaacaaggaaataaaggct

SEQ ID NO: 89 - DNA, 100 bp, Homo sapiens
  source 1..100: mol_type = other DNA
tactgaagaatggagagagaattgaaaaagtggagcattcagacttgtctttcagcaaggactggtctttctatctcttgtactacactgaattcacccc

SEQ ID NO: 90 - DNA, 100 bp, Homo sapiens
  source 1..100: mol_type = other DNA
ttaatagtcattccaaatatgagatgcgttgttacaggaagtcccttgccatcctaaaagccaccccacttctctctaaggagaatggcccagtcctctc

SEQ ID NO: 91 - DNA, 100 bp, Homo sapiens
  source 1..100: mol_type = other DNA
atgcccgaatgccagagaaggtcacatggatgaggagaatgaggattttgcgccggctgctcagaagataccgtgaatctaagaagatcgatcgccacat

SEQ ID NO: 92 - DNA, 100 bp, Homo sapiens
  source 1..100: mol_type = other DNA
actgaatctcccctcctcacagttgccatgtagaccccttgaagaggggaggggcctagggagccgcaccttgtcatgtaccatcaataaagtaccctgt

SEQ ID NO: 93 - DNA, 100 bp, Homo sapiens
  source 1..100: mol_type = other DNA
gtccttggggaacatggagattccagtgtgcctgtatggagtggaatgaatgttgctggtgtctctctgaagactctgcacccagatttagggactgata

SEQ ID NO: 94 - DNA, 100 bp, Homo sapiens
  source 1..100: mol_type = other DNA
agattcagctagtggccaagagatgcagtgccaggaacccttaaacagttgcacagcatctcagctcatcttcactgcaccctggatttgcatacattct

SEQ ID NO: 95 - DNA, 100 bp, Homo sapiens
  source 1..100: mol_type = other DNA
ttctaagtatgtccatttcccatctcagcttcaagggaggtgtcagcagtattatctccactttcaatctccctccaagctctactctggaggagtctgt

SEQ ID NO: 96 - DNA, 100 bp, Homo sapiens
  source 1..100: mol_type = other DNA
tcggtacatggtgccaaccgcctcggggcaaactcgctcttggacctggttgtctttggtcgggcatgtgccctgagcatcgaagagtcatgcaggcctg

SEQ ID NO: 97 - DNA, 100 bp, Homo sapiens
  source 1..100: mol_type = other DNA
gcgaaagtgatgagtctaaaaaagtagtccctccggttcctctgtttccccccatgtggcgtcaggaagcgaggcaccgggcgcactgcgccccgaattc

SEQ ID NO: 98 - DNA, 100 bp, Homo sapiens
  source 1..100: mol_type = other DNA
gagcctatagactatcagttccctttgggcggattgttgtttaacttgtaaatgaaaaaattctcttaaaccacagcactattgagtgaaacattgaact

SEQ ID NO: 99 - DNA, 100 bp, Homo sapiens
  source 1..100: mol_type = other DNA
gtggagtgtgaccttgatgtgaccgggatcccactctgattgcatccatttctctgaaagacttgtttgttctgcttctcttcatataactgagctggcc

SEQ ID NO: 100 - DNA, 100 bp, Homo sapiens
  source 1..100: mol_type = other DNA
ccgatttcatgactgaacagtcaccgacgagagtgctggggaataaaaaggggatcttcactcggcagagacaaccaaaaagtgcagcgttccttttgcg

SEQ ID NO: 101 - DNA, 100 bp, Homo sapiens
  source 1..100: mol_type = other DNA
gcaggcaacacagggaacctcaggccaggcaccacagctcttccactcacagactctcacaactgcacccttgccgggcaccactccactgtatccctcc

SEQ ID NO: 102 - DNA, 100 bp, Homo sapiens
  source 1..100: mol_type = other DNA
acaagggcaacgagcgtttcgtttggacttctcgacttgagtgcccgcctccttcgccgccgcctctgcagtcctcagcgcagttatgcccagttcttcc